(19) United States
(12) Patent Application Publication
(10) Pub. No.: US 2019/0310269 A1
(43) Pub. Date: Oct. 10, 2019

(54) SYSTEMS AND METHODS FOR MEASURING OBESITY USING METABOLOME ANALYSIS

(71) Applicant: Human Longevity, Inc., San Diego, CA (US)

(72) Inventors: Elizabeth CIRULLI, San Diego, CA (US); Amalio TELENTI, La Jolla, CA (US)

(21) Appl. No.: 16/375,834

(22) Filed: Apr. 4, 2019

Related U.S. Application Data
(60) Provisional application No. 62/652,864, filed on Apr. 4, 2018.
(60) Provisional application No. 62/724,515, filed on Aug. 29, 2018.

(51) Int. Cl. G01N 33/92; G01N 33/53; G01N 33/74; G01N 33/66

(52) U.S. Cl. CPC G01N 33/92; G01N 33/5308; G01N 2800/52; G01N 33/66; G01N 33/743

(57) ABSTRACT

The disclosure relates to systems, software and methods for diagnosis or prognosis of subjects for obesity or a disease related thereto, including, classification and treatment of subjects who have been diagnosed with or deemed at risk of having obesity. The methods are based, in part, on the detection of the levels or activities of a plurality of metabolites or their derivatives, such as levels of amino acids, carbohydrates, lipids, nucleic acids, and/or cofactors, in the subject's biological sample, e.g., blood.

### CROSS-REFERENCE TO RELATED APPLICATIONS

This application claims the benefit of priority under 35 U.S.C. § 119 from U.S. Provisional Application No. 62/652,864, filed Apr. 4, 2018; and from U.S. Provisional Application No. 62/724,515, filed Aug. 29, 2018, which are hereby incorporated by reference in their entirety as set forth in full.

### FIELD

The embodiments disclosed herein are generally directed towards systems and methods for identifying obesity risk for individuals. More specifically, there is a need for systems and methods for analyzing an individual's metabolome to make more precise assessments of its risk for health effects associated with obesity.

### BACKGROUND

Obesity is one of the most widespread problems facing our society's health today. Excessive weight significantly increases an individual's risk for conditions like diabetes mellitus and cardiovascular disease. Worldwide, the prevalence of obesity has nearly tripled since 1975, with 39% of the world's adults being overweight and 13% being obese. The high prevalence can be attributed to increasing consumption of hypercaloric foods and sedentary lifestyles. While BMI (body mass index, kg/(m²)) is generally used to characterize obesity, it is a crude measure that does not capture the complexity of a person's state of health. Because of the importance of having a healthy body, better methods of measuring health are needed, and the underlying biology of obesity needs to be better understood. Previous studies have identified metabolic signatures associated with obesity, including increased levels of branched-chain and aromatic amino acids as well as glycerol and glycerophosphocholines. However, conventional approaches to identifying metabolic signatures of obesity have been limited by a focus on a relatively small number of metabolites, individuals, or phenotypes.

With the advent of artificial intelligence and machine learning techniques, it is now possible to process large stores of health data documenting the natural weight gain and loss of large cohorts of individuals over time to identify metabolome changes that can be predictive indicators of obesity as well as identify metabolic biomarkers for different types of obesity (e.g., biomarkers of so-called healthy obesity, diabetes-prone obesity, and cardiovascular disease-prone obesity, etc.).

The ability to measure phenotypic indicators of people with obesity allows for a better understanding of factors that make people susceptible to (or protected from) obesity, accompanied by better elucidation of the factors that account for variability in success of different obesity treatments. As such, there is a need for techniques and/or assays that can provide more accurate predictions of an individual's obesity state and/or health effects associated with it.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

FIGS. 1A-1C show pathway categories of metabolites associated with BMI. FIG. 1A shows pathway categories of the 307 metabolites significantly associated with BMI and FIG. 1B shows pathway categories of the 49-metabolite signature. FIG. 1C shows the values of each of the 49 BMI-associated metabolites are plotted with a Loess curve against the BMI for time point 1 in Twins UK. Only unrelated individuals of European ancestry are included, and the small number of individuals with BMI below 20 (n=31) or above 40 (n=10) are removed to keep the ends of the graphs from being skewed.

FIG. 2 shows changes in BMI between visits. The x axis shows the change in BMI from visit 1 to visit 2, and the y axis shows the change in BMI from visit 2 to visit 3. For analyses of overall BMI change during the study, quantitative change values were calculated by identifying the slope of the changes in BMI over time for each person. For the analysis of BMI recovery, participants were split into 4 groups (or excluded) based on being at least 1 SD above or below the mean for the BMI change at that time point. Those who gained >1 SDs of the mean BMI change at both visits 2 and 3 were classified as “steady gain” (n=27, red); those who lost at both visits were classified “steady loss” (n=19, blue); those who gained and then lost were classified “gain then loss” (n=41, purple); and those who lost and then gained were classified “loss then gain” (n=42, orange).

FIGS. 3A-3C show variables associated with BMI and predicted BMI from the metabolome. FIG. 3A shows correlation between ridge regression model prediction of BMI and actual BMI for all unrelated individuals of European ancestry in the TWINSUK and HN dataset. The identification of outliers is defined below: the pink box shows individuals with a much lower predicted BMI (mBMI) than actual BMI, and the yellow box shows individuals with a much higher mBMI than actual BMI. FIG. 3B shows factors associated with being an mBMI outlier. Participants were split into 5 groups: those whose metabolome accurately predicted their BMI (residual after accounting for age, sex and BMI between −0.5 and 0.5) whose BMIs were either normal (18.5-25), overweight (25-30), or obese (>30); and those whose metabolome predicted a substantially higher mBMI than the actual BMI (residual <−0.5) or a substantially lower mBMI than the actual BMI (residual >0.5). All y-axis values are scaled to a range from 0-1 to allow comparison across groups. FIG. 3C, the results of which were obtained using the same above process, shows DEXA imaging values associated with metabolic BMI outliers. The unexpectedly low mBMI and unexpectedly high mBMI groups had a comparable measured BMI; however, these two groups were statistically significantly different from each other (p<0.01) for all modalities except blood pressure.

FIG. 4 shows heat map of 49 BMI-associated metabolites vs. obesity. This plot compares 1,209 unrelated individuals of European ancestry (rows): 215 are obese (BMI>30; red); 438 are overweight (BMI=25-30; orange); 545 are normal weight (BMI=18.5-25; white); and 11 are underweight (BMI<18.5; blue). Columns are the 49 BMI-associated metabolites, colored as in FIG. 1: lavender is amino acid, green is lipid, purple is peptide, dark red is nucleotide, orange is energy, yellow is cofactors and vitamins, light blue is carbohydrate, and dark blue is xenobiotics. There is an obvious cluster of obese individuals with a distinct metabolic signature.

FIGS. 5A-5C show body composition profiles from Dixon Magnetic Resonance Imaging for four outlier individuals: FIG. 5A shows correlation between ridge regression model prediction of BMI and actual BMI for all unrelated individuals of European ancestry in the TWINSUK and HN dataset. Outliers highlighted in panels B and C are marked with corresponding colors. All individuals highlighted are from the outlier mBMI>>BMI or mBMI<<BMI categories shown in FIG. 3A. FIG. 5B shows body composition profiles (Red=Visceral Adipose Tissue, Yellow=Subcutaneous Adipose Tissue, Cyan=Muscle). FIG. 5C shows waist to hip cross sections (Hip=Mid femoral head; Waist=Top of ASIS). Identity of the individuals depicted in panels A and B.

FIGS. 6A and 6B shows receiver operating characteristic (ROC) curve for the BMI prediction model. Shown is the ability to distinguish A) obese (BMI>=30) from normal weight (BMI 18.5-25) and B) overweight or obese (BMI>=25) from normal weight (BMI 18.5-25). The train (black) AUC were 0.918 FIG. 6A and 0.795 FIG. 6B, and the test (blue) AUC were 0.926 FIG. 6A and 0.804 FIG. 6B. The test specificities were 89.7% FIG. 6A and 68.7% FIG. 6B, with 80.2% FIG. 6A and 80.7% FIG. 6B sensitivity.

FIGS. 7A-7C show progression of different mBMI/BMI categories. FIG. 7A shows alluvial plot showing the proportion of participants who remained in the same weight category or transitioned to a different weight category over the course of the 8-18 years of the TWINSUK study. Red individuals have an obese metabolome, orange individuals have an overweight metabolome, and grey individuals have a normal metabolome. FIG. 7B shows alluvial plot showing the proportion of participants who remained in the same mBMI category or transitioned to a different mBMI category over the course of the 8-18 years of the TWINSUK study. Red individuals begin the study with an obese BMI, orange overweight, and grey normal weight. FIG. 7C shows a survival plot showing age until cardiac event (infarction, angina, or angioplasty). The plot is divided into those whose mBMI corresponds with their BMI (normal weight, overweight, and obese categories) as well as the two outlier groups: those with mBMI<<BMI and those with mBMI>>BMI (p=0.02 for a difference between these categories in cardiovascular outcomes).

FIGS. 8A and 8B show factors associated with having a metabolic BMI different from actual BMI. In FIG. 8A, participants were split into 9 groups: normal weight, metabolically healthy (gray; BMI 18.5-25, BMI prediction below overweight cutoff from FIG. 6B; overweight, metabolically overweight (orange; BMI 25-30, BMI prediction above overweight cutoff but below obese cutoff from FIG. 6A; obese, metabolically obese (red; BMI>=30, BMI prediction above obese cutoff from FIG. 6A; obese, metabolically healthy (pink 1; BMI>=30, BMI prediction below overweight cutoff); obese, metabolically overweight (pink 2; BMI>=30, BMI prediction below obese cutoff); overweight, metabolically healthy (pink 3; BMI 25-30, BMI prediction below overweight cutoff); normal, metabolically obese (yellow 1; BMI 18.5-25, BMI prediction above obese cutoff); normal, metabolically overweight (yellow 2; BMI 18.5-25, BMI prediction above overweight cutoff); and overweight, metabolically obese (yellow 3; BMI 25-30, BMI prediction above obese cutoff). All y-axis values are scaled to a range from 0-1 to allow comparison across groups. The same process is used in FIG. 8B to show imaging (DEXA or MRI) values associated with metabolic BMI outliers (legend: BMI: basal metabolic rate; IR: insulin resistance; WH: waist-to-hip ratio; SYSBP: systolic blood pressure; DIABP: diastolic blood pressure; PG: BMI polygenetic risk score; AG: android/gynoid; PFAT: % fat; VAT: % visceral fat; SAT: % subcutaneous fat).

FIGS. 9A and 9B show genetic risk compared to BMI-relevant variables. FIG. 9A shows correlation between polygenic risk score (PG) category, MC4R carrier status, and BMI and anthropomorphic and clinical measurements for all unrelated individuals of European ancestry in the TWINSUK and HN dataset. All y-axis values are scaled to a range from 0-1 to allow comparison across groups. The same process is used in FIG. 9B to show DEXA imaging values. While there was a trend for genetic risk to be associated with various measurements, the polygenic risk score only achieved p<0.05 for BMI, waist/hip ratio and android/gynoid ratio, and MC4R carrier status only achieved p<0.05 for BMI.

FIG. 10 shows polygenic risk score as a function of BMI. The plot shows the mean polygenic risk score at each BMI for time points 1, 2 and 3 in TWINSUK in red, green and blue, respectively.

FIG. 11 shows representative clinical phenotypes of mBMI/BMI outliers. While there is a continuum of obesity and metabolic perturbations, there are four representative extant phenotypes that are schematically represented in the figure. Indicated are salient features of these groups: rates of insulin resistance (IR), high BMI genetic risk (GR, top decile of polygenic risk or MC4R carrier), and rates of cardiovascular events (CV) during the study follow up.

FIGS. 12A and 12B show obesity prediction and actual obesity status of 350 sets of twins. Shown is the BMI model prediction for each individual plotted against his or her twin's prediction. The heavier twin is always on the x axis, and twins are color-coded to indicate their actual BMI status. FIG. 12A shows the 144 monozygotic twins, and FIG. 12B shows the 206 dizygotic twins. When both twins were obese, they both generally had high BMI model predictions, and when both twins were normal weight, they both generally had low BMI predictions. When only one twin was obese (green, X axis) and the other was normal weight (green, Y axis), the obese twin usually had the higher BMI prediction.

FIGS. 13A-13C show change in metabolic BMI/actual BMI status over time. Included are 1,458 individuals from TWINSUK who had weight data available at all three time points. FIG. 13A shows metabolic BMI categories as defined in FIG. 3. FIG. 13B shows metabolic categories as defined in FIG. 8. FIG. 13C shows proportion of TWINSUK individuals who transitioned to obesity by time point 3. The categories shown on the X axis are the mBMI/BMI category at time point 1. The Y axis shows the proportion of participants in that category who became obese by time point 3. As in FIG. 3 and FIG. 8, gray represents normal weight with healthy metabolome, orange represents overweight with overweight metabolome, yellow colors represent individuals who have mBMI>>BMI and pink colors represent individuals who have mBMI<<BMI.

FIGS. 14A and 14B show MC4R variant carriers, obesity status and polygenic risk score. FIG. 14A shows the carrier frequency of individuals with rare (MAF<0.001%) coding variants in MC4R broken down by obesity status and having a low (first quartile) polygenic risk score (PG). FIG. 14B shows polygenic risk scores of the twin pairs in the TWINSUK cohort, broken down by whether both twins were obese (BMI>30) or normal weight (BMI 18.5-25) and predicted by the metabolome to be obese or normal weight. Twin pairs where both twins were obese and carried MC4R variants are shown in red.

FIG. 15 shows heat map of metabolite loadings for principal component analyses. The loadings of each of the main 49 BMI-associated metabolites are plotted for principal component (PC) analyses performed on the values from each visit (v1, v2, and v3) for the TWINSUK cohort and for the Health Nucleus (HN) cohort. For consistency, the negative values of axis 1 for visit 1, axes 4 and 5 for visit 3, and axes 1 and 5 were used for Health Nucleus.

FIG. 16 shows results of principal component analysis (PCA) of 1 vs. 2. PCA was performed on the 49 BMI-associated metabolites, and here is shown PC1 vs. PC2 in 950 unrelated individuals of European ancestry from the TWINSUK cohort.

FIG. 17 shows cardiovascular events and stroke during follow-up for the different mBMI/BMI categories. During a median 13 years of follow up, 53 of 1573 individuals (3.4%) in the TWINSUK cohort had a cardiovascular or stroke event recorded.

FIG. 18 shows a schematic representation of a computer system of the disclosure.

FIGS. 19A-19D show schematic representations of the system(s) of the disclosure. FIG. 19A shows a schematic representation of an integrated system. FIG. 19B shows a schematic representation of a semi-integrated system. FIG. 19C shows a schematic representation of a semi-discrete system. FIG. 19D shows a schematic representation of a discrete system.

FIG. 20 shows a flowchart of a representative diagnostic method of the disclosure.

### SUMMARY

Obesity is currently identified using the body mass index (BMI) of an individual. This metric (which is derived from the mass and height of an individual) is imprecise, but it is commonly used for health (and medical) recommendations and clinical decisions. A more precise assessment of a person's obesity may also involve the use of anthropomorphic measurements (e.g., waist circumference, waist-height ratio, waist-hip ratio, etc.), biological (hyper-triglyceridemic waist, metabolites, genomic markers, etc.), and imaging (e.g., CT, MRI, DXA, etc.). There are a number of individual metabolites that are known to be associated with BMI and obesity. These include branched chain amino acids (leucine, isoleucine, valine), aromatic amino acids (tyrosine, tryptophan), uric acid, phospholipids, glucose, mannose, asparagine, glycerol, and glycerophosphocholines. However, these metabolites are not currently considered singly or in aggregate to calculate a person's metabolic BMI (mBMI).

Various aspects and embodiments are disclosed herein for analyzing an individual's metabolome to make more precise assessments of his/her risk for obesity and/or health effects associated with obesity. Specifically, the systems and methods disclosed herein relate to detecting, measuring and analyzing an individual's (blood, plasma, serum or some combination thereof) metabolite signature (metabolite profile) to accurately predict an individual's mBMI. Importantly, this signature can identify individuals whose predicted mBMI can be very different from their conventional BMI (determined using conventional weight and height measurements). That is, individuals with different mBMIs can have very different health outcomes even though they are in the same conventional BMI class.

Using linear regression, the levels of an initial set of 901 distinct metabolites were compared to the conventional BMIs of overlapping sets of unrelated individuals in a first population cohort at three time points spanning a total range of 8-18 years. Of that initial set of metabolites, a first subset of 284 metabolites were significantly associated (p<5.5×10⁻⁵) with conventional BMI at one or more time points. From that first subset, 110 metabolites were identified as being significantly associated with BMI at all 3 time points.

These 110 metabolites were further studied in an additional set of unrelated individuals in a second population cohort in order to replicate the initial associations. Of the 84 metabolites that had been measured in both the first and the second cohorts, 83 showed directions of effect that were consistent between the two cohorts, and 49 were shown to be statistically significant replications.

In addition to these 49 strongly associated metabolites, there were an additional 23 metabolites that were statistically significantly associated with BMI in the second population cohort. Overall, 307 metabolites were identified as showing statistically significant associations in at least cohort at one time point, and 49 metabolites with overwhelmingly significant signals, which were used to build a metabolic signature of obesity.

The 307 metabolites that exhibit at least some statistically significant association with obesity are shown in Table 1 below:

TABLE 1
Metabolite

Urate
5-methylthioadenosine (MTA)
Glutamate
N2,N2-dimethylguanosine
1-nonadecanoyl-GPC (19:0)
N-acetylglycine
1-arachidoyl-GPC (20:0)
1-stearoyl-2-dihomo-linolenoyl-GPC (18:0/20:3n3 or 6)
1-(1-enyl-palmitoyl)-2-oleoyl-GPC (P-16:0/18:1)
1-oleoyl-2-linoleoyl-GPC (18:1/18:2)
Valine
1-(1-enyl-stearoyl)-2-docosahexaenoyl-GPC (P-18:0/22:6)
Succinylcarnitine
Kynurenate
1-(1-enyl-palmitoyl)-2-linoleoyl-GPC (P-16:0/18:2)
gamma-glutamylphenylalanine
N-acetylcarnosine
1-eicosenoyl-GPC (20:1)
Mannose
sphingomyelin (d18:1/18:1, d18:2/18:0)
gamma-glutamyltyrosine
N-acetylalanine
1-(1-enyl-stearoyl)-2-oleoyl-GPC (P-18:0/18:1)
N6-carbamoylthreonyladenosine
1-linoleoyl-GPC (18:2)
Propionylcarnitine
1,2-dilinoleoyl-GPC (18:2/18:2)
1-palmitoyl-2-dihomo-linolenoyl-GPC (16:0/20:3n3 or 6)
1-palmitoleoyl-2-oleoyl-glycerol (16:1/18:1)
Alanine
Aspartate
1-palmitoyl-3-linoleoyl-glycerol (16:0/18:2)
Asparagine
N-acetylvaline
N-acetyltyrosine
Leucine
1-palmitoleoyl-3-oleoyl-glycerol (16:1/18:1)
Tyrosine
Cinnamoylglycine
1-oleoyl-2-linoleoyl-glycerol (18:1/18:2)
1-palmitoyl-2-linoleoyl-glycerol (16:0/18:2)
1-oleoyl-3-linoleoyl-glycerol (18:1/18:2)
Carnitine
1-palmitoyl-2-adrenoyl-GPC (16:0/22:4)
Quinolinate
2-methylbutyrylcarnitine (C5)
Glucose
Cortisone
gulonic acid
Adenine
sphingomyelin (d18:2/14:0, d18:1/14:1)
Pseudouridine
sphingomyelin (d18:2/16:0, d18:1/16:1)
Kynurenine
3-phenylpropionate (hydrocinnamate)
arachidate (20:0)
Glycerol
1-oleoyl-2-docosahexaenoyl-GPC (18:1/22:6)
hydantoin-5-propionic acid
2-aminoadipate
1-margaroyl-2-linoleoyl-GPC (17:0/18:2)
1-oleoyl-GPC (18:1)
palmitoleoyl-linoleoyl-glycerol (16:1/18:2) [1]
N1-methyladenosine
2-linoleoyl-GPC (18:2)
1-margaroyl-GPC (17:0)
3-hydroxy-3-methylglutarate
beta-cryptoxanthin
1-(1-enyl-palmitoyl)-GPC (P-16:0)
1-(1-enyl-palmitoyl)-2-palmitoyl-GPC (P-16:0/16:0)
N6-acetyllysine
N-acetylleucine
1-stearoyl-2-oleoyl-GPE (18:0/18:1)
Phenylalanine
1-(1-enyl-stearoyl)-2-docosahexaenoyl-GPE (P-18:0/22:6)
erucate (22:1n9)
Hypotaurine
N-acetylphenylalanine
Orotidine
docosahexaenoate (DHA; 22:6n3)
Lactate
N-acetylserine
1-palmitoyl-2-arachidonoyl-GPI (16:0/20:4)
1-docosahexaenoyl-GPC (22:6)
3-(4-hydroxyphenyl)lactate
N-acetylisoleucine
1,3,7-trimethylurate
Proline
1-palmitoyl-2-linoleoyl-GPI (16:0/18:2)
linoleoyl-arachidonoyl-glycerol (18:2/20:4) [1]
1-palmitoyl-2-oleoyl-GPE (16:0/18:1)
2-docosahexaenoyl-GPC (22:6)
Glycine
Isovalerylcarnitine
1-palmitoyl-2-oleoyl-GPI (16:0/18:1)
Ribitol
1-methylhistidine
1-stearoyl-2-docosapentaenoyl-GPC (18:0/22:5n6)
1,7-dimethylurate
gamma-CEHC glucuronide
Butyrylcarnitine
lactosyl-N-palmitoyl-sphingosine
Glutamine
1-linolenoylglycerol (18:3)
4-androsten-3beta,17beta-diol monosulfate (1)
1-stearoyl-2-meadoyl-GPC (18:0/20:3n9)
1-palmitoyl-2-arachidonoyl-GPC (16:0/20:4)
l-stearoyl-2-arachidonoyl-GPC (18:0/20:4)
cyclo(leu-pro)
gamma-tocopherol/beta-tocopherol
indolepropionate
glucuronate
1-stearoyl-2-arachidonoyl-GPE (18:0/20:4)
bilirubin (E,Z or Z,E)
1-stearoyl-2-docosahexaenoyl-GPE (18:0/22:6)
1-palmitoyl-2-palmitoleoyl-GPC (16:0/16:1)
methyl indole-3-acetate
2-linoleoyl-GPE (18:2)
1-(1-enyl-stearoyl)-GPE (P-18:0)
1-oleoylglycerol (18:1)
dimethylglycine
1-stearoyl-2-linoleoyl-GPE (18:0/18:2)
bilirubin (Z,Z)
creatine
argininate
N-acetyltryptophan
homoarginine
ribonate
glycohyocholate
7-alpha-hydroxy-3-oxo-4-cholestenoate (7-Hoca)
glycerate
sulfate
X - 12100
X - 22822
X - 11787
X - 15492
1-carboxyethylvaline
X - 15503
X - 11299
X - 11452
1-carboxyethylphenylalanine
X - 12040
hydroxy-CMPF
X - 15486
5,6-dihydrouridine
3-methylglutarylcarnitine (1)
X - 11372
X - 12847
X - 12329
X - 13835
X - 18901
X - 17166
glycine conjugate of C10H14O2 (1)
X - 12206
X - 23026
X - 11522
X - 23639
X - 21752
X - 11905
X - 18249
X - 17299
X - 11838
X - 24435
X - 12101
X - 17145
X - 21736
X - 16580
5-methylthioribose
X - 16944
X - 17179
X - 17337
bradykinin, des-arg(9)
X - 12846
X - 12221
octadecenedioate (C18:1-DC)
X - 23593
X - 11429
X - 14056
X - 14838
X - 16123
X - 21626
X - 16132
1-palmityl-2-oleoyl-GPC (O-16:0/18:1)
1-myristoyl-2-arachidonoyl-GPC (14:0/20:4)
1-pentadecanoyl-2-arachidonoyl-GPC (15:0/20:4)
4-hydroxyglutamate
1-(1-enyl-stearoyl)-2-linoleoyl-GPC (P-18:0/18:2)
1-(1-enyl-palmitoyl)-2-palmitoleoyl-GPC (P-16:0/16:1)
gamma-glutamyltryptophan
S-adenosylhomocysteine (SAH)
1-linoleoyl-2-docosahexaenoyl-GPC (18:2/22:6)
1-oleoyl-2-dihomo-linoleoyl-GPC (18:1/20:2)
C-glycosyltryptophan
guanidinoacetate
isoleucine
gamma-glutamylisoleucine
gamma-glutamylleucine
nonadecanoate (19:0)
beta-alanine
1-(1-enyl-palmitoyl)-2-docosahexaenoyl-GPC (P-16:0/22:6)
N1-Methyl-2-pyridone-5-carboxamide
urea
pyruvate
1-stearyl-GPC (O-18:0)
gamma-glutamylvaline
2-hydroxyphenylacetate
1-palmitoleoylglycerol (16:1)
palmitoyl sphingomyelin (d18:1/16:0)
1-oleoyl-2-dihomo-linolenoyl-GPC (18:1/20:3)
allantoin
N-acetylneuraminate
1-palmitoyl-2-stearoyl-GPC (16:0/18:0)
pipecolate
1-methylimidazoleacetate
5alpha-androstan-3alpha,17beta-diol monosulfate (1)
7-methylguanine
sphingosine
1-palmitoyl-2-docosahexaenoyl-GPC (16:0/22:6)
1-stearoyl-GPC (18:0)
erythritol
1-dihomo-linoleoyl-GPC (20:2)
2-oleoyl-GPC (18:1)
1-dihomo-linolenylglycerol (20:3)
2-palmitoyl-GPE (16:0)
1-myristoylglycerol (14:0)
gamma-glutamylalanine
2-docosahexaenoyl-GPE (22:6)
1-(1-enyl-oleoyl)-GPC (P-18:1)
mannitol/sorbitol
alpha-ketoglutarate
1-palmitoyl-GPE (16:0)
hexadecadienoate (16:2n6)
1-(1-enyl-stearoyl)-GPC (P-18:0)
3-methyladipate
1-dihomo-linolenoyl-GPC (20:3n3 or 6)
erythronate
1,2-dipalmitoyl-GPC (16:0/16:0)
palmitoyl dihydrosphingomyelin (d18:0/16:0)
5-methyluridine (ribothymidine)
2-hydroxybutyrate/2-hydroxyisobutyrate
1-eicosapentaenoyl-GPE (20:5)
1-palmitoyl-GPC (16:0)
N-acetylcitrulline
2-aminoheptanoate
indoleacetylglutamine
eicosapentaenoate (EPA; 20:5n3)
phenylalanylphenylalanine
ergothioneine
gluconate
1-myristoyl-2-linoleoyl-GPC (14:0/18:2)
stearoyl sphingomyelin (d18:1/18:0)
gamma-glutamyl-epsilon-lysine
oxalate (ethanedioate)
glutarylcarnitine (C5)
N-acetylmethionine
dihydroorotate
palmitoleate (16:1n7)
deoxycholate
1-methylurate
2-oxoarginine
tartronate (hydroxymalonate)
1-stearoyl-2-arachidonoyl-GPI (18:0/20:4)
2-hydroxypalmitate
N-formylphenylalanine
isobutyrylglycine
1-(1-enyl-stearoyl)-2-arachidonoyl-GPC (P-18:0/20:4)
leucylleucine
1-docosahexaenoyl-GPE (22:6)
gamma-glutamyl-alpha-lysine
serotonin
1-stearoyl-GPE (18:0)
caprate (10:0)
succinate
thyroxine
phosphocholine (16:0/22:5n3, 18:1/20:4)
cysteine sulfinic acid
1-(1-enyl-palmitoyl)-2-arachidonoyl-GPE (P-16:0/20:4)
7-methylurate
sphingomyelin (d18:1/20:1, d18:2/20:0)
1-arachidonylglycerol (20:4)
2-hydroxyadipate
3-methyl-2-oxobutyrate
6-oxopiperidine-2-carboxylic acid
4-hydroxyphenylacetate
1-linoleoyl-GPE (18:2)
xanthine
1-docosapentaenoyl-GPC (22:5n3)
1-margaroyl-2-oleoyl-GPC (17:0/18:1)
1-palmityl-GPC (O-16:0)
3,7-dimethylurate
choline phosphate
dodecanedioate
2-methylbutyrylglycine
2-hydroxystearate
N-acetyltaurine
N-acetylglutamate
3-methyl-2-oxovalerate
X - 15245
2-methylcitrate/homocitrate
PC(O-16:0/16:0)
X - 21339
lysoPE(O-16:0)
X - 11537
X - 11530
1-oleoyl-2-eicosapentaenoyl-GPC (18:1/20:5)
X - 13737
prolylproline

While all 307 of these metabolites are associated with obesity, not all were used in the final metabolic signature to determine mBMI due to either missing data or insufficient evidence. Many of the 307 metabolites have strong correlations with one or more of the subset of 49 strongly associated metabolites and would be expected to show significant associations in a larger study and make similar contributions to the final model as their respective proxies in the subset of 49.

As discussed above, 49 metabolites were identified to have consistent and strong signals associated with conventional BMI. In various embodiments, the levels of the 49 metabolites were measured to calculate each person's metabolic BMI (mBMI) using ridge regression in R's glmnet package. The formula for the calculation was identified using machine learning and artificial intelligence techniques and is as follows:

mBMI=sum((coefficient)×(metabolite value))+Intercept Eq. 1:

The 49 metabolites that are the most strongly associated with obesity are shown in Table 2 below in rank of correlation:

TABLE 2

| | Rank of relative |
|---|---|
| | correlation to |
| Metabolite | metabolic obesity |
| Urate | 1 |
| Glutamate | 2 |
| 1-(1-enyl-palmitoyl)-2-oleoyl-GPC (P-16:0/18:1) | 3 |
| 1-stearoyl-2-dihomo-linolenoyl-GPC (18:0/20:3n3 | 4 |
| or 6) | |
| 1-eicosenoyl-GPC (20:1) | 5 |
| N2,N2-dimethylguanosine | 6 |
| 1-arachidoyl-GPC (20:0) | 7 |
| 1-(1-enyl-stearoyl)-2-oleoyl-GPC (P-18:0/18:1) | 8 |
| N-acetylglycine | 9 |
| 5-methylthioadenosine (MTA) | 10 |
| Valine | 11 |
| Propionylcarnitine | 12 |
| Succinylcarnitine | 13 |
| 1-nonadecanoyl-GPC (19:0) | 14 |
| 1-linoleoyl-GPC (18:2) | 15 |
| Aspartate | 16 |
| Mannose | 17 |
| N-acetylvaline | 18 |
| Kynurenate | 19 |
| sphingomyelin (d18:1/18:1, d18:2/18:0) | 20 |
| 1-palmitoyl-2-dihomo-linolenoyl-GPC (16:0/20:3n3 | 21 |
| or 6) | |
| 1-(1-enyl-palmitoyl)-2-linoleoyl-GPC (P-16:0/18:2) | 22 |
| Alanine | 23 |
| 1-palmitoyl-3-linoleoyl-glycerol (16:0/18:2) | 24 |
| N-acetylcarnosine | 25 |
| Asparagine | 26 |
| 1-oleoyl-2-linoleoyl-GPC (18:1/18:2) | 27 |
| N6-carbamoylthreonyladenosine | 28 |
| 1-(1-enyl-stearoyl)-2-docosahexaenoyl-GPC (P- | 29 |
| 18:0/22:6) | |
| 1-oleoyl-3-linoleoyl-glycerol (18:1/18:2) | 30 |
| N-acetylalanine | 31 |
| gamma-glutamylphenylalanine | 32 |
| Carnitine | 33 |
| Tyrosine | 34 |
| gamma-glutamyltyrosine | 35 |
| 1-palmitoyl-2-linoleoyl-glycerol (16:0/18:2) | 36 |
| Leucine | 37 |
| 1-oleoyl-2-linoleoyl-glycerol (18:1/18:2) | 38 |
| 1,2-dilinoleoyl-GPC (18:2/18:2) | 39 |
| N-acetyltyrosine | 40 |
| 2-methylbutyrylcarnitine (C5) | 41 |
| 1-palmitoleoyl-2-oleoyl-glycerol (16:1/18:1) | 42 |
| Cinnamoylglycine | 43 |
| Quinolinate | 44 |
| 1-palmitoleoyl-3-oleoyl-glycerol (16:1/18:1) | 45 |
| gulonic acid | 46 |
| 1-palmitoyl-2-adrenoyl-GPC (16:0/22:4) | 47 |
| Glucose | 48 |
| Cortisone | 49 |

Before the coefficients are applied, the metabolite data is rank-ordered and forced to a normal distribution with a mean of 0 and standard deviation of 1. After applying the coefficients, the sum of the 49 metabolite level values for each person is taken, and the intercept is added. This final value is the metabolic BMI (mBMI) or the metabolic signature of obesity.

The various embodiments of the disclosure are provided below:

In some embodiments, the disclosure relates to a method of diagnosing obesity or a disease related thereto in a subject, comprising, obtaining a biological sample from the subject; detecting, in the biological sample, levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7; calculating a metabolomic body mass index (mBMI) value for the subject based on the detection, wherein the metabolites of the Tables are listed in the order of relative correlation to the subject's calculated mBMI value; and diagnosing the subject as having obesity if the mBMI value of the subject is modulated compared to a reference standard.

In some embodiments, the disclosure relates to a method of diagnosing obesity or a disease related thereto in a subject, comprising, obtaining a biological sample from the subject; detecting, in the biological sample, levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 1 or Table 2; calculating a metabolomic body mass index (mBMI) value for the subject based on the detection, wherein the metabolites of the Tables are listed in order of effect on the mBMI value or the order of relative correlation to the subject's calculated mBMI value; and diagnosing the subject as having obesity if the mBMI value of the subject is modulated compared to a reference standard.

In some embodiments, the disclosure relates to a method of diagnosing obesity or a disease related thereto in a subject in accordance with the foregoing, wherein the biological sample comprises a blood sample (e.g., whole blood, plasma, serum, or a combination thereof).

In some embodiments, the disclosure relates to a method of diagnosing obesity or a disease related thereto in a subject in accordance with the foregoing, wherein the levels and/or activities of the metabolites or derivatives thereof is determined using a chemical analytical method selected from the group consisting of HPLC, thin layer chromatography (TLC), electrochemical analysis, Mass Spectroscopy (MS), refractive index spectroscopy (RI), Ultra-Violet spectroscopy (UV), fluorescent analysis, radiochemical analysis, Near-Infra Red spectroscopy (Near-IR), Nuclear Magnetic Resonance spectroscopy (NMR), fluorescence spectroscopy, dual polarization interferometry, computational methods, Light Scattering analysis (LS), gas chromatography (GC), GC coupled with MS, and direct injection (DI) coupled with LC-MS/MS or a combination thereof.

In some embodiments, the disclosure relates to a method of diagnosing obesity or a disease related thereto in a subject in accordance with the foregoing, wherein the disease related to obesity is selected from coronary artery disease, hypertension, stroke, peripheral vascular disease, insulin resistance, glucose intolerance, diabetes mellitus, hyperglycemia, hyperlipidemia, hypercholesteremia, hypertriglyceridemia, hyperinsulinemia, atherosclerosis, cellular proliferation and endothelial dysfunction, diabetic dyslipidemia, lipodystrophy and metabolic syndrome, type II diabetes, diabetic complications including diabetic neuropathy, nephropathy, retinopathy or cataracts, heart failure, inflammation, thrombosis, congestive heart failure, asthmatic or pulmonary disease related to obesity, and cardiovascular disease related to obesity or a combination thereof.

In some embodiments, the disclosure relates to a method of diagnosing obesity or a disease related thereto in a subject in accordance with the foregoing, wherein the derivative of the metabolite is selected from salts, amides, esters, enol ethers, enol esters, acetals, ketals, acids, bases, solvates, hydrates, and polymorphs or a combination thereof.

In some embodiments, the disclosure relates to a method of diagnosing obesity or a disease related thereto in a subject in accordance with the foregoing, wherein the modulation of mBMI comprises an increase or a decrease in mBMI compared to a reference standard. Preferably, if the subject's mBMI is increased compared to a reference standard, then the subject is diagnosed as having obesity with metabolic repercussions (e.g., predictive of metabolic syndrome and cardiovascular risk). Particularly, if mBMI>threshold obesity BMI of 30, then the subject is diagnosed as having obesity with severe metabolic repercussions.

In some embodiments, the disclosure relates to a method of diagnosing obesity or a disease related thereto in a subject, comprising, obtaining a biological sample from the subject; detecting, in the biological sample, levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7; calculating a metabolomic body mass index (mBMI) value for the subject based on the detection, wherein the metabolites of the Tables are listed in the order of relative correlation to (or effect on) the subject's calculated mBMI value; and diagnosing the subject as having obesity if the mBMI value of the subject is modulated compared to a reference standard comprising the subject's BMI. Preferably, if the subject's mBMI is increased compared to the subject's BMI, then the subject is diagnosed as having obesity with metabolic repercussions (e.g., predictive of metabolic syndrome and cardiovascular risk). Particularly, if mBMI>threshold obesity BMI of 30, then the subject is diagnosed as having obesity with severe metabolic repercussions.

In some embodiments, the disclosure relates to a method of diagnosing obesity or a disease related thereto in a subject, comprising, obtaining a biological sample from the subject; detecting, in the biological sample, levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7; calculating a metabolomic body mass index (mBMI) value for the subject based on the detection, wherein the metabolites of the Tables are listed in the order of relative correlation to (or effect on) the subject's calculated mBMI value; further determining a secondary parameter selected from blood pressure, waist/hip ratio, android/gynoid ratio, % body fat, % visceral fat, % subcutaneous fat and insulin resistance or a combination thereof; and diagnosing the subject as having obesity if the mBMI value and the level of at least 1, 2, 3, 4, 5, 6 or 7 secondary parameter is increased compared to a reference standard. Particularly, the reference standard comprises a subject whose BMI>30. Under this embodiment, preferably, the method comprises generating a composite score of the mBMI and the secondary parameter and comparing the composite score to a reference standard. Particularly, the reference standard comprises a positive reference standard comprising a composite score of the mBMI and the secondary parameter for an obese subject and/or a negative reference standard comprising a composite score of the mBMI and the secondary parameter for a non-obese or healthy subject.

In some embodiments, the disclosure relates to a method for diagnosis of healthy obesity or unhealthy obesity or a disease related thereto by carrying out the foregoing methods. Preferably healthy obesity comprises a subject whose BMI>threshold obesity BMI of 30 but whose mBMI≤30; and the unhealthy obesity comprises a subject whose BMI≤threshold obesity BMI of 30 but whose mBMI>30.

In some embodiments, the disclosure relates to a method of diagnosing and treating obesity or a disease related thereto in a subject, comprising, (a) detecting, in a biological sample obtained from the subject, levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7 and calculating a metabolomic body mass index (mBMI) value for the subject based on the detection, wherein the metabolites of the Tables are listed in the order of relative correlation to (or effect on) the subject's calculated mBMI value; (b) diagnosing subject with obesity if the mBMI value of the subject is modulated compared to a reference standard; and (c) administering an effective amount of a therapy selected from the group consisting of anti-obesity pharmacotherapy, surgery, and lifestyle therapy to the subject diagnosed with obesity. Preferably under this embodiment, if the subject's mBMI is greater than a reference standard, e.g., a threshold obesity BMI of 30, then the subject is diagnosed as having obesity or a disease related thereto with metabolic repercussions (e.g., predictive of metabolic syndrome and cardiovascular risk). Particularly, if mBMI>>threshold obesity BMI of 30, then the subject is diagnosed as having obesity with severe metabolic repercussions.

In some embodiments, the disclosure relates to a method of diagnosing and treating obesity or a disease related thereto in a subject, comprising, (a) detecting levels and/or activities of at least three markers of Table 1 or derivatives thereof in a biological sample obtained from the subject and computing a metabolomic body mass index (mBMI) value for the subject based on the detection, wherein the at least 3 metabolites comprises: urate, 5-methylthioadenosine, and glutamate; (b) diagnosing subject with obesity if the mBMI value of the subject is modulated compared to a reference standard; and (c) administering an effective amount of a therapy selected from the group consisting of anti-obesity pharmacotherapy, surgery, and lifestyle therapy. Preferably under this embodiment, if the subject's mBMI is greater than a reference standard, e.g., a threshold obesity BMI of 30, then the subject is diagnosed as having obesity or a disease related thereto with metabolic repercussions (e.g., predictive of metabolic syndrome and cardiovascular risk). Particularly, if mBMI>>threshold obesity BMI of 30, then the subject is diagnosed as having obesity with severe metabolic repercussions.

In some embodiments, the disclosure relates to a method of diagnosing and treating obesity in a subject, comprising, (a) detecting levels and/or activities of at least three markers of Table 2 or derivatives thereof in a biological sample obtained from the subject and computing a metabolomic body mass index (mBMI) value for the subject based on the detection, wherein the at least 3 metabolites comprises, urate, glutamate and 1-(1-enyl-palmitoyl)-2-oleoyl-GPC (P-16:0/18:1); (b) diagnosing subject with obesity if the mBMI value of the subject is modulated compared to a reference standard; and (c) administering an effective amount of a therapy selected from the group consisting of anti-obesity pharmacotherapy, surgery, and lifestyle therapy. Preferably under this embodiment, if the subject's mBMI is greater than a reference standard, e.g., a threshold obesity BMI of 30, then the subject is diagnosed as having obesity or a disease related thereto with metabolic repercussions (e.g., predictive of metabolic syndrome and cardiovascular risk). Particularly, if mBMI>>threshold obesity BMI of 30, then the subject is diagnosed as having obesity with severe metabolic repercussions.

In some embodiments, the disclosure relates to diagnosing and optionally treating obesity in a subject in accordance with the foregoing methods, comprising further detecting at least one secondary parameter and further optionally detecting at least one genetic parameter. Preferably, the secondary parameter is selected from the group consisting of android/gynoid ratio; total triglycerides; waist/hip ratio; subcutaneous fat; visceral fat; insulin resistance; HDL; percent fat; diastolic blood pressure; systolic blood pressure; total cholesterol; and LDL, or a combination thereof, particularly preferably, android/gynoid ratio; total triglycerides; waist/hip ratio; subcutaneous fat; visceral fat; insulin resistance; and HDL. Preferably, the genetic parameter is selected from genetic variants of melanocortin 4 receptor gene (MC4R) or a lipdystrophy gene selected from zinc metallopeptidase STE24 (ZMPSTE24) gene or the 1-acylglycerol-3-phosphate O-acyltransferase 2 (AGPAT2) gene or lipase E, hormone sensitive type (LIPE) gene or Bernardinelli-Seip congenital lipodystrophy type 2 (BSCL2) gene, or any combination thereof; especially an MC4R variant selected from M292fs, R236C, S180P, A175T, and T11A, but not I170V; and/or a genetic variant of a lipodystrophy gene selected from ZMPSTE24, AGPAT2, LIPE gene, BSCL2, or any combination thereof. In some embodiments, the disclosure relates to a method for screening a test agent for treating obesity, comprising, (a) detecting, in a biological sample obtained from the subject, levels and/or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7 and computing a first metabolomic body mass index (mBMI) value; (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject. Preferably under this embodiment, if the subject's second mBMI is reduced compared to the first mBMI, e.g., to a value below a threshold obesity BMI of 30, then the test agent is selected for treating obesity.

In some embodiments, the disclosure relates to a method for screening a test agent for treating obesity, comprising, (a) detecting levels and/or activities of at least three metabolites of Table 1 or derivatives thereof in a biological sample obtained from the subject to compute a first metabolomic body mass index (mBMI) value, wherein the at least 3 metabolites comprises, in the order of rank of relative correlation to the subject's obesity, urate, 5-methylthioadenosine, and glutamate; (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject.

In some embodiments, the disclosure relates to a method for screening a test agent for treating obesity, comprising, (a) detecting levels and/or activities of at least three metabolites of Table 2 or derivatives thereof in a biological sample obtained from the subject, wherein the at least 3 metabolites comprises, in the order of rank of relative correlation to the subject's obesity, urate, glutamate and 1-(1-enyl-palmitoyl)-2-oleoyl-GPC (P-16:0/18:1); (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject.

In some embodiments, the disclosure relates to a method for screening a test agent for treating unhealthy or healthy obesity, preferably unhealthy obesity, comprising, (a) detecting levels and/or activities of at least three metabolites of Table 2 or derivatives thereof in a biological sample obtained from the subject, wherein the at least 3 metabolites comprises, in the order of rank of relative correlation to the subject's obesity, urate, glutamate and 1-(1-enyl-palmitoyl)-2-oleoyl-GPC (P-16:0/18:1); (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject. In some embodiments, the healthy obesity comprises a subject whose BMI>threshold obesity BMI of 30 but whose mBMI≤30; and the unhealthy obesity comprises a subject whose BMI≤threshold obesity BMI of 30 but whose mBMI>30.

In some embodiments, the disclosure relates to a method for screening a test agent for treating unhealthy or healthy obesity, preferably unhealthy obesity, comprising, (a) detecting levels and/or activities of at least three metabolites of Table 2 or derivatives thereof in a biological sample obtained from the subject, wherein the at least 3 metabolites comprises, in the order of rank of relative correlation to the subject's obesity, urate, glutamate and 1-(1-enyl-palmitoyl)-2-oleoyl-GPC (P-16:0/18:1); (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject, wherein the method further comprises (e) detecting a secondary parameter selected from the group consisting of android/gynoid ratio; total triglycerides; waist/hip ratio; subcutaneous fat; visceral fat; insulin resistance; HDL; percent fat; diastolic blood pressure; systolic blood pressure; total cholesterol; and LDL, or a combination thereof, preferably, android/gynoid ratio; total triglycerides; waist/hip ratio; subcutaneous fat; visceral fat; insulin resistance; and HDL.

In some embodiments, the disclosure relates to a method for screening a test agent for treating unhealthy or healthy obesity, preferably unhealthy obesity, comprising, (a) detecting levels and/or activities of at least three metabolites of Table 2 or derivatives thereof in a biological sample obtained from the subject, wherein the at least 3 metabolites comprises, in the order of rank of relative correlation to the subject's obesity, urate, glutamate and 1-(1-enyl-palmitoyl)-2-oleoyl-GPC (P-16:0/18:1); (b) administering a composition comprising the test agent to the subject; (c) detecting levels and/or activities of the metabolites of step (a) in the biological sample obtained from the subject to compute a second mBMI value; and (d) selecting a test agent if the second mBMI value is modulated compared to the first mBMI value for the subject, wherein the method further comprises (e) detecting a genetic parameter selected from a rare (MAF<0.01%) coding variant in the melanocortin 4 receptor gene (MC4R), preferably an MC4R variant selected from M292fs, R236C, S180P, A175T, and T11A, but not I170V; genetic variants of lipodystrophy genes selected from ZMPSTE24 gene or AGPAT2 gene or LIPE gene or BSCL2 gene, or any combination thereof; or a combination of a rare coding variant of MC4R gene and a variant of a gene selected from ZMPSTE24, AGPAT2, LIPE and BSCL2.

In some embodiments, the disclosure relates to a computer readable medium comprising computer-executable instructions, which, when executed by a processor, cause the processor to carry out a method or a set of steps for diagnosing obesity in a subject, comprising detecting a metabolite profile in a metabolome dataset received from a subject's sample, wherein the metabolite profile comprises levels or activities of at least 3, 4, 5, 6, 8, 10, 12, 13, 15, 20, 25, 30, 35, 40, 45, 49, 50, 60, 75, 100, 125, 150, 200, 250, 300, or 307 metabolites or derivatives thereof, wherein the metabolites are selected from the metabolites of Table 11, Table 12A, Table 12B, or Tables 1-7, preferably Tables 2-7, especially Table 2 or Tables 4-7; and the computer readable medium comprises machine learning techniques to determine obesity of subject based on the metabolite profile.

In some embodiments, the disclosure relates to a computer readable medium comprising computer-executable instructions, which, when executed by a processor, cause the processor to carry out a method or a set of steps for diagnosing obesity in a subject, comprising detecting a metabolite profile in a metabolome dataset received from a subject's sample, wherein the metabolite profile comprises levels or activities of at least 3 metabolites of Table 1 or derivatives thereof and the computer readable medium comprises machine learning techniques to determine obesity of subject based on the metabolite profile, wherein the at least 3 metabolites comprises, in the order of rank of relative correlation to the subject's obesity, urate, 5-methylthioadenosine, and glutamate.

In some embodiments, the disclosure relates to a computer readable medium comprising computer-executable instructions, which, when executed by a processor, cause the processor to carry out a method or a set of steps for diagnosing obesity in a subject, comprising detecting a metabolite profile in a metabolome dataset received from a subject's sample, wherein the metabolite profile comprises levels or activities of at least three metabolites of Table 2 or derivatives thereof and the computer readable medium comprises machine learning techniques to determine obesity of subject based on the metabolite profile, wherein the at least 3 metabolites comprises, in the order of rank of relative correlation to the subject's obesity, urate, glutamate and 1-(1-enyl-palmitoyl)-2-oleoyl-GPC (P-16:0/18:1).

Preferably, in the foregoing embodiments, the computer readable medium comprising computer-executable instructions, comprises an algorithm that is trained with a compendium of metabolite profiles each of which are associated with obesity and the algorithm computes the predictive power of each metabolite using a rigorous mathematical algorithm.

In some embodiments, the disclosure relates to an obesity profiling system, comprising: (a) a metabolome detector/analyzer configured to detect/analyze levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a subject's biological sample; (b) an obesity determining engine configured to determine obesity based on levels and/or activities of metabolites or derivatives thereof; (c) an optional data source (e.g., human metabolome database); and (d) a display communicatively connected to a computing device and configured to display a report containing the subject's obesity profile, wherein each of components (a), (b), (c) and (d) is communicatively connected to each other either directly or via indirectly (e.g., via the internet).

In some embodiments, the disclosure relates to an obesity profiling system, comprising: (a) a metabolome detector/analyzer configured to detect/analyze levels or activities of at least 3 metabolites of Table 1 or derivatives thereof in a subject's biological sample, wherein the at least 3 metabolites comprises, in the order of rank of relative correlation to the subject's obesity, urate; 5-methylthioadenosine; and glutamate; (b) an obesity determining engine configured to determine obesity based on levels and/or activities of metabolites of (a) or derivatives thereof; (c) a data source (e.g., human metabolome database); and (d) a display communicatively connected to a computing device and configured to display a report containing the subject's obesity profile, wherein each of components (a), (b), (c) and (d) is communicatively connected to each other either directly or via indirectly (e.g., via the internet).

In some embodiments, the disclosure relates to an obesity profiling system, comprising: (a) a metabolome detector/analyzer configured to detect/analyze levels or activities of at least 3 metabolites of Table 2 or derivatives thereof in a subject's biological sample, wherein the at least 3 metabolites comprises, in the order of rank of relative correlation to the subject's obesity, urate, glutamate and 1-(1-enyl-palmitoyl)-2-oleoyl-GPC (P-16:0/18:1); (b) an obesity determining engine configured to determine obesity based on levels and/or activities of metabolites of (a) or derivatives thereof; (c) a data source (e.g., human metabolome database); and (d) a display communicatively connected to a computing device and configured to display a report containing the subject's obesity profile, wherein each of components (a), (b), (c) and (d) is communicatively connected to each other either directly or via indirectly (e.g., via the internet).

In some embodiments, the disclosure relates to an obesity profiling system of the foregoing, comprising: (a) a detector/analyzer configured to detect levels or activities of at least 3 metabolites of Table 1 or derivatives thereof in a subject's biological sample, wherein the at least 3 metabolites comprises, in the order of rank of relative correlation to the subject's obesity, urate, 5-methylthioadenosine, and glutamate.

In some embodiments, the disclosure relates to an obesity profiling system, comprising: (a) a detector/analyzer configured to detect metabolic profile comprising at least 3 metabolites of Table 2 or derivatives thereof in a subject's biological sample, wherein the at least 3 metabolites comprises, in the order of rank of relative correlation to the subject's obesity, glutamate and 1-(1-enyl-palmitoyl)-2-oleoyl-GPC (P-16:0/18:1).

In some embodiments, the disclosure relates to a kit for determining a lipid or fat content of a biological sample, comprising, a plurality of probes for detecting a metabolite profile of the biological sample; vessels for holding the biological sample; optionally together with instructions for performing the detection, wherein the metabolite profile comprises at least three of the metabolites of Table 1 or derivatives thereof, wherein the at least 3 metabolites comprises: urate, 5-methylthioadenosine, and glutamate or derivatives thereof.

In some embodiments, the disclosure relates to a kit for determining a lipid or fat content of a biological sample, comprising, a plurality of probes for detecting a metabolite profile of the biological sample; vessels for holding the biological sample; optionally together with instructions for performing the detection, wherein the metabolite profile comprises at least three of the metabolites of Table 2 or derivatives thereof, wherein the at least 3 metabolites comprises: urate, glutamate and 1-(1-enyl-palmitoyl)-2-oleoyl-GPC (P-16:0/18:1) or derivatives thereof.

### DETAILED DESCRIPTION

The disclosure relates to various exemplary embodiments of systems and methods to make precise predictions for individuals by measuring certain biomarkers in his/her metabolome. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

### I. Definitions

As used herein, the term “diagnosis” refers to methods by which a determination can be made as to whether a subject is likely to be suffering from a given disease or condition, including but not limited diseases or conditions characterized by genetic variations. The skilled artisan often makes a diagnosis on the basis of one or more diagnostic indicators, e.g., a marker such as a metabolome, the presence, absence, amount, or change in amount, level or activity of which is indicative of the presence, severity, or absence of the disease or condition. Other diagnostic indicators can include patient history; physical symptoms (e.g., breathlessness, increased sweating, snoring, inability to cope with sudden physical activity, tiredness, lethargy, back and joint pains, etc.); psychological symptoms (e.g., low self-confidence and/or self-esteem, feeling isolated, depression, etc.); phenotype changes (large waistline, unhealthy fat distribution); metabolic syndrome (e.g., high regular body mass index, high triglyceride levels, low HDL cholesterol levels, high fasting blood sugar, type 2 diabetes, diseases of heart and/or blood vessels such as, e.g., deregulated blood pressure, atherosclerosis, heart attacks, or strokes; etc.); diseases of organs such as liver (e.g., non-alcoholic fatty liver disease; NAFLD), gall bladder, urinary bladder (e.g., urinary incontinence) and bone (e.g., osteoarthritis); genotype; or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

As used herein, “metabolome” refers to the collection of all metabolites in a biological cell, tissue, organ or organism, which are the end products of cellular processes. Metabolome includes lipidome, sugars, nucleotides, amino acids, xenobiotics, carbohydrates, peptides, cofactors, vitamins, and cell process intermediates. As used herein, “lipidome” is the complete lipid profile in a biological cell, tissue, organ or organism.

As used herein, “metabolomic profiling” refers to the characterization and/or measurement of the small molecule metabolites in biological specimen or sample, including cells, tissue, organs, organisms, or any derivative fraction thereof and fluids such as blood, blood plasma, blood serum, saliva, synovial fluid, spinal fluids, urine, bronchoalveolar lavage, tissue extracts and so forth.

The “metabolite profile” or “metabolite signature” may include information such as the quantity and/or type of small molecules present in the sample. The ordinarily skilled artisan would know that the information, which is necessary and/or sufficient, will vary depending on the intended use of the metabolite profile. For example, the metabolite profile, can be determined using a single technique for an intended use but may require the use of several different techniques for another intended use depending on such factors as the disease state involved, the types of small molecules present in a particular targeted cellular compartment, the cellular compartment being assayed per se, and so forth.

The relevant information in a metabolite profile may also vary depending on the intended use of the compiled information, e.g., spectrum. For example for some intended uses, the amounts of a particular metabolite or a particular class of metabolite may be relevant, but for other uses the distribution of types of metabolites may be relevant.

Metabolite profiles may be generated by several methods, e.g., HPLC, thin layer chromatography (TLC), electrochemical analysis, Mass Spectroscopy (MS), refractive index spectroscopy (RI), Ultra-Violet spectroscopy (UV), fluorescent analysis, radiochemical analysis, Near-Infrared spectroscopy (Near-IR), Nuclear Magnetic Resonance spectroscopy (NMR), fluorescence spectroscopy, dual polarization interferometry, computational methods, Light Scattering analysis (LS), gas chromatography (GC), or GC coupled with MS, direct injection (DI) coupled with LC-MS/MS and/or other methods or combination of methods known in the art.

The term “small molecule metabolites” includes organic and inorganic molecules which are present in the cell, cellular compartment, or organelle, usually having a molecular weight under 2,000, or 1,500. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecule metabolites of the cell are generally found free in solution in the cytoplasm or in other organelles, such as the mitochondria, where they form a pool of intermediates which can be metabolized further or used to generate large molecules, called macromolecules.

The term “small molecule metabolites” includes signaling molecules and intermediates in the chemical reactions that transform energy derived from food into usable forms. Examples of small molecule metabolites include phospholipids, glycerophospholipids, lipids, plasmalogens, sugars, fatty acids, amino acids, nucleotides, intermediates formed during cellular processes, isomers and other small molecules found within the cell. In one embodiment, the small molecules of the invention are isolated.

As used herein, the term “a significant number” denotes at least 5%, at least 10%, least 15%, least 20%, least 25%, at least 40%, at least 50%, at least 60%, at least 70%, at least 80%, at least 90%, or 100% (e.g., all) of a set (e.g., the metabolites of the Tables).

As used herein, the term “cell” denotes a basic structural, functional, and biological unit. The term includes biological cells of living organisms and also artificial or synthetic cells. Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

As used herein, the term “sample” refers to a composition that is obtained or derived from a subject of interest that contains a cellular and/or other molecular entity that is to be characterized and/or identified, for example based on physical, biochemical, chemical and/or physiological characteristics. The source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Preferably, the sample is obtained from blood or blood components, including, e.g., whole blood, plasma, serum, lymph, and the like.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within 10%, or within 5% or less, e.g., with 2%.

As used herein, the term “detecting” refers to the process of determining a value or set of values associated with a sample by measurement of one or more parameters in a sample, and may further comprise comparing a test sample against a reference sample. In accordance with the present disclosure, the detection step includes identification, assaying, measuring and/or quantifying one or more markers or activities thereof.

As used herein, the term “level” is defined herein as including any information related to, for example, the amount, relative concentration and absolute concentration. The term also includes changes in the amount, relative and absolute concentrations, whether in a percentage or absolute context. These “level” changes may be used over a selected duration of time such as, for example, a time change in amount or concentration. The “level” may refer to a time change in amount or concentration, and compared to a later time change. The amount and rate of change of the metabolites are powerful tools in assessing the physiological state of the individual.

As used herein, the term “activity” relates to a functional property of a molecule (e.g., a metabolite). For the small molecule compounds, the term “activity” may relate to an adhesive property, e.g., binding to its binding partner such as a protein (e.g., enzyme, receptor, or antibody). Binding activity may be studied using Fourier transform spectroscopy (FTS), Raman spectroscopy, fluorescence spectroscopy (FS), circular dichroism (CD), nuclear magnetic resonance (NMR), mass spectrometry (MS), atomic force microscope (AFM), paramagnetic probes, dual polarization interferometry, surface plasmon resonance (SPR), fluorescence intensity, bimolecular fluorescence complementation, fluorescent resonance energy transfer (FRET), bio-layer interferometry, co-immunopreciptation, ELISA, equilibrium dialysis, gel electrophoresis, far western blot, fluorescence polarization anisotropy, electron paramagnetic resonance, or microscale thermophoresis. “Activity” of a molecule may also relate to a “functional activity” e.g., pharmacological activity (e.g., agonist, partial agonist or antagonist activity on a receptor or ligand), catalytic activity (e.g., allosteric regulation of an enzyme), toxicity (e.g., apoptotic or necrotic activity), or chemical activity (e.g., pigmentation). Functional activities may be determined using routine functional assays, e.g., pharmacological assays, toxicity assays, enzyme kinetics, colorimetric or fluorescence assays, etc. The term “activity” is used broadly to include a binary definition, e.g., a definition of a compound, as a whole, being either active or inactive. Additionally, the present systems and methods can provide finer binning, ranges of percentile IC₅₀ or raw IC₅₀ values, including grouping (e.g., quantile or standard deviations) based on statistical weights corresponding to functional profile or other molecular parameter. Probabilities for a compound to be active may also be reflected in the activity profile. For example, the present systems and methods can correlate molecular parameters with experimental data, so that a user can be provided with an estimation about the activity. Some implementations can use a linear regression model.

As used herein, the term “marker” refers to a characteristic that can be objectively measured as an indicator of normal biological processes, pathogenic processes or a pharmacological response to a therapeutic intervention, e.g., treatment with an anti-obesity agent. Representative types of marker characteristics include, for example, molecular changes in the structure (e.g., changes in the chemical composition of a metabolite) or level (e.g., changes in concentration of a metabolite) or activity (e.g., changes in pharmacological activity, enzymatic activity, metabolic activity, or any other biological activity). Marker characteristics may further include, e.g., a plurality of differences, such as changes in the levels of molecular markers and activities thereof.

As used herein, the term “metabolite” refers to the end product that remains after metabolism. In some embodiments, these metabolites leach out into the biological fluid, e.g., blood, sweat, urine, saliva, pleural fluid, tears, over time. Preferably, metabolites are compound derived from the metabolism of a biological macromolecule, e.g., fats, lipids, carbohydrates, polysaccharides, polynucleotides, etc.

As used herein, the term “derivative” includes salts, amides, esters, enol ethers, enol esters, acetals, ketals, acids, bases, solvates, hydrates, or polymorphs of the individual metabolites. Derivatives may include precursors or products (e.g., glutamate is a derivative of glutamine and vice versa). Derivatives may be readily prepared by those of skill in this art using known methods for such derivatization. The derivatives suitable for use in the methods described herein may be detected using methods that are used for detecting parent metabolites. Derivatives include solvent addition forms, e.g., a solvates or alcoholates, which may be synthesized to facilitate detection. Derivatives further include amides or esters of the amino acids and/or isomers (e.g., stereoisomers).

As used herein, the term “salt” includes salts derived from any suitable of organic and inorganic counter ions well known in the art and include, by way of example, hydrochloric acid salt or a hydrobromic acid salt or an alkaline or an acidic salt of the metabolites.

As used herein, the term “solvate” refers to compounds containing either stoichiometric or non-stoichiometric amounts of a solvent such as water, ethanol, and the like. “Hydrates” are formed when the solvent is water; alcoholates are formed when the solvent is alcohol.

As used herein, the term “metabolite profile” or “metabolomics profile” includes an inventory of metabolites (in tangible form or computer readable form) within a sample from a subject, or any derivative fraction thereof, that is necessary and/or sufficient to provide information to a user for its intended use within the methods described herein. The inventory may include the quantity, levels, activities and/or types of small molecules present. The information, which is necessary and/or sufficient, will vary depending on the intended use of the “metabolite profile.” For example, the “metabolite profile,” can be determined using a single technique for an intended use but may require the use of several different techniques for another intended use depending on such factors as genotypic or phenotypic traits of the subject, the disease state involved, the types of small molecules present in a particular sample, etc. In a further embodiment, the small molecule profile comprises information regarding at least 3, at least 5, at least 10, at least 20, at least 25, at least 35, at least 50, at least 75, at least 100, at least 150, at least 200, at least 250, at least 300, at least 350, or more, e.g., at least 400, metabolites. In some instances the term “profile” may be used to refer to said inventory of small molecules.

As used herein, “reference standard” refers to a sample of tissue or cells that may or may not have the disorder (e.g., obesity) or a trait thereof that are used for comparisons. Thus a “reference” standard thereby provides a basis to which another sample, for example plasma sample containing metabolite markers, e.g., metabolites of Table 1, that can be compared. In contrast, a “test sample” refers to a sample compared to a reference standard or control sample.

As used herein, the term “reference metabolic profile” or “reference metabolomic profile” refers to the resulting profile generated using the “reference sample.” The term includes information regarding the small molecules of the profile that is necessary and/or sufficient to provide information to a user for its intended use within the methods described herein. The reference profile would include the quantity and/or type of small molecules present.

As used herein, “test sample” refers to a sample obtained from the individual subject to be analyzed. The term “control,” as used herein, refers to a reference for a test sample, such as control cells obtained from healthy or normal subjects, wherein the subjects are not suffering from or are otherwise predisposed to obesity. In some aspects, controls include samples obtained from the same subject at different points in time, during which, the subject may be going through a clinically-approved therapy or experimental therapy, e.g., with drugs or surgical intervention or both.

The term “modulate” as used herein refers to an increase or decrease. The change (e.g., increase or decrease) may be qualitative or quantitative in nature. For example, the term modulate may refer to a post-therapy reduction in BMI values (e.g., quantitative modulation) or drops in mood swings (e.g., qualitative modulation) or reduction in a composite qualitative-quantitative score such as Patient Health Questionnaire-9 (PHQ9) in obese patients.

The term “enhance” or “increase” refers to an increase in the specified parameter of, e.g., at least about 1.25-fold, 1.5-fold, 2-fold, 3-fold, 4-fold, 5-fold, 6-fold, 8-fold, 10-fold, twelve-fold, or fifteen-fold, or greater, e.g., 25-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more, e.g., 99%. In particular embodiments, the inhibition or reduction results in little or essentially no detectible levels or activity of the parameter being measured. Herein, non-detectable level/activity typically represents an insignificant level/activity, e.g., <about 10% or even <about 5% of the initial level/activity.

As used herein, the term “treating” refers to curative action, palliative action (e.g., control or mitigate a disease or disease symptoms) or prophylactic action (e.g., reduce the frequency of, or delay the onset of a pathologic condition or symptoms of the condition in a subject receiving the therapy relative to a subject not receiving therapy. This can include reversing, reducing, or arresting the symptoms, clinical signs, and underlying pathology of a condition in a manner to improve or stabilize a subject's condition (e.g., regress rapid weight gain in obese subjects).

As used herein, the term “lifestyle therapy” includes, dietary management (e.g., reduce intake of high calorie diet), exercise management (e.g., increase frequency and/or rigor of exercise), stress management (e.g., reduce emotional or mental stress) and/or behavior management (e.g., quit smoking).

As used herein, the term “administering” is used in the broadest sense as giving or providing to a subject in need of the treatment, a composition such as a pharmaceutical agent (e.g., drug) or a pharmaceutical composition containing the pharmaceutical agent. For instance, in the pharmaceutical sense, “administering” means applying as a remedy, such as by the placement of a drug in a manner in which such drug would be received, e.g., intravenous, oral, topical, buccal (e.g., sub-lingual), vaginal, parenteral (e.g., subcutaneous; intramuscular including skeletal muscle, cardiac muscle, diaphragm muscle and smooth muscle; intradermal; intravenous; or intraperitoneal), topical (e.g., skin or mucosal surfaces), intranasal, transdermal, intraarticular, intrathecal, inhalation, intraportal delivery, organ injection (e.g., eye or blood, etc.), or ex vivo (e.g., via immunoapheresis).

As used herein, “contacting” means that the composition comprising the pharmaceutical agent or a pharmaceutical composition comprising the agent is introduced into a sample containing a target, e.g., cell target, in a test tube, flask, tissue culture, chip, array, plate, microplate, capillary, or the like, and incubated at a temperature and time sufficient to permit binding of the agent to the target (e.g., cells) or vice versa (e.g., blood cells coming into contact with the agent). In the in vivo context, “contacting” means that the therapeutic or diagnostic molecule is introduced into a patient or a subject for the treatment of a disease, and the molecule is allowed to come in contact with the patient's target tissue, e.g., blood tissue, in vivo or ex vivo.

As used herein, the term “therapeutically effective amount” refers to an amount that provides some improvement or benefit to the subject. Alternatively stated, a “therapeutically effective” amount is an amount that will provide some alleviation, mitigation, or decrease in at least one clinical symptom in the subject. Methods for determining therapeutically effective amount of the therapeutic molecules, e.g., anti-obesity drugs, are described below.

As used herein, the term “subject” means an individual. In one aspect, a subject is a mammal such as a human. In one aspect a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., cows, pigs, goats), laboratory animals (e.g., mouse, rabbit, rat, gerbil, guinea pig, etc.) and avian species (e.g., chickens, turkeys, ducks, etc.). Subjects can also include, but are not limited to fish (for example, zebrafish, goldfish, tilapia, salmon, and trout), amphibians and reptiles. Preferably, the subject is a human subject. Especially, the subject is a human patient.

As used herein, the term “obesity” generally refers to a condition, temporary or chronic, which is defined by an excess amount body fat. The normal amount of body fat (expressed as percentage of body weight) is between about 25-30% in women and about 18-23% in men. Women with over 30% body fat and men with over 25% body fat are characterized as being obese.

As used herein, the term “healthy obesity” denotes a condition which would normally be classified as overweight or obese under a clinically acceptable metric, e.g., a body mass index (BMI) score of at least about 25 (overweight) or 30 (obesity), but which is extricated from the health complications that are normally linked with obesity.

In contrast, the term “metabolic obesity” denotes a condition which can be classified as non-obese under a clinically acceptable metric, e.g., a body mass index (BMI) score of less than about 25 (overweight) or 30 (obese), but which is nonetheless implicated with the health complications that are normally linked with obesity. “Unhealthy obesity” includes, but is not limited to, metabolic syndrome (a cluster of metabolic disorders that is characterized by obesity, high blood lipid levels, high blood pressure, and/or insulin resistance/high blood sugar) and cardiovascular disease consequences. The level of unhealthiness may be qualitative or quantitative, preferably quantitative. Cutoffs between healthy and unhealthy may be made based on statistical measurements, e.g., using a parametric or a non-parametric mBMI distribution and confidence estimates. Alternately, a regression residual for the difference between two parameters (e.g., BMI and mBMI, optionally adjusted for age and sex) may be used. Individuals in the top 5%, top 10%, top 20%, top 25%, or top 40%, preferably top 10% of the residual distribution may be classified as being obese.

“Body Mass Index, (or BMI)” refers to a calculation that uses the height and weight of an individual to estimate the amount of the individual's body fat. Too much body fat (e.g. obesity) can lead to illnesses and other health problems. BMI is the measurement of choice for many physicians and researchers studying obesity. BMI is calculated using a mathematical formula that takes into account both height and weight of the individual. BMI equals a person's weight in kilograms divided by height in meters squared. (BMI=kg/m2). Subjects having a BMI less than 18.5 are considered to be underweight, while those with a BMI of between 18.5 and 25 are considered to be of normal weight, while a BMI of between 25 to 30 are generally considered overweight, while individuals with a BMI of 30 or more are typically considered obese. Morbid obesity refers to a subject having a BMI of 40 or greater.

As used herein, an “obesity-related disease or condition” includes, but is not limited to, coronary artery disease, hypertension, stroke, peripheral vascular disease, insulin resistance, glucose intolerance, diabetes mellitus, hyperglycemia, hyperlipidemia, hypercholesteremia, hypertriglyceridemia, hyperinsulinemia, atherosclerosis, cellular proliferation and endothelial dysfunction, diabetic dyslipidemia, lipodystrophy and metabolic syndrome, type II diabetes, diabetic complications including diabetic neuropathy, nephropathy, retinopathy or cataracts, heart failure, inflammation, thrombosis, congestive heart failure, asthmatic or pulmonary disease related to obesity, and cardiovascular disease related to obesity.

As used herein, the term “screen” refers to a specific biological or biochemical assay which is directed to measurement of a specific condition or phenotype that a molecule induces in a target, e.g., target cell-free system, target cells, tissues, organs, organ systems, or organisms.

As used herein, the term “selecting” in the context of screening compounds or libraries includes both (a) choosing compounds from a group previously unknown to be modulators of a condition or phenotype (e.g., obesity); and (b) testing compounds that are known to be inhibitors or activators of the condition or phenotype (e.g., obesity). Both types of compounds are generally referred to herein as “test compounds.” The test compounds may include, by way of example, polypeptides (e.g., small peptides, artificial or natural proteins, antibodies), polynucleotides (e.g., DNA or RNA), carbohydrates (small sugars, oligosaccharides, and complex sugars), lipids (e.g., fatty acids, glycerolipids, sphingolipids, etc.), mimetics and analogs thereof, and small organic molecules having a molecular weight of less than about 10 KDa, preferably less than about 5 KDa, especially less than about 1 KDa (e.g., about 300 daltons to about 800 daltons). Preferably, the test compounds are provided in library formats known in the art, e.g., in chemically synthesized libraries, recombinantly-expressed libraries (e.g., phage display libraries), and in vitro translation-based libraries (e.g., ribosome display libraries).

### II. Methods

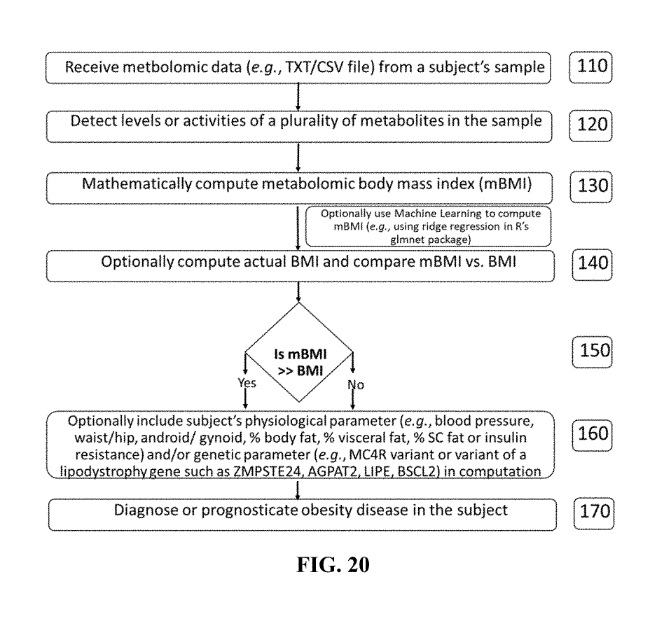

In some embodiments, the disclosure relates to a method of diagnosis of obesity in subjects. FIG. 20 is a representative flow chart illustrating a method 100 for diagnosing obesity or a disorder related thereto (e.g., diabetes) in accordance with the various embodiments of the present disclosure. Method 100 is illustrative only and embodiments can use variations of method 100. Method 100 can include steps for receiving a metabolic profile (e.g., data on the composition and/or activity of the metabolites in a subject's sample, e.g., blood or serum).

In step 110 of method 100 of FIG. 20, metabolomic data is received from a subject. In some embodiments, the metabolomic data comprising the markers, e.g., levels or activities of the various metabolites or derivatives thereof, is received in a comma separated value (CSV) file or text (TXT) file. As is understood in the art, CSV files are used in metabolomics for storing information about metabolites. Alternately, the subject's metabolomics data is received in situ by processing the subject's sample using HPLC, TLC, electrochemical analysis, mass spectroscopy, refractive index spectroscopy (RI), Ultra-Violet spectroscopy (UV), fluorescent analysis, radiochemical analysis, Near-Infrared spectroscopy (Near-IR), Nuclear Magnetic Resonance spectroscopy (NMR), and Light Scattering analysis (LS), preferably, HPLC (Kristal et al., Anal. Biochem. 263: 18-25, 1998), thin layer chromatography (TLC), or electrochemical separation techniques (see, WO 99/27361, WO 92/13273, U.S. Pat. Nos. 5,290,420, 5,284,567, 5,104,639, 4,863,873, and U.S. RE 32,920). A combination of the aforementioned techniques may be used, e.g., ultra-high performance liquid chromatography-tandem mass spectrometry (UPLC-MS/MS).

In step 120 of method 100 of FIG. 20, levels or activities of the metabolites are detected. As outlined previously, levels of metabolites may be determined using routine chemical detection techniques such as UPLC-MS/MS. Levels of metabolites may be expressed in mass units (e.g., μg or pg), mole units (e.g., micromoles or picomoles), or concentration units (e.g., μM or pM). Activities of metabolites may be measured using functional assays. For e.g., as is known in the art, many metabolites serve as substrates of enzymes and/or regulators of informational molecules such as proteins and nucleic acids. As such, abundance of metabolites is decisive to the biological roles. When metabolite levels are modulated, enzyme activity or regulation of proteins will be modulated, which affect the metabolic pathways and networks. Differential activation or suppression of one or more metabolic pathways could be a critical feature of the response (stress or disease) phenotype. Thus, alternately, phenotypic changes at the cellular, tissue, organ or organism level, which are triggered by the metabolites of the disclosure, may also be used in the computation of the functional parameter of the disclosure (mBMI values). Further, in step 120, the received metabolomic data may be optionally analyzed using toolkit, e.g., METACORE, METABOANALYST, INCROMAP and 3OMICS (see, Cambiaghi et al., Briefings in Bioinformatics, 18, 498-510, 2017).

The metabolomic data, which are optionally analyzed with a toolkit, may be processed to generate standardized data, which ensures non-redundancy and/or integrity of data. The processing step may comprise normalization and/or standardization. Thus, the process of encoding categorical data and normalizing numeric data (sometimes called data standardization) can be carried out in accordance with the methods of the present disclosure. For example, values from multiple experimental batches may be normalized into Z-scores based on a reference cohort of n self-reported healthy individuals run with each batch, which normalized batches are converted to the same scale using linear transformation based on the values obtained from the runs that include the controls. Samples with metabolite measurements that are below the detection threshold are imputed as the minimum value for that metabolite and any batch that does not meet this threshold requirement may be purged or rerun. This process may be carried out for each metabolite of interest.

In embodiments wherein the metabolomic data is received in situ, any biological sample may be used to obtain the metabolomics profile. Preferably, the sample is a biological fluid sample, containing, w/w, at least about 20%, 30%, 40%, 50%, 60%, 70%, 80%, 90%, or 99% of an aqueous agent compared to particulate matter in solution, dispersion, colloid, or sol-form. Representative examples include, e.g., blood (including whole blood), blood plasma, blood serum, hemolysate, lymph, synovial fluid, spinal fluid, urine, cerebrospinal fluid, stool, sputum, mucus, amniotic fluid, lacrimal fluid, cyst fluid, sweat gland secretion, bile, milk, tears, saliva, or earwax. Blood-based samples, e.g., plasma, serum, hemolysate, lymph, are preferred.

In step 130 of method 100 of FIG. 20, the subject's metabolomic body mass index (mBMI) is mathematically computed. A variety of methods may be used in computing mBMI values based on the levels or activities of the metabolites of the disclosure that are detected in a subject's sample, including, e.g., machine learning (ML). ML may be incorporated as an add-on to the computational methods to systemically eliminate or reduce noise. The approach may be applied at any step of the method, although it may be advantageous to implement the ML after the markers have been detected in step 120 and their levels or activities have been determined. In this regard, in the purely illustrative method of FIG. 20, an ML algorithm is optionally applied at step 130 to build the model. The ML algorithm may comprise employing a deep learning algorithm such as, e.g., using neural networks to analyze actual patient samples to identify signatures that discriminate between true markers and noise. In some embodiments, the ML method comprises use of linear regression to compute mBMI values. Purely as a representative example, mBMI values may be computed using ridge regression in R's glmnet package. The formula for the calculation may be identified using machine learning and artificial intelligence techniques and is provided in Equation 1 above, e.g., mBMI=sum((coefficient)×(metabolite value))+Intercept.

As can be seen from Equation 1, the metabolite value (e.g., levels or concentration) exerts an effect on mBMI. Accordingly, in some embodiments, the metabolites of Tables 1-7, Table 11, Table 12A, Table 12B; preferably the metabolites of Tables 2-7; and especially the metabolites of Table 2 or Tables 4-7, exert an effect on mBMI. Particularly, in the case of the metabolites of Tables 1, 2, and 4-7, the relative effect of each metabolite on mBMI is associated with the order in which they are listed (i.e., metabolites that are listed at the top exert an effect on mBMI that is greater than metabolites that are listed in the bottom). Moreover, in the case of the metabolites of Table 3, the relative effect of each metabolite on mBMI is associated with its rank (in parenthesis).

In some embodiments, the ML is trained with an in silico metabolomic dataset. For example, the in silico dataset may include tissue samples (e.g., from subjects, both male and female, who are between 12 and 95 years of age. The association between specific metabolites and obesity is identified using a robust mathematical regression. The markers that are highly specific and also tightly associated with specific conditions, e.g., cardiovascular diseases (e.g., heart disease such as heart attack, angina, heart failure, arrhythmia), cerebrovascular diseases (e.g., stroke), vascular disease (e.g., high blood pressure), and/or diabetes, may be further identified using the robust mathematical regression, are then studied for the features, including, association with any obesity-related genes or signatures. A representative method is described in the Examples.

The architecture of the machine learning approach will be discussed in detail below.

Not being bound to a single embodiment and purely for the purpose of illustration, a machine-learning algorithm was integrated into the existing methodology at an individual, or combination of individual steps, in accordance with various embodiments herein. ML can be incorporated to optimize the results coming out of the algorithm (e.g., neural network, ML algorithm, etc.), by utilization of inputted training data sets, cross reference of output to known answers, backpropagation, and adjustment of weighting factors and parameters associated with the given ML algorithm in a repeating loop to arrive at a threshold quality of data output. For instance, in the process described here, machine earning (ML) is used to identify the best weights to assign to metabolites associated with BMI when building the mBMI model. The specific algorithm used is the glmnet package in R, specifically the cvglmnet function, which performs 10-fold cross validation. For training, we took a random half of our sample. The cvglmnet function performed 10-fold cross validation in this half of the dataset to assign the weights to each metabolite. We then tested the resulting model in the other half of the dataset by applying those weights. Other methods that could be used to achieve similar results would include random forest regression and linear regression. In subsequent steps, the prediction power of the model on the test dataset may be validated, e.g., using a probability model such as logistic regression. Optionally, a resampling may be performed to obtain an unbiased appraisal of the model's likely future performance. Features of ROC curve, such as, area-under-the curve (also called c-index) or concordance probability from a statistical test such as the Wilcoxon-Mann-Whitney test, may provide a good summary measure of pure predictive discrimination.

Generally in method 100 of FIG. 20, a machine learning approach may be incorporated to systemically determine, for example, the relative weights of various metabolites. The approach may be applied at any step of the method, although it may be advantageous to implement the machine learning at step 130. In this regard, in the purely illustrative method of FIG. 20, a machine learning (ML) algorithm is optionally applied at step 130 to build the model. The ML algorithm may comprise employing a deep learning algorithm such as, e.g., using neural networks, with applicable training data sets and specific weighting factors optimized by backpropogation, to analyze variations in levels and/or activities of metabolites (or derivatives thereof) and deduce the functional significance thereof.

In step 140 of method 100 of FIG. 20, the subject's actual body mass index (BMI) is optionally computed and may be used in comparative assessment. BMI may be calculated using the formula BMI=[weight (lb)/[height (in)]²×703 (English system) or BMI=[weight (kg)/height (cm)/height (cm)]×10,000 (metric system).

It should be noted that use of actual BMI for comparative assessment is optional because the subject's mBMI value may be directly compared with a reference standard (e.g., control). In some embodiments, control mBMI values may be determined using an identical sample obtained from a non-obese individual, which values are computed using steps 110, 120 and 130 of the aforementioned method 100). In some embodiments, the control mBMI values may be based on statistically determined value (e.g., mean or median) in a population of non-obese subjects. Control mBMI may be adjusted for age, gender, race, and any other variable that may influence the physiology of the subject.

In step 150 of method 100 of FIG. 20, the subject's metabolomic body mass index (mBMI) is compared with the actual BMI. Subject's whose mBMI≈BMI are not classified as outliers because their actual BMI serves as a reliable predictor of obesity and/or related diseases.

In step 160 of method 100 of FIG. 20, a secondary parameter is optionally detected and included in the final analytical step 170. Step 160 may include a secondary parameter such as android/gynoid ratio; total triglycerides; waist/hip ratio; subcutaneous fat; visceral fat; insulin resistance; HDL; percent fat; diastolic blood pressure; systolic blood pressure; total cholesterol; and LDL, or a combination thereof, preferably, android/gynoid ratio; total triglycerides; waist/hip ratio; subcutaneous fat; visceral fat; insulin resistance; and HDL. Step 160 may include a genetic parameter selected from whether the subject is a carrier or a melanocortin 4 receptor gene (MC4R) variant, preferably an MC4R variant selected from M292fs, R236C, S180P, A175T, and T11A, but not I170V; and/or whether the subject is a carrier of a genetic variant of a lipodystrophy gene selected from ZMPSTE24, AGPAT2, LIPE, BSCL2 or any combination thereof. Preferably, the final analytical step includes at least inclusion of a secondary parameter and/or a genetic parameter (preferably both), as it was found to improve the accuracy of diagnosis or prognosis (e.g., correlation between mBMI and actual BMI). In some embodiments, outlier subjects whose mBMI<<BMI (e.g., false positive obese based on BMI) may be subjected to additional body composition tests (e.g., waist circumference, waist-to-hip ratio, body fatness, lipedema) or biochemical tests (e.g., for high triglyceride levels, high LDL cholesterol, low HDL cholesterol levels, high fasting blood sugar, glycemia, insulin resistance or a combination thereof). Similarly, false negative outlier subjects (e.g., subjects whose mBMI>>BMI) may be classified as “at risk” and therefore be subjected to additional tests, e.g., measurement of blood pressure, waist/hip ratio, android/gynoid ratio, % body fat, % visceral fat, % subcutaneous fat or insulin resistance, the results of which may be used in the final prognostication step 170. Blood total, HDL and LDL cholesterol, triglycerides, urates, creatinine, sodium and potassium concentrations, ALAT, ASAT, GGT, glucose, non-esterified fatty acids, insulin and mean arterial blood pressure (MAP) may be determined using routine laboratory methods (U.S. Pat. No. 9,261,520). Insulin resistance status may be assessed as homeostasis model assessment of insulin resistance (HOMA-IR) according to the previously described formula (Matthews et al., Diabetologia 28:412-419, 1985): insulin (μU/mL)×glucose (mmol/L)/22.5. Preferred types of secondary parameters included in the computational methods and/or algorithms of the disclosure are listed in Table 8. Preferred types of genetic parameters included in the computational methods and/or algorithms of the disclosure are listed in Tables 9 and 10.

In step 170 of method 100 of FIG. 20, the obesity disease is diagnosed or prognosticated in the subject by comparing mBMI values, optionally together with the additional obesity parameters outlined above, to that of a reference standard. In a representative mBMI model, values above about −0.073 are considered overweight (range from about −0.073 to about 0.314), and values above about 0.314 (e.g., 0.32, 0.4, 0.5, 0.6, 0.7, 0.8, 0.9, 1.0) are considered obese. For BMI, values between 18.5 and 25 are considered normal, 25-30 is considered overweight, and >30 is considered obese.

In some embodiments, the reference standard comprises a BMI score for the subject and the subject is deemed at risk of obesity or a disease associated therewith if the mBMI>>a BMI of about 18.5 to about 24.9 kg/m² (normal BMI); particularly if mBMI>a BMI of about 25 to about 30 kg/m² (overweight BMI); and especially if the mBMI>a BMI of about 30 kg/m² (obese BMI).

Preferably, determinations of normal weight, overweight, obesity or morbid obesity are made via statistical analysis. In a representative embodiment, a residual score may be used. For instance, if the residual of mBMI regressed on BMI, age and sex is greater than about 0.4, 0.5, 0.6, 0.7, or more, e.g., 0.8 (preferably, >0.5) then they are put into the high risk category.

In some embodiments, the methods of the disclosure may be further carried out by detecting one or more signatures. Such signatures may comprise, for example, a plurality of metabolites (e.g., about 2, 3, 4, 5, 6, 8, 10, 12, 13, 15, 20, 25, 30, 35, 40, 45, 49, 50, 60, 75, 100, 125, 150, 200, 250, 300, 307 or more metabolites). Representative signatures including a significant number, e.g., at least 50%, at least 65%, at least 80%, at least 90%, or more, e.g., 100% of the metabolites of Table 11, Table 12A, Table 12B, or Tables 1-7 (preferably Tables 2-7), or derivatives thereof.

In some embodiments, the methods of the disclosure are carried out by detecting one or more signatures comprising the broad classes of metabolites recited in Table 3, or a derivative thereof.

TABLE 3
Metabolite signature associated with BMI.

| | | | Direction | |
|---|---|---|---|---|
| Super | | Sub pathway (Correlated | of effect | BMI |
| pathway | Metabolite | blood lipids^{†}) | (rank*) | r²** |
| Nucleotide | urate | Purine Metabolism, | ↑ (1) | 16.4% |
| | | (Hypo)Xanthine/Inosine | | |
| | | containing | | |
| | N2,N2-dimethylguanosine | Purine Metabolism, Guanine | ↑ (6) | 8.8% |
| | | containing | | |
| | N6- | Purine Metabolism, Adenine | ↑ (28) | 7.3% |
| | carbamoylthreonyladenosine | containing | | |
| Amino Acid | glutamate | Glutamate Metabolism | ↑ (2) | 11.5% |
| | N-acetylglycine | Glycine, Serine and Threonine | ↓ (9) | 9.0% |
| | | Metabolism | | |
| | 5-methylthioadenosine (MTA) | Polyamine Metabolism | ↑ (10) | 7.5% |
| | valine | Leucine, Isoleucine and Valine | ↑ (11) | 8.8% |
| | | Metabolism | | |
| | aspartate | Alanine and Aspartate | ↑ (16) | 7.0% |
| | | Metabolism | | |
| | N-acetylvaline | Leucine, Isoleucine and Valine | ↑ (18) | 7.3% |
| | | Metabolism | | |
| | kynurenate | Tryptophan Metabolism | ↑ (19) | 6.0% |
| | alanine | Alanine and Aspartate | ↑ (23) | 5.3% |
| | | Metabolism | | |
| | asparagine | Alanine and Aspartate | ↓ (26) | 3.7% |
| | | Metabolism | | |
| | N-acetylalanine | Alanine and Aspartate | ↑ (31) | 6.6% |
| | | Metabolism | | |
| | tyrosine | Phenylalanine and Tyrosine | ↑ (34) | 1.8% |
| | | Metabolism | | |
| | leucine | Leucine, Isoleucine and Valine | ↑ (37) | 6.8% |
| | | Metabolism | | |
| | N-acetyltyrosine | Phenylalanine and Tyrosine | ↑ (40) | 4.2% |
| | | Metabolism | | |
| | 2-methylbutyrylcarnitine (C5) | Leucine, Isoleucine and Valine | ↑ (41) | 8.3% |
| | | Metabolism | | |
| Lipid | 1-(1-enyl-palmitoyl)-2-oleoyl- | Plasmalogen (HDL, TG) | ↓ (3) | 7.1% |
| | GPC (P-16:0/18:1) | | | |
| | 1-stearoyl-2-dihomo-linolenoyl- | Phospholipid Metabolism (TG, | ↑ (4) | 9.8% |
| | GPC (18:0/20:3n3 or 6) | Chol) | | |
| | 1-eicosenoyl-GPC (20:1) | Lysolipid | ↓ (5) | 6.2% |
| | 1-arachidoyl-GPC (20:0) | Lysolipid | ↓ (7) | 8.6% |
| | 1-(1-enyl-stearoyl)-2-oleoyl- | Phospholipid (HDL) | ↓ (8) | 6.5% |
| | GPC (P-18:0/18:1) | | | |
| | propionylcarnitine | Fatty Acid Metabolism (also | ↑ (12) | 9.9% |
| | | BCAA Metabolism) | | |
| | 1-nonadecanoyl-GPC (19:0) | Lysolipid | ↓ (14) | 4.2% |
| | 1-linoleoyl-GPC (18:2) | Lysolipid | ↓ (15) | 4.9% |
| | sphingomyelin (d18:1/18:1, | Sphingolipid Metabolism (Chol) | ↑ (20) | 6.8% |
| | d18:2/18:0) | | | |
| | 1-palmitoyl-2-dihomo- | Phospholipid Metabolism (TG, | ↑ (21) | 5.1% |
| | linolenoyl-GPC (16:0/20:3n3 or | Chol) | | |
| | 6) | | | |
| | 1-(1-enyl-palmitoyl)-2- | Phospholipid Metabolism | ↓ (22) | 5.7% |
| | linoleoyl-GPC (P-16:0/18:2) | (HDL) | | |
| | 1-palmitoyl-3-linoleoyl- | Phospholipid Metabolism (TG) | ↑ (24) | 7.6% |
| | glycerol (16:0/18:2) | | | |
| | 1-oleoyl-2-linoleoyl-GPC | Phospholipid Metabolism | ↓ (27) | 5.6% |
| | (18:1/18:2) | | | |
| | 1-(1-enyl-stearoyl)-2- | Phospholipid Metabolism | ↓ (29) | 2.5% |
| | docosahexaenoyl-GPC (P- | | | |
| | 18:0/22:6) | | | |
| | 1-oleoyl-3-linoleoyl-glycerol | Diacylglycerol (TG, HDL) | ↑ (30) | 6.3% |
| | (18:1/18:2) | | | |
| | carnitine | Carnitine Metabolism | ↑ (33) | 7.5% |
| | 1-palmitoyl-2-linoleoyl- | Phospholipid Metabolism (TG, | ↑ (36) | 7.2% |
| | glycerol (16:0/18:2) | HDL) | | |
| | 1-oleoyl-2-linoleoyl-glycerol | Diacylglycerol (TG, HDL) | ↑ (38) | 5.9% |
| | (18:1/18:2) | | | |
| | 1,2-dilinoleoyl-GPC (18:2/18:2) | Phospholipid Metabolism | ↓ (39) | 4.2% |
| | 1-palmitoleoyl-2-oleoyl- | Phospholipid (TG) | ↑ (42) | 5.6% |
| | glycerol (16:1/18:1) | | | |
| | 1-palmitoleoyl-3-oleoyl- | Phospholipid (TG) | ↑ (45) | 6.0% |
| | glycerol (16:1/18:1) | | | |
| | 1-palmitoyl-2-adrenoyl-GPC | Phospholipid Metabolism (TG) | ↑ (47) | 2.9% |
| | (16:0/22:4) | | | |
| | cortisone | Steroid | ↓ (49) | 2.5% |
| Energy | succinylcarnitine | TCA Cycle | ↑ (13) | 9.8% |
| Carbohydrate | mannose | Fructose, Mannose and | ↑ (17) | 6.6% |
| | | Galactose Metabolism | | |
| | glucose | Glycolysis, Gluconeogenesis, | ↑ (48) | 6.3% |
| | | and Pyruvate Metabolism | | |
| Xenobiotics | cinnamoylglycine | Food Component/Plant | ↓ (43) | 3.5% |
| Cofactors and | gulonic acid | Ascorbate and Aldarate | ↑ (46) | 3.2% |
| Vitamins | | Metabolism | | |
| | quinolinate | Nicotinate and Nicotinamide | ↑ (44) | 8.4% |
| | | Metabolism | | |
| Peptide | N-acetylcarnosine | Dipeptide Derivative | ↑ (25) | 6.9% |
| | gamma-glutamylphenylalanine | Gamma-glutamyl Amino Acid | ↑ (32) | 6.0% |
| | gamma-glutamyltyrosine | Gamma-glutamyl Amino Acid | ↑ (35) | 4.6% |

*Rank indicates order of significance of association with BMI.
**Mean r² indicates the percent variation in BMI explained by each metabolite in univariate analysis for a combined analysis of the first time point of the TWINSUK cohort and the Health Nucleus data.
^{†}Blood labs for TG (triglycerides), Chol (cholesterol), HDL (high-density lipoprotein) or LDL (low-density lipoprotein) that had an r2 > 0.1 with the metabolite are indicated in parentheses.

Metabolites may be included/excluded in a signature based on a variety of criteria, including, inclusion or exclusion of metabolites (or derivatives) from the same class, e.g., amino acids, carbohydrates, lipids, co-factors, nucleotides, peptides, xenobiotics, etc.; inclusion or exclusion of metabolites (or derivatives) based on whether they belong to the same or different sub-pathway, e.g., amino acid metabolism, sugar metabolism, purine metabolism, phospholipid metabolism, steroid metabolism, fatty acid or TCA metabolism, etc.; inclusion or exclusion of metabolites (or derivatives) based on directionality of correlation with BMI, e.g., signatures comprising metabolites that are only positively or negatively correlated with BMI.

Owing partly due to enhanced prognostic significance of signatures compared to unitary markers, it may be preferable to group markers into distinct subgroups based on one or more statistical parameters. For instance, metabolites that are uncorrelated with each other (individually) may be grouped together so that changes in the levels/activities of individual markers are guided by factors other than other components of the composite. On this basis, a linear regression model was used to generate a three metabolite base signature comprising (a) 1-(1-enyl-stearoyl)-2-docosahexaenoyl-GPC (P-18:0/22:6); (b) sphingomyelin (d18:1/18:1, d18:2/18:0); and (c) urate; or derivatives thereof. Using a slightly more expansive linear regression model, a similar methodology was used to generate a six marker signature comprising: (a) N-acetylglycine, (b) 1-(1-enyl-stearoyl)-2-docosahexaenoyl-GPC (P-18:0/22:6), (c) sphingomyelin (d18:1/18:1, d18:2/18:0), (d) cortisone, (e) mannose, and (f) urate; or a derivative thereof.

TABLE 4
Subset of metabolites that are uncorrelated to one another,
which are included in a three-member signature.

| S/N | Metabolite |
|---|---|
| 1 | 1-(1-enyl-stearoyl)-2-docosahexaenoyl-GPC (P-18:0/22:6) |
| 2 | sphingomyelin (d18:1/18:1, d18:2/18:0) |
| 3 | urate |

TABLE 5
Subset of metabolites that are uncorrelated to one another,
which are included in a six-member signature.

| S/N | Metabolite |
|---|---|
| 1 | N-acetylglycine |
| 2 | 1-(1-enyl-stearoyl)-2-docosahexaenoyl-GPC (P-18:0/22:6) |
| 3 | sphingomyelin (d18:1/18:1, d18:2/18:0) |
| 4 | cortisone |
| 5 | mannose |
| 6 | urate |

In some embodiments, prognostic metabolomic signatures may be identified using coefficients from an mBMI model. Such signatures may comprise, in reverse order of strength, metabolite markers selected from: urate, 1-stearoyl-2-dihomo-linolenoyl-GPC (18:0/20:3n3 or 6)*, alanine, N-acetyltyrosine glutamate, 1-palmitoleoyl-3-oleoyl-glycerol (16:1/18:1)*, 1-(1-enyl-palmitoyl)-2-oleoyl-GPC (P-16:0/18:1)*, 1-(1-enyl-stearoyl)-2-oleoyl-GPC (P-18:0/18:1), 1-(1-enyl-stearoyl)-2-docosahexaenoyl-GPC (P-18:0/22:6)*, 1-arachidoyl-GPC (20:0), N-acetylglycine, sphingomyelin (d18:1/18:1, d18:2/18:0), mannose, and cortisone; or a derivative thereof.

TABLE 6
Subset of metabolites that are grouped
based on co-efficient of mBMI.

| S/N | Metabolite |
|---|---|
| 1 | cortisone |
| 2 | N-acetylglycine |
| 3 | 1-nonadecanoyl-GPC (19:0) |
| 4 | asparagine |
| 5 | glucose |
| 6 | mannose |
| 7 | sphingomyelin (d18:1/18:1, d18:2/18:0) |
| 8 | aspartate |
| 9 | alanine |
| 10 | 1-stearoyl-2-dihomo-linolenoyl-GPC (18:0/20:3n3 or 6) |
| 11 | glutamate |
| 12 | kynurenate |
| 13 | urate |

In some embodiments, prognostic metabolomic signatures may be identified using lasso regression. Signatures identified by such methods may comprise, in reverse order of strength, metabolite markers selected from: urate, 1-stearoyl-2-dihomo-linolenoyl-GPC (18:0/20:3n3 or 6)*, alanine, N-acetyltyrosine glutamate, 1-palmitoleoyl-3-oleoyl-glycerol (16:1/18:1)*, 1-(1-enyl-palmitoyl)-2-oleoyl-GPC (P-16:0/18:1)*, 1-(1-enyl-stearoyl)-2-oleoyl-GPC (P-18:0/18:1), 1-(1-enyl-stearoyl)-2-docosahexaenoyl-GPC (P-18:0/22:6)*, 1-arachidoyl-GPC (20:0), N-acetylglycine, sphingomyelin (d18:1/18:1, d18:2/18:0), mannose, and cortisone; or a derivative thereof.

TABLE 7
Subset of metabolites that are grouped based on lasso regression.

| S/N | Metabolite |
|---|---|
| 1 | urate |
| 2 | 1-stearoyl-2-dihomo-linolenoyl-GPC (18:0/20:3n3 or 6)* |
| 3 | alanine |
| 4 | N-acetyltyrosine |
| 5 | glutamate |
| 6 | 1-palmitoleoyl-3-oleoyl-glycerol (16:1/18:1)* |
| 7 | 1-(1-enyl-palmitoyl)-2-oleoyl-GPC (P-16:0/18:1)* |
| 8 | 1-(1-enyl-stearoyl)-2-oleoyl-GPC (P-18:0/18:1) |
| 9 | 1-(1-enyl-stearoyl)-2-docosahexaenoyl-GPC (P-18:0/22:6)* |
| 10 | 1-arachidoyl-GPC (20:0) |
| 11 | N-acetylglycine |
| 12 | sphingomyelin (d18:1/18:1, d18:2/18:0) |
| 13 | mannose |
| 14 | cortisone |

Epidemiological Analysis

Events of interest, e.g., disease onset, disease progression, morbidity and mortality (termed disease variable), which occur due to an explanatory variable, such as high mBMI, are generally measured by analyzing the effect of the explanatory variable on the disease variable. Generally speaking, this is done by comparing rates of disease in an explanatory group versus a control group. There are a number of ways of comparing the explanatory and control groups, using different measures of association. A measure of association is any mathematical or statistical measure that used to quantify the association between two or more variables. In the context of epidemiology, a measure of association is any such mathematical or statistical relationship used to measure disease frequency relative to other factors, and is an indication of how more or less likely one is to develop disease as compared to another. Measures of association focus on risk factors, which are found to be associated with a health condition, and may be thought of as an attribute or exposure that increases the probability of occurrence of disease (e.g., behavior, genetic, environmental or social factors, time, person or place).

Epidemiological measures of association can broadly be divided into absolute and relative comparisons. Thus, a study of the rate of a disease phenotype (e.g., heart attack) may yield a rate of 2 per 100 in obese subjects and 1 per 100 in non-obese (normal weight) subjects. An absolute comparison such as (2 per 100)−(1 per 100)=(1 per 100), meaning there is one additional case per 100 obese subjects. A relative comparison such as (2 per 100)/(1 per 100)=2, means that obese subjects are at twice the risk of control subjects (subjects with normal weight or normal BMI). Both metrics, including, statistical classifications thereof, e.g., in percentile or quantile, may be used.

A variety of different measures of association is routinely used in epidemiology. The most common are relative risk (RR; risk ratio) and odds ratio (OR). Risk ratio is often used in cohort studies and may be defined as the relative risk associated with a risk factor, e.g., RR=R1/R0, where R1 is the rate in an exposed group versus RO, the rate in a non-exposed group. RR is thus a risk multiplier on top of a baseline risk RO, where the segment of the RR above 1 represents elevation in risk. Thus, a RR of 1.0 or greater indicates an increased risk, a RR of less than 1.0 indicates decreased risk, and a RR of 2 represents a 100% increase in risk. OR is an epidemiological measure of association expressing disease frequency in terms of odds, and is defined as the odds of disease in the exposed population divided by the odds of disease in the unexposed population. OR is more often used in case-controlled studies, and may involve a comparison of disease cases with the prevalence among non-cases for controls. Both RR and OR characterize the association between the exposure and the disease in relative terms, and both reflect the frequency of disease occurrence among exposed subjects as a multiple of the rate among unexposed subjects.

Absolute or difference measures of association are also used in epidemiology, and are generally referred to as attributable risk and population attributable risk percent. Attributable risk is defined as the incidents of disease in an exposed population minus the incidents of disease in the unexposed population, and generally is thought of as the number of cases among the exposed that could be eliminated if the exposure were removed. Population attributable risk percent is defined as the incidents of the disease in the total population minus the incidents in the unexposed population, divided by the incidents of disease in the total population. It measures the excess risk of disease in the total population attributable to exposure and the reduction in risk, which would be achieved if the population were entirely unexposed. Epidemiological measures of association are further defined and explained in Epidemiology: Beyond the Basics, by Xavier Nieto et al., Jones & Bartlett Learning; 4th Ed. (2018); and Nguyen et al., Gastroenterol Clin North Am. 39(1): 1-7 (2010).

In the present disclosure, patients whose mBMI values are significantly elevated compared to BMI values are, at least, 20%, 30%, 40%, 50%, 60%, 80%, 100%, 125%, 150%, 175%, 180%, 200%, 250%, 300%, or a greater %, e.g., 400%, more likely to suffer from an adverse events compared to controls. For instance, the number of events per 100 patients with a healthy metabolome (normal BMI) was increased by more than 80% in outliner patients with obese metabolic profile (normal BMI) (i.e., about 2.0 events in 100 patients in healthy subjects versus about 3.7 events in 100 patients in outlier subjects). In obese individuals, the number of events was elevated even more (about 4.2 events in 100 patients or increase of about 110% compared to healthy subjects). Separated analysis of the various endpoints reveals that the association was much more pronounced and accentuated for subjects with cardiovascular diseases than patients who had or who were at risk for developing stroke.

Additional Steps for Improving Robustness of Analysis

In some embodiments, the disclosure includes improving prognostic significance of the methods of the disclosure by analyzing a variety of environmental and/or genetic factors that may play in the predisposition, initiation, development, and pathophysiology of the obesity phenotype or diseases related thereto. Representative examples of such factors include, e.g., android/gynoid ratio, total triglycerides, waist/hip ratio, subcutaneous fat, visceral fat, insulin resistance, high density lipoprotein (HDL) levels, percent fat, diastolic blood pressure, systolic blood pressure, total cholesterol, low density lipoprotein (LDL), insulin resistance, dual-energy X-ray absorptiometry (DEXA) scores, and other anthropomorphic traits (larger-framed individuals).

TABLE 8
Association between different phenotypes with mBMI and BMI.
r² indicates the percent variation in BMI explained by each
metabolite in univariate analysis for a combined analysis
of the first time point of the TWINSUK cohort and the
Health Nucleus data. Improvement is calculated as mBMI r² − BMI r².

| Phenotype | r² with mBMI | r² with BMI | Improvement |
|---|---|---|---|
| BMI | 42.6% | — | — |
| Android/gynoid ratio | 34.1% | 23.7% | 10.4% |
| Total triglycerides | 28.8% | 9.2% | 19.6% |
| Waist/hip ratio | 24.9% | 14.8% | 10.1% |
| Subcutaneous fat | 23.3% | 16.2% | 7.1% |
| Visceral fat | 23.3% | 16.2% | 7.1% |
| Insulin resistance | 22.9% | 17.0% | 5.9% |
| HDL | 19.2% | 11.9% | 7.3% |
| Percent fat | 8.8% | 14.9% | −6.1% |
| Diastolic blood pressure | 7.4% | 8.4% | −1.0% |
| Systolic blood pressure | 7.2% | 6.8% | 0.4% |
| Total cholesterol | 1.8% | 1.1% | 0.7% |
| LDL | 1.4% | 1.9% | −0.5% |

In some embodiments, the disclosure includes improving the prognostic significance of the methods of the disclosure by analyzing the sample for the presence or absence of one or more genetic factors. In one specific embodiment, the prognostic methods include analysis of whether the subject is a carrier of a rare (MAF<0.01%) coding variants in the melanocortin 4 receptor gene (MC4R), including, variants thereof, e.g., M292fs, R236C, S180P, A175T, and T11A, but not I170V. Table 9 provides a general overview on the association of these MC4R variants with obesity in participants of European ancestry.

TABLE 9
Variants identified in MC4R in unrelated participants of European ancestry.

| | | | | | | | Twin | |
|---|---|---|---|---|---|---|---|---|
| | | | Global | Known | | Carrier | non- | |
| | Protein | Study | gnomad | obesity | Carrier | twin | carrier | Twin |
| Variant | change | MAF | MAF | annotation | BMI | BMI | BMI | zygosity |
| chr18: 60371541 G/A | p.Ser270Phe | 0.036% | 0.003% | None | 25.7 | 24.8 | N/A | MZ |
| chr18: 60372307 G/A | p.Leu15Phe | 0.036% | 0.000% | None | 23 | 22.6 | N/A | MZ |
| chr18: 60371474 CA/C | p.Met292fs | 0.036% | <0.003% | None | 32.8 | N/A | 28.8 | DZ |
| chr18: 60371644 G/A | p.Arg236Cys | 0.036% | 0.003% | HGMD | 34.5 | 34.5 | N/A | DZ |
| | | | | highC DM | | | | |
| chr18: 60371812 A/G | p.Ser180Pro | 0.036% | <0.003% | ClinVar LP | 34.2 | 34.4 | N/A | DZ |
| chr18: 60371827 C/T | p.Ala175Thr | 0.036% | 0.019% | ClinVar P | 29 | 28.5 | N/A | MZ |
| | | | | and HGMD | | | | |
| | | | | highC DM | | | | |
| chr18: 60371842 T/C | p.Ile170Val | 0.036% | 0.013% | ClinVar P | 22.6 | N/A | 21.3 | DZ |
| | | | | and HGMD | | | | |
| | | | | highC DM | | | | |
| chr18: 60372319 T/C | p.Thr11Ala | 0.036% | <0.003% | HGMD | 36 | N/A | N/A | N/A |
| | | | | lowC DM | | | | |

MAF = minor allele frequency; HGMD highC DM = Human Gene Mutation Database high-confidence disease- causing mutation; lowC = low-confidence; LP = likely pathogenic; P = pathogenic; MZ = monozygotic; DZ = dizygotic. Each variant was only seen once in the unrelated participants of this study.

In some embodiments, the methods of the disclosure include supplemental genetic analysis data comprising annotations of risk genes and linkages thereof to obesity phenotypes in the human genome mutation database (HGMD; accessible via the world-wide-web at hgmd(dot)cf(dot)ac(dot)uk) or clinically relevant variant archive (CLINVAR; accessible via the world-wide-web at www(dot)ncbi(dot)nlm(dot)nih(dot)gov/clinvar). As outlined in detail in the Examples section, MC4R carriers had significantly higher BMI (p=0.02) and a positive statistical correlation than non-carriers. MC4R carriers also generally had higher diastolic blood pressure, insulin resistance, and percent body fat. The BMI data in the participants supported a pathogenic role for five of the variants (Met292fs, Arg236Cys, Ser180Pro, Ala175T, and Thr11Ala), but did not Ile170V (identified in HGMD and ClinVar as being pathogenically associated with obesity). Overall, MC4R variant carriers are observed with greatest frequency (about 6.1%) in obese patients with polygenic risk scores in the lowest quartile compared to subjects with normal weight (only about 0.3%).

In some embodiments, the methods of the disclosure include supplemental genetic analysis data comprising annotations of risk genes and linkages thereof, e.g., lipodystrophy genes, to obesity phenotypes. Particularly, the disclosure includes analysis of one or more of the Table 10 genes or linkages thereof:

TABLE 10
Lipodystrophy genes that are analyzed in accordance with the present disclosure

| | | | | Global | Known | Carrier | |
|---|---|---|---|---|---|---|---|
| | | Protein | Study | gnomad | lipodystrophy | BMI | |
| Gene | Variant | change | MAF | MAF | annotation | (mBMI) | |
| ZMPSTE24 | chr1: 40290870 G/GT | p.Leu362fs | 0.11% | 0.03% | ClinVar P | 18 | (18.9) |
| ZMPSTE24 | chr1: 40290870 G/GT | p.Leu362fs | 0.11% | 0.03% | ClinVar P | 22 | (20.3) |
| ZMPSTE24 | chr1: 40290870 G/GT | p.Leu362fs | 0.11% | 0.03% | ClinVar P | 22.4 | (26.1)** |
| ZMPSTE24 | chr1: 40290870 GT/G | p.Leu362fs | 0.04% | <0.003% | Not | 30.7 | (27.5) |
| | | | | | annotated† | | |
| AGPAT2 | chr9: 136673876 G/C | p.Ala238Gly | 0.04% | 0.00% | HGMD highC | 20 | (23.3) |
| | | | | | DM | | |
| LIPE | chr19: 42401821 | p.Val1068fs | 0.04% | 0.07% | ClinVar P | 23 | (27.4) |
| | CCCCCCGCAGCCCCCGTCTA/C | | | | | | |
| BSCL2 | chr11: 62692371 C/T | c.863 + 5G > A | 0.04% | <0.003% | ClinVar P | 24 | (29.9) |

MAF = minor allele frequency; HGMD highC DM = Human Gene Mutation Database high-confidence disease-causing mutation, and lowC = low confidence; ClinVar LP = likely pathogenic, P = pathogenic, and DZ = dizygotic. Each variant was only seen once in the participants of this study.
**Non-carrier DZ twin BMI = 22.6 (25.1). No other carriers of lipodystrophy variants had twins.
†This deletion at the same site as a lipodystrophy insertion has not previously been annotated.

In particular, the disclosure relates to analysis of at least 1, 2, 3, 4 or more, e.g., all genetic variants of the zinc metallopeptidase STE24 (ZMPSTE24) gene or the 1-acylglycerol-3-phosphate O-acyltransferase 2 (AGPAT2) gene or lipase E, hormone sensitive type (LIPE) gene or Bernardinelli-Seip congenital lipodystrophy type 2 (BSCL2) gene, or any combination thereof. The resulting variation may result in a change (e.g., mutation) in an amino acid sequence encoded by the gene. In some embodiments, the genetic variants include, one or more variations in the ZMPSTE24 gene comprising variation at chr1:40290870 G/GT (e.g., resulting in p.Leu362fs). In some embodiments, the genetic variants include, one or more variations in the AGPAT2 gene comprising variation chr9:136673876 G/C (e.g., resulting in p.Ala238Gly). In some embodiments, the genetic variants include one or more variations in the LIPE gene comprising variation chr19:42401821 CCCCCCGCAGCCCCCGTCTA/C (e.g., resulting in p.Val1068fs). In some embodiments, the genetic variants include one or more variations in the BSCL2 gene comprising variation chr11:62692371 C/T (e.g., resulting in c.863+5G>A). Preferably, the genetic variants include at least 2, 3, 4 or all of the aforementioned variations. Information on the genetic variants can be obtained from known databases, e.g., Varsome (varsome(dot)com) or Clinvar database (ncbi(dot)nlm(dot)nih(dot)gov/clinvar/).

Methods of Diagnosis

Methods for diagnosing, or aiding in diagnosing, whether a subject has obesity or a disease or condition related thereto, such as diabetes, metabolic syndrome, atherosclerosis, or cardiomyopathy, may performed using one or more of the biomarkers identified in the respective tables provided herein. A method of diagnosing (or aiding in diagnosing) includes (a) analyzing a biological sample from a subject to determine the levels or activities of one or more biomarkers in the sample and (b) comparing the levels or activities of one or more biomarkers in the sample to disease-positive or condition-positive reference levels (e.g., positive control) and/or disease-negative or condition-negative reference levels (e.g., negative control) of the one or more biomarkers to diagnose (or aid in the diagnosis of) whether the subject has the disease or condition. For example, a method of diagnosing whether a subject is obese may include the steps of (a) analyzing a biological sample (e.g., serum or blood) from a subject to determine the levels or activities of one or more metabolites of Table 11, Table 12A, Table 12B, or Tables 1-7 (preferably Tables 2-7) in the sample to compute an mBMI score; (b) optionally comparing the mBMI score to the actual BMI score; and (c) diagnosing obesity or a disease related thereto by comparing the mBMI score to a reference standard.

The diagnostic methods of the disclosure may be used along with other methods that are useful in the clinical determination of whether a subject has obesity or a disease related thereto. Methods useful in the clinical determination of whether a subject has a disease or condition related to obesity, such as, diabetes, metabolic syndrome, atherosclerosis, or cardiomyopathy are known in the art. For example, methods useful in the clinical determination of whether a subject has diabetes include, e.g., glucose disposal rates (Rd), body weight measurements, waist circumference measurements, BMI determinations, peptide YY measurements, hemoglobin A1C measurements, adiponectin measurements, fasting plasma glucose measurements, free fatty acid measurements, fasting plasma insulin measurements, and the like. Methods useful for the clinical determination of atherosclerosis and/or cardiomyopathy in a subject include angiography, stress-testing, blood tests (e.g., to measure homocysteine, fibrinogen, lipoprotein A, small LDL particles, and C-reactive protein levels), electrocardiography, echocardiography, computed tomography (CT) scans, ankle/brachial index, and intravascular ultrasounds.

In the context of diagnosing or treating obesity-related disease such as diabetes, the methods of the disclosure may be combined with methods for diagnosing diabetes, e.g., measurement of glucose disposal rate (Rd) as measured by the HI clamp. Similarly, insulin sensitivity of the individual can be determined using appropriate in vitro or in vivo assays. In some embodiments, such methods include use of oral glucose tolerance tests (OGTT) for use in categorizing subjects as having normal glucose tolerance (NGT), impaired fasting glucose levels (IFG), or impaired glucose tolerance (IGT). Methods for determining level of insulin resistance using a calibrated insulin resistance score (IR score) are known in the art. See, Shalaurova et al., Metab Syndr Relat Disord., 12(8): 422-429, 2014. The IR Score can be used to monitor disease progression or remission, response to therapeutic intervention and also for evaluating drug efficacy.

After the levels or activities of the one or more metabolites (or derivatives) is determined, the level(s) may be compared to disease or condition reference levels or activities of the one or more metabolites (or derivatives) to determine a rating for each of the one or more metabolites (or derivatives) in the sample. Preferably, the ratings are aggregated using any algorithm to create a score, for example, an mBMI score, for the subject. The algorithm may take into account any factors relating to a particular disease or condition related to obesity, such as cardiomyopathy or diabetes, including the number of biomarkers, the correlation of the biomarkers to the particular disease or condition, etc.

### III. Monitoring Disease or Condition Progression/Regression

The identification of biomarkers herein allows for monitoring progression/regression of obesity or a disease related thereto (e.g. diabetes, metabolic syndrome, atherosclerosis, cardiomyopathy, insulin resistance, etc.) in a subject. A method of monitoring the progression/regression of obesity or a disease related thereto, such as diabetes, metabolic syndrome, atherosclerosis, and cardiomyopathy, in a subject comprises (a) analyzing a first biological sample from a subject to determine the levels or activities of one or more metabolites selected from Table 11, Table 12A, Table 12B, or Tables 1-7 (preferably Tables 2-7) or a derivative thereof, or a combination thereof in the first sample obtained from the subject at a first time point, (b) analyzing a second biological sample from a subject to determine the levels or activities of the one or more metabolites of (a) or a derivative thereof, the second sample obtained from the subject at a second time point, and (c) comparing the levels or activities of one or more metabolites in the first sample to the levels or activities of one or more metabolites in the second sample in order to monitor the progression/regression of the disease or condition in the subject. The results of the method are indicative of the course of the disease or condition (i.e., progression or regression, if any change) in the subject.

In some particular embodiments, progression or regression of obesity or a disease related thereto may be based on metabolomics BMI (mBMI) score which is indicative of the obesity (particularly unhealthy obesity) in the subject and which can be monitored over time. By comparing the mBMI score from a first time point sample to the mBMI score from at least a second time point sample the progression or regression of obesity or a disease related thereto can be determined. Such a method of monitoring the progression/regression of obesity or a disease related thereto in a subject comprises (a) analyzing a first biological sample from a subject for metabolites of Table 11, Table 12A, Table 12B, or Tables 1-7 (preferably Tables 2-7) to determine an mBMI score for the first sample obtained from the subject at a first time point, (b) analyzing a second biological sample from a subject for the metabolites of Table 11, Table 12A, Table 12B, or Tables 1-7 (preferably Tables 2-7) to determine a second mBMI score, the second sample obtained from the subject at a second time point, and (c) comparing the mBMI score in the first sample to the mBMI score in the second sample in order to monitor the progression/regression of obesity or a disease related thereto in the subject.

The markers and algorithms of the instant disclosure, which are useful for progression monitoring, may be further used to guide or assist physicians to make decisions about preventative or therapeutic measures such as dietary restrictions, exercise, or early-stage drug treatment.

### IV. Determining Predisposition to or Risk of Developing Obesity

The biomarkers identified herein may also be used in the determination of whether a subject who is not exhibiting any symptoms of a disease or condition, such as obesity or a disease related thereto, may nonetheless be at risk. Such methods are particularly useful, e.g., in determining whether a subject is predisposed to developing obesity or a disease related thereto, e.g., diabetes, metabolic syndrome, atherosclerosis, or cardiomyopathy. Such methods include (a) analyzing a first biological sample from a subject to determine the levels or activities of one or more metabolites selected from Table 11, Table 12A, Table 12B, or Tables 1-7 (preferably Tables 2-7) or a derivative thereof in the first sample obtained from the subject at a first time point, (b) analyzing a second biological sample from a subject to determine the levels or activities of the one or more metabolites of (a) or a derivative thereof, the second sample obtained from the subject at a second time point, and (c) comparing the levels or activities of one or more metabolites in the first sample to the levels or activities of one or more metabolites in the second sample in order to determine the subject's predisposition to or risk of developing obesity or a disease related thereto. The results of the method may be used along with other methods (e.g., biochemical assays, physiological measurements, and/or lifestyle evaluations) to clinically determine whether a subject is predisposed to or at risk of developing obesity or a disease related thereto.

After the levels or activities of the one or more metabolites or derivatives thereof in the sample are determined, the levels or activities may be compared to disease-positive or condition-positive and/or disease-negative or condition-negative reference levels in order to predict whether the subject is predisposed to or at risk of developing obesity or a disease related thereto, such as, diabetes, metabolic syndrome, atherosclerosis, or cardiomyopathy. Levels of the one or more metabolites (or derivatives thereof) in a sample corresponding to the disease-positive or condition-positive reference levels (e.g., levels that are the same as the reference levels, substantially the same as the reference levels, above and/or below the minimum and/or maximum of the reference levels, and/or within the range of the reference levels) are indicative of the subject being predisposed to or at risk of developing obesity or a disease related thereto. Levels of the one or more metabolites (or derivatives thereof) in a sample corresponding to disease-negative or condition-negative reference levels (e.g., levels that are the same as the reference levels, substantially the same as the reference levels, above and/or below the minimum and/or maximum of the reference levels, and/or within the range of the reference levels) are indicative of the subject not being predisposed to or at risk of developing obesity or a disease related thereto. In addition, levels of the one or more metabolites (or derivatives thereof) that are differentially present (especially at a level that is statistically significant) in the sample as compared to disease- or condition-negative reference levels may be indicative of the subject being predisposed to developing obesity or a disease related thereto. Levels of the one or more metabolites (or derivatives thereof) that are differentially present (especially at a level that is statistically significant) in the sample as compared to disease- or condition-positive reference levels may be indicative of the subject not being predisposed to developing the disease or condition.

Preferably, in carrying out the prognostic methods of the disclosure, the levels or activities of the one or more metabolites (or derivatives thereof) of Table 11, Table 12A, Table 12B, or Tables 1-7 (preferably Tables 2-7) may be outputted as a metabolomics BMI (mBMI) score which is indicative of the obesity (particularly unhealthy obesity) in the subject and which can be used to prognosticate obesity or a disease related thereto. By comparing the mBMI score of a subject's sample to the mBMI score of a reference standard (e.g., obtained by analyzing the levels or activities of the same metabolites in one or more healthy subjects), a determination can be made regarding whether the subject is predisposed to or at risk of developing obesity or a disease related thereto. Such a method of determining predisposition to or risk of developing obesity or a disease related thereto can be made by (a) analyzing a first biological sample from a subject to determine levels or activities of the one or more metabolites (or derivatives thereof) of Table 11, Table 12A, Table 12B, or Tables 1-7 (preferably Tables 2-7) and computing a first mBMI score for the first sample obtained from the subject, (b) analyzing an identical biological sample from a reference (e.g., healthy subjects) to determine levels or activities of the one or more metabolites of step (a) and computing a second mBMI score, and (c) comparing the mBMI score in the first sample to the mBMI score in the second sample in order to determine whether the subject is predisposed to or at risk of developing obesity or a disease related thereto. Herein, if the mBMI score of the test sample exceeds the mBMI score in the second sample, then the subject is evaluated as being predisposed to or at risk of developing obesity or a disease related thereto.

Purely by way of example, after the levels or activities of the one or more metabolites (or derivatives thereof) in the sample are determined, the levels or activities are used to compute an mBMI score for the subject, and the subject's mBMI score compared to mBMI scores of obesity-positive and/or obesity-negative reference samples in order to predict whether the subject is predisposed to or at risk of developing obesity or a disease related thereto. If the subject's mBMI scores correspond to the mBMI scores of obesity-positive reference standards (e.g., scores that are the same as the reference levels, substantially the same as the reference levels, above and/or below the minimum and/or maximum of the reference levels, and/or within the range of the reference levels), then the result indicates that the subject is predisposed to or at risk of developing obesity or a disease related thereto. If the subject's mBMI scores correspond to the mBMI scores of obesity-negative reference standards (e.g., scores that are the same as the reference levels, substantially the same as the reference levels, above and/or below the minimum and/or maximum of the reference levels, and/or within the range of the reference levels), then the result indicates that the subject is not predisposed to or not at risk of developing obesity or a disease related thereto. If the subject's mBMI score is elevated compared to the mBMI score of a negative sample (especially at a level that is statistically significant), then the results are indicative of the subject being predisposed to developing obesity or a disease related thereto. If the subject's mBMI score is attenuated compared to the mBMI score of a positive sample (especially at a level that is statistically significant), then the results are indicative of the subject not being predisposed to developing obesity or a disease related thereto. Although obesity is discussed in this example, predisposition to or risk of developing related diseases, e.g., diabetes, metabolic syndrome, atherosclerosis, or cardiomyopathy, may also be determined in accordance with the instant methods.

Predisposition to or risk of developing obesity or a disease related thereto may be computed using methods outlined above. For instance, for parametric continuous variables such as mBMI, means along with standard deviations (SD) may be used. For categorical data such as % body fat, % visceral fat, % subcutaneous fat or insulin resistance, counts or percentages may be used. Non-parametric Spearman's rank correlation may be used to assess the associations between anthropometric measurements (e.g., waist-to-height ratio (WHtR), waist to hip ratio (WHR), waist circumference, and BMI) of obesity with risk factors (e.g., mortality, morbidity, survival, etc.). Anthropometric measurements may also be converted to z-scores (original value subtracted by the mean and result divided by the SD) to represent the number of SDs above and below the mean for each subject. Logistic regression may be used to assess the effects of each standardized anthropometric measurement of being above the recommended treatment thresholds for various risk score models (computed for each SD increment above the mean for each anthropometric measure of obesity). Odds ratio (OR) and associated 95% confidence intervals (CI) may be further used to compute the chance of being above the recommended thresholds for the specific risk score model (e.g., Framingham model). Sensitivity, specificity and area under the receiver operating characteristic (ROC) curve may be computed for each metric using software packages such as SPSS.

### IV. Monitoring Therapeutic Efficacy

The biomarkers provided also allow for the assessment of the efficacy of a composition for treating obesity or a disease related thereto, e.g., diabetes, metabolic syndrome, atherosclerosis, or cardiomyopathy. For example, depending on the modulation of the levels or activities of the metabolites of the disclosure, which is brought about by a pharmaceutical composition (or drug) for treating obesity, determinations can be made regarding whether the composition (or drug) is effective in treating obesity or a disease related thereto. Similar methodology can be used in determining the relative efficacy of two or more compositions (or drugs) for treating obesity. Such assessments may be used, for example, in efficacy studies as well as in lead selection of compositions for treating obesity.

Representative examples of anti-obesity drugs include, but are not limited to, e.g., orlistat, locaserin, sibutramine, rimonabant, metformin, exenatide, pramlintide, phentermine, topiramate; insulin, acetylsalicylic acid, acarbose, miglitol, alogliptin, linagliptin, pioglitazone, saxagliptin, sitagliptin, simivastin, albiglutide, dulaglutide, liraglutide, nateglinide, repaglinide, dapagliflozin, canagliflozin, empagliflozin, glimepiride, rosiglitazone, gliclazide, glipizide, glyburide, chlorpropamide, tolazamide, tolbutamide or a combination thereof.

Accordingly, the instant disclosure provides methods of assessing the efficacy of a composition for treating obesity or a disease related thereto, e.g., diabetes, metabolic syndrome, atherosclerosis, or cardiomyopathy, comprising determining levels or activities of at least one metabolite of Table 11, Table 12A, Table 12B, or Tables 1-7 (preferably Tables 2-7) in a sample obtained from a subject having obesity or a disease related thereto, wherein the determination is made before and after administration of the composition to the subject, wherein a modulation in the activities or levels of the metabolites in the subject post-administration of the composition compared to the activities or levels of the metabolites in the subject pre-administration of the composition indicates that the composition is effective in treating obesity or a disease related thereto.

In some embodiments, there is provided a method for assessing the efficacy of a composition for treating obesity or a disease related thereto by monitoring the directionality of changes in the levels or activities of the metabolites of the disclosure compared to a reference standard. As noted in Table 3, the levels or activities of a subset of metabolites is increased in obese subjects compared to control (e.g., healthy subjects). When an effective anti-obesity composition is administered to such subjects in need, the levels or activities of such metabolites may attenuate and reach threshold levels (e.g., control) or even sub-threshold levels. Similarly, if the levels or activities of a subset of metabolites is decreased in obese subjects compared to controls (e.g., healthy subjects), administration of an anti-obesity composition to such subjects may increase the levels or activities of such metabolites in the subject such that a threshold level (e.g., control) or even supra-threshold level is attained. In short, an effective anti-obesity composition may reverse the directionality of changes in the levels or activities of the metabolites of the disclosure in the subject's sample compared to the levels or activities of the metabolites in healthy subject(s).

In some embodiments, there is provided a method for assessing the efficacy of a composition for treating obesity or a disease related thereto by monitoring changes in mBMI levels post-administration of the composition. When an effective anti-obesity composition is administered to such subjects in need, the subject's mBMI scores may attenuate and reach threshold levels (e.g., control) or even sub-threshold levels. Similarly, if the obese subject's baseline mBMI scores is lower prior to administration of an anti-obesity composition, such that intake of the anti-obesity composition increases mBMI score for the subject, and then the composition is deemed not be effective for treating obesity.

As with the other methods described herein, the comparisons made in the methods of monitoring progression/regression of obesity or a disease related thereto, e.g., diabetes, metabolic syndrome, atherosclerosis, or cardiomyopathy, may be carried out using various techniques, including simple comparisons, statistical analyses (e.g., regression), and combinations thereof.

The results of the determinations may be used along with other methods for clinical monitoring of progression/regression of the disease or condition in a subject.

### V. Identification of Responders and Non-Responders to Therapeutic

The metabolites (or derivatives thereof) provided in Table 11, Table 12A, Table 12B, or Tables 1-7 (preferably Tables 2-7) also allow for the identification of subjects in whom the composition for treating obesity or a disease related thereto such as diabetes, metabolic syndrome, atherosclerosis, or cardiomyopathy, is efficacious (i.e., patient responds to the therapeutic agent). For example, the identification of metabolites (or derivatives thereof) for obesity also allows for assessment of the subject response to a composition for treating obesity as well as the assessment of the relative patient response to two or more compositions for treating obesity. Such assessments may be used, for example, in targeted therapy of obesity or diseases related thereto. For instance, based on the results of the aforementioned tests, certain types of anti-obesity drugs may be favored over other types of anti-obesity drugs in certain subjects based on whether the subject is known to respond to the particular anti-obesity drug.

Thus, also provided are methods of predicting the response of a patient to a composition for treating obesity or a disease related thereto, e.g., diabetes, metabolic syndrome, atherosclerosis, or cardiomyopathy. The predictive method comprises (a) analyzing in a biological sample obtained from a subject having obesity or a disease related thereto, e.g., diabetes, metabolic syndrome, atherosclerosis, or cardiomyopathy, which subject is currently or previously being treated with a composition, the levels or activities of one or more metabolites (or derivatives thereof) of Table 11, Table 12A, Table 12B, or Tables 1-7 (preferably Tables 2-7); and (b) comparing the levels or activities of one or more metabolites of (a) in the sample to the levels or activities of one or more metabolites of (a) in a previously-taken biological sample from the subject, wherein the previously-taken biological sample was obtained from the subject before being treated with the composition. The results of the comparison are indicative of the response of the patient to the composition for treating the respective disease or condition. Preferably, the methods of predicting the response (i.e., measuring responsiveness) is carried out by measuring mBMI scores of the subject prior to and after administration of the composition for treating obesity or a disease related thereto.

The aforementioned methods can be used to monitor whether or not a patient is responding to an agent for treating obesity or a disease related thereto. If the comparisons indicate that the levels or activities of one or more metabolites (or derivatives thereof) of Table 11, Table 12A, Table 12B, or Tables 1-7 (preferably Tables 2-7) are increasing or decreasing over time to become more similar to the disease- or condition-negative reference levels (or less similar to the disease- or condition-positive reference levels), then the results are indicative of the patient responding to the anti-obesity agent.

It should be noted that responsiveness to the test agent or clinically-approved therapeutic agent can be made at any time after the first sample is obtained. In one aspect, the second sample (for measuring the responsiveness to a test agent or clinically-approved agent) is obtained 1, 2, 3, 4, 5, 6, or more days after the first sample. In another aspect, the second sample is obtained 1, 2, 3, 4, 5, 6, 7, 8, 9, 10, or more weeks after the first sample or after the initiation of treatment with the composition. In another aspect, the second sample may be obtained 1, 2, 3, 4, 5, 6, 7, 8, 9, 10, 11, 12, or more months after the first sample or after the initiation of treatment with the composition.

As with the other methods described herein, in the aforementioned methods for determining the subject's responsiveness to a test agent or clinically-approved therapeutic agent for treating obesity or a disease related thereto, e.g., diabetes, metabolic syndrome, atherosclerosis, or cardiomyopathy, may be carried out using various techniques, including simple comparisons, one or more statistical analyses, including combinations thereof.

The aforementioned methods are useful in identifying responders and/or non-responders to novel therapeutic agents that may at various stages of clinical testing. In particular, the aforementioned methods allow clinicians to stratify high-risk obese individuals and to assess the efficacy of therapeutic candidates more effectively and safely. A new diagnostic test that discriminates non-responding from responding patients to a therapeutic would enable pharmaceutical companies to identify and stratify patients that are likely to respond to the therapeutic agent and target specific therapeutics for certain cohorts that are likely to respond to the therapeutic. Accordingly, the methods of the disclosure not only provide cost-saving measures to pharmaceutical companies but also enable hospitals and dispensaries to deliver individualized and targeted therapy to patients by improving drug efficacy and concomitantly reducing the side effects.

### VI. Methods of Screening a Composition for Activity in Modulating Biomarker

The biomarkers provided herein also allow for the screening of compositions for activity in modulating metabolites (or derivatives thereof) that are associated with obesity or a disease related thereto, such as diabetes, metabolic syndrome, atherosclerosis, and cardiomyopathy, which may be useful in treating the disease or condition. Such methods comprise assaying test compounds for activity in modulating the levels or activities of one or more metabolites (or derivatives thereof) of Table 11, Table 12A, Table 12B, or Tables 1-7 (preferably Tables 2-7). Such screening assays may be conducted in vitro and/or in vivo, and may be in any form known in the art useful for assaying modulation of such metabolites (or derivatives thereof) in the presence of a test composition such as, for example, cell culture assays, organ culture assays, and in vivo assays (e.g., assays involving animal models). For example, the identification of metabolites (or derivatives thereof) associated with obesity also allows for the screening of compositions for activity in modulating metabolites (or derivatives thereof) associated with obesity, which may be useful in treating obesity. Methods of screening compositions useful for treatment of obesity (or a disease related thereto) comprise assaying test compositions for activity in modulating the levels of one or more metabolites (or derivatives thereof) of Table 11, Table 12A, Table 12B, or Tables 1-7 (preferably Tables 2-7). Although obesity is discussed in this example, the other diseases and conditions, such as diabetes, metabolic syndrome, atherosclerosis, and cardiomyopathy, may also be diagnosed in accordance with this method.

### VII. Method of Identifying Potential Drug Targets

The disclosure also provides methods of identifying potential drug targets for diseases or conditions such as obesity or a disease related thereto, such as, diabetes, metabolic syndrome, atherosclerosis, and cardiomyopathy, using the biomarkers listed in Table 11, Table 12A, Table 12B, or Tables 1-7 (preferably Tables 2-7). A method for identifying a potential drug target for obesity or a disease related thereto, such as, diabetes, metabolic syndrome, atherosclerosis, and cardiomyopathy, comprises (a) identifying one or more biochemical pathways associated with one or more metabolites (or derivatives thereof) of Table 11, Table 12A, Table 12B, or Tables 1-7 (preferably Tables 2-7); and (b) identifying a protein (e.g., an enzyme) affecting at least one of the one or more identified biochemical pathways, the protein being a potential drug target for the disease or condition. For example, the identification of biomarkers for obesity also allows for the identification of potential drug targets for obesity. Representative pathways implicated in obesity are provided in Table 3 and include, e.g., (a) alanine and aspartate metabolism; (b) glutamate metabolism; (c) leucine, isoleucine and valine metabolism; (d) phenylalanine and tyrosine metabolism; (e) polyamine metabolism; (f) tryptophan metabolism; (g) glycine, serine and threonine metabolism; (h) fructose, mannose and galactose metabolism; (i) glycolysis, gluconeogenesis, and pyruvate metabolism; (j) ascorbate and aldarate metabolism; (k) nicotinate and nicotinamide metabolism; (l) TCA cycle; (m) carnitine metabolism (k) diacylglycerol fatty acid metabolism (also BCAA Metabolism); (l) phospholipid metabolism; (m) sphingolipid metabolism; (n) lysolipid metabolism; (o) plasmalogen; (p) steroid; (q) purine metabolism or (Hypo)xanthine/inosine containing; (r) purine metabolism adenine containing; (s) purine metabolism, guanine containing; (t) dipeptide derivative; (u) gamma-glutamyl amino acid (v) food component/plant-based xenobiotics metabolism.

Accordingly, the disclosure relates to one or more biochemical pathways (e.g., biosynthetic and/or metabolic (catabolic) pathway) that are associated with one or more metabolites (or derivatives thereof) which in turn are associated with obesity or a disease related thereto.

As is known in the art, pathway analysis is useful in drug discovery. For instance, a build-up of one metabolite (e.g., a pathway intermediate) may indicate the presence of a ‘block’ downstream of the metabolite and the block may result in a low/absent level of a downstream metabolite (e.g. product of a biosynthetic pathway). In a similar manner, the absence of a metabolite could indicate the presence of a ‘block’ in the pathway upstream of the metabolite resulting from inactive or non-functional enzyme(s) or from unavailability of biochemical intermediates that are required substrates to produce the product. Alternatively, an increase in the level of a metabolite could indicate a genetic mutation that produces an aberrant protein which results in the over-production and/or accumulation of a metabolite which then leads to an alteration of other related biochemical pathways and result in dysregulation of the normal flux through the pathway; further, the build-up of the biochemical intermediate metabolite may be toxic or may compromise the production of a necessary intermediate for a related pathway. It is possible that the relationship between pathways is currently unknown and this data could reveal such a relationship.

The proteins identified as potential drug targets may then be used to identify compositions that may be potential candidates for treating a particular disease or condition, such as obesity, including compositions for gene therapy.

### VII. Methods of Treatment

In another aspect, the disclosure relates to methods for treating obesity or a disease related thereto such as diabetes, metabolic syndrome, atherosclerosis, and cardiomyopathy. The methods generally involve treating a subject obesity or a disease related thereto, e.g., with an effective amount of a pharmaceutical composition (e.g., an anti-obesity drug), or with surgery or lifestyle therapy, until the levels or activities of metabolites of Table 1-7 are modulated. More specifically, the disclosure provides methods for treating obesity or a disease related thereto comprising (a) detecting levels and/or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a biological sample obtained from the subject; (b) diagnosing subject with obesity or a disease related thereto if the levels or activities of the metabolites of (a) are modulated compared to a reference standard; and (c) administering an effective amount of a therapy selected from the group consisting of anti-obesity pharmacotherapy, surgery, and lifestyle therapy to the subjects of (b) who are diagnosed with obesity or a disease related thereto.

Particularly, the disclosure provides methods for treating obesity or a disease related thereto comprising (a) detecting levels and/or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a biological sample obtained from the subject and computing a metabolomic body mass index (mBMI) value for the subject based on the detection; (b) diagnosing subject with obesity or a disease related thereto if the mBMI value of the subject is modulated compared to a reference standard; and (c) administering an effective amount of a therapy selected from the group consisting of anti-obesity pharmacotherapy, surgery, and lifestyle therapy to the subjects of (b) who are diagnosed with obesity or a disease related thereto. In some embodiments, the reference standard includes a subject's BMI, wherein if mBMI>>BMI, then the subject is administered an anti-obesity drug. Optionally, the method may comprise determining an additional feature (e.g., blood pressure, waist/hip ratio, android/gynoid ratio, % body fat, % visceral fat, % subcutaneous fat or insulin resistance) and using that determination, together with mBMI values, regarding whether the subject should take the anti-obesity drug.

In the therapeutic embodiments described above, subjects whose mBMI exceeds BMI by at least 20%, 30%, 40%, 50%, 60%, 80%, 100% (i.e., 1-fold increase), 150%, 200%, 250%, 300%, or more, e.g., 500%, are treated with the anti-obesity drug.

### IX. Methods of Using the Biomarkers for Other Diseases or Conditions

In some embodiments, the metabolites (or derivatives thereof), as disclosed in Table 11, Table 12A, Table 12B, or Tables 1-7 (preferably Tables 2-7), may also serve as markers for genetic deficiency (e.g., leptin deficiency) or diseases such as hypothyroidism, insulin resistance, polycystic ovary syndrome, Cushing's syndrome and Prader-Willi syndrome, which may lead to obesity. For example, it is believed that at least some of the metabolites that are biomarkers of obesity may also serve as markers for one or more of these underlying causes of obesity. That is, the methods described herein with respect to obesity (or a disease related thereto) may also be used for diagnosing underlying conditions of obesity. Similarly, methods of assessing efficacy of compositions for treating obesity (or a disease related thereto), methods of screening a composition for activity in modulating metabolites associated with obesity (or a disease related thereto), methods of identifying potential drug targets for treating obesity (or a disease related thereto), and methods of treating obesity (or a disease related thereto) may be conducted in the context of diagnosis, evaluation, therapy, maintenance of underlying conditions and also for screening agents for the same purpose.

### X. Other Methods

Other methods of using the biomarkers discussed herein are also contemplated. For example, the methods described in U.S. Pat. Nos. 7,005,255; 7,635,556; 7,329,489, 7,682,783; 7,682,784 and 7,550,258 may be conducted using a small molecule profile comprising one or more of the biomarkers disclosed herein.

Kits

The disclosure also relates to kits for detecting the presence of metabolite(s) or their derivatives, e.g., the metabolites (or derivatives thereof) disclosed in Table 11, Table 12A, Table 12B, or Tables 1-7 (preferably Tables 2-7), in a biological sample (a test sample). Such kits can be used to determine if a subject is suffering from or is at increased risk of developing a disorder associated with the metabolite (e.g., obesity or a disease related thereto). For example, the kit can comprise a labeled compound or agent capable of detecting the metabolite (or derivative thereof) in a biological sample and reagent/equipment for determining the amount of the metabolite (or derivative thereof) in the sample (e.g., an antibody against the metabolite or its derivative). Preferably, kits comprise one or more reagents to preserve the analyte (i.e., metabolites or derivatives thereof) and prevent contamination thereof. Typically, sodium azide (10%) may be used to prevent bacterial contamination. Kits may also include reagents for extraction of metabolites, e.g., acetonitrile, methanol, or chloroform, etc. Perchloric acid (PCA) may be included in metabolites are to be extracted from adherent cell culture and mammalian tissues.

Kits may also include instruction for observing that the tested subject is suffering from or is at risk of developing obesity or a disease related thereto if the amount of the metabolite is above or below a normal level. The kit may also comprise, e.g., a buffering agent, a preservative, or a stabilizing agent. The kit may also comprise components necessary for detecting the detectable agent (e.g., a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing obesity or a disease related thereto.

Especially, provided herein is a kit for determining a lipid or fat content of a biological sample, comprising, a plurality of probes for detecting a metabolite profile of the biological sample; vessels for holding the biological sample; optionally together with instructions for performing the detection, wherein the metabolite profile comprises at least three of the metabolites of Table 1 or derivatives thereof, wherein the at least 3 metabolites comprises, in the order of rank of relative correlation to the lipid or fat content, urate, 5-methylthioadenosine, and glutamate or derivatives thereof.

Further, provided herein is a kit for determining a lipid or fat content of a biological sample, comprising, a plurality of probes for detecting a metabolite profile of the biological sample; vessels for holding the biological sample; optionally together with instructions for performing the detection, wherein the metabolite profile comprises at least three of the metabolites of Table 2 or derivatives thereof, wherein the at least 3 metabolites comprises, in the order of rank of relative correlation to the lipid or fat content, urate, glutamate and 1-(1-enyl-palmitoyl)-2-oleoyl-GPC (P-16:0/18:1) or derivatives thereof.

Computer-Implemented Systems

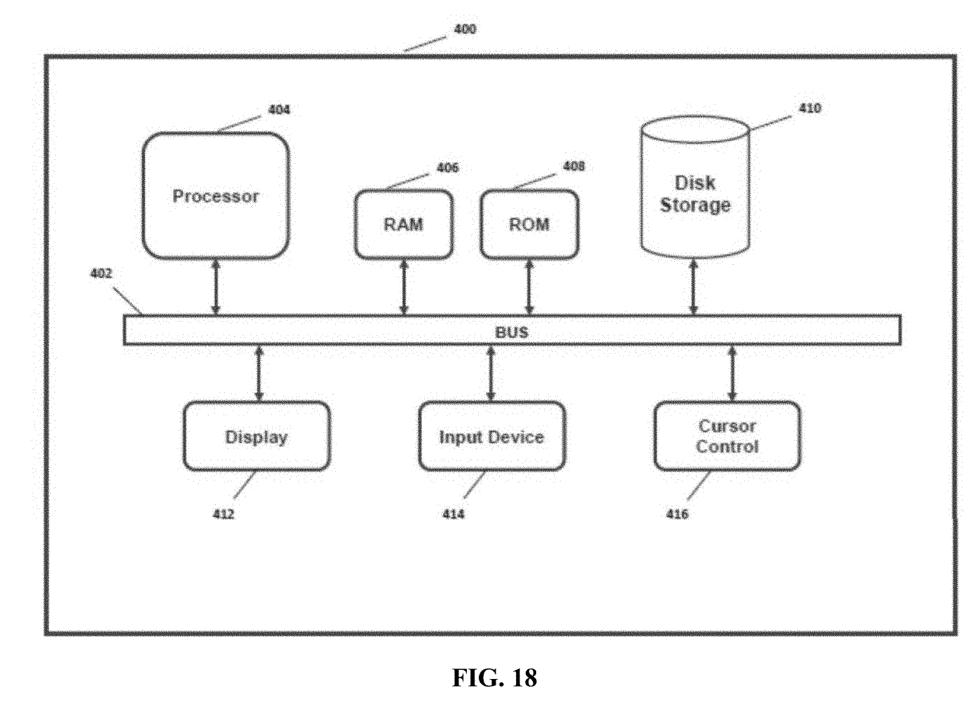

FIG. 18 is a block diagram that illustrates a computer system 400, upon which embodiments of the present teachings may be implemented. In various embodiments of the present teachings, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3 dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively hard-wired circuitry can be used in place of or in combination with software instructions to implement the present teachings. Thus, implementations of the present teachings are not limited to any specific combination of hardware circuitry and software. The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, memory components 406/4008/410 and user input provided via input device 414.

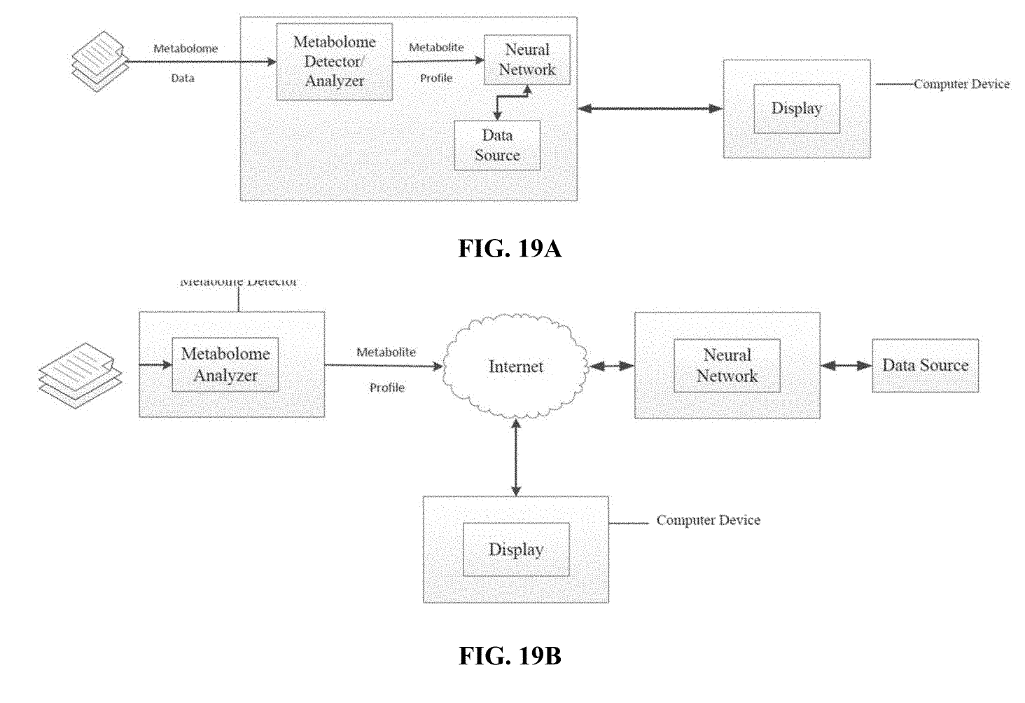

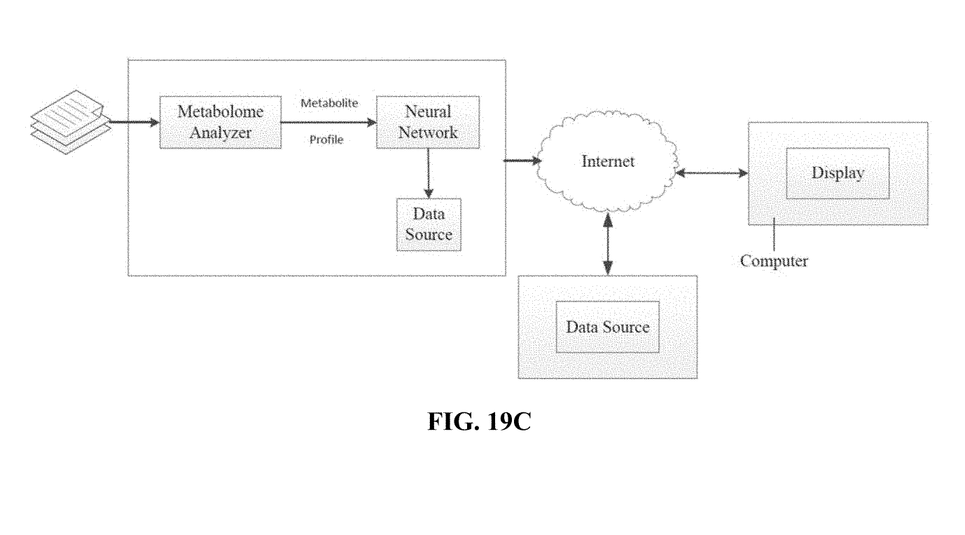

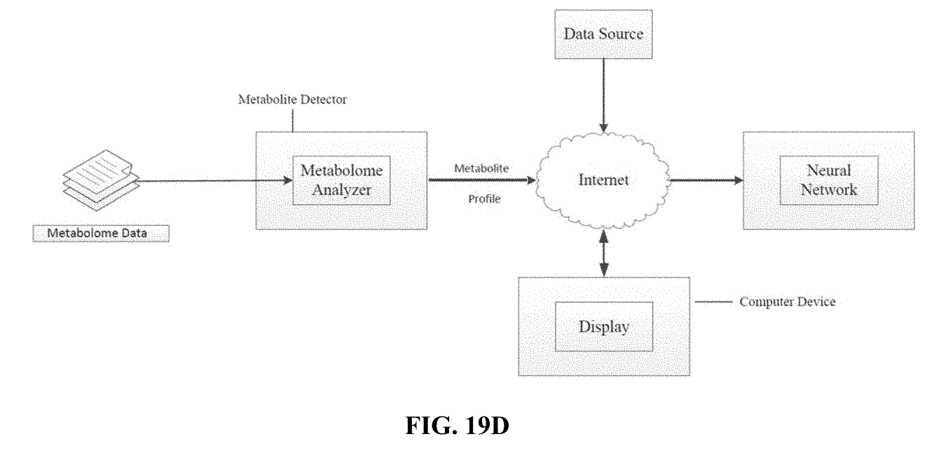

FIG. 19 provides schematic representations of various system architectures that can be employed to practice the methods of the disclosure.

FIG. 19A provides a schematic representation of an integrated system. Metabolome data, which can be made available on point (e.g., via a standalone sequence) or via a database (e.g., as TXT or CSV file), is received by the metabolome detector/analyzer. The metabolome analyzer is capable of determining a level (e.g., via counting concentration or amount of metabolites) or activity of metabolites in the received dataset. The metabolome analyzer may communicate with a neural network to filter noise contained in the data and/or to improve search for markers that are associated with the disease (e.g., obesity). The neural network may be trained with a training dataset comprising actual biological samples (e.g., tissue sample) of patients, which are further phenotypically annotated, e.g., for obesity profile. Listings of markers that have the highest predictive significance are provided in Table 1 and Table 2. Metabolite signatures are further provided in Tables 3-7. Accordingly, in some embodiments, the output of the analyzer may be matched with the markers that are recited in Table 1 (preferably Table 2) and a result of process be displayed in the display monitor. Optionally, the display monitor is a part of a computer device that receives the outputs of the analyzer and/or the neural network and performs mathematical analyses (e.g., regression analysis) to output a metabolome body mass index (mBMI), e.g., using Equation 1 (described above). The display may further indicate whether results of the analyses permit reliable and/or accurate inferences about the sample/subject's trait (e.g., obesity) to be made. Such a computer system may also allow a user (e.g., a scientist or a clinician) to evaluate the results (e.g., based on statistical output of confidence intervals) and input recommendations and other notes based on such evaluations.

FIG. 19B provides a schematic representation of a semi-integrated system. A difference between the semi-integrated system and the integrated system of FIG. 19A is that the output of the analyzer (which has been filtered and optionally weighed based on a dynamic neural network-mediated filtering/weighing process or a static matching process with the top 5%, top 10%, top 20%, top 50% or top 80% of metabolite markers listed in Table 1 or Table 2) is analyzed in real time over an internet (or cloud) and assessments are made in real time by comparing to existing datasets. The results of the analyses are outputted via a computer display that may be located distally from the marker analyzer module.

FIG. 19C provides a schematic representation of a semi-discrete system. A difference between the semi-discrete system and the semi-integrated system of FIG. 19B is that neural network (or even a static listing of prominent metabolite markers, e.g., Table 1 or Table 2) need not be housed within or in close proximity to the methylation analyzer. In fact, the methylation data processed by the methylation analyzer may be continuously processed, in real time, to dynamically provide information about associations between the metabolite markers and the traits of interest (e.g., obesity).

FIG. 19D provides a schematic representation of a completely discrete system. A difference between the fully discrete system and the semi-discrete system of FIG. 19C is the central location of the cloud/internet, which contains metabolome data from not only the subject in question, but also an entire database of other subjects (who may be optionally matched to the subject in question based on other phenotypic traits (e.g., blood pressure, insulin resistance) and/or anthropometric traits (e.g., waist-to-hip ratio, waist to hip ratio (WHR), waist circumference, and/or BMI). The patient's obesity status, as determined by the analyzer, including other subjects (as inputted by the database) is analyzed by a neural network, which has been trained by a data source. The output of the network, as applied on the patient's dataset, may optionally be compared to the output of the network on an in silico dataset, and the predictive accuracy of the system and also the subject's metabolome dataset, may be outputted onto a display monitor via a computer.

In various embodiments, provided herein is an obesity profiling system, comprising: (a) a metabolome detector/analyzer configured to detect/analyze levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a subject's biological sample; (b) an obesity determining engine configured to determine obesity based on levels and/or activities of metabolites or derivatives thereof, wherein the engine is optionally communicatively connected to a data source (e.g., human metabolome database); and (c) a display communicatively connected to a computing device and configured to display a report containing the subject's obesity profile.

In some embodiments, provided herein is an obesity profiling system, comprising: (a) a metabolome detector/analyzer configured to detect/analyze levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B,9 or Tables 1-7 (preferably Tables 2-7) in a subject's biological sample; (b) an obesity determining engine configured to determine obesity based on levels and/or activities of metabolites, wherein the engine is optionally communicatively connected to a data source (e.g., human metabolome database); and (c) a display communicatively connected to a computing device and configured to display a report containing the subject's obesity profile, wherein components (a), (b) and (c) are communicatively connected to each other, e.g., via the internet.

In some embodiments, provided herein is an obesity profiling system, comprising: (a) a metabolome detector/analyzer configured to detect/analyze levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a subject's biological sample, wherein the analyzer is communicatively connected to an obesity determining engine configured to determine obesity based on levels and/or activities of metabolites; (b) a data source (e.g., human metabolome database); and (c) a display communicatively connected to a computing device and configured to display a report containing the subject's obesity profile, wherein components (a), (b) and (c) are communicatively connected to each other, e.g., via the internet.

In some embodiments, provided herein is an obesity profiling system, comprising: (a) a metabolome detector/analyzer configured to detect/analyze levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a subject's biological sample; (b) an obesity determining engine configured to determine obesity based on levels and/or activities of metabolites; (c) a data source (e.g., human metabolome database); and (d) a display communicatively connected to a computing device and configured to display a report containing the subject's obesity profile, wherein components (a), (b), (c) and (d) are communicatively connected to each other, e.g., via the internet.

In some embodiments, provided herein are obesity profiling systems of the foregoing, comprising a metabolome detector/analyzer configured to detect/analyze levels or activities of at least 3 metabolites of Table 1 or derivatives thereof in a subject's biological sample, wherein the at least 3 metabolites comprises, in the order of rank of relative correlation to the subject's obesity, urate, 5-methylthioadenosine, and glutamate.

In some embodiments, herein are obesity profiling systems of the foregoing, comprising a metabolome detector/analyzer configured to detect/analyze levels or activities of at least 3 metabolites of Table 2 or derivatives thereof in a subject's biological sample, wherein the at least 3 metabolites comprises, in the order of rank of relative correlation to the subject's obesity, urate, glutamate and 1-(1-enyl-palmitoyl)-2-oleoyl-GPC (P-16:0/18:1).

In some embodiments, provided herein is an obesity profiling system of the foregoing, further comprising an analyzer for detecting a secondary parameter in the subject's sample optionally together with a genetic parameter. Preferably, the secondary parameter is selected from the group consisting of android/gynoid ratio; total triglycerides; waist/hip ratio; subcutaneous fat; visceral fat; insulin resistance; HDL; percent fat; diastolic blood pressure; systolic blood pressure; total cholesterol; and LDL, or a combination thereof, particularly preferably, android/gynoid ratio; total triglycerides; waist/hip ratio; subcutaneous fat; visceral fat; insulin resistance; and HDL. Preferably, the genetic parameter is selected from genetic variants of melanocortin 4 receptor gene (MC4R) or a lipdystrophy gene selected from zinc metallopeptidase STE24 (ZMPSTE24), 1-acylglycerol-3-phosphate O-acyltransferase 2 (AGPAT2), lipase E, hormone sensitive type (LIPE), Bernardinelli-Seip congenital lipodystrophy type 2 (BSCL2), or a combination thereof. Especially, the analyzer analyzes, whether the subject's sample comprises an MC4R variant selected from M292fs, R236C, S180P, A175T, and T11A, but not I170V; and/or whether the subject's sample comprises a genetic variant of ZMPSTE24, AGPAT2, LIPE, BSCL2, or a combination thereof.

The disclosure further relates to computer readable medium comprising computer-executable instructions, which, when executed by a processor, cause the processor to carry out a method or a set of steps for diagnosing obesity in a subject. In some embodiments, the computer readable media carry out a method or a set of steps for diagnosing obesity in a subject, comprising detecting levels or activities of a plurality of metabolites (or derivatives thereof) (e.g., at least 2, 3, 4, 5, 6, 7, 8, 9, 10, 11, 12, 13, 14, 15, 20, 25, 30, 39, 40, 50, 80, 100, 150, 200, 250, 300, 307, or more, e.g., 500 metabolites or derivatives thereof) from Table 11, Table 12A, Table 12B, or Tables 1-7 (preferably Tables 2-7) in a subject's biological sample, wherein the computer readable medium comprises machine learning techniques to determine obesity of subject based on the metabolite profile.

In some embodiments, the computer readable media carry out a method or a set of steps for diagnosing obesity in a subject, comprising detecting a metabolite profile in a metabolome dataset received from a subject's sample, wherein the metabolite profile comprises levels or activities of at least three metabolites of Table 1 or derivatives thereof and the computer readable medium comprises machine learning techniques to determine obesity of subject based on the metabolite profile, wherein the at least 3 metabolites comprises, in the order of rank of relative correlation to the subject's obesity, urate, 5-methylthioadenosine, and glutamate.

In some embodiments, the computer readable media carry out a method or a set of steps for diagnosing obesity in a subject, comprising detecting a metabolite profile in a metabolome dataset received from a subject's sample, wherein the metabolite profile comprises levels or activities of at least three metabolites of Table 2 or derivatives thereof and the computer readable medium comprises machine learning techniques to determine obesity of subject based on the metabolite profile, wherein the at least 3 metabolites comprises, in the order of rank of relative correlation to the subject's obesity, urate, glutamate and 1-(1-enyl-palmitoyl)-2-oleoyl-GPC (P-16:0/18:1).

The aforementioned embodiments of the disclosure are further described in view of the following non-limiting examples.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

There are recent calls to improve phenotyping in very large numbers of people with obesity with the goals of understanding factors that make people susceptible to (or protected from) obesity, accompanied by a better elucidation of the factors that account for variability in success of different obesity treatments. Here, longitudinal body mass index (BMI), anthropomorphic measurements, whole body DXA scans and genetic risk and metabolite data were analyzed from 2,601 individuals. The metabolome assay covered up to 1,007 metabolites at up to three time-points per person. Associations between nearly a third of the metabolome and BMI were identified, and it was revealed that metabolite levels can explain ˜40% of the variation in BMI and can predict obesity status with ˜80-90% specificity and sensitivity. The metabolome profile is a strong indicator of metabolic health compared to the polygenic risk and anthropomorphic measurement of BMI.

Methods

Samples and study design—The study included 1,969 European ancestry twins enrolled in the TWINSUK registry, a British national register of adult twins. A detailed study of the genetic variants influencing the human metabolome in this cohort has been previously reported in Long et al. (Nature Genetics, 49, 568-578, 2017). Serum samples were collected at three visits, 8-18 (median 13) years apart. The cohort is mainly composed of females (96.7%), and the sample set included 388 monozygotic twin pairs, 519 dizygotic twin pairs, and 155 unrelated individuals. The age of participants at the first time point ranged from 33 to 74 years old (median 51); 36 to 81 years old (median 59) at the second time point; and 42 to 88 years old (median 65) at the third time point. The BMI values measured at each metabolome time point were taken within two years of the blood draw date. At baseline, 36.3% of the female participants and 53.8% of the male participants were overweight, and 16.9% of the females and 10.8% of the males were obese. The twins study was approved by Ethics Committee, and all participants provided informed written consent. BMI data were available for 1743 participants within two years of the time point for metabolome time point 1, 1834 for within two years of time point 2, and 1777 for up to 2 years before time point 3 or 4 years after this time point; 1,458 individuals had all three data points. For independent validation and studies of phenotypes correlated with metabolic BMI outliers, 617 unselected adults more than 18 years old who were available for a clinical research protocol were enrolled. Participants underwent a verbal review of the institutional review board-approved consent. Participants ranged in age from 18-89 years old (median 53), were 32.9% female, and had BMI data measured at one time point: 16.7% of the female participants and 47.5% of the male participants were overweight, and 7.2% of the females and 23.7% of the males were obese.

Phenotyping—Individuals in the TWINSUK cohort and Health Nucleus both underwent DEXA imaging. The data from these scans were used to calculate android/gynoid ratio, percent body fat, visceral fat, and subcutaneous fat. DEXA is very accurate in the measurement abdominal visceral adipose tissue (VAT). High levels of VAT are associated with atherogenic dyslipidemia, hyperinsulinemia, and glucose intolerance (Neeland et al., Circulation, 137, 1391-1406, 2018). TWINSUK cohort participants were additionally measured for circumference at the waist and hip using a measuring tip to calculate the waist/hip ratio. TWINSUK participants self-reported information about whether they were taking high blood pressure medication at their first visit and about cardiovascular events and their timing via a survey at the final visit. MRI images were available for a selected number of Health Nucleus participants. Insulin resistance was defined by HOMA score >3 (available on the world-wide-web at gihep(dot)com/calculators/other/homa/).

Metabolite Profiling—The non-targeted metabolomics analysis of 901 metabolites in the TWINSUK cohort and 1,007 metabolites in the Health Nucleus cohort was performed at Metabolon, Inc. (Durham, N.C., USA) on a platform consisting of four independent ultra-high performance liquid chromatography-tandem mass spectrometry (UPLC-MS/MS) methods. The detailed descriptions of the platform can be found in previous publications (Long et al., Nature Genetics, 49, 568-578, 2017). For the TWINSUK cohort, blood serum after fasting was used for analysis, and the resulting raw values were transformed to z scores using the mean and standard deviation. For the Health Nucleus cohort, blood plasma after fasting was used for analysis, and values from multiple experimental batches were normalized into Z-scores based on a reference cohort of either 42 (n=457) or 300 (n=176) self-reported healthy individuals run with each batch. The 42 and 300-normalized batches were converted to the same scale using linear transformation based on the values obtained from 7 runs that included both the 42 and 300 controls. Samples with metabolite measurements that were below the detection threshold were imputed as the minimum value for that metabolite.

Genome sequencing and analysis—As previously described, DNA samples were sequenced on an Illumina HISEQX sequencer utilizing a 150 base paired-end single index read format. Reads were mapped to the human reference sequence build HG38. Variants were called using ISIS Analysis Software (v. 2.5.26.13; Illumina). A linear mixed model was applied to account for family structure in the cohort while testing for associations between genetic variants and the different phenotypes: BMI; BMI prediction model values and residuals after accounting for BMI, age, sex; and levels of the 49 BMI-associated metabolites. A genetic similarity matrix (GSM) was constructed from 301,556 variants that represented a random 20% of all common (MAF>5%) variants genome-wide after linkage-disequilibrium (LD) pruning (r² less than 0.6, window size 200 kb) and was used to model the random effect in the linear mixed model via a “leave-out-one-chromosome” method for each tested variant. Each of 97 known BMI-associated variants was tested independently using customized Python scripts wrapping the FAST-LMM package. Principal component axes were calculated to check ethnicity using plink, and the first principal component for those of European ancestry was used as a covariate in analyses of unrelated individuals in R described below. Polygenic risk scores were calculated using genotypes for 97 variants whose associations and betas had been published previously. For rare variant analysis in the gene MC4R, coding and splice variants with MAF<0.1% were analyzed with a gene-based collapsing analysis where all qualifying variants in the gene were grouped together, again using customized Python scripts wrapping the FAST-LMM package and the same GSM described above to account for relatedness. Rare lipodystrophy variants were defined as those achieving a pathogenic or likely pathogenic categorization in ClinVar or HGMD.

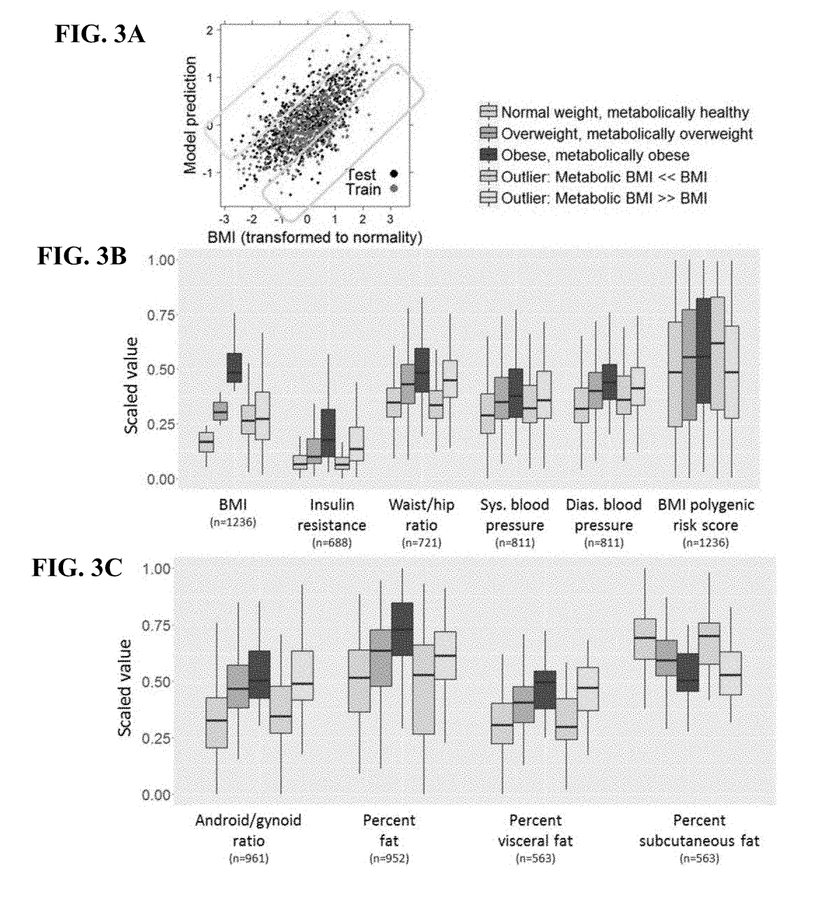

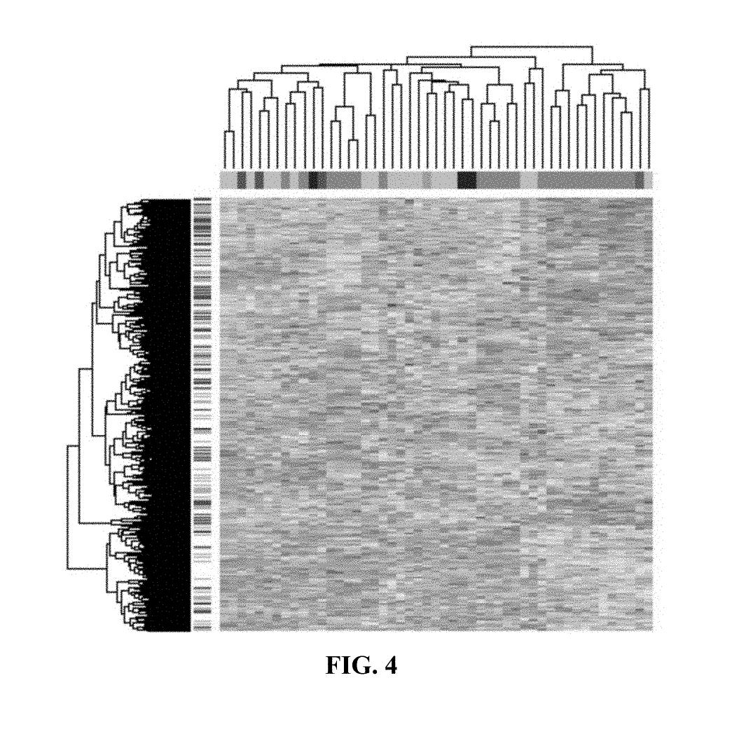

Statistical analysis—R was used for the analysis and data manipulation. Bonferroni correction was used for all analyses, and most statistical analyses were restricted to unrelated individuals of European ancestry, in accordance with field standards for ensuring that ancestry differences do not cause bias or skew in the results. For each quantitative analysis of BMI or other traits, the subset of BMI values or other outcome variables used were rank-ordered and forced to a normal distribution. Analyses comparing metabolites to BMI were performed in R using the 1 m function, and age, sex, and the first genetic principal component were included as covariates. The obesity prediction model was built using ridge regression (alpha=0) with glmnet in R. The residuals used to separate participants into the five categories shown in FIG. 3 were calculated using age, sex, and initial BMI. Heat maps were generated in R using the pheatmap package. Survival analysis was performed using coxph in R with age at first visit included as a covariate.

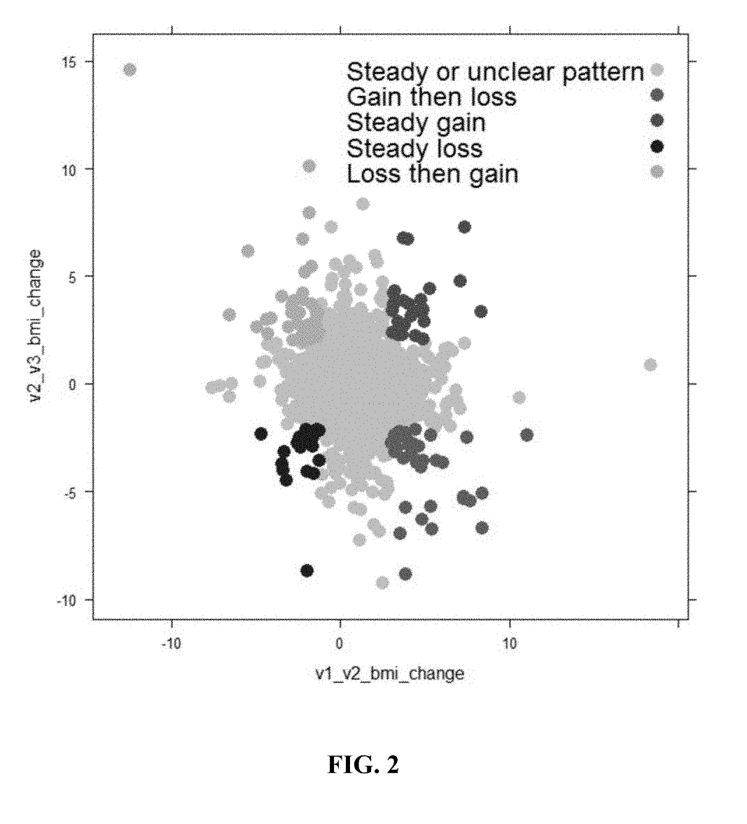

For the analysis of change in BMI across visits 1, 2 and 3, the slope of the change for each person was calculated (change in BMI vs. change in years). For the analyses of BMI recovery, participants were separated into four categories based on having values that were at least one standard deviation above or below the mean for the BMI change at that time point (FIG. 2). Analyses comparing metabolites to change in BMI were performed in R using the 1 m function, and age, sex, initial BMI and the first genetic principal component were included as covariates. For principal component analysis, the metabolite normalized Z-scores were rank-ordered and forced to a normal distribution, and missing data were imputed using the missForest R package. Principal components were calculated using the prcomp command in R. Data files are presented in Tables 11, 12A and 12B.

Results

Profound Perturbation of Metabolome by Obesity (Metabolites Associated with Body Mass Index).

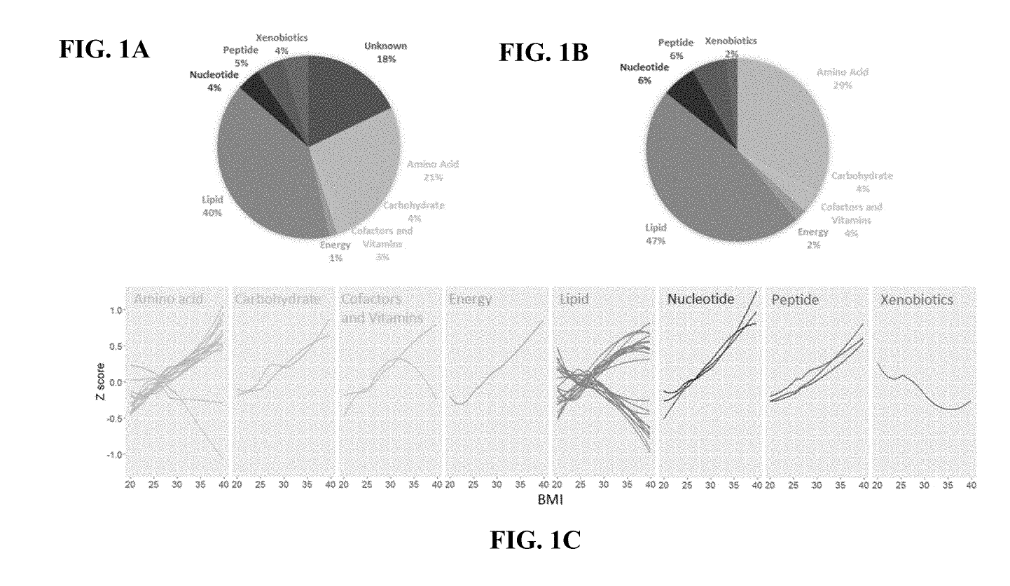

The levels of 901 metabolites to the BMIs of 832, 882, and 861 unrelated individuals of European ancestry in the TWINSUK cohort at three time points spanning a total range of 8-18 years were compared. Initially, 284 metabolites that were significantly associated (p<5.5×10-5) with BMI at one or more time points were identified (Table 11, Table 12A and Table 12B). The study focused on 110 metabolites that were significantly associated with BMI at all 3 time points and sought to replicate the associations in an independent sample of 427 unrelated individuals of European ancestry out of the 617 participants in the Health Nucleus cohort. Of the 84 metabolites that had been measured in both cohorts, 83 showed directions of effect that were consistent between the two cohorts, and 49 were statistically significant replications (FIG. 1). While this set of 49 metabolites were the most stringently associated with BMI, the majority of the implicated metabolites (292 of 307, 95%) had directions of effect that were consistent between timepoints/cohorts, indicating that many of the remaining metabolites may reach our stringent cutoffs in a larger study.

The 49 metabolites that associated with BMI were primarily lipids (n=23, accounting for 7.5% of all lipids assayed across both cohorts) and amino acids (n=14, 9.3% of all amino acids) as well as nucleotides (n=3, 12.0% of all nucleotides), peptides (n=3, 12% of all peptides), and other categories (n=6, see FIG. 1 and Table 1). The most significantly associated metabolite was urate (uric acid; p-value 1.2×10-40 for combined analysis of TWINSUK time point 1 and Health Nucleus data). In summary, the analyses identified 307 metabolites (Table 1) that were significantly associated with BMI in at least one cohort and time point (Table 11, Table 12A and Table 12B), and a signature of 49 (Table 2) metabolites that were consistently significantly associated with BMI.

Patterns in Metabolite Change According to BMI.

The majority of the 49 BMI-associated metabolites increased with increasing BMI (n=35) (FIG. 1, Table 11, Table 12A and Table 12B). Notably, this included glucose and mannose, which has recently been highlighted as playing a role in insulin resistance. Most metabolites change linearly with BMI, though some appeared to have a tapering off of the association at higher BMIs, especially 2-methylbutyrylcarnitine (see cofactors panel in FIG. 1C). Branched-chain and aromatic amino acids as well as metabolites related to nucleotide metabolism like urate had the most rapid increases. Those that decreased (n=14) included phospholipids and lysolipids, as well as the amino acids asparagine and N-acetylglycine and the xenobiotic cinnamoylglycine, which has been identified as a product of the microbiome. The negatively associated lipids tended to reflect HDL (high-density lipoprotein) levels, while the positively correlated lipids were more representative of triglyceride levels (Table 1, Table 11 and Table 12).

TABLE 11

| | | | | | 49 | 307 | |
|---|---|---|---|---|---|---|---|
| | | | | | definitive | ever sig- | |
| | | | | | BMI- | nificantly | direc- |
| Metabolite | other | | Super | Sub | associated | associated | tion of |
| ID | ID | Metabolite | pathway | pathway | metabolites | with BMI | effect |
| 1134 | | urate | Nucleotide | Purine | 1 | 1 | pos |
| | | | | Metabolism, | | | |
| | | | | (Hypo)Xanthine/ | | | |
| | | | | Inosine | | | |
| | | | | containing | | | |
| 100001412 | | N2,N2- | Nucleotide | Purine | 1 | 1 | pos |
| | | dimethyl- | | Metabolism, | | | |
| | | guanosine | | Guanine | | | |
| | | | | containing | | | |
| 100009051 | | 1-stearoyl- | Lipid | Phospholipid | 1 | 1 | pos |
| | | 2-dihomo- | | Metabolism | | | |
| | | linolenoyl- | | | | | |
| | | GPC | | | | | |
| | | (18:0/20:3n3 | | | | | |
| | | or 6)* | | | | | |
| 561 | | glutamate | Amino | Glutamate | 1 | 1 | pos |
| | | | Acid | Metabolism | | | |
| 212 | | 5-methyl- | Amino | Polyamine | 1 | 1 | pos |
| | | thioadenosine | Acid | Metabolism | | | |
| | | (MTA) | | | | | |
| 100001384 | | 1-arachidoyl- | Lipid | Lysolipid | 1 | 1 | neg |
| | | GPC (20:0) | | | | | |
| 100001006 | | N- | Amino | Glycine, | 1 | 1 | neg |
| | | acetylglycine | Acid | Serine and | | | |
| | | | | Threonine | | | |
| | | | | Metabolism | | | |
| 100005353 | | 1-nonadecanoyl- | Lipid | Lysolipid | 1 | 1 | neg |
| | | GPC (19:0) | | | | | |
| 566 | | valine | Amino | Leucine, | 1 | 1 | pos |
| | | | Acid | Isoleucine | | | |
| | | | | and Valine | | | |
| | | | | Metabolism | | | |
| 100009007 | | 1-(1-enyl- | Lipid | Plasmalogen | 1 | 1 | neg |
| | | palmitoyl)- | | | | | |
| | | 2-oleoyl-GPC | | | | | |
| | | (P-16:0/18:1)* | | | | | |
| 100005352 | | 1-eicosenoyl- | Lipid | Lysolipid | 1 | 1 | neg |
| | | GPC (20:1)* | | | | | |
| 100001948 | | succinyl- | Energy | TCA Cycle | 1 | 1 | pos |
| | | carnitine | | | | | |
| 100008917 | | 1-(1-enyl-stearoyl)- | Lipid | phospholipid | 1 | 1 | neg |
| | | 2-oleoyl-GPC | | | | | |
| | | (P-18:0/18:1) | | | | | |
| 100001162 | | propionyl- | Lipid | Fatty Acid | 1 | 1 | pos |
| | | carnitine | | Metabolism | | | |
| | | | | (also BCAA | | | |
| | | | | Metabolism) | | | |
| 98 | | kynurenate | Amino | Tryptophan | 1 | 1 | pos |
| | | | Acid | Metabolism | | | |
| 803 | | mannose | Carbo- | Fructose, | 1 | 1 | pos |
| | | | hydrate | Mannose and | | | |
| | | | | Galactose | | | |
| | | | | Metabolism | | | |
| 1084 | | N- | Amino | Leucine, | 1 | 1 | pos |
| | | acetylvaline | Acid | Isoleucine | | | |
| | | | | and Valine | | | |
| | | | | Metabolism | | | |
| 100008981 | | 1-oleoyl-2- | Lipid | Phospholipid | 1 | 1 | neg |
| | | linoleoyl-GPC | | Metabolism | | | |
| | | (18:1/18:2)* | | | | | |
| 100001395 | | 1-linoleoyl- | Lipid | Lysolipid | 1 | 1 | neg |
| | | GPC (18:2) | | | | | |
| 100004046 | | N- | Peptide | Dipeptide | 1 | 1 | pos |
| | | acetylcarnosine | | Derivative | | | |
| 100002106 | | sphingomyelin | Lipid | Sphingolipid | 1 | 1 | pos |
| | | (d18:1/18:1, | | Metabolism | | | |
| | | d18:2/18:0) | | | | | |
| 100001415 | | N6-carbamoyl- | Nucleotide | Purine | 1 | 1 | pos |
| | | threonyl- | | Metabolism, | | | |
| | | adenosine | | Adenine | | | |
| | | | | containing | | | |
| 100009009 | | 1-(1-enyl-palmitoyl)- | Lipid | Phospholipid | 1 | 1 | neg |
| | | 2-linoleoyl- | | Metabolism | | | |
| | | GPC (P- | | | | | |
| | | 16:0/18:2)* | | | | | |
| 100008985 | | 1-palmitoyl- | Lipid | Phospholipid | 1 | 1 | pos |
| | | 2-dihomo- | | Metabolism | | | |
| | | linolenoyl-GPC | | | | | |
| | | (16:0/20:3n3 | | | | | |
| | | or 6)* | | | | | |
| 1110 | | N- | Amino | Alanine and | 1 | 1 | pos |
| | | acetylalanine | Acid | Aspartate | | | |
| | | | | Metabolism | | | |
| 811 | | alanine | Amino | Alanine and | 1 | 1 | pos |
| | | | Acid | Aspartate | | | |
| | | | | Metabolism | | | |
| 100009015 | | 1-(1-enyl- | Lipid | Phospholipid | 1 | 1 | neg |
| | | stearoyl)-2- | | Metabolism | | | |
| | | docosahexa- | | | | | |
| | | enoyl-GPC | | | | | |
| | | (P-18:0/22:6)* | | | | | |
| 100000491 | | gamma- | Peptide | Gamma- | 1 | 1 | pos |
| | | glutamylphenyl- | | glutamyl | | | |
| | | alanine | | Amino Acid | | | |
| 100009055 | | 1-palmitoyl- | Lipid | Phospholipid | 1 | 1 | pos |
| | | 3-linoleoyl- | | Metabolism | | | |
| | | glycerol | | | | | |
| | | (16:0/18:2)* | | | | | |
| 917 | | asparagine | Amino | Alanine and | 1 | 1 | neg |
| | | | Acid | Aspartate | | | |
| | | | | Metabolism | | | |
| 1102 | | gamma- | Peptide | Gamma- | 1 | 1 | pos |
| | | glutamyl- | | glutamyl | | | |
| | | tyrosine | | Amino Acid | | | |
| 815 | | tyrosine | Amino | Phenylalanine | 1 | 1 | pos |
| | | | Acid | and Tyrosine | | | |
| | | | | Metabolism | | | |
| 100002990 | | 1-oleoyl-3- | Lipid | Diacylglycerol | 1 | 1 | pos |
| | | linoleoyl- | | | | | |
| | | glycerol | | | | | |
| | | (18:1/18:2) | | | | | |
| 100008903 | | 1,2-dilinoleoyl- | Lipid | Phospholipid | 1 | 1 | neg |
| | | GPC (18:2/18:2) | | Metabolism | | | |
| 397 | | leucine | Amino | Leucine, | 1 | 1 | pos |
| | | | Acid | Isoleucine | | | |
| | | | | and Valine | | | |
| | | | | Metabolism | | | |
| 100009053 | | 1-palmitoleoyl- | Lipid | phospholipid | 1 | 1 | pos |
| | | 2-oleoyl- | | | | | |
| | | glycerol | | | | | |
| | | (16:1/18:1)* | | | | | |
| 100009052 | | 1-palmitoyl- | Lipid | Phospholipid | 1 | 1 | pos |
| | | 2-linoleoyl- | | Metabolism | | | |
| | | glycerol | | | | | |
| | | (16:0/18:2)* | | | | | |
| 100001104 | | N- | Amino | Phenylalanine | 1 | 1 | pos |
| | | acetyltyrosine | Acid | and Tyrosine | | | |
| | | | | Metabolism | | | |
| 100000007 | | carnitine | Lipid | Carnitine | 1 | 1 | pos |
| | | | | Metabolism | | | |
| 100002989 | | 1-oleoyl-2- | Lipid | Diacylglycerol | 1 | 1 | pos |
| | | linoleoyl- | | | | | |
| | | glycerol | | | | | |
| | | (18:1/18:2) | | | | | |
| 234 | | aspartate | Amino | Alanine and | 1 | 1 | pos |
| | | | Acid | Aspartate | | | |
| | | | | Metabolism | | | |
| 100002253 | | cinnamoyl- | Xeno- | Food | 1 | 1 | neg |
| | | glycine | biotics | Component/ | | | |
| | | | | Plant | | | |
| 100009054 | | 1-palmitoleoyl- | Lipid | phospholipid | 1 | 1 | pos |
| | | 3-oleoyl- | | | | | |
| | | glycerol | | | | | |
| | | (16:1/18:1)* | | | | | |
| 182 | | quinolinate | Cofactors | Nicotinate and | 1 | 1 | pos |
| | | | and | Nicotinamide | | | |
| | | | Vitamins | Metabolism | | | |
| 100001509 | | 2-methyl- | Amino | Leucine, | 1 | 1 | pos |
| | | butyryl- | Acid | Isoleucine | | | |
| | | carnitine | | and Valine | | | |
| | | (C5) | | Metabolism | | | |
| 572 | | glucose | Carbo- | Glycolysis, | 1 | 1 | pos |
| | | | hydrate | Gluconeo- | | | |
| | | | | genesis, and | | | |
| | | | | Pyruvate | | | |
| | | | | Metabolism | | | |
| 100009143 | | 1-palmitoyl- | Lipid | Phospholipid | 1 | 1 | pos |
| | | 2-adrenoyl- | | Metabolism | | | |
| | | GPC | | | | | |
| | | (16:0/22:4)* | | | | | |
| 100001586 | | gulonic | Cofactors | Ascorbate and | 1 | 1 | pos |
| | | acid* | and | Aldarate | | | |
| | | | Vitamins | Metabolism | | | |
| 273 | | cortisone | Lipid | Steroid | 1 | 1 | neg |
| X - 12063 | | X - 12100 | 0 | . | 0 | 1 | pos |
| X - 22822 | | X - 22822 | 0 | . | 0 | 1 | pos |
| X - 11564 | | X - 11787 | 0 | . | 0 | 1 | pos |
| X - 15492 | | X - 15492 | 0 | . | 0 | 1 | pos |
| X - 13529 | | 1-carboxy- | 0 | . | 0 | 1 | pos |
| | | ethylvaline | | | | | |
| X - 15497 | | 1-carboxy- | 0 | . | 0 | 1 | pos |
| | | ethylphenyl- | | | | | |
| | | alanine | | | | | |
| 100009020 | | 1-palmityl- | Lipid | Plasmalogen | 0 | 1 | neg |
| | | 2-oleoyl-GPC | | | | | |
| | | (O- | | | | | |
| | | 16:0/18:1) | | | | | |
| X - 15503 | | X - 15503 | 0 | . | 0 | 1 | pos |
| X - 11444 | | X - 11452 | 0 | . | 0 | 1 | pos |
| X - 12026 | | X - 12040 | 0 | . | 0 | 1 | pos |
| 100005985 | | sphingomyelin | Lipid | Sphingolipid | 0 | 1 | pos |
| | | (d18:2/14:0, | | Metabolism | | | |
| | | d18:1/14:1)* | | | | | |
| 821 | | pseudouridine | Nucleotide | Pyrimidine | 0 | 1 | pos |
| | | | | Metabolism, | | | |
| | | | | Uracil | | | |
| | | | | containing | | | |
| X - 11261 | | X - 11299 | 0 | . | 0 | 1 | pos |
| 100000265 | | kynurenine | Amino | Tryptophan | 0 | 1 | pos |
| | | | Acid | Metabolism | | | |
| 100006379 | | C-glycosyl- | Amino | Tryptophan | 0 | 1 | pos |
| | | tryptophan | Acid | Metabolism | | | |
| 100002514 | | hydantoin- | Amino | Histidine | 0 | 1 | pos |
| | | 5-propionic | Acid | Metabolism | | | |
| | | acid | | | | | |
| 344 | | guanidino- | Amino | Creatine | 0 | 1 | neg |
| | | acetate | Acid | Metabolism | | | |
| 381 | | 2-amino- | Amino | Lysine | 0 | 1 | pos |
| | | adipate | Acid | Metabolism | | | |
| 100000010 | | 3-phenyl- | Amino | Phenylalanine | 0 | 1 | neg |
| | | propionate | Acid | and Tyrosine | | | |
| | | (hydrocinnamate) | | Metabolism | | | |
| 1254 | | glycerol | Lipid | Glycerolipid | 0 | 1 | pos |
| | | | | Metabolism | | | |
| 100019794 | X - 02269 | hydroxy- | 0 | . | 0 | 1 | neg |
| | | CMPF* | | | | | |
| 880 | | adenine | Nucleotide | Purine | 0 | 1 | pos |
| | | | | Metabolism, | | | |
| | | | | Adenine | | | |
| | | | | containing | | | |
| 100010930 | X - 24125 | palmitoleoyl- | Lipid | Diacylglycerol | 0 | 1 | pos |
| | | linoleoyl- | | | | | |
| | | glycerol | | | | | |
| | | (16:1/18:2) | | | | | |
| | | [1]* | | | | | |
| 100001425 | X - 11429 | 5,6- | 0 | . | 0 | 1 | pos |
| | | dihydrouridine | | | | | |
| X - 17166 | | X - 17166 | 0 | . | 0 | 1 | pos |
| 376 | | isoleucine | Amino | Leucine, | 0 | 1 | pos |
| | | | Acid | Isoleucine | | | |
| | | | | and Valine | | | |
| | | | | Metabolism | | | |
| 100005849 | | 3-methyl- | Amino | Lysine | 0 | 1 | pos |
| | | glutaryl- | Acid | Metabolism | | | |
| | | carnitine (1) | | | | | |
| 1242 | | N1-methyl- | Nucleotide | Purine | 0 | 1 | pos |
| | | adenosine | | Metabolism, | | | |
| | | | | Adenine | | | |
| | | | | containing | | | |
| X - 12846 | | X - 12847 | 0 | . | 0 | 1 | pos |
| 893 | | arachidate | Lipid | Long Chain | 0 | 1 | neg |
| | | (20:0) | | Fatty Acid | | | |
| X - 15486 | | X - 15486 | 0 | . | 0 | 1 | pos |
| 100001264 | | 1-margaroyl- | Lipid | Lysolipid | 0 | 1 | neg |
| | | GPC (17:0) | | | | | |
| 100001272 | | 1-oleoyl- | Lipid | Lysolipid | 0 | 1 | neg |
| | | GPC (18:1) | | | | | |
| X - 12170 | | X - 12206 | 0 | . | 0 | 1 | pos |
| 892 | | nonadeca- | Lipid | Long Chain | 0 | 1 | neg |
| | | noate (19:0) | | Fatty Acid | | | |
| 100009130 | | 1-oleoyl-2- | Lipid | Phospholipid | 0 | 1 | neg |
| | | docosahexa- | | Metabolism | | | |
| | | enoyl-GPC | | | | | |
| | | (18:1/22:6)* | | | | | |
| 100009037 | | 1-margaroyl- | Lipid | Phospholipid | 0 | 1 | neg |
| | | 2-linoleoyl- | | Metabolism | | | |
| | | GPC | | | | | |
| | | (17:0/18:2)* | | | | | |
| 1082 | | N-acetyl- | Amino | Leucine, | 0 | 1 | pos |
| | | leucine | Acid | Isoleucine | | | |
| | | | | and Valine | | | |
| | | | | Metabolism | | | |
| 244 | | beta-alanine | Nucleotide | Pyrimidine | 0 | 1 | pos |
| | | | | Metabolism, | | | |
| | | | | Uracil | | | |
| | | | | containing | | | |
| 100001557 | | 2-linoleoyl- | Lipid | Lysolipid | 0 | 1 | neg |
| | | GPC (18:2)* | | | | | |
| X - 13835 | | X - 13835 | 0 | . | 0 | 1 | pos |
| 100001977 | X - 23765 | beta- | Xeno- | Food | 0 | 1 | neg |
| | | cryptoxanthin | biotics | Component/ | | | |
| | | | | Plant | | | |
| X - 17299 | | X - 17299 | 0 | . | 0 | 1 | pos |
| 100009139 | | 1-myristoyl-2- | Lipid | Phospholipid | 0 | 1 | pos |
| | | arachidonoyl- | | Metabolism | | | |
| | | GPC | | | | | |
| | | (14:0/20:4)* | | | | | |
| X - 18901 | | X - 18901 | 0 | . | 0 | 1 | neg |
| 100004329 | | sphingomyelin | Lipid | Sphingolipid | 0 | 1 | pos |
| | | (d18:2/16:0, | | Metabolism | | | |
| | | d18:1/16:1)* | | | | | |
| 358 | | hypotaurine | Amino | Methionine, | 0 | 1 | neg |
| | | | Acid | Cysteine, | | | |
| | | | | SAM and | | | |
| | | | | Taurine | | | |
| | | | | Metabolism | | | |
| 533 | | urea | Amino | Urea cycle; | 0 | 1 | pos |
| | | | Acid | Arginine | | | |
| | | | | and Proline | | | |
| | | | | Metabolism | | | |
| X - 23639 | | X - 23639 | 0 | . | 0 | 1 | neg |
| 460 | | phenylalanine | Amino | Phenylalanine | 0 | 1 | pos |
| | | | Acid | and Tyrosine | | | |
| | | | | Metabolism | | | |
| 100009122 | | 1-pentadec- | Lipid | Phospholipid | 0 | 1 | pos |
| | | anoyl-2- | | Metabolism | | | |
| | | arachidonoyl- | | | | | |
| | | GPC | | | | | |
| | | (15:0/20:4)* | | | | | |
| 100020254 | X - 21849 | glycine | 0 | . | 0 | 1 | pos |
| | | conjugate of | | | | | |
| | | C10H14O2 | | | | | |
| | | (1)* | | | | | |
| 100002544 | | 4-hydroxy- | Amino | Glutamate | 0 | 1 | pos |
| | | glutamate | Acid | Metabolism | | | |
| 100001468 | | N1-Methyl- | Cofactors | Nicotinate | 0 | 1 | pos |
| | | 2-pyridone-5- | and | and | | | |
| | | carboxamide | Vitamins | Nicotinamide | | | |
| | | | | Metabolism | | | |
| 100001856 | | 1-stearoyl- | Lipid | Phospholipid | 0 | 1 | pos |
| | | 2-oleoyl-GPE | | Metabolism | | | |
| | | (18:0/18:1) | | | | | |
| 100002875 | | 1-(1-enyl- | Lipid | Lysoplasmalogen | 0 | 1 | neg |
| | | palmitoyl)- | | | | | |
| | | GPC (P-16:0)* | | | | | |
| 100008952 | | 1-palmitoleoyl | Lipid | Monoacylglycerol | 0 | 1 | pos |
| | | glycerol | | | | | |
| | | (16:1)* | | | | | |
| 100001256 | | N-acetyl- | Amino | Phenylalanine | 0 | 1 | pos |
| | | phenylalanine | Acid | and Tyrosine | | | |
| | | | | Metabolism | | | |
| 100001734 | | N6- | Amino | Lysine | 0 | 1 | pos |
| | | acetyllysine | Acid | Metabolism | | | |
| X - 23026 | | X - 23026 | 0 | . | 0 | 1 | pos |
| 240 | | 3-(4-hydroxy- | Amino | Phenylalanine | 0 | 1 | pos |
| | | phenyl)lactate | Acid | and Tyrosine | | | |
| | | | | Metabolism | | | |
| 100001485 | | gamma- | Peptide | Gamma- | 0 | 1 | pos |
| | | glutamyl- | | glutamyl | | | |
| | | isoleucine* | | Amino Acid | | | |
| 100001566 | | 1-docosahexa- | Lipid | Lysolipid | 0 | 1 | neg |
| | | enoyl-GPC | | | | | |
| | | (22:6)* | | | | | |
| 100009135 | | 1-(1-enyl- | Lipid | Plasmalogen | 0 | 1 | neg |
| | | stearoyl)-2- | | | | | |
| | | linoleoyl-GPC | | | | | |
| | | (P-18:0/18:2) | | | | | |
| 482 | | lactate | Carbo- | Glycolysis, | 0 | 1 | pos |
| | | | hydrate | Gluconeo- | | | |
| | | | | genesis, and | | | |
| | | | | Pyruvate | | | |
| | | | | Metabolism | | | |
| X - 11315 | | X - 11372 | 0 | . | 0 | 1 | neg |
| 1087 | | erucate | Lipid | Long Chain | 0 | 1 | neg |
| | | (22:1n9) | | Fatty Acid | | | |
| 100001397 | | 1,3,7-tri- | Xeno- | Xanthine | 0 | 1 | pos |
| | | methylurate | biotics | Metabolism | | | |
| X - 11491 | | X - 11522 | 0 | . | 0 | 1 | pos |
| 100001393 | | isovaleryl- | Amino | Leucine, | 0 | 1 | pos |
| | | carnitine | Acid | Isoleucine | | | |
| | | | | and Valine | | | |
| | | | | Metabolism | | | |
| X - 17145 | | X - 17145 | 0 | . | 0 | 1 | neg |
| 100001292 | | bradykinin, | Peptide | Polypeptide | 0 | 1 | pos |
| | | des-arg(9) | | | | | |
| X - 11880 | | X - 11905 | 0 | . | 0 | 1 | pos |
| 100009162 | | 1-(1-enyl- | Lipid | phospholipid | 0 | 1 | neg |
| | | palmitoyl)- | | | | | |
| | | 2-palmitoyl- | | | | | |
| | | GPC (P- | | | | | |
| | | 16:0/16:0)* | | | | | |
| 100009148 | | 1-oleoyl-2- | Lipid | Phospholipid | 0 | 1 | pos |
| | | dihomo- | | Metabolism | | | |
| | | linolenoyl- | | | | | |
| | | GPC | | | | | |
| | | (18:1/20:3)* | | | | | |
| 100009160 | | 1-(1-enyl- | Lipid | Plasmalogen | 0 | 1 | neg |
| | | palmitoyl)-2- | | | | | |
| | | palmitoleoyl- | | | | | |
| | | GPC (P- | | | | | |
| | | 16:0/16:1) | | | | | |
| X - 18249 | | X - 18249 | 0 | . | 0 | 1 | pos |
| X - 11381 | | X - 11429 | 0 | . | 0 | 1 | pos |
| X - 21752 | | X - 21752 | 0 | . | 0 | 1 | neg |
| X - 16944 | | X - 16944 | 0 | . | 0 | 1 | pos |
| 235 | | 2-hydroxy- | Amino | Phenylalanine | 0 | 1 | pos |
| | | phenylacetate | Acid | and Tyrosine | | | |
| | | | | Metabolism | | | |
| 100001276 | | N-acetyl- | Amino | Leucine, | 0 | 1 | pos |
| | | isoleucine | Acid | Isoleucine | | | |
| | | | | and Valine | | | |
| | | | | Metabolism | | | |
| X - 24435 | | X - 24435 | 0 | . | 0 | 1 | neg |
| 823 | | pyruvate | Carbo- | Glycolysis, | 0 | 1 | pos |
| | | | hydrate | Gluconeo- | | | |
| | | | | genesis, and | | | |
| | | | | Pyruvate | | | |
| | | | | Metabolism | | | |
| 100009008 | | 1-(1-enyl- | Lipid | Plasmalogen | 0 | 1 | neg |
| | | palmitoyl)-2- | | | | | |
| | | docosahexa- | | | | | |
| | | enoyl-GPC (P- | | | | | |
| | | 16:0/22:6)* | | | | | |
| 100009147 | | 1-stearyl- | Lipid | Phospholipid | 0 | 1 | neg |
| | | GPC (0-18:0)* | | Metabolism | | | |
| X - 12306 | | X - 12329 | 0 | . | 0 | 1 | neg |
| 1526 | | 1-palmitoyl- | Lipid | Phospholipid | 0 | 1 | pos |
| | | 2-oleoyl-GPE | | Metabolism | | | |
| | | (16:0/18:1) | | | | | |
| 1162 | | N-acetyl- | Carbo- | Aminosugar | 0 | 1 | pos |
| | | neuraminate | hydrate | Metabolism | | | |
| 112 | | 3-hydroxy- | Lipid | Mevalonate | 0 | 1 | pos |
| | | 3-methyl- | | Metabolism | | | |
| | | glutarate | | | | | |
| 100001851 | | N-acetylserine | Amino | Glycine, | 0 | 1 | pos |
| | | | Acid | Serine and | | | |
| | | | | Threonine | | | |
| | | | | Metabolism | | | |
| 100001399 | | 1,7- | Xeno- | Xanthine | 0 | 1 | pos |
| | | dimethylurate | biotics | Metabolism | | | |
| 480 | | proline | Amino | Urea cycle; | 0 | 1 | pos |
| | | | Acid | Arginine | | | |
| | | | | and Proline | | | |
| | | | | Metabolism | | | |
| 1268 | | gamma- | Peptide | Gamma- | 0 | 1 | pos |
| | | glutamyl- | | glutamyl | | | |
| | | leucine | | Amino Acid | | | |
| 100010922 | X - 24278 | linoleoyl- | Lipid | Diacylglycerol | 0 | 1 | pos |
| | | arachidonoyl- | | | | | |
| | | glycerol | | | | | |
| | | (18:2/20:4) | | | | | |
| | | [1]* | | | | | |
| X - 12844 | | X - 12846 | 0 | . | 0 | 1 | pos |
| 340 | | glycine | Amino | Glycine, | 0 | 1 | neg |
| | | | Acid | Serine and | | | |
| | | | | Threonine | | | |
| | | | | Metabolism | | | |
| X - 16580 | | X - 16580 | 0 | . | 0 | 1 | pos |
| 100009142 | | 1-stearoyl-2 | Lipid | Phospholipid | 0 | 1 | pos |
| | | docosapenta- | | Metabolism | | | |
| | | enoyl-GPC | | | | | |
| | | (18:0/22:5n6)* | | | | | |
| X - 21736 | | X - 21736 | 0 | . | 0 | 1 | pos |
| 100000406 | | ribitol | Carbo- | Pentose | 0 | 1 | pos |
| | | | hydrate | Metabolism | | | |
| 100001051 | | 1-methyl- | Amino | Histidine | 0 | 1 | pos |
| | | histidine | Acid | Metabolism | | | |
| 100001565 | | 2-docosahexa- | Lipid | Lysolipid | 0 | 1 | neg |
| | | enoyl-GPC | | | | | |
| | | (22:6)* | | | | | |
| X - 11805 | | X - 11838 | 0 | . | 0 | 1 | pos |
| 563 | | glutamine | Amino | Glutamate | 0 | 1 | neg |
| | | | Acid | Metabolism | | | |
| 100001295 | | gamma- | Peptide | Gamma- | 0 | 1 | pos |
| | | glutamyl- | | glutamyl | | | |
| | | tryptophan | | Amino Acid | | | |
| 100001416 | | orotidine | Nucleotide | Pyrimidine | 0 | 1 | pos |
| | | | | Metabolism, | | | |
| | | | | Orotate | | | |
| | | | | containing | | | |
| 100001083 | | indolepro- | Amino | Tryptophan | 0 | 1 | neg |
| | | pionate | Acid | Metabolism | | | |
| 100008993 | | 1-palmitoyl-2- | Lipid | Phospholipid | 0 | 1 | pos |
| | | arachidonoyl- | | Metabolism | | | |
| | | GPI (16:0/20:4)* | | | | | |
| 1002 | | allantoin | Nucleotide | Purine | 0 | 1 | pos |
| | | | | Metabolism, | | | |
| | | | | (Hypo)Xanthine/ | | | |
| | | | | Inosine | | | |
| | | | | containing | | | |
| 100001054 | | butyryl- | Lipid | Fatty Acid | 0 | 1 | pos |
| | | carnitine | | Metabolism | | | |
| | | | | (also BCAA | | | |
| | | | | Metabolism) | | | |
| 100009030 | X - 24065 | lactosyl-N- | Lipid | Sphingolipid | 0 | 1 | neg |
| | | palmitoyl- | | Metabolism | | | |
| | | sphingosine | | | | | |
| 197 | | S-adenosyl- | Amino | Methionine, | 0 | 1 | pos |
| | | homocysteine | Acid | Cysteine, | | | |
| | | (SAH) | | SAM and | | | |
| | | | | Taurine | | | |
| | | | | Metabolism | | | |
| 100001556 | | 2-oleoyl- | Lipid | Lysolipid | 0 | 1 | neg |
| | | GPC (18:1)* | | | | | |
| 100000665 | | docosahexa- | Lipid | Polyun- | 0 | 1 | neg |
| | | enoate (DHA; | | saturated | | | |
| | | 22:6n3) | | Fatty Acid | | | |
| | | | | (n3 and n6) | | | |
| 297 | | sphingosine | Lipid | Sphingolipid | 0 | 1 | pos |
| | | | | Metabolism | | | |
| 100006121 | | 1-dihomo- | Lipid | Monoacylglycerol | 0 | 1 | incon- |
| | | linolenyl- | | | | | sistent |
| | | glycerol (20:3) | | | | | |
| 100000924 | | 1-oleoyl- | Lipid | Monoacylglycerol | 0 | 1 | pos |
| | | glycerol (18:1) | | | | | |
| 100002028 | | 4-androsten- | Lipid | Steroid | 0 | 1 | pos |
| | | 3beta,17beta-diol | | | | | |
| | | monosulfate (1) | | | | | |
| 100008984 | | 1-palmitoyl-2- | Lipid | Phospholipid | 0 | 1 | pos |
| | | palmitoleoyl- | | Metabolism | | | |
| | | GPC | | | | | |
| | | (16:0/16:1)* | | | | | |
| 100019978 | X - 11538 | octadecene | 0 | . | 0 | 1 | neg |
| | | dioate | | | | | |
| | | (C18:1-DC) | | | | | |
| 100009132 | | 1-linoleoyl- | Lipid | Phospholipid | 0 | 1 | neg |
| | | 2-docosahexa- | | Metabolism | | | |
| | | enoyl-GPC | | | | | |
| | | (18:2/22:6)* | | | | | |
| 100009350 | | 1-oleoyl-2- | Lipid | Phospholipid | 0 | 1 | neg |
| | | dihomo- | | Metabolism | | | |
| | | linoleoyl- | | | | | |
| | | GPC | | | | | |
| | | (18:1/20:2)* | | | | | |
| X - 15245 | | X - 15245 | 0 | . | 0 | 1 | pos |
| 100002107 | | palmitoyl | Lipid | Sphingolipid | 0 | 1 | neg |
| | | sphingomyelin | | Metabolism | | | |
| | | (d18:1/16:0) | | | | | |
| 100008928 | | 2-hydroxy- | Amino | Methionine, | 0 | 1 | pos |
| | | butyrate/ | Acid | Cysteine, | | | |
| | | 2-hydroxy- | | SAM and | | | |
| | | isobutyrate | | Taurine | | | |
| | | | | Metabolism | | | |
| 100002103 | X - 12686 | 5-methyl- | 0 | . | 0 | 1 | pos |
| | | thioribose** | | | | | |
| 100009066 | | 1-palmitoyl- | Lipid | Phospholipid | 0 | 1 | pos |
| | | 2-oleoyl-GPI | | Metabolism | | | |
| | | (16:0/18:1)* | | | | | |
| 100002018 | | 5alpha- | Lipid | Steroid | 0 | 1 | pos |
| | | androstan- | | | | | |
| | | 3alpha,17beta-diol | | | | | |
| | | monosulfate (1) | | | | | |
| 1528 | | 1-palmitoyl- | Lipid | Phospholipid | 0 | 1 | pos |
| | | 2-linoleoyl- | | Metabolism | | | |
| | | GPI | | | | | |
| | | (16:0/18:2) | | | | | |
| 100001456 | | 7- | Nucleotide | Purine | 0 | 1 | pos |
| | | methylguanine | | Metabolism, | | | |
| | | | | Guanine | | | |
| | | | | containing | | | |
| X - 17179 | | X - 17179 | 0 | . | 0 | 1 | pos |
| 100001869 | | 1-stearoyl-2- | Lipid | Phospholipid | 0 | 1 | pos |
| | | arachidonoyl- | | Metabolism | | | |
| | | GPC | | | | | |
| | | (18:0/20:4) | | | | | |
| 100004542 | | 2-amino- | Lipid | Fatty Acid, | 0 | 1 | pos |
| | | heptanoate | | Amino | | | |
| 100001552 | | 1-dihomo- | Lipid | Lysolipid | 0 | 1 | pos |
| | | linolenoyl- | | | | | |
| | | GPC | | | | | |
| | | (20:3n3 or 6)* | | | | | |
| 1025 | | pipecolate | Amino | Lysine | 0 | 1 | neg |
| | | | Acid | Metabolism | | | |
| 100001435 | | 1-linolenoyl- | Lipid | Monoacylglycerol | 0 | 1 | pos |
| | | glycerol (18:3) | | | | | |
| 100000257 | | glucuronate | Carbo- | Aminosugar | 0 | 1 | pos |
| | | | hydrate | Metabolism | | | |
| 100001618 | | 1-myristoyl- | Lipid | Monoacylglycerol | 0 | 1 | pos |
| | | glycerol (14:0) | | | | | |
| 100001925 | | cyclo(leu-pro) | Peptide | Dipeptide | 0 | 1 | pos |
| 100009153 | | 1-stearoyl- | Lipid | phospholipid | 0 | 1 | pos |
| | | 2-meadoyl-GPC | | | | | |
| | | (18:0/20:3n9)* | | | | | |
| 100008977 | | 1-stearoyl-2- | Lipid | Phospholipid | 0 | 1 | pos |
| | | arachidonoyl- | | Metabolism | | | |
| | | GPE | | | | | |
| | | (18:0/20:4) | | | | | |
| 100001126 | | gamma- | Peptide | Gamma- | 0 | 1 | pos |
| | | glutamylvaline | | glutamyl | | | |
| | | | | Amino Acid | | | |
| X - 14838 | | X - 14838 | 0 | . | 0 | 1 | pos |
| 338 | | gluconate | Xeno- | Food | 0 | 1 | pos |
| | | | biotics | Component/ | | | |
| | | | | Plant | | | |
| 100008992 | | 1-stearoyl-2- | Lipid | Phospholipid | 0 | 1 | pos |
| | | docosahexa- | | Metabolism | | | |
| | | enoyl-GPE | | | | | |
| | | (18:0/22:6)* | | | | | |
| 100001254 | | N-acetyl- | Amino | Tryptophan | 0 | 1 | pos |
| | | tryptophan | Acid | Metabolism | | | |
| 100009004 | | 1-(1-enyl- | Lipid | Phospholipid | 0 | 1 | neg |
| | | stearoyl)-2- | | Metabolism | | | |
| | | docosahexa- | | | | | |
| | | enoyl-GPE | | | | | |
| | | (P-18:0/22:6)* | | | | | |
| 100001652 | | 2-palmitoyl- | Lipid | Lysolipid | 0 | 1 | neg |
| | | GPE (16:0)* | | | | | |
| 100004243 | | gamma-CEHC | Cofactors | Tocopherol | 0 | 1 | pos |
| | | glucuronide* | and | Metabolism | | | |
| | | | Vitamins | | | | |
| 100000961 | | homoarginine | Amino | Urea cycle; | 0 | 1 | pos |
| | | | Acid | Arginine | | | |
| | | | | and Proline | | | |
| | | | | Metabolism | | | |
| 100002769 | X - 12681 | argininate* | Amino | Urea cycle; | 0 | 1 | pos |
| | | | Acid | Arginine | | | |
| | | | | and Proline | | | |
| | | | | Metabolism | | | |
| X - 23593 | | X - 23593 | 0 | . | 0 | 1 | pos |
| 100008914 | | 1-palmitoyl-2- | Lipid | Phospholipid | 0 | 1 | pos |
| | | arachidonoyl- | | Metabolism | | | |
| | | GPC (16:0/20:4) | | | | | |
| 1090 | | bilirubin | Cofactors | Hemoglobin | 0 | 1 | neg |
| | | (Z,Z) | and | and Porphyrin | | | |
| | | | Vitamins | Metabolism | | | |
| X - 21626 | | X - 21626 | 0 | . | 0 | 1 | pos |
| 100001208 | | 1-methyl- | Amino | Histidine | 0 | 1 | pos |
| | | imidazole- | Acid | Metabolism | | | |
| | | acetate | | | | | |
| 93 | | alpha- | Energy | TCA Cycle | 0 | 1 | pos |
| | | ketoglutarate | | | | | |
| 100001567 | | 1-palmitoyl- | Lipid | Lysolipid | 0 | 1 | neg |
| | | GPE (16:0) | | | | | |
| 1104 | | methyl | Xeno- | Food | 0 | 1 | pos |
| | | indole-3- | biotics | Component/ | | | |
| | | acetate | | Plant | | | |
| 100008998 | | gamma-tocopherol/ | Cofactors | Tocopherol | 0 | 1 | pos |
| | | beta-tocopherol | and | Metabolism | | | |
| | | | Vitamins | | | | |
| X - 12100 | | X - 12101 | 0 | . | 0 | 1 | pos |
| X - 14056 | | X - 14056 | 0 | . | 0 | 1 | pos |
| 100008915 | | 1-palmitoyl-2- | Lipid | Phospholipid | 0 | 1 | neg |
| | | docosahexa- | | Metabolism | | | |
| | | enoyl-GPC | | | | | |
| | | (16:0/22:6) | | | | | |
| 100008976 | | 1-stearoyl- | Lipid | Phospholipid | 0 | 1 | pos |
| | | 2-linoleoyl- | | Metabolism | | | |
| | | GPE | | | | | |
| | | (18:0/18:2)* | | | | | |
| X - 21339 | | X - 21339 | 0 | . | 0 | 1 | pos |
| 100003001 | | 1-(1-enyl- | Lipid | Lysolipid | 0 | 1 | neg |
| | | stearoyl)-GPE | | | | | |
| | | (P-18:0)* | | | | | |
| 100002876 | | 1-(1-enyl- | Lipid | Lysolipid | 0 | 1 | neg |
| | | oleoyl)-GPC | | | | | |
| | | (P-18:1)* | | | | | |
| 504 | | serotonin | Amino | Tryptophan | 0 | 1 | incon- |
| | | | Acid | Metabolism | | | sistent |
| 100008929 | | 2- | Energy | TCA Cycle | 0 | 1 | pos |
| | | methylcitrate/ | | | | | |
| | | homocitrate | | | | | |
| X - 16132 | | X - 16132 | 0 | . | 0 | 1 | pos |
| X - 11530 | | X - 11537 | 0 | . | 0 | 1 | neg |
| X - 12216 | | X - 12221 | 0 | . | 0 | 1 | neg |
| X - 17337 | | X - 17337 | 0 | . | 0 | 1 | pos |
| 100002063 | | 1-docosapenta- | Lipid | Lysolipid | 0 | 1 | neg |
| | | enoyl-GPC | | | | | |
| | | (22:5n3)* | | | | | |
| 1538 | | stearoyl | Lipid | Sphingolipid | 0 | 1 | pos |
| | | sphingomyelin | | Metabolism | | | |
| | | (d18:1/18:0) | | | | | |
| 100008921 | | 1-palmitoyl- | Lipid | Phospholipid | 0 | 1 | neg |
| | | 2-stearoyl- | | Metabolism | | | |
| | | GPC (16:0/18:0) | | | | | |
| 100001577 | | N-acetyl- | Amino | Urea cycle; | 0 | 1 | pos |
| | | citrulline | Acid | Arginine | | | |
| | | | | and Proline | | | |
| | | | | Metabolism | | | |
| X - 16123 | | X - 16123 | 0 | . | 0 | 1 | pos |
| 100000846 | | erythritol | Xeno- | Food | 0 | 1 | pos |
| | | | biotics | Component/ | | | |
| | | | | Plant | | | |
| 100004083 | | glyco- | Lipid | Secondary | 0 | 1 | neg |
| | | hyocholate | | Bile Acid | | | |
| | | | | Metabolism | | | |
| 1221 | | creatine | Amino | Creatine | 0 | 1 | pos |
| | | | Acid | Metabolism | | | |
| 100001951 | | bilirubin | Cofactors | Hemoglobin | 0 | 1 | neg |
| | | (E,Z or Z,E)* | and | and Porphyrin | | | |
| | | | Vitamins | Metabolism | | | |
| 806 | | dimethyl- | Amino | Glycine, | 0 | 1 | pos |
| | | glycine | Acid | Serine and | | | |
| | | | | Threonine | | | |
| | | | | Metabolism | | | |
| X - 24061 | | PC(O- | 0 | . | 0 | 1 | neg |
| | | 16:0/16:0) | | | | | |
| 100001553 | | 1-dihomo- | Lipid | Lysolipid | 0 | 1 | neg |
| | | linoleoyl- | | | | | |
| | | GPC (20:2)* | | | | | |
| 100002293 | | phenylalanyl- | Peptide | Dipeptide | 0 | 1 | pos |
| | | phenylalanine | | | | | |
| 100001320 | | erythronate* | Carbo- | Aminosugar | 0 | 1 | pos |
| | | | hydrate | Metabolism | | | |
| 100000616 | | 1-stearoyl-2- | Lipid | Phospholipid | 0 | 1 | pos |
| | | arachidonoyl- | | Metabolism | | | |
| | | GPI | | | | | |
| | | (18:0/20:4) | | | | | |
| 100001590 | | isobutyryl- | Amino | Leucine, | 0 | 1 | pos |
| | | glycine | Acid | Isoleucine | | | |
| | | | | and Valine | | | |
| | | | | Metabolism | | | |
| 100001765 | | 3- | Lipid | Fatty Acid, | 0 | 1 | neg |
| | | methyladipate | | Dicarboxylate | | | |
| X - 11522 | | X - 11530 | 0 | . | 0 | 1 | neg |
| 100001776 | | 2-linoleoyl- | Lipid | Lysolipid | 0 | 1 | neg |
| | | GPE (18:2)* | | | | | |
| 100000840 | | tartronate | Xeno- | Bacterial/ | 0 | 1 | neg |
| | | (hydroxy- | biotics | Fungal | | | |
| | | malonate) | | | | | |
| 923 | | dihydroorotate | Nucleotide | Pyrimidine | 0 | 1 | incon- |
| | | | | Metabolism, | | | sistent |
| | | | | Orotate | | | |
| | | | | containing | | | |
| 100001446 | | 5- | Nucleotide | Pyrimidine | 0 | 1 | incon- |
| | | methyluridine | | Metabolism, | | | sistent |
| | | (ribothymidine) | | Uracil | | | |
| | | | | containing | | | |
| 100001271 | | 1-stearoyl- | Lipid | Lysolipid | 0 | 1 | incon- |
| | | GPC (18:0) | | | | | sistent |
| 100001731 | | indoleacetyl | Amino | Tryptophan | 0 | 1 | pos |
| | | glutamine | Acid | Metabolism | | | |
| 100002061 | | 2-docosahexa- | Lipid | Lysolipid | 0 | 1 | neg |
| | | enoyl-GPE | | | | | |
| | | (22:6)* | | | | | |
| 100000282 | | N- | Amino | Glutamate | 0 | 1 | pos |
| | | acetylglutamate | Acid | Metabolism | | | |
| 100000841 | | oxalate | Cofactors | Ascorbate | 0 | 1 | neg |
| | | (ethanedioate) | and | and | | | |
| | | | Vitamins | Aldarate | | | |
| | | | | Metabolism | | | |
| 100002154 | | ergothioneine | Xeno- | Food | 0 | 1 | neg |
| | | | biotics | Component/ | | | |
| | | | | Plant | | | |
| 100008954 | | palmitoyl | Lipid | Sphingolipid | 0 | 1 | incon- |
| | | dihydro- | | Metabolism | | | sistent |
| | | sphingomyelin | | | | | |
| | | (d18:0/16:0)* | | | | | |
| 100001609 | | 7-alpha- | Lipid | Sterol | 0 | 1 | pos |
| | | hydroxy- | | | | | |
| | | 3-oxo-4- | | | | | |
| | | cholestenoate | | | | | |
| | | (7-Hoca) | | | | | |
| 100001102 | | dodecanedioate | Lipid | Fatty Acid, | 0 | 1 | neg |
| | | | | Dicarboxylate | | | |
| 100001263 | | 1-palmitoyl- | Lipid | Lysolipid | 0 | 1 | incon- |
| | | GPC (16:0) | | | | | sistent |
| 2050 | | eicosapenta- | Lipid | Polyun- | 0 | 1 | neg |
| | | enoate | | saturated | | | |
| | | (EPA; 20:5n3) | | Fatty Acid | | | |
| | | | | (n3 and n6) | | | |
| 100002784 | X - 12339 | 2- | Amino | Urea cycle; | 0 | 1 | pos |
| | | oxoarginine* | Acid | Arginine | | | |
| | | | | and Proline | | | |
| | | | | Metabolism | | | |
| 100002877 | | 1-(1-enyl- | Lipid | Phospholipid | 0 | 1 | neg |
| | | stearoyl)-GPC | | Metabolism | | | |
| | | (P-18:0)* | | | | | |
| 100001007 | | ribonate | Carbo- | Pentose | 0 | 1 | pos |
| | | | hydrate | Metabolism | | | |
| 100005466 | | N- | Amino | Methionine, | 0 | 1 | pos |
| | | acetyltaurine | Acid | Cysteine, | | | |
| | | | | SAM and | | | |
| | | | | Taurine | | | |
| | | | | Metabolism | | | |
| 100001593 | | glutaryl- | Amino | Lysine | 0 | 1 | pos |
| | | carnitine (C5) | Acid | Metabolism | | | |
| 100001740 | | mannitol/ | Carbo- | Fructose, | 0 | 1 | pos |
| | | sorbitol | hydrate | Mannose and | | | |
| | | | | Galactose | | | |
| | | | | Metabolism | | | |
| 100001398 | | 3,7- | Xeno- | Xanthine | 0 | 1 | pos |
| | | dimethylurate | biotics | Metabolism | | | |
| 100006056 | | N- | Amino | Phenylalanine | 0 | 1 | pos |
| | | formylphenyl- | Acid | and Tyrosine | | | |
| | | alanine | | Metabolism | | | |
| 100001579 | | 2-hydroxy- | Lipid | Fatty Acid, | 0 | 1 | neg |
| | | palmitate | | Monohydroxy | | | |
| 100002528 | | sulfate* | Xeno- | Chemical | 0 | 1 | pos |
| | | | biotics | | | | |
| 100000657 | | 1,2-dipalmitoyl- | Lipid | Phospholipid | 0 | 1 | neg |
| | | GPC (16:0/16:0) | | Metabolism | | | |
| 100009394 | X - 12450 | hexadecadienoate | Lipid | Polyun- | 0 | 1 | incon- |
| | | (16:2n6) | | saturated | | | sistent |
| | | | | Fatty Acid | | | |
| | | | | (n3 and n6) | | | |
| 302 | | deoxycholate | Lipid | Secondary | 0 | 1 | pos |
| | | | | Bile Acid | | | |
| | | | | Metabolism | | | |
| 1052 | | glycerate | Carbo- | Glycolysis, | 0 | 1 | neg |
| | | | hydrate | Gluconeo- | | | |
| | | | | genesis, and | | | |
| | | | | Pyruvate | | | |
| | | | | Metabolism | | | |
| 888 | | caprate | Lipid | Medium | 0 | 1 | neg |
| | | (10:0) | | Chain Fatty | | | |
| | | | | Acid | | | |
| X - 24021 | | lysoPE(O-16:0) | 0 | . | 0 | 1 | neg |
| 100000611 | | 1-palmityl- | Lipid | Lysolipid | 0 | 1 | neg |
| | | GPC (O- | | | | | |
| | | 16:0) | | | | | |
| 1094 | | thyroxine | Amino | Phenylalanine | 0 | 1 | pos |
| | | | Acid | and Tyrosine | | | |
| | | | | Metabolism | | | |
| 100001433 | | 1-arachidonyl | Lipid | Monoacylglycerol | 0 | 1 | pos |
| | | glycerol (20:4) | | | | | |
| 100001710 | | leucylleucine | Peptide | Dipeptide | 0 | 1 | pos |
| 452 | | palmitoleate | Lipid | Long Chain | 0 | 1 | pos |
| | | (16:1n7) | | Fatty Acid | | | |
| 100009002 | | 1-(1-enyl- | Lipid | Plasmalogen | 0 | 1 | pos |
| | | palmitoyl)-2- | | | | | |
| | | arachidonoyl- | | | | | |
| | | GPE (P- | | | | | |
| | | 16:0/20:4)* | | | | | |
| 100001570 | | 1-linoleoyl- | Lipid | Lysolipid | 0 | 1 | neg |
| | | GPE (18:2)* | | | | | |
| 100004552 | | 1-eicosapenta- | Lipid | Lysolipid | 0 | 1 | incon- |
| | | enoyl-GPE | | | | | sistent |
| | | (20:5)* | | | | | |
| 252 | | succinate | Energy | TCA Cycle | 0 | 1 | incon- |
| | | | | | | | sistent |
| 100002113 | | cysteine | Amino | Methionine, | 0 | 1 | pos |
| | | sulfinic acid | Acid | Cysteine, | | | |
| | | | | SAM and | | | |
| | | | | Taurine | | | |
| | | | | Metabolism | | | |
| 100001155 | | 2-methyl- | Amino | Leucine, | 0 | 1 | pos |
| | | butyrylglycine | Acid | Isoleucine | | | |
| | | | | and Valine | | | |
| | | | | Metabolism | | | |
| 144 | | 4-hydroxy- | Amino | Phenylalanine | 0 | 1 | pos |
| | | phenylacetate | Acid | and Tyrosine | | | |
| | | | | Metabolism | | | |
| 100001843 | | gamma- | Peptide | Gamma- | 0 | 1 | pos |
| | | glutamylalanine | | glutamyl | | | |
| | | | | Amino Acid | | | |
| 100002241 | | 7- | Xeno- | Xanthine | 0 | 1 | pos |
| | | methylurate | biotics | Metabolism | | | |
| 1004 | | xanthine | Nucleotide | Purine | 0 | 1 | pos |
| | | | | Metabolism, | | | |
| | | | | (Hypo)Xanthine/ | | | |
| | | | | Inosine | | | |
| | | | | containing | | | |
| 100009149 | | 1-oleoyl-2- | Lipid | phospholipid | 0 | 1 | incon- |
| | | eicosapenta- | | | | | sistent |
| | | enoyl-GPC | | | | | |
| | | (18:1/20:5)* | | | | | |
| 1083 | | N-acetyl- | Amino | Methionine, | 0 | 1 | pos |
| | | methionine | Acid | Cysteine, | | | |
| | | | | SAM and | | | |
| | | | | Taurine | | | |
| | | | | Metabolism | | | |
| 100006292 | | sphingomyelin | Lipid | Sphingolipid | 0 | 1 | pos |
| | | (d18:1/20:1, | | Metabolism | | | |
| | | d18:2/20:0)* | | | | | |
| 100009138 | | 1-myristoyl- | Lipid | Phospholipid | 0 | 1 | pos |
| | | 2-linoleoyl- | | Metabolism | | | |
| | | GPC | | | | | |
| | | (14:0/18:2)* | | | | | |
| 100002060 | | 1-docosahexa- | Lipid | Lysolipid | 0 | 1 | neg |
| | | enoyl-GPE | | | | | |
| | | (22:6)* | | | | | |
| 100003678 | | prolylproline | Peptide | Dipeptide | 0 | 1 | pos |
| X - 13737 | | X - 13737 | 0 | . | 0 | 1 | pos |
| 100001461 | | 1-stearoyl- | Lipid | Lysolipid | 0 | 1 | incon- |
| | | GPE (18:0) | | | | | sistent |
| 100009166 | | phosphocholine | Lipid | phospholipid | 0 | 1 | incon- |
| | | (16:0/22:5n3, | | | | | sistent |
| | | 18:1/20:4)* | | | | | |
| 100001153 | | 2- | Lipid | Fatty Acid, | 0 | 1 | pos |
| | | hydroxyadipate | | Dicarboxylate | | | |
| 100004499 | | 6- | Xeno- | Drug | 0 | 1 | pos |
| | | oxopiperidine- | biotics | | | | |
| | | 2-carboxylic | | | | | |
| | | acid | | | | | |
| 1239 | | 2- | Lipid | Fatty Acid, | 0 | 1 | neg |
| | | hydroxystearate | | Monohydroxy | | | |
| 100001400 | | 1- | Xeno- | Xanthine | 0 | 1 | pos |
| | | methylurate | biotics | Metabolism | | | |
| 100009036 | | 1-margaroyl- | Lipid | Phospholipid | 0 | 1 | neg |
| | | 2-oleoyl-GPC | | Metabolism | | | |
| | | (17:0/18:1)* | | | | | |
| 100000936 | | 3-methyl-2- | Amino | Leucine, | 0 | 1 | pos |
| | | oxobutyrate | Acid | Isoleucine | | | |
| | | | | and Valine | | | |
| | | | | Metabolism | | | |
| 100001262 | | gamma- | Peptide | Gamma- | 0 | 1 | pos |
| | | glutamyl- | | glutamyl | | | |
| | | epsilon- | | Amino Acid | | | |
| | | lysine | | | | | |
| 100008918 | | 1-(1-enyl- | Lipid | Phospholipid | 0 | 1 | incon- |
| | | stearoyl)-2- | | Metabolism | | | sistent |
| | | arachidonoyl- | | | | | |
| | | GPC (P- | | | | | |
| | | 18:0/20:4) | | | | | |
| 100010901 | X - 24240 | gamma- | Peptide | Gamma- | 0 | 1 | incon- |
| | | glutamyl- | | glutamyl | | | sistent |
| | | alpha- | | Amino Acid | | | |
| | | lysine | | | | | |
| 100000036 | | 3-methyl-2- | Amino | Leucine, | 0 | 1 | pos |
| | | oxovalerate | Acid | Isoleucine | | | |
| | | | | and Valine | | | |
| | | | | Metabolism | | | |
| 267 | | choline | Lipid | Phospholipid | 0 | 1 | neg |
| | | phosphate | | Metabolism | | | |
| 100009331 | | oleoylcholine | Lipid | Phospholipid | 0 | 0 | neg |
| | | | | Metabolism | | | |
| 100003677 | | prolylphenyl- | Peptide | Dipeptide | 0 | 0 | pos |
| | | alanine | | | | | |
| 100009134 | | 1-palmityl- | Lipid | Plasmalogen | 0 | 0 | neg |
| | | 2-linoleoyl- | | | | | |
| | | GPC (O-16:0/18:2) | | | | | |
| X - 11787 | | X - 11795 | 0 | . | 0 | 0 | pos |
| 100015684 | | myristoyl- | Lipid | Diacylglycerol | 0 | 0 | pos |
| | | linoleoyl- | | | | | |
| | | glycerol | | | | | |
| | | (14:0/18:2) | | | | | |
| | | [2]* | | | | | |
| 100002873 | | 1-lignoceroyl- | Lipid | Lysolipid | 0 | 0 | neg |
| | | GPC (24:0) | | | | | |
| 100010935 | | diacylglycerol | Lipid | Diacylglycerol | 0 | 0 | pos |
| | | (14:0/18:1, | | | | | |
| | | 16:0/16:1) | | | | | |
| | | [2]* | | | | | |
| 100009361 | | 1-oleoyl-2- | Lipid | Phospholipid | 0 | 0 | pos |
| | | docosapenta- | | Metabolism | | | |
| | | enoyl-GPC | | | | | |
| | | (18:1/22:5n6)* | | | | | |
| X - 24309 | | X - 24309 | 0 | . | 0 | 0 | pos |
| 100002749 | | S- | Amino | Methionine, | 0 | 0 | neg |
| | | methylcysteine | Acid | Cysteine, | | | |
| | | | | SAM and | | | |
| | | | | Taurine | | | |
| | | | | Metabolism | | | |
| 189 | | N6,N6,N6- | Amino | Lysine | 0 | 0 | pos |
| | | trimethyllysine | Acid | Metabolism | | | |
| 100015838 | | eicosenoyl- | Lipid | Fatty Acid | 0 | 0 | neg |
| | | carnitine | | Metabolism | | | |
| | | (C20:1)* | | (Acyl | | | |
| | | | | Carnitine) | | | |
| 100005396 | | 5alpha-androstan- | Lipid | Steroid | 0 | 0 | pos |
| | | 3alpha,17beta-diol- | | | | | |
| | | 17-glucosiduronate | | | | | |
| 100010940 | | diacylglycerol | Lipid | Diacylglycerol | 0 | 0 | pos |
| | | (16:1/18:2 | | | | | |
| | | [2], | | | | | |
| | | 16:0/18:3 | | | | | |
| | | [1])* | | | | | |
| 100009347 | | phosphatidyl- | Lipid | Phospholipid | 0 | 0 | pos |
| | | choline | | Metabolism | | | |
| | | (15:0/18:1, | | | | | |
| | | 17:0/16:1)* | | | | | |
| 100006614 | | adipoylcarnitine | Lipid | Fatty Acid | 0 | 0 | pos |
| | | (C6- | | Metabolism | | | |
| | | DC) | | (Acyl | | | |
| | | | | Carnitine) | | | |
| X - 21628 | | X - 21628 | 0 | . | 0 | 0 | pos |
| 100001127 | | pyroglutamyl- | Peptide | Dipeptide | 0 | 0 | pos |
| | | glycine | | | | | |
| 100001413 | | N4- | Nucleotide | Pyrimidine | 0 | 0 | pos |
| | | acetylcytidine | | Metabolism, | | | |
| | | | | Cytidine | | | |
| | | | | containing | | | |
| 100009026 | | behenoyl | Lipid | Sphingolipid | 0 | 0 | pos |
| | | dihydro- | | Metabolism | | | |
| | | sphingomyelin | | | | | |
| | | (d18:0/22:0)* | | | | | |
| 100010937 | | oleoyl- | Lipid | Diacylglycerol | 0 | 0 | pos |
| | | arachidonoyl- | | | | | |
| | | glycerol | | | | | |
| | | (18:1/20:4) | | | | | |
| | | [2]* | | | | | |
| 100010936 | | oleoyl- | Lipid | Diacylglycerol | 0 | 0 | pos |
| | | arachidonoyl- | | | | | |
| | | glycerol | | | | | |
| | | (18:1/20:4) | | | | | |
| | | [1]* | | | | | |
| 310 | | cystathionine | Amino | Methionine, | 0 | 0 | pos |
| | | | Acid | Cysteine, | | | |
| | | | | SAM and | | | |
| | | | | Taurine | | | |
| | | | | Metabolism | | | |
| 100000943 | | 2- | Lipid | Monoacylglycerol | 0 | 0 | pos |
| | | oleoylglycerol | | | | | |
| | | (18:1) | | | | | |
| 100003926 | | hydroxybutyryl- | Lipid | Fatty Acid | 0 | 0 | pos |
| | | carnitine* | | Metabolism | | | |
| | | | | (Acyl | | | |
| | | | | Carnitine) | | | |
| 141 | | gamma- | Amino | Glutamate | 0 | 0 | neg |
| | | aminobutyrate | Acid | Metabolism | | | |
| | | (GABA) | | | | | |
| X - 11441 | | X - 11442 | 0 | . | 0 | 0 | neg |
| 100002927 | | S- | Amino | Methionine, | 0 | 0 | neg |
| | | methylcysteine | Acid | Cysteine, | | | |
| | | sulfoxide | | SAM and | | | |
| | | | | Taurine | | | |
| | | | | Metabolism | | | |
| 100000269 | | glycerophos- | Lipid | Phospholipid | 0 | 0 | neg |
| | | phorylcholine | | Metabolism | | | |
| | | (GPC) | | | | | |
| 100009027 | | sphingomyelin | Lipid | Sphingolipid | 0 | 0 | pos |
| | | (d18:0/18:0, | | Metabolism | | | |
| | | d19:0/17:0)* | | | | | |
| 100010917 | | palmitoyl- | Lipid | Diacylglycerol | 0 | 0 | pos |
| | | oleoyl- | | | | | |
| | | glycerol | | | | | |
| | | (16:0/18:1) | | | | | |
| | | [2]* | | | | | |
| 1001 | | trans-4- | Amino | Urea cycle; | 0 | 0 | pos |
| | | hydroxyproline | Acid | Arginine | | | |
| | | | | and Proline | | | |
| | | | | Metabolism | | | |
| 100000453 | | paraxanthine | Xeno- | Xanthine | 0 | 0 | pos |
| | | | biotics | Metabolism | | | |
| 100000827 | | 1-palmitoyl- | Lipid | Monoacylglycerol | 0 | 0 | pos |
| | | glycerol (16:0) | | | | | |
| 882 | | thymine | Nucleotide | Pyrimidine | 0 | 0 | pos |
| | | | | Metabolism, | | | |
| | | | | Thymine | | | |
| | | | | containing | | | |
| 100001033 | | beta- | Lipid | Sterol | 0 | 0 | neg |
| | | sitosterol | | | | | |
| 100001327 | | HWESASXX* | Peptide | Polypeptide | 0 | 0 | pos |
| 100001193 | | adrenate | Lipid | Polyun- | 0 | 0 | pos |
| | | (22:4n6) | | saturated | | | |
| | | | | Fatty Acid | | | |
| | | | | (n3 and n6) | | | |
| 100010916 | | palmitoyl- | Lipid | Diacylglycerol | 0 | 0 | pos |
| | | oleoyl- | | | | | |
| | | glycerol | | | | | |
| | | (16:0/18:1) | | | | | |
| | | [1]* | | | | | |
| 849 | | caffeine | Xeno- | Xanthine | 0 | 0 | pos |
| | | | biotics | Metabolism | | | |
| 100001452 | | isovaleryl- | Amino | Leucine, | 0 | 0 | incon- |
| | | glycine | Acid | Isoleucine | | | sistent |
| | | | | and Valine | | | |
| | | | | Metabolism | | | |
| 100009375 | | 1-linoleoyl- | Lipid | Phospholipid | 0 | 0 | neg |
| | | 2-docosapenta- | | Metabolism | | | |
| | | enyol-GPC | | | | | |
| | | (18:2/22:5n3)* | | | | | |
| 1140 | | gamma- | Peptide | Gamma- | 0 | 0 | neg |
| | | glutamyl- | | glutamyl | | | |
| | | glutamine | | Amino Acid | | | |
| X - 24241 | | X - 24241 | 0 | . | 0 | 0 | pos |
| X - 16935 | | X - 16935 | 0 | . | 0 | 0 | pos |
| 100015786 | | sphingomyelin | Lipid | Sphingolipid | 0 | 0 | pos |
| | | (d18:0/20:0, | | Metabolism | | | |
| | | d16:0/22:0)* | | | | | |
| 100000445 | | theobromine | Xeno- | Xanthine | 0 | 0 | pos |
| | | | biotics | Metabolism | | | |
| X - 12822 | | X - 12824 | 0 | . | 0 | 0 | pos |
| X - 21737 | | X - 21737 | 0 | . | 0 | 0 | neg |
| X - 15728 | | X - 15728 | 0 | . | 0 | 0 | neg |
| 100015620 | | lactosyl-N- | Lipid | Ceramides | 0 | 0 | neg |
| | | nervonoyl- | | | | | |
| | | sphingosine | | | | | |
| | | (d18:1/24:1)* | | | | | |
| X - 12798 | | X - 12816 | 0 | . | 0 | 0 | pos |
| 1547 | | N-stearoyl- | Lipid | Ceramides | 0 | 0 | pos |
| | | sphingosine | | | | | |
| | | (d18:1/18:0)* | | | | | |
| 275 | | creatinine | Amino | Creatine | 0 | 0 | pos |
| | | | Acid | Metabolism | | | |
| 100015683 | | myristoyl- | Lipid | Diacylglycerol | 0 | 0 | pos |
| | | linoleoyl- | | | | | |
| | | glycerol | | | | | |
| | | (14:0/18:2) | | | | | |
| | | [1]* | | | | | |
| 100000715 | | 1- | Amino | Guanidino and | 0 | 0 | pos |
| | | methylguanidine | Acid | Acetamido | | | |
| | | | | Metabolism | | | |
| 100004299 | | N-acetyl-1- | Amino | Histidine | 0 | 0 | pos |
| | | methylhistidine* | Acid | Metabolism | | | |
| X - 18914 | | X - 18914 | 0 | . | 0 | 0 | pos |
| 100000015 | | xanthurenate | Amino | Tryptophan | 0 | 0 | pos |
| | | | Acid | Metabolism | | | |
| 2048 | | 3-(N-acetyl- | Xeno- | Drug | 0 | 0 | pos |
| | | L-cystein-S-yl) | biotics | | | | |
| | | acetaminophen | | | | | |
| 100003637 | | valylarginine | Peptide | Dipeptide | 0 | 0 | neg |
| X - 11372 | | X - 11381 | 0 | . | 0 | 0 | pos |
| 100002254 | | stearoyl | Lipid | Endocannabinoid | 0 | 0 | pos |
| | | ethanolamide | | | | | |
| 100000580 | | 1,5- | Carbo- | Glycolysis, | 0 | 0 | pos |
| | | anhydroglucitol | hydrate | Gluconeo- | | | |
| | | (1,5-AG) | | genesis, and | | | |
| | | | | Pyruvate | | | |
| | | | | Metabolism | | | |
| 100009028 | | N- | Lipid | Sphingolipid | 0 | 0 | pos |
| | | palmitoyl- | | Metabolism | | | |
| | | sphinganine | | | | | |
| | | (d18:0/16:0) | | | | | |
| 100001510 | | phenol | Amino | Phenylalanine | 0 | 0 | pos |
| | | sulfate | Acid | and Tyrosine | | | |
| | | | | Metabolism | | | |
| 100003520 | | serylalanine | Peptide | Dipeptide | 0 | 0 | pos |
| X - 11442 | | X - 11444 | 0 | . | 0 | 0 | neg |
| X - 11838 | | 3-(Methylthio) | 0 | . | 0 | 0 | pos |
| | | acetaminophen | | | | | |
| | | sulfate | | | | | |
| 100000776 | | palmitoyl- | Lipid | Fatty Acid | 0 | 0 | pos |
| | | carnitine | | Metabolism | | | |
| | | | | (Acyl | | | |
| | | | | Carnitine) | | | |
| 100001278 | | 10- | Lipid | Long Chain | 0 | 0 | pos |
| | | heptadecenoate | | Fatty Acid | | | |
| | | (17:1n7) | | | | | |
| 100000711 | | 4-acetylphenol | Xeno- | Drug | 0 | 0 | neg |
| | | sulfate | biotics | | | | |
| 100002717 | | hydroxycotinine | Xeno- | Tobacco | 0 | 0 | incon- |
| | | | biotics | Metabolite | | | sistent |
| 100009035 | | 1- | Lipid | Phospholipid | 0 | 0 | incon- |
| | | pentadecanoyl- | | Metabolism | | | sistent |
| | | 2-linoleoyl- | | | | | |
| | | GPC | | | | | |
| | | (15:0/18:2)* | | | | | |
| X - 14364 | | pyr-phe* | 0 | . | 0 | 0 | pos |
| 100006370 | | 3beta-hydroxy- | Lipid | Sterol | 0 | 0 | neg |
| | | 5-cholestenoate | | | | | |
| 100009360 | | 1-oleoyl-2- | Lipid | Phospholipid | 0 | 0 | neg |
| | | docosapenta- | | Metabolism | | | |
| | | enoyl-GPC | | | | | |
| | | (18:1/22:5n3)* | | | | | |
| 100002462 | | 5- | Amino | Lysine | 0 | 0 | pos |
| | | (galactosyl | Acid | Metabolism | | | |
| | | hydroxy)-L- | | | | | |
| | | lysine | | | | | |
| 100001768 | | N6-carboxy- | Carbo- | Advanced | 0 | 0 | pos |
| | | methyllysine | hydrate | Glycation | | | |
| | | | | End-product | | | |
| 100000054 | | 5- | Amino | Lysine | 0 | 0 | pos |
| | | hydroxylysine | Acid | Metabolism | | | |
| 100003434 | | imidazole | Amino | Histidine | 0 | 0 | pos |
| | | propionate | Acid | Metabolism | | | |
| 100001739 | | dihomo- | Lipid | Polyun- | 0 | 0 | pos |
| | | linolenate | | saturated | | | |
| | | (20:3n3 or | | Fatty Acid | | | |
| | | n6) | | (n3 and n6) | | | |
| 100001872 | | 1-stearoyl-2- | Lipid | Phosphatidyl- | 0 | 0 | pos |
| | | arachidonoyl- | | serine (PS) | | | |
| | | GPS | | | | | |
| | | (18:0/20:4) | | | | | |
| 100000966 | | pyroglutamyl- | Peptide | Dipeptide | 0 | 0 | incon- |
| | | glutamine | | | | | sistent |
| 100009343 | | 1-linoleoyl- | Lipid | Phospholipid | 0 | 0 | neg |
| | | 2-linolenoyl- | | Metabolism | | | |
| | | GPC | | | | | |
| | | (18:2/18:3)* | | | | | |
| X - 22162 | | X - 22162 | 0 | . | 0 | 0 | pos |
| 100002173 | | 1-pentadecanoyl- | Lipid | Lysolipid | 0 | 0 | neg |
| | | GPC (15:0)* | | | | | |
| 100001274 | | N- | Amino | Glycine, | 0 | 0 | pos |
| | | acetylthreonine | Acid | Serine and | | | |
| | | | | Threonine | | | |
| | | | | Metabolism | | | |
| X - 11847 | | X - 11849 | 0 | . | 0 | 0 | neg |
| 391 | | citrulline | Amino | Urea cycle; | 0 | 0 | neg |
| | | | Acid | Arginine | | | |
| | | | | and Proline | | | |
| | | | | Metabolism | | | |
| 100010925 | | palmitoyl- | Lipid | Diacylglycerol | 0 | 0 | pos |
| | | arachidonoyl- | | | | | |
| | | glycerol | | | | | |
| | | (16:0/20:4) | | | | | |
| | | [2]* | | | | | |
| 100015962 | X - 21365 | N-trimethyl | Amino | Lysine | 0 | 0 | pos |
| | | 5- | Acid | Metabolism | | | |
| | | aminovalerate | | | | | |
| 331 | | gamma- | Peptide | Gamma- | 0 | 0 | incon- |
| | | glutamyl- | | glutamyl | | | sistent |
| | | glutamate | | Amino Acid | | | |
| 100006430 | | arabitol/ | Carbo- | Pentose | 0 | 0 | pos |
| | | xylitol | hydrate | Metabolism | | | |
| 100000258 | | glycerol 3- | Lipid | Glycerolipid | 0 | 0 | neg |
| | | phosphate | | Metabolism | | | |
| 179 | | 9,10- | Lipid | Fatty Acid, | 0 | 0 | neg |
| | | DiHOME | | Dihydroxy | | | |
| X - 16946 | | X - 16946 | 0 | . | 0 | 0 | incon- |
| | | | | | | | sistent |
| 100009397 | | 1-linoleoyl- | Lipid | Phospholipid | 0 | 0 | neg |
| | | 2-eicosapenta- | | Metabolism | | | |
| | | enoyl-GPC | | | | | |
| | | (18:2/20:5)* | | | | | |
| 100009346 | | phosphatidyl- | Lipid | Phospholipid | 0 | 0 | pos |
| | | choline | | Metabolism | | | |
| | | (14:0/14:0, | | | | | |
| | | 16:0/12:0) | | | | | |
| 100000802 | | acetylcarnitine | Lipid | Fatty Acid | 0 | 0 | pos |
| | | | | Metabolism | | | |
| | | | | (Acyl | | | |
| | | | | Carnitine) | | | |
| 100015759 | X - 11871 | stearoylcholine* | Lipid | Fatty Acid | 0 | 0 | neg |
| | | | | Metabolism | | | |
| | | | | (Acyl | | | |
| | | | | Choline) | | | |
| 100015593 | | 1-stearoyl- | Lipid | Phosphatidyl- | 0 | 0 | pos |
| | | 2-docosapenta- | | ethanolamine | | | |
| | | enoyl-GPE | | (PE) | | | |
| | | (18:0/22:5n6)* | | | | | |
| 100001178 | | 3-carboxy- | Lipid | Fatty Acid, | 0 | 0 | neg |
| | | 4-methyl-5- | | Dicarboxylate | | | |
| | | propyl-2- | | | | | |
| | | furanpropanoate | | | | | |
| | | (CMPF) | | | | | |
| 100001580 | | docosapenta- | Lipid | Polyun- | 0 | 0 | pos |
| | | enoate | | saturated | | | |
| | | (n6 DPA; | | Fatty Acid | | | |
| | | 22:5n6) | | (n3 and n6) | | | |
| 100005351 | | 1-eicosapenta- | Lipid | Lysolipid | 0 | 0 | incon- |
| | | enoyl-GPC | | | | | sistent |
| | | (20:5)* | | | | | |
| 100009376 | | 1-(1-enyl- | Lipid | Plasmalogen | 0 | 0 | neg |
| | | oleoyl)-2- | | | | | |
| | | docosahexa- | | | | | |
| | | enoyl-GPE (P- | | | | | |
| | | 18:1/22:6)* | | | | | |
| 100005850 | X - 12855 | 3-methyl- | Amino | Lysine | 0 | 0 | pos |
| | | glutaryl- | Acid | Metabolism | | | |
| | | carnitine | | | | | |
| | | (2) | | | | | |
| X - 21448 | | X - 21448 | 0 | . | 0 | 0 | incon- |
| | | | | | | | sistent |
| 503 | | serine | Amino | Glycine, | 0 | 0 | neg |
| | | | Acid | Serine and | | | |
| | | | | Threonine | | | |
| | | | | Metabolism | | | |
| 100006260 | X - 21810 | 6- | Xeno- | Chemical | 0 | 0 | pos |
| | | hydroxyindole | biotics | | | | |
| | | sulfate | | | | | |
| 100001615 | | octadecane | Lipid | Fatty Acid, | 0 | 0 | neg |
| | | dioate | | Dicarboxylate | | | |
| 100004541 | | acisoga | Amino | Polyamine | 0 | 0 | pos |
| | | | Acid | Metabolism | | | |
| 356 | | cortisol | Lipid | Steroid | 0 | 0 | neg |
| 100001511 | | 1- | Lipid | Lysolipid | 0 | 0 | incon- |
| | | palmitoleoyl- | | | | | sistent |
| | | GPC (16:1)* | | | | | |
| 100001562 | | 2-palmitoyl- | Lipid | Lysolipid | 0 | 0 | incon- |
| | | GPC (16:0)* | | | | | sistent |
| 100001810 | | dimethyl- | Amino | Urea cycle; | 0 | 0 | incon- |
| | | arginine | Acid | Arginine | | | sistent |
| | | (SDMA + | | and Proline | | | |
| | | ADMA) | | Metabolism | | | |
| 100006651 | | 3,4-methyl- | Xeno- | Food | 0 | 0 | pos |
| | | eneheptanoate | biotics | Component/ | | | |
| | | | | Plant | | | |
| 100003179 | | leucylalanine | Peptide | Dipeptide | 0 | 0 | neg |
| X - 14939 | | X - 14939 | 0 | . | 0 | 0 | pos |
| 100001613 | | tetradecane | Lipid | Fatty Acid, | 0 | 0 | neg |
| | | dioate | | Dicarboxylate | | | |
| 100002356 | | 17- | Lipid | Fatty Acid, | 0 | 0 | incon- |
| | | methylstearate | | Branched | | | sistent |
| 100009333 | | docosahexa- | Lipid | Phospholipid | 0 | 0 | neg |
| | | enoylcholine | | Metabolism | | | |
| 100001040 | | 1-linoleoyl- | Lipid | Monoacylglycerol | 0 | 0 | pos |
| | | glycerol (18:2) | | | | | |
| 100001597 | | tiglylcarnitine | Amino | Leucine, | 0 | 0 | pos |
| | | | Acid | Isoleucine | | | |
| | | | | and Valine | | | |
| | | | | Metabolism | | | |
| 100005864 | | methyl | Xeno- | Food | 0 | 0 | neg |
| | | glucopyranoside | biotics | Component/ | | | |
| | | (alpha + | | Plant | | | |
| | | beta) | | | | | |
| 100000467 | | 3-indoxyl | Amino | Tryptophan | 0 | 0 | pos |
| | | sulfate | Acid | Metabolism | | | |
| 100009332 | | arachidonoyl- | Lipid | Phospholipid | 0 | 0 | neg |
| | | choline | | Metabolism | | | |
| 100002397 | X - 12695 | alpha-keto- | 0 | . | 0 | 0 | pos |
| | | glutaramate** | | | | | |
| 100001182 | | docosadienoate | Lipid | Polyun- | 0 | 0 | neg |
| | | (22:2n6) | | saturated | | | |
| | | | | Fatty Acid | | | |
| | | | | (n3 and n6) | | | |
| 100000963 | | homocitrulline | Amino | Urea cycle; | 0 | 0 | pos |
| | | | Acid | Arginine | | | |
| | | | | and Proline | | | |
| | | | | Metabolism | | | |
| X - 16071 | | X - 16071 | 0 | . | 0 | 0 | pos |
| 171 | | hypoxanthine | Nucleotide | Purine | 0 | 0 | pos |
| | | | | Metabolism, | | | |
| | | | | (Hypo)Xanthine/ | | | |
| | | | | Inosine | | | |
| | | | | containing | | | |
| X - 09789 | | X - 10358 | 0 | . | 0 | 0 | neg |
| 100015681 | | palmitoleoyl- | Lipid | Diacylglycerol | 0 | 0 | pos |
| | | arachidonoyl- | | | | | |
| | | glycerol | | | | | |
| | | (16:1/20:4) | | | | | |
| | | [2]* | | | | | |
| X - 12442 | | X - 12450 | 0 | . | 0 | 0 | neg |
| 100001055 | | isobutyryl- | Amino | Leucine, | 0 | 0 | pos |
| | | carnitine | Acid | Isoleucine | | | |
| | | | | and Valine | | | |
| | | | | Metabolism | | | |
| 100009209 | | 1-palmitoyl- | Lipid | Phospholipid | 0 | 0 | neg |
| | | 2-eicosapenta- | | Metabolism | | | |
| | | enoyl-GPE | | | | | |
| | | (16:0/20:5)* | | | | | |
| 100015610 | | palmitoyl- | Lipid | Diacylglycerol | 0 | 0 | pos |
| | | myristoyl- | | | | | |
| | | glycerol | | | | | |
| | | (16:0/14:0) | | | | | |
| | | [2] | | | | | |
| X - 23782 | | X - 23782 | 0 | . | 0 | 0 | neg |
| 100009145 | | 1-palmitoyl- | Lipid | Phospholipid | 0 | 0 | pos |
| | | 2-meadoyl- | | Metabolism | | | |
| | | GPC | | | | | |
| | | (16:0/20:3n9)* | | | | | |
| 1080 | | 5-KETE | Lipid | Eicosanoid | 0 | 0 | pos |
| 209 | | adenosine 5′- | Nucleotide | Purine | 0 | 0 | pos |
| | | monophosphate | | Metabolism, | | | |
| | | (AMP) | | Adenine | | | |
| | | | | containing | | | |
| X - 11308 | | X - 11315 | 0 | . | 0 | 0 | pos |
| 100002126 | | 16a-hydroxy | Lipid | Steroid | 0 | 0 | incon- |
| | | DHEA 3- | | | | | sistent |
| | | sulfate | | | | | |
| 1123 | | chenodeoxy- | Lipid | Primary | 0 | 0 | pos |
| | | cholate | | Bile Acid | | | |
| | | | | Metabolism | | | |
| 100006116 | | methyl-4- | Xeno- | Benzoate | 0 | 0 | neg |
| | | hydroxybenzoate | biotics | Metabolism | | | |
| | | sulfate | | | | | |
| 913 | | maltose | Carbo- | Glycogen | 0 | 0 | pos |
| | | | hydrate | Metabolism | | | |
| 100002869 | | 1-erucoyl- | Lipid | Lysophospholipid | 0 | 0 | neg |
| | | GPC (22:1)* | | | | | |
| X - 11478 | | X - 11483 | 0 | . | 0 | 0 | pos |
| 827 | | cytidine | Nucleotide | Pyrimidine | 0 | 0 | incon- |
| | | | | Metabolism, | | | sistent |
| | | | | Cytidine | | | |
| | | | | containing | | | |
| 62 | | 12,13- | Lipid | Fatty Acid, | 0 | 0 | neg |
| | | DiHOME | | Dihydroxy | | | |
| 100000707 | | maleate | Lipid | Fatty Acid, | 0 | 0 | pos |
| | | | | Dicarboxylate | | | |
| 407 | | lysine | Amino | Lysine | 0 | 0 | pos |
| | | | Acid | Metabolism | | | |
| 932 | | caprylate | Lipid | Medium | 0 | 0 | neg |
| | | (8:0) | | Chain Fatty | | | |
| | | | | Acid | | | |
| 100015967 | | carotene | Xeno- | Food | 0 | 0 | neg |
| | | diol (2) | biotics | Component/ | | | |
| | | | | Plant | | | |
| 250 | | biliverdin | Cofactors | Hemoglobin | 0 | 0 | incon- |
| | | | and | and Porphyrin | | | sistent |
| | | | Vitamins | Metabolism | | | |
| 100009233 | | palmitoyl- | Lipid | Fatty Acid | 0 | 0 | neg |
| | | choline | | Metabolism | | | |
| | | | | (Acyl | | | |
| | | | | Choline) | | | |
| 100008957 | | sphingomyelin | Lipid | Sphingolipid | 0 | 0 | incon- |
| | | (d18:2/24:1, | | Metabolism | | | sistent |
| | | d18:1/24:2)* | | | | | |
| 100004646 | | cyclo(ala- | Peptide | Dipeptide | 0 | 0 | pos |
| | | pro) | | | | | |
| 272 | | corticosterone | Lipid | Steroid | 0 | 0 | neg |
| 100001466 | | 3- | Nucleotide | Pyrimidine | 0 | 0 | pos |
| | | methylcytidine | | Metabolism, | | | |
| | | | | Cytidine | | | |
| | | | | containing | | | |
| X - 17178 | | X - 17178 | 0 | . | 0 | 0 | neg |
| 537 | | trans- | Amino | Histidine | 0 | 0 | neg |
| | | urocanate | Acid | Metabolism | | | |
| 100015839 | | dihomo- | Lipid | Fatty Acid | 0 | 0 | neg |
| | | linoleoyl- | | Metabolism | | | |
| | | carnitine | | (Acyl | | | |
| | | (C20:2)* | | Carnitine) | | | |
| 100000016 | | suberate | Lipid | Fatty Acid, | 0 | 0 | incon- |
| | | (octanedioate) | | Dicarboxylate | | | sistent |
| X - 21796 | | X - 21796 | 0 | . | 0 | 0 | pos |
| 100004575 | | N2,N5- | Amino | Urea cycle; | 0 | 0 | neg |
| | | diacetylornithine | Acid | Arginine | | | |
| | | | | and Proline | | | |
| | | | | Metabolism | | | |
| 100000774 | | phenyllactate | Amino | Phenylalanine | 0 | 0 | pos |
| | | (PLA) | Acid | and Tyrosine | | | |
| | | | | Metabolism | | | |
| 100009335 | | dihomo- | Lipid | Phospholipid | 0 | 0 | neg |
| | | linolenoyl- | | Metabolism | | | |
| | | choline | | | | | |
| 1506 | | N- | Lipid | Fatty Acid | 0 | 0 | neg |
| | | linoleoyl- | | Metabolism | | | |
| | | glycine | | (Acyl | | | |
| | | | | Glycine) | | | |
| 100001594 | | beta-hydroxy- | Amino | Leucine, | 0 | 0 | pos |
| | | isovaleroyl- | Acid | Isoleucine | | | |
| | | carnitine | | and Valine | | | |
| | | | | Metabolism | | | |
| 2049 | | 4- | Xeno- | Drug | 0 | 0 | incon- |
| | | acetaminophen | biotics | | | | sistent |
| | | sulfate | | | | | |
| 100001756 | | 4- | Xeno- | Benzoate | 0 | 0 | neg |
| | | ethylphenyl | biotics | Metabolism | | | |
| | | sulfate | | | | | |
| 100001391 | | stearoyl- | Lipid | Fatty Acid | 0 | 0 | incon- |
| | | carnitine | | Metabolism | | | sistent |
| | | | | (Acyl | | | |
| | | | | Carnitine) | | | |
| 100015968 | | carotene | Xeno- | Food | 0 | 0 | neg |
| | | diol (3) | biotics | Component/ | | | |
| | | | | Plant | | | |
| 194 | | N- | Amino | Methionine, | 0 | 0 | pos |
| | | formylmethionine | Acid | Cysteine, | | | |
| | | | | SAM and | | | |
| | | | | Taurine | | | |
| | | | | Metabolism | | | |
| 100001992 | | 4-androsten- | Lipid | Steroid | 0 | 0 | pos |
| | | 3beta,17beta-diol | | | | | |
| | | disulfate (1) | | | | | |
| 254 | | 3-hydroxybutyrate | Lipid | Ketone | 0 | 0 | neg |
| | | (BHBA) | | Bodies | | | |
| 100004089 | | 2-hydroxydecanoate | Lipid | Fatty Acid, | 0 | 0 | neg |
| | | | | Monohydroxy | | | |
| 100001541 | | 2-hydroxy- | Amino | Leucine, | 0 | 0 | pos |
| | | 3-methyl- | Acid | Isoleucine | | | |
| | | valerate | | and Valine | | | |
| | | | | Metabolism | | | |
| 100009150 | | 1-myristoyl-2- | Lipid | Phosphatidyl- | 0 | 0 | pos |
| | | palmitoleoyl- | | choline (PC) | | | |
| | | GPC | | | | | |
| | | (14:0/16:1)* | | | | | |
| 100001994 | | 4-androsten- | Lipid | Steroid | 0 | 0 | pos |
| | | 3beta,17beta-diol | | | | | |
| | | disulfate (2) | | | | | |
| 100020004 | X - 02249 | 3-Cmpfp** | 0 | . | 0 | 0 | pos |
| 100005371 | | 1-dihomo- | Lipid | Lysolipid | 0 | 0 | pos |
| | | linolenoyl- | | | | | |
| | | GPE | | | | | |
| | | (20:3n3 or | | | | | |
| | | 6)* | | | | | |
| 100015745 | | glycosyl | Lipid | Ceramides | 0 | 0 | neg |
| | | ceramide | | | | | |
| | | (d18:2/24:1, | | | | | |
| | | d18:1/24:2)* | | | | | |
| 100004182 | | 3b-hydroxy- | Lipid | Secondary | 0 | 0 | neg |
| | | 5-cholenoic | | Bile Acid | | | |
| | | acid | | Metabolism | | | |
| 100000743 | | 2-hydroxy- | Lipid | Fatty Acid, | 0 | 0 | incon- |
| | | octanoate | | Monohydroxy | | | sistent |
| 100015845 | | docosahexa- | Lipid | Fatty Acid | 0 | 0 | neg |
| | | enoylcarnitine | | Metabolism | | | |
| | | (C22:6)* | | (Acyl | | | |
| | | | | Carnitine) | | | |
| X - 21353 | | X - 21353 | 0 | . | 0 | 0 | neg |
| X - 12206 | | X - 12212 | 0 | . | 0 | 0 | pos |
| 100010918 | | oleoyl-oleoyl- | Lipid | Diacylglycerol | 0 | 0 | pos |
| | | glycerol | | | | | |
| | | (18:1/18:1) | | | | | |
| | | [1]* | | | | | |
| X - 16570 | | X - 16570 | 0 | . | 0 | 0 | pos |
| 100000463 | | indolelactate | Amino | Tryptophan | 0 | 0 | pos |
| | | | Acid | Metabolism | | | |
| 100005372 | | 1-(1-enyl- | Lipid | Lysolipid | 0 | 0 | neg |
| | | oleoyl)- | | | | | |
| | | GPE (P- | | | | | |
| | | 18:1)* | | | | | |
| X - 21834 | | X - 21834 | 0 | . | 0 | 0 | neg |
| 100000011 | | phenylacetate | Amino | Phenylalanine | 0 | 0 | neg |
| | | | Acid | and Tyrosine | | | |
| | | | | Metabolism | | | |
| 100001403 | | 5- | Xeno- | Xanthine | 0 | 0 | pos |
| | | acetylamino- | biotics | Metabolism | | | |
| | | 6-amino-3- | | | | | |
| | | methyluracil | | | | | |
| 100002171 | | 1- | Lipid | Lysolipid | 0 | 0 | neg |
| | | margaroyl- | | | | | |
| | | GPE | | | | | |
| | | (17:0)* | | | | | |
| 100004555 | | benzoyl- | Xeno- | Chemical | 0 | 0 | pos |
| | | carnitine | biotics | | | | |
| 100002152 | | andro | Lipid | Steroid | 0 | 0 | pos |
| | | steroid | | | | | |
| | | monosulfate | | | | | |
| | | (1)* | | | | | |
| X - 22816 | | X - 22816 | 0 | . | 0 | 0 | neg |
| X - 12127 | | X - 12170 | 0 | . | 0 | 0 | pos |
| 100001022 | | threonate | Cofactors | Ascorbate and | 0 | 0 | neg |
| | | | and | Aldarate | | | |
| | | | Vitamins | Metabolism | | | |
| 100001445 | | 1-palmitoyl- | Lipid | Lysolipid | 0 | 0 | pos |
| | | GPA (16:0) | | | | | |
| 100001596 | | 2- | Lipid | Fatty Acid | 0 | 0 | pos |
| | | methylmalonyl | | Synthesis | | | |
| | | carnitine | | | | | |
| 100010950 | | stearoyl- | Lipid | Diacylglycerol | 0 | 0 | pos |
| | | arachidonoyl- | | | | | |
| | | glycerol | | | | | |
| | | (18:0/20:4) | | | | | |
| | | [2]* | | | | | |
| 100001106 | | 1,3- | Xeno- | Xanthine | 0 | 0 | pos |
| | | dimethylurate | biotics | Metabolism | | | |
| 100003674 | | prolylglycine | Peptide | Dipeptide | 0 | 0 | pos |
| 100002183 | | S- | Amino | Methionine, | 0 | 0 | neg |
| | | methylmethionine | Acid | Cysteine, | | | |
| | | | | SAM and | | | |
| | | | | Taurine | | | |
| | | | | Metabolism | | | |
| 100000014 | | hippurate | Xeno- | Benzoate | 0 | 0 | neg |
| | | | biotics | Metabolism | | | |
| 100002167 | | 12-HETE | Lipid | Eicosanoid | 0 | 0 | pos |
| 100002204 | | N-acetyl-3- | Amino | Histidine | 0 | 0 | incon- |
| | | methylhistidine* | Acid | Metabolism | | | sistent |
| 100000862 | | 4- | Xeno- | Benzoate | 0 | 0 | incon- |
| | | hydroxybenzoate | biotics | Metabolism | | | sistent |
| 100001064 | | glycolithocholate | Lipid | Secondary | 0 | 0 | incon- |
| | | | | Bile Acid | | | sistent |
| | | | | Metabolism | | | |
| 100005986 | | sphingomyelin | Lipid | Sphingolipid | 0 | 0 | neg |
| | | (d18:1/24:1, | | Metabolism | | | |
| | | d18:2/24:0)* | | | | | |
| X -12459 | | X - 12462 | 0 | . | 0 | 0 | neg |
| 100003686 | | N-palmitoyl | Lipid | Fatty Acid | 0 | 0 | incon- |
| | | glycine | | Metabolism | | | sistent |
| | | | | (Acyl | | | |
| | | | | Glycine) | | | |
| 100001784 | | 1-palmitoleoyl- | Lipid | Lysolipid | 0 | 0 | pos |
| | | GPE (16:1)* | | | | | |
| 409 | | malate | Energy | TCA Cycle | 0 | 0 | incon- |
| | | | | | | | sistent |
| 1442 | | beta-hydroxy- | Amino | Leucine, | 0 | 0 | pos |
| | | isovalerate | Acid | Isoleucine | | | |
| | | | | and Valine | | | |
| | | | | Metabolism | | | |
| 100001604 | | hydroquinone | Xeno- | Drug | 0 | 0 | pos |
| | | sulfate | biotics | | | | |
| 100003200 | | phenylalanyl- | Peptide | Dipeptide | 0 | 0 | pos |
| | | leucine | | | | | |
| 100000706 | | alpha- | Amino | Leucine, | 0 | 0 | pos |
| | | hydroxy- | Acid | Isoleucine | | | |
| | | isocaproate | | and Valine | | | |
| | | | | Metabolism | | | |
| 100000437 | | theophylline | Xeno- | Xanthine | 0 | 0 | pos |
| | | | biotics | Metabolism | | | |
| 1135 | | ursodeoxycholate | Lipid | Secondary | 0 | 0 | pos |
| | | | | Bile Acid | | | |
| | | | | Metabolism | | | |
| X - 12212 | | X - 12216 | 0 | . | 0 | 0 | neg |
| 100001251 | | decanoyl- | Lipid | Fatty Acid | 0 | 0 | neg |
| | | carnitine | | Metabolism | | | |
| | | | | (Acyl | | | |
| | | | | Carnitine) | | | |
| 1492 | | linoleamide | Lipid | Fatty Acid, | 0 | 0 | neg |
| | | (18:2n6) | | Amide | | | |
| 158 | | 5,6- | Nuclotide | Pyrimidine | 0 | 0 | pos |
| | | dihydrothymine | | Metabolism, | | | |
| | | | | Thymine | | | |
| | | | | containing | | | |
| 100002405 | | metformin | Xeno- | Drug | 0 | 0 | pos |
| | | | biotics | | | | |
| 279 | | cystine | Amino | Methionine, | 0 | 0 | pos |
| | | | Acid | Cysteine, | | | |
| | | | | SAM and | | | |
| | | | | Taurine | | | |
| | | | | Metabolism | | | |
| 100005384 | | O-sulfo-L- | Xeno- | Chemical | 0 | 0 | pos |
| | | tyrosine | biotics | | | | |
| 100008956 | | sphingomyelin | Lipid | Sphingolipid | 0 | 0 | incon- |
| | | (d18:2/23:0), | | Metabolism | | | sistent |
| | | d18:1/23:1, | | | | | |
| | | d17:1/24:1)* | | | | | |
| X - 21729 | | X - 21729 | 0 | . | 0 | 0 | pos |
| 100009271 | | 3- | Lipid | Fatty Acid | 0 | 0 | pos |
| | | hydroxybutyryl- | | Metabolism | | | |
| | | carnitine (2) | | (Acyl | | | |
| | | | | Carnitine) | | | |
| 100010958 | | diacylglycerol | Lipid | Diacylglycerol | 0 | 0 | pos |
| | | (12:0/18:1, | | | | | |
| | | 14:0/16:1, | | | | | |
| | | 16:0/14:1) | | | | | |
| | | [1]* | | | | | |
| 100001501 | | oleoylcarnitine | Lipid | Fatty Acid | 0 | 0 | incon- |
| | | | | Metabolism | | | sistent |
| | | | | (Acyl | | | |
| | | | | Carnitine) | | | |
| 100000584 | | 2- | Lipid | Monoacylglycerol | 0 | 0 | incon- |
| | | arachidonoyl- | | | | | sistent |
| | | glycerol | | | | | |
| | | (20:4) | | | | | |
| 100002137 | | 5-HETE | Lipid | Eicosanoid | 0 | 0 | pos |
| X - 11440 | | X - 11441 | 0 | . | 0 | 0 | pos |
| 848 | | cotinine | Xeno- | Tobacco | 0 | 0 | incon- |
| | | | biotics | Metabolite | | | sistent |
| 100001437 | | cysteine- | Amino | Glutathione | 0 | 0 | neg |
| | | glutathione | Acid | Metabolism | | | |
| | | disulfide | | | | | |
| 362 | | inosine 5′- | Nucleotide | Purine | 0 | 0 | pos |
| | | monophosphate | | Metabolism, | | | |
| | | (IMP) | | (Hypo)Xanthine/ | | | |
| | | | | Inosine | | | |
| | | | | containing | | | |
| 100000660 | | 1,2-distearoyl- | Lipid | Phospholipid | 0 | 0 | neg |
| | | GPC (18:0/18:0) | | Metabolism | | | |
| 100001658 | | taurolithocholate | Lipid | Secondary | 0 | 0 | neg |
| | | 3-sulfate | | Bile Acid | | | |
| | | | | Metabolism | | | |
| 100010919 | | oleoyl- | Lipid | Diacylglycerol | 0 | 0 | pos |
| | | oleoyl- | | | | | |
| | | glycerol | | | | | |
| | | (18:1/18:1) | | | | | |
| | | [2]* | | | | | |
| 1053 | | 3- | Nucleotide | Pyrimidine | 0 | 0 | neg |
| | | ureidopropionate | | Metabolism, | | | |
| | | | | Uracil | | | |
| | | | | containing | | | |
| 100008980 | | 1-stearoyl- | Lipid | Phospholipid | 0 | 0 | neg |
| | | 2-linoleoyl- | | Metabolism | | | |
| | | GPC | | | | | |
| | | (18:0/18:2)* | | | | | |
| 1235 | | gamma- | Peptide | Gamma- | 0 | 0 | incon- |
| | | glutamylhistidine | | glutamyl | | | sistent |
| | | | | Amino Acid | | | |
| 100006125 | | vanillic | Amino | Phenylalanine | 0 | 0 | pos |
| | | alcohol | Acid | and Tyrosine | | | |
| | | sulfate | | Metabolism | | | |
| 100001409 | | N1- | Nucleotide | Purine | 0 | 0 | pos |
| | | methylinosine | | Metabolism, | | | |
| | | | | (Hypo)Xanthine/ | | | |
| | | | | Inosine | | | |
| | | | | containing | | | |
| 100000101 | | pimelate | Lipid | Fatty Acid, | 0 | 0 | pos |
| | | (heptanedioate) | | Dicarboxylate | | | |
| 825 | | uracil | Nucleotide | Pyrimidine | 0 | 0 | pos |
| | | | | Metabolism, | | | |
| | | | | Uracil | | | |
| | | | | containing | | | |
| 100006314 | | sphingomyelin | Lipid | Sphingolipid | 0 | 0 | incon- |
| | | (d18:1/15:0, | | Metabolism | | | sistent |
| | | d16:1/17:0)* | | | | | |
| 100015915 | | 1-nervonoyl- | Lipid | Phosphatidyl- | 0 | 0 | neg |
| | | 2-arachidonoyl- | | choline | | | |
| | | GPC | | (PC) | | | |
| | | (24:1/20:4)* | | | | | |
| 1105 | | alpha- | Cofactors | Tocopherol | 0 | 0 | neg |
| | | tocopherol | and | Metabolism | | | |
| | | | Vitamins | | | | |
| 100001870 | | 1-palmitoyl- | Lipid | Phospholipid | 0 | 0 | pos |
| | | 2-linoleoyl- | | Metabolism | | | |
| | | GPE | | | | | |
| | | (16:0/18:2) | | | | | |
| 100001527 | | hexanoylglycine | Lipid | Fatty Acid | 0 | 0 | neg |
| | | | | Metabolism | | | |
| | | | | (Acyl | | | |
| | | | | Glycine) | | | |
| 100002761 | X - 22379 | androsterone | 0 | . | 0 | 0 | pos |
| | | glucuronide | | | | | |
| X - 23705 | | PC(O- | 0 | . | 0 | 0 | neg |
| | | 18:0/20:4)* | | | | | |
| X - 21411 | | X - 21411 | 0 | . | 0 | 0 | neg |
| 100015587 | | 1-stearyl-2- | Lipid | Plasmalogen | 0 | 0 | neg |
| | | docosapenta- | | | | | |
| | | enoyl-GPC (O- | | | | | |
| | | 18:0/22:5n3)* | | | | | |
| 100003549 | | histidyltryptophan | Peptide | Dipeptide | 0 | 0 | pos |
| 100003696 | | succinimide | Xeno- | Chemical | 0 | 0 | neg |
| | | | biotics | | | | |
| 424 | | palmitate | Lipid | Long Chain | 0 | 0 | pos |
| | | (16:0) | | Fatty Acid | | | |
| 799 | | betaine | Amino | Glycine, | 0 | 0 | neg |
| | | | Acid | Serine and | | | |
| | | | | Threonine | | | |
| | | | | Metabolism | | | |
| 100001300 | | alpha- | Amino | Leucine, | 0 | 0 | incon- |
| | | hydroxy- | Acid | Isoleucine | | | sistent |
| | | isovalerate | | and Valine | | | |
| | | | | Metabolism | | | |
| 100001795 | | 2- | Xeno- | Drug | 0 | 0 | pos |
| | | methoxy- | biotics | | | | |
| | | acetaminophen | | | | | |
| | | glucuronide* | | | | | |
| X - 24097 | | PC(14:0/16:1)* | 0 | . | 0 | 0 | pos |
| 100000626 | | sphingosine 1- | Lipid | Sphingolipid | 0 | 0 | pos |
| | | phosphate | | Metabolism | | | |
| 100000042 | | 3- | Amino | Histidine | 0 | 0 | incon- |
| | | methylhistidine | Acid | Metabolism | | | sistent |
| 100000096 | | 4- | Amino | Guanidino and | 0 | 0 | pos |
| | | guanidinobutanoate | Acid | Acetamido | | | |
| | | | | Metabolism | | | |
| 878 | | fructose | Carbo- | Fructose, | 0 | 0 | incon- |
| | | | hydrate | Mannose and | | | sistent |
| | | | | Galactose | | | |
| | | | | Metabolism | | | |
| 100006190 | | 2- | Xeno- | Drug | 0 | 0 | neg |
| | | acetamidophenol | biotics | | | | |
| | | sulfate | | | | | |
| 100000803 | | aspartylphenylalanine | Peptide | Dipeptide | 0 | 0 | pos |
| 100001383 | | 1-myristoyl- | Lipid | Lysolipid | 0 | 0 | pos |
| | | GPC (14:0) | | | | | |
| X - 13866 | | X - 13866 | 0 | . | 0 | 0 | incon- |
| | | | | | | | sistent |
| 100002945 | | 15- | Lipid | Fatty Acid, | 0 | 0 | incon- |
| | | methylpalmitate | | Branched | | | sistent |
| 100003901 | | 2-stearoyl- | Lipid | Lysolipid | 0 | 0 | incon- |
| | | GPE | | | | | sistent |
| | | (18:0)* | | | | | |
| 100008904 | | 1-stearoyl- | Lipid | Phospholipid | 0 | 0 | pos |
| | | 2-oleoyl- | | Metabolism | | | |
| | | GPC | | | | | |
| | | (18:0/18:1) | | | | | |
| 100010944 | | oleoyl- | Lipid | Diacylglycerol | 0 | 0 | pos |
| | | linolenoyl- | | | | | |
| | | glycerol | | | | | |
| | | (18:1/18:3) | | | | | |
| | | [2]* | | | | | |
| 100015966 | | carotene | Xeno- | Food | 0 | 0 | neg |
| | | diol (1) | biotics | Component/ | | | |
| | | | | Plant | | | |
| 100000487 | | glycylvaline | Peptide | Dipeptide | 0 | 0 | pos |
| 100001335 | | eicosenoate | Lipid | Long Chain | 0 | 0 | neg |
| | | (20:1) | | Fatty Acid | | | |
| 100001620 | | glycerophospho- | Lipid | Phospholipid | 0 | 0 | incon- |
| | | ethanolamine | | Metabolism | | | sistent |
| 100001614 | | hexadecanedioate | Lipid | Fatty Acid, | 0 | 0 | neg |
| | | | | Dicarboxylate | | | |
| 1113 | | 4- | Amino | Polyamine | 0 | 0 | pos |
| | | acetamidobutanoate | Acid | Metabolism | | | |
| 100001791 | | 2-hydroxy- | Xeno- | Drug | 0 | 0 | incon- |
| | | acetaminophen | biotics | | | | sistent |
| | | sulfate* | | | | | |
| 100006115 | | arabonate/ | Carbo- | Pentose | 0 | 0 | incon- |
| | | xylonate | hydrate | Phosphate | | | sistent |
| | | | | Pathway | | | |
| X - 23293 | | X - 23293 | 0 | . | 0 | 0 | neg |
| X - 11850 | | X - 11852 | 0 | . | 0 | 0 | neg |
| X - 21286 | | X - 21286 | 0 | . | 0 | 0 | pos |
| 100008999 | | 1-(1-enyl- | Lipid | Phospholipid | 0 | 0 | incon- |
| | | stearoyl)-2- | | Metabolism | | | sistent |
| | | arachidonoyl- | | | | | |
| | | GPE (P- | | | | | |
| | | 18:0/20:4)* | | | | | |
| 100009146 | | 1-stearoyl- | Lipid | Phosphatidyl- | 0 | 0 | pos |
| | | 2-adrenoyl- | | choline (PC) | | | |
| | | GPC | | | | | |
| | | (18:0/22:4)* | | | | | |
| 339 | | glutarate | Amino | Lysine | 0 | 0 | incon- |
| | | (pentanedioate) | Acid | Metabolism | | | sistent |
| 100003700 | | diglycerol | Xeno- | Chemical | 0 | 0 | pos |
| | | | biotics | | | | |
| 100001148 | | 5- | Lipid | Fatty Acid, | 0 | 0 | incon- |
| | | hydroxyhexanoate | | Monohydroxy | | | sistent |
| 1258 | | anthranilate | Amino | Tryptophan | 0 | 0 | pos |
| | | | Acid | Metabolism | | | |
| 100000870 | | saccharin | Xeno- | Food | 0 | 0 | pos |
| | | | biotics | Component/ | | | |
| | | | | Plant | | | |
| 1128 | | 2- | Amino | Methionine, | 0 | 0 | pos |
| | | aminobutyrate | Acid | Cysteine, | | | |
| | | | | SAM and | | | |
| | | | | Taurine | | | |
| | | | | Metabolism | | | |
| 100001392 | | laurylcarnitine | Lipid | Fatty Acid | 0 | 0 | neg |
| | | | | Metabolism | | | |
| | | | | (Acyl | | | |
| | | | | Carnitine) | | | |
| 100009234 | | 3,4- | Lipid | Fatty Acid | 0 | 0 | pos |
| | | methyleneheptanoyl- | | Metabolism | | | |
| | | carnitine | | (Acyl | | | |
| | | | | Carnitine) | | | |
| 100002021 | | 5alpha- | Lipid | Steroid | 0 | 0 | incon- |
| | | androstan- | | | | | sistent |
| | | 3beta,17alpha-diol | | | | | |
| | | disulfate | | | | | |
| 100002122 | | 3- | Xeno- | Benzoate | 0 | 0 | incon- |
| | | hydroxyhippurate | biotics | Metabolism | | | sistent |
| 100004284 | | dimethyl | Xeno- | Chemical | 0 | 0 | neg |
| | | sulfone | biotics | | | | |
| X - 18899 | | X - 18899 | 0 | . | 0 | 0 | pos |
| 100001569 | | 1-oleoyl- | Lipid | Lysolipid | 0 | 0 | neg |
| | | GPE (18:1) | | | | | |
| 100010934 | | diacylglycerol | Lipid | Diacylglycerol | 0 | 0 | pos |
| | | (14:0/18:1, | | | | | |
| | | 16:0/16:1) | | | | | |
| | | [1]* | | | | | |
| 100002094 | | gamma- | Cofactors | Tocopherol | 0 | 0 | pos |
| | | CEHC | and | Metabolism | | | |
| | | | Vitamins | | | | |
| X - 11452 | | sulfate of | 0 | . | 0 | 0 | pos |
| | | piperine | | | | | |
| | | metabolite | | | | | |
| | | C16H19NO3 (2)* | | | | | |
| 100004328 | | sphingomyelin | Lipid | Sphingolipid | 0 | 0 | incon- |
| | | (d18:1/14:0, | | Metabolism | | | sistent |
| | | d16:1/16:0)* | | | | | |
| 100001621 | | glycero- | Lipid | Phospholipid | 0 | 0 | incon- |
| | | phosphoinositol* | | Metabolism | | | sistent |
| 100010949 | | stearoyl- | Lipid | Diacylglycerol | 0 | 0 | pos |
| | | arachidonoyl- | | | | | |
| | | glycerol | | | | | |
| | | (18:0/20:4) | | | | | |
| | | [1]* | | | | | |
| 1537 | | 1-palmitoyl- | Lipid | Phospholipid | 0 | 0 | incon- |
| | | 2-linoleoyl- | | Metabolism | | | sistent |
| | | GPC | | | | | |
| | | (16:0/18:2) | | | | | |
| 100001987 | | 5alpha-androstan- | Lipid | Steroid | 0 | 0 | incon- |
| | | 3beta,17beta-diol | | | | | sistent |
| | | disulfate | | | | | |
| 100009345 | | 1-palmitoleoyl- | Lipid | Phospholipid | 0 | 0 | pos |
| | | 2-linolenoyl- | | Metabolism | | | |
| | | GPC (16:1/18:3)* | | | | | |
| 100006092 | | tyramine O- | Amino | Phenylalanine | 0 | 0 | incon- |
| | | sulfate | Acid | and Tyrosine | | | sistent |
| | | | | Metabolism | | | |
| 100015760 | X - 11537 | linoleoylcholine* | Lipid | Fatty Acid | 0 | 0 | neg |
| | | | | Metabolism | | | |
| | | | | (Acyl | | | |
| | | | | Choline) | | | |
| 100009344 | | 1,2- | Lipid | Phospholipid | 0 | 0 | pos |
| | | dilinolenoyl- | | Metabolism | | | |
| | | GPC | | | | | |
| | | (18:3/18:3)* | | | | | |
| 1628 | | glycocheno- | Lipid | Primary | 0 | 0 | incon- |
| | | deoxycholate | | Bile Acid | | | sistent |
| | | | | Metabolism | | | |
| 100006290 | | sphingomyelin | Lipid | Sphingolipid | 0 | 0 | incon- |
| | | (d18:1/20:0, | | Metabolism | | | sistent |
| | | d16:1/22:0)* | | | | | |
| 100001462 | | 1-stearoyl- | Lipid | Lysophospholipid | 0 | 0 | pos |
| | | GPG (18:0) | | | | | |
| 100001247 | | octanoylcarnitine | Lipid | Fatty Acid | 0 | 0 | neg |
| | | | | Metabolism | | | |
| | | | | (Acyl | | | |
| | | | | Carnitine) | | | |
| X - 17438 | | X - 17438 | 0 | . | 0 | 0 | neg |
| 100001417 | | phenylacetyl- | Amino | Phenylalanine | 0 | 0 | incon- |
| | | glutamine | Acid | and Tyrosine | | | sistent |
| | | | | Metabolism | | | |
| 100006642 | | glycodeoxycholate | Lipid | Secondary | 0 | 0 | neg |
| | | sulfate | | Bile Acid | | | |
| | | | | Metabolism | | | |
| 100009220 | | 1-oleoyl-2- | Lipid | Phosphatidyl- | 0 | 0 | neg |
| | | docosahexaenoyl- | | ethanolamine | | | |
| | | GPE | | (PE) | | | |
| | | (18:1/22:6)* | | | | | |
| 100001250 | | tauro-beta- | Lipid | Primary | 0 | 0 | neg |
| | | muricholate | | Bile Acid | | | |
| | | | | Metabolism | | | |
| 100001777 | | 1-oleoyl- | Lipid | Lysolipid | 0 | 0 | neg |
| | | GPI (18:1)* | | | | | |
| 100008990 | | 1-palmitoyl- | Lipid | Phospholipid | 0 | 0 | pos |
| | | 2-arachidonoyl- | | Metabolism | | | |
| | | GPE | | | | | |
| | | (16:0/20:4)* | | | | | |
| 100001868 | | 4- | Xeno- | Food | 0 | 0 | neg |
| | | allylphenol | biotics | Component/ | | | |
| | | sulfate | | Plant | | | |
| 100003892 | | lanthionine | Xeno- | Chemical | 0 | 0 | incon- |
| | | | biotics | | | | sistent |
| 500 | | riboflavin | Cofactors | Riboflavin | 0 | 0 | pos |
| | | (Vitamin | and | Metabolism | | | |
| | | B2) | Vitamins | | | | |
| 100020492 | X - 01911 | glucuronide | 0 | . | 0 | 0 | pos |
| | | of piperine | | | | | |
| | | metabolite | | | | | |
| | | C17H21NO3 (4)* | | | | | |
| 100002049 | | 4- | Xeno- | Drug | 0 | 0 | neg |
| | | hydroxycoumarin | biotics | | | | |
| 100001170 | | 3-hydroxy- | Amino | Leucine, | 0 | 0 | pos |
| | | 2-ethylpropionate | Acid | Isoleucine | | | |
| | | | | and Valine | | | |
| | | | | Metabolism | | | |
| X - 22776 | | X - 22776 | 0 | . | 0 | 0 | pos |
| 800 | | cysteine | Amino | Methionine, | 0 | 0 | pos |
| | | | Acid | Cysteine, | | | |
| | | | | SAM and | | | |
| | | | | Taurine | | | |
| | | | | Metabolism | | | |
| 100003271 | X - 12748 | beta- | Amino | Glutamate | 0 | 0 | pos |
| | | citrylglutamate | Acid | Metabolism | | | |
| 266 | | cholesterol | Lipid | Sterol | 0 | 0 | incon- |
| | | | | | | | sistent |
| 100009364 | | phosphatidyl- | Lipid | Phospholipid | 0 | 0 | neg |
| | | choline | | Metabolism | | | |
| | | (18:0/20:2, | | | | | |
| | | 20:0/18:2)* | | | | | |
| 100002105 | | stearamide | Lipid | Fatty Acid, | 0 | 0 | neg |
| | | | | Amide | | | |
| 100020204 | X - 12511 | N-acetyl-2- | 0 | . | 0 | 0 | incon- |
| | | aminoctanoate | | | | | sistent |
| 100015641 | | N- | Lipid | Endocannabinoid | 0 | 0 | neg |
| | | oleoylserine | | | | | |
| 399 | | leukotriene | Lipid | Eicosanoid | 0 | 0 | incon- |
| | | B4 | | | | | sistent |
| 100006264 | | propyl 4- | Xeno- | Benzoate | 0 | 0 | neg |
| | | hydroxybenzoate | biotics | Metabolism | | | |
| | | sulfate | | | | | |
| 100001313 | | gamma- | Peptide | Gamma- | 0 | 0 | incon- |
| | | glutamyl- | | glutamyl | | | sistent |
| | | methionine | | Amino Acid | | | |
| 100010959 | | diacylglycerol | Lipid | Diacylglycerol | 0 | 0 | pos |
| | | (12:0/18:1, | | | | | |
| | | 14:0/16:1, | | | | | |
| | | 16:0/14:1) | | | | | |
| | | [2]* | | | | | |
| 100001651 | | 2-oleoyl- | Lipid | Lysolipid | 0 | 0 | incon- |
| | | GPE (18:1)* | | | | | sistent |
| X - 14314 | | pyr-leu* | 0 | . | 0 | 0 | pos |
| 100005391 | | 3-(3-hydroxy- | Amino | Phenylalanine | 0 | 0 | neg |
| | | phenyl)propionate | Acid | and Tyrosine | | | |
| | | sulfate | | Metabolism | | | |
| 330 | | fumarate | Energy | TCA Cycle | 0 | 0 | incon- |
| | | | | | | | sistent |
| 100001048 | | 2- | Lipid | Monoacylglycerol | 0 | 0 | incon- |
| | | palmitoylglycerol | | | | | sistent |
| | | (16:0) | | | | | |
| 100002029 | | 4-androsten- | Lipid | Steroid | 0 | 0 | pos |
| | | 3beta,17beta-diol | | | | | |
| | | monosulfate (2) | | | | | |
| 100006126 | | 4- | Xeno- | Food | 0 | 0 | incon- |
| | | vinylguaiacol | biotics | Component/ | | | sistent |
| | | sulfate | | Plant | | | |
| 100006191 | | p-cresol- | Amino | Phenylalanine | 0 | 0 | pos |
| | | glucuronide* | Acid | and Tyrosine | | | |
| | | | | Metabolism | | | |
| 100020497 | X - 12231 | sulfate of | 0 | . | 0 | 0 | pos |
| | | piperine | | | | | |
| | | metabolite | | | | | |
| | | C16H19NO3 (3)* | | | | | |
| 100006171 | | eugenol | Xeno- | Food | 0 | 0 | incon- |
| | | sulfate | biotics | Component/ | | | sistent |
| | | | | Plant | | | |
| 100002488 | X - 21666 | isoursodeoxy- | Lipid | Secondary | 0 | 0 | pos |
| | | cholate | | Bile Acid | | | |
| | | | | Metabolism | | | |
| 100009338 | | 5- | Amino | Tryptophan | 0 | 0 | neg |
| | | bromotryptophan | Acid | Metabolism | | | |
| 100000447 | | gentisate | Amino | Phenylalanine | 0 | 0 | neg |
| | | | Acid | and Tyrosine | | | |
| | | | | Metabolism | | | |
| 100008989 | | 1-palmitoyl- | Lipid | Phospholipid | 0 | 0 | incon- |
| | | 2-eicosapentaenoyl- | | Metabolism | | | sistent |
| | | GPC | | | | | |
| | | (16:0/20:5)* | | | | | |
| 100003432 | | dihydroferulic | Xeno- | Food | 0 | 0 | pos |
| | | acid | biotics | Component/ | | | |
| | | | | Plant | | | |
| 100002719 | | cotinine N- | Xeno- | Tobacco | 0 | 0 | incon- |
| | | oxide | biotics | Metabolite | | | sistent |
| 55 | | 1- | Cofactors | Nicotinate | 0 | 0 | neg |
| | | methylnicotinamide | and | and | | | |
| | | | Vitamins | Nicotinamide | | | |
| | | | | Metabolism | | | |
| 100002249 | | N-acetyl- | Amino | Lysine | 0 | 0 | pos |
| | | cadaverine | Acid | Metabolism | | | |
| 100001786 | | 1- | Lipid | Lysophospholipid | 0 | 0 | pos |
| | | palmitoleoyl- | | | | | |
| | | GPI (16:1)* | | | | | |
| 100006726 | | linoleoyl | Lipid | Endocannabinoid | 0 | 0 | pos |
| | | ethanolamide | | | | | |
| 1869 | | 2- | Xeno- | Benzoate | 0 | 0 | pos |
| | | hydroxyhippurate | biotics | Metabolism | | | |
| | | (salicylurate) | | | | | |
| 100009161 | | 1-(1-enyl- | Lipid | Plasmalogen | 0 | 0 | neg |
| | | palmitoyl)- | | | | | |
| | | 2-myristoyl- | | | | | |
| | | GPC (P- | | | | | |
| | | 16:0/14:0) | | | | | |
| 100001793 | | 2-methoxy- | Xeno- | Drug | 0 | 0 | pos |
| | | acetaminophen | biotics | | | | |
| | | sulfate* | | | | | |
| X - 21258 | | X - 21258 | 0 | . | 0 | 0 | neg |
| X - 11438 | | X - 11440 | 0 | . | 0 | 0 | neg |
| 100001150 | | propionylglycine | Lipid | Fatty Acid | 0 | 0 | neg |
| | | | | Metabolism | | | |
| | | | | (also BCAA | | | |
| | | | | Metabolism) | | | |
| 1141 | | 4- | Amino | Phenylalanine | 0 | 0 | pos |
| | | hydroxyphenyl- | Acid | and Tyrosine | | | |
| | | pyruvate | | Metabolism | | | |
| 1539 | | 1-palmitoyl- | Lipid | Phospholipid | 0 | 0 | incon- |
| | | 2-oleoyl- | | Metabolism | | | sistent |
| | | GPC | | | | | |
| | | (16:0/18:1) | | | | | |
| 100001294 | | gamma- | Peptide | Gamma- | 0 | 0 | neg |
| | | glutamyl- | | glutamyl | | | |
| | | glycine | | Amino Acid | | | |
| 100009125 | | 1-margaroyl- | Lipid | Phospholipid | 0 | 0 | neg |
| | | 2-docosahexa- | | Metabolism | | | |
| | | enoyl-GPC | | | | | |
| | | (17:0/22:6)* | | | | | |
| 1224 | | cys-gly, | Amino | Glutathione | 0 | 0 | incon- |
| | | oxidized | Acid | Metabolism | | | sistent |
| 100001212 | | guanidinosuccinate | Amino | Guanidino and | 0 | 0 | neg |
| | | | Acid | Acetamido | | | |
| | | | | Metabolism | | | |
| X - 22764 | | X - 22764 | 0 | . | 0 | 0 | neg |
| 100005389 | | ferulic acid | Xeno- | Food | 0 | 0 | incon- |
| | | 4-sulfate | biotics | Component/ | | | sistent |
| | | | | Plant | | | |
| X - 07765 | | X - 09789 | 0 | . | 0 | 0 | pos |
| 1384 | | naproxen | Xeno- | Drug | 0 | 0 | pos |
| | | | biotics | | | | |
| 100008930 | | oleate/ | Lipid | Long Chain | 0 | 0 | pos |
| | | vaccenate | | Fatty Acid | | | |
| | | (18:1) | | | | | |
| 100003119 | | N- | Lipid | Endocannabinoid | 0 | 0 | neg |
| | | oleoyltaurine | | | | | |
| X - 24242 | | X - 24242 | 0 | . | 0 | 0 | pos |
| 100002185 | | indole-3- | Amino | Tryptophan | 0 | 0 | pos |
| | | carboxylic | Acid | Metabolism | | | |
| | | acid | | | | | |
| 439 | | stearate | Lipid | Long Chain | 0 | 0 | incon- |
| | | (18:0) | | Fatty Acid | | | sistent |
| 100003000 | | 1-(1-enyl- | Lipid | Lysoplasmalogen | 0 | 0 | incon- |
| | | palmitoyl)- | | | | | sistent |
| | | GPE (P-16:0)* | | | | | |
| 100001806 | | o-cresol | Amino | Phenylalanine | 0 | 0 | incon- |
| | | sulfate | Acid | and Tyrosine | | | sistent |
| | | | | Metabolism | | | |
| 100001859 | | chiro- | Lipid | Inositol | 0 | 0 | incon- |
| | | inositol | | Metabolism | | | sistent |
| 100020478 | X - 21343 | dodecadienoate | 0 | . | 0 | 0 | neg |
| | | (12:2)* | | | | | |
| 100009337 | X - 12040 | caffeic acid | Xeno- | Xanthine | 0 | 0 | incon- |
| | | sulfate | biotics | Metabolism | | | sistent |
| 100002726 | | atenolol | Xeno- | Drug | 0 | 0 | incon- |
| | | | biotic | | | | sistent |
| 100001310 | | nicotinamide | Cofactors | Nicotinate | 0 | 0 | neg |
| | | riboside | and | and | | | |
| | | | Vitamins | Nicotinamide | | | |
| | | | | Metabolism | | | |
| 100000436 | | glycodeoxycholate | Lipid | Secondary | 0 | 0 | incon- |
| | | | | Bile Acid | | | sistent |
| | | | | Metabolism | | | |
| 100010947 | | palmitoyl- | Lipid | Diacylglycerol | 0 | 0 | pos |
| | | palmitoyl- | | | | | |
| | | glycerol | | | | | |
| | | (16:0/16:0) | | | | | |
| | | [1]* | | | | | |
| 100009021 | | 1-palmityl- | Lipid | Plasmalogen | 0 | 0 | pos |
| | | 2-arachidonoyl- | | | | | |
| | | GPC (O- | | | | | |
| | | 16:0/20:4) | | | | | |
| 100008920 | | sphingomyelin | Lipid | Sphingolipid | 0 | 0 | incon- |
| | | (d18:1/17:0, | | Metabolism | | | sistent |
| | | d17:1/18:0, | | | | | |
| | | d19:1/16:0) | | | | | |
| 536 | | 2′- | Nucleotide | Pyrimidine | 0 | 0 | incon- |
| | | deoxyuridine | | Metabolism, | | | sistent |
| | | | | Uracil | | | |
| | | | | containing | | | |
| 1383 | | 4- | Xeno- | Drug | 0 | 0 | incon- |
| | | acetamidophenol | biotics | | | | sistent |
| 100001657 | | glycolithocholate | Lipid | Secondary | 0 | 0 | incon- |
| | | sulfate* | | Bile Acid | | | sistent |
| | | | | Metabolism | | | |
| 100000043 | | 4- | Xeno- | Drug | 0 | 0 | pos |
| | | acetamidophenyl- | biotics | | | | |
| | | glucuronide | | | | | |
| 100009043 | | 7- | Amino | Tryptophan | 0 | 0 | neg |
| | | hydroxyindole | Acid | Metabolism | | | |
| | | sulfate | | | | | |
| 100002773 | | solanidine | Xeno- | Food | 0 | 0 | pos |
| | | | biotics | Component/ | | | |
| | | | | Plant | | | |
| 100001755 | | 4- | Xeno- | Benzoate | 0 | 0 | incon- |
| | | vinylphenol | biotics | Metabolism | | | sistent |
| | | sulfate | | | | | |
| 1161 | | tigloylglycine | Amino | Leucine, | 0 | 0 | neg |
| | | | Acid | Isoleucine | | | |
| | | | | and Valine | | | |
| | | | | Metabolism | | | |
| 100001296 | | stachydrine | Xeno- | Food | 0 | 0 | incon- |
| | | | biotics | Component/ | | | sistent |
| | | | | Plant | | | |
| 100005999 | | 7-hydroxy- | Lipid | Sterol | 0 | 0 | incon- |
| | | cholesterol | | | | | sistent |
| | | (alpha or | | | | | |
| | | beta) | | | | | |
| 1489 | | palmitoyl- | Lipid | Endocannabinoid | 0 | 0 | incon- |
| | | ethanolamide | | | | | sistent |
| 100005350 | | 1-linolenoyl- | Lipid | Lysolipid | 0 | 0 | neg |
| | | GPC (18:3)* | | | | | |
| 100002026 | | 4-androsten- | Lipid | Steroid | 0 | 0 | pos |
| | | 3alpha,17alpha-diol | | | | | |
| | | monosulfate (2) | | | | | |
| X - 22771 | | X - 22771 | 0 | . | 0 | 0 | neg |
| 798 | | adenosine | Nucleotide | Purine | 0 | 0 | incon- |
| | | | | Metabolism, | | | sistent |
| | | | | Adenine | | | |
| | | | | containing | | | |
| 100002024 | | 5alpha-androstan- | Lipid | Steroid | 0 | 0 | pos |
| | | 3beta,17beta-diol | | | | | |
| | | monosulfate (2) | | | | | |
| 181 | | laurate | Lipid | Medium | 0 | 0 | incon- |
| | | (12:0) | | Chain Fatty | | | sistent |
| | | | | Acid | | | |
| 100003594 | | phenylalanyl- | Peptide | Dipeptide | 0 | 0 | pos |
| | | tryptophan | | | | | |
| 1024 | | pantothenate | Cofactors | Pantothenate | 0 | 0 | incon- |
| | | | and | and CoA | | | sistent |
| | | | Vitamins | Metabolism | | | |
| 100003640 | | valylglutamine | Peptide | Dipeptide | 0 | 0 | pos |
| X - 12230 | | X - 12231 | 0 | . | 0 | 0 | neg |
| 100009000 | | 1-(1-enyl- | Lipid | Plasmalogen | 0 | 0 | incon- |
| | | palmitoyl)-2- | | | | | sistent |
| | | docosahexa- | | | | | |
| | | enoyl-GPE (P- | | | | | |
| | | 16:0/22:6)* | | | | | |
| 100001405 | | 1- | Xeno- | Xanthine | 0 | 0 | incon- |
| | | methylxanthine | biotics | Metabolism | | | sistent |
| 100009025 | | sphingomyelin | Lipid | Sphingolipid | 0 | 0 | incon- |
| | | (d18:1/21:0, | | Metabolism | | | sistent |
| | | d17:1/22:0, | | | | | |
| | | d16:1/23:0)* | | | | | |
| 100015723 | | hexadeca- | Lipid | Sphingolipid | 0 | 0 | pos |
| | | sphingosine | | Metabolism | | | |
| | | (d16:1)* | | | | | |
| 100001655 | | 1-palmitoyl- | Lipid | Lysolipid | 0 | 0 | incon- |
| | | GPI (16:0)* | | | | | sistent |
| 100000987 | | 2- | Lipid | Monoacylglycerol | 0 | 0 | incon- |
| | | linoleoylglyerol | | | | | sistent |
| | | (18:2) | | | | | |
| 796 | | alpha- | Amino | Methionine, | 0 | 0 | pos |
| | | ketobutyrate | Acid | Cysteine, | | | |
| | | | | SAM and | | | |
| | | | | Taurine | | | |
| | | | | Metabolism | | | |
| 100006438 | | citraconate/ | Energy | TCA Cycle | 0 | 0 | pos |
| | | glutaconate | | | | | |
| X - 12411 | | X - 12442 | 0 | . | 0 | 0 | pos |
| 100010941 | | linoleoyl- | Lipid | Diacylglycerol | 0 | 0 | pos |
| | | linoleoyl- | | | | | |
| | | glycerol | | | | | |
| | | (18:2/18:2) | | | | | |
| | | [1]* | | | | | |
| X - 11858 | | X - 11871 | 0 | . | 0 | 0 | neg |
| 100019975 | X - 11905 | hexadecenedioate | 0 | . | 0 | 0 | neg |
| | | (C16:1-DC)* | | | | | |
| 100001723 | | alpha- | Lipid | Fatty Acid, | 0 | 0 | pos |
| | | hydroxycaproate | | Monohydroxy | | | |
| X - 21442 | | X - 21442 | 0 | . | 0 | 0 | neg |
| 100001797 | | 3-(cystein-S- | Xeno- | Drug | 0 | 0 | pos |
| | | yl)acetaminophen* | biotics | | | | |
| X - 19141 | | X - 19141 | 0 | . | 0 | 0 | pos |
| 355 | | histidine | Amino | Histidine | 0 | 0 | incon- |
| | | | Acid | Metabolism | | | sistent |
| 100009141 | | 1-stearoyl-2- | Lipid | Phospholipid | 0 | 0 | incon- |
| | | docosapenta- | | Metabolism | | | sistent |
| | | enoyl-GPC | | | | | |
| | | (18:0/22:5n3)* | | | | | |
| 100002911 | | glycoursodeoxy- | Lipid | Secondary | 0 | 0 | pos |
| | | cholate | | Bile Acid | | | |
| | | | | Metabolism | | | |
| 100001472 | | mead acid | Lipid | Polyun- | 0 | 0 | neg |
| | | (20:3n9) | | saturated | | | |
| | | | | Fatty Acid | | | |
| | | | | (n3 and n6) | | | |
| 100009131 | | 1-linoleoyl- | Lipid | Phospholipid | 0 | 0 | incon- |
| | | 2-arachidonoyl- | | Metabolism | | | sistent |
| | | GPC | | | | | |
| | | (18:2/20:4)* | | | | | |
| 100001334 | | N- | Amino | Urea cycle; | 0 | 0 | pos |
| | | acetylproline | Acid | Arginine | | | |
| | | | | and Proline | | | |
| | | | | Metabolism | | | |
| 100009126 | | 1-arachidoyl- | Lipid | Phosphatidyl- | 0 | 0 | neg |
| | | 2-arachidonoyl- | | choline (PC) | | | |
| | | GPC | | | | | |
| | | (20:0/20:4)* | | | | | |
| 564 | | threonine | Amino | Glycine, | 0 | 0 | neg |
| | | | Acid | Serine and | | | |
| | | | | Threonine | | | |
| | | | | Metabolism | | | |
| 1518 | | N-palmitoyl- | Lipid | Sphingolipid | 0 | 0 | incon- |
| | | sphingosine | | Metabolism | | | sistent |
| | | (d18:1/16:0) | | | | | |
| 100004110 | | 3-methyl- | Xeno- | Benzoate | 0 | 0 | incon- |
| | | catechol | biotics | Metabolism | | | sistent |
| | | sulfate (2) | | | | | |
| 1256 | | choline | Lipid | Phospholipid | 0 | 0 | incon- |
| | | | | Metabolism | | | sistent |
| 100009272 | | glycosyl-N- | Lipid | Sphingolipid | 0 | 0 | neg |
| | | palmitoyl- | | Metabolism | | | |
| | | sphingosine | | | | | |
| 100000808 | | cysteine | Amino | Methionine, | 0 | 0 | incon- |
| | | s-sulfate | Acid | Cysteine, | | | sistent |
| | | | | SAM and | | | |
| | | | | Taurine | | | |
| | | | | Metabolism | | | |
| 100001181 | | docosapenta- | Lipid | Polyun- | 0 | 0 | incon- |
| | | enoate | | saturated | | | sistent |
| | | (n3 DPA; | | Fatty Acid | | | |
| | | 22:5n3) | | (n3 and n6) | | | |
| 100001564 | | 2-margaroyl- | Lipid | Lysophospholipid | 0 | 0 | neg |
| | | GPC (17:0)* | | | | | |
| 2029 | | azelate | Lipid | Fatty Acid, | 0 | 0 | incon- |
| | | (nonanedioate) | | Dicarboxylate | | | sistent |
| 100001654 | | 1- | Lipid | Lysolipid | 0 | 0 | pos |
| | | arachidonoyl- | | | | | |
| | | GPI (20:4)* | | | | | |
| 100006005 | | 5alpha-androstan- | Lipid | Steroid | 0 | 0 | pos |
| | | 3alpha,17beta-diol | | | | | |
| | | monosulfate (2) | | | | | |
| 498 | | retinol | Cofactors | Vitamin A | 0 | 0 | pos |
| | | (Vitamin A) | and | Metabolism | | | |
| | | | Vitamins | | | | |
| 100001481 | | 1-docosahexa- | Lipid | Monoacylglycerol | 0 | 0 | incon- |
| | | enoylglycerol | | | | | sistent |
| | | (22:6) | | | | | |
| 100015846 | | nervonoylcarnitine | Lipid | Fatty Acid | 0 | 0 | neg |
| | | (C24:1)* | | Metabolism | | | |
| | | | | (Acyl | | | |
| | | | | Carnitine) | | | |
| 100001314 | | gamma- | Peptide | Gamma- | 0 | 0 | incon- |
| | | glutamylthreonine* | | glutamyl | | | sistent |
| | | | | Amino Acid | | | |
| 100001605 | | catechol | Xeno- | Benzoate | 0 | 0 | neg |
| | | sulfate | biotics | Metabolism | | | |
| X - 21341 | | X - 21341 | 0 | . | 0 | 0 | incon- |
| | | | | | | | sistent |
| 100001635 | | ectoine | Xeno- | Chemical | 0 | 0 | neg |
| | | | biotics | | | | |
| 100002129 | | pregnenolone | Lipid | Steroid | 0 | 0 | neg |
| | | sulfate | | | | | |
| 2054 | | ethylmalonate | Amino | Leucine, | 0 | 0 | incon- |
| | | | Acid | Isoleucine | | | sistent |
| | | | | and Valine | | | |
| | | | | Metabolism | | | |
| X - 12847 | | X - 12849 | 0 | . | 0 | 0 | incon- |
| | | | | | | | sistent |
| 1114 | | 3- | Nucleotide | Pyrimidine | 0 | 0 | neg |
| | | aminoisobutyrate | | Metabolism, | | | |
| | | | | Thymine | | | |
| | | | | containing | | | |
| 100001502 | | gamma- | Peptide | Gamma- | 0 | 0 | pos |
| | | glutamyl-2- | | glutamyl | | | |
| | | aminobutyrate | | Amino Acid | | | |
| 100009123 | | 1- | Lipid | Phospholipid | 0 | 0 | neg |
| | | pentadecanoyl- | | Metabolism | | | |
| | | 2-docosahexa- | | | | | |
| | | enoyl-GPC | | | | | |
| | | (15:0/22:6)* | | | | | |
| 100016069 | X - 11540 | 5- | 0 | . | 0 | 0 | pos |
| | | dodecenoyl- | | | | | |
| | | carnitine | | | | | |
| 313 | | sphinganine | Lipid | Sphingolipid | 0 | 0 | incon- |
| | | | | Metabolism | | | sistent |
| X - 17010 | | X - 17010 | 0 | . | 0 | 0 | pos |
| 1629 | | taurochenodeoxy- | Lipid | Primary | 0 | 0 | incon- |
| | | cholate | | Bile Acid | | | sistent |
| | | | | Metabolism | | | |
| 100002568 | | L-urobilin | Cofactors | Hemoglobin and | 0 | 0 | pos |
| | | | and | Porphyrin | | | |
| | | | Vitamins | Metabolism | | | |
| 100002008 | | 5alpha-androstan- | Lipid | Steroid | 0 | 0 | neg |
| | | 3alpha,17alpha-diol | | | | | |
| | | monosulfate | | | | | |
| X - 11470 | | X - 11478 | 0 | . | 0 | 0 | incon- |
| | | | | | | | sistent |
| 100002732 | | diphenhydramine | Xeno- | Drug | 0 | 0 | incon- |
| | | | biotics | | | | sistent |
| 100004251 | | dimethylmalonic | Lipid | Fatty Acid, | 0 | 0 | pos |
| | | acid | | Dicarboxylate | | | |
| 100001323 | | DSGEGDFXAEGGGVR* | Peptide | Fibrinogen | 0 | 0 | incon- |
| | | | | Cleavage | | | sistent |
| | | | | Peptide | | | |
| 444 | | ornithine | Amino | Urea cycle; | 0 | 0 | incon- |
| | | | Acid | Arginine | | | sistent |
| | | | | and Proline | | | |
| | | | | Metabolism | | | |
| 100009076 | | 1-palmitoyl- | Lipid | Phospholipid | 0 | 0 | incon- |
| | | 2-linolenoyl- | | Metabolism | | | sistent |
| | | GPC | | | | | |
| | | (16:0/18:3)* | | | | | |
| 100002196 | | 13-HODE + | Lipid | Fatty Acid, | 0 | 0 | incon- |
| | | 9-HODE | | Monohydroxy | | | sistent |
| X - 21319 | | X - 21319 | 0 | . | 0 | 0 | incon- |
| | | | | | | | sistent |
| 100001550 | | homostachydrine | Xeno- | Food | 0 | 0 | pos |
| | | | biotics | Component/ | | | |
| | | | | Plant | | | |
| 100015751 | | glycosyl- | Lipid | Ceramides | 0 | 0 | neg |
| | | ceramide | | | | | |
| | | (d18:1/23:1, | | | | | |
| | | d17:1/24:1)* | | | | | |
| 100010962 | | 1-palmityl-GPE | Lipid | Lysoplasmalogen | 0 | 0 | neg |
| | | (O-16:0)* | | | | | |
| 100000787 | | N- | Amino | Alanine and | 0 | 0 | incon- |
| | | acetylaspartate | Acid | Aspartate | | | sistent |
| | | (NAA) | | Metabolism | | | |
| 445 | | orotate | Nucleotide | Pyrimidine | 0 | 0 | incon- |
| | | | | Metabolism, | | | sistent |
| | | | | Orotate | | | |
| | | | | containing | | | |
| 100003240 | | N- | Lipid | Endocannabinoid | 0 | 0 | neg |
| | | stearoyltaurine | | | | | |
| 100001866 | | 1-stearoyl- | Lipid | Phosphatidyl- | 0 | 0 | neg |
| | | 2-oleoyl-GPG | | glycerol (PG) | | | |
| | | (18:0/18:1) | | | | | |
| X - 14568 | | X - 14568 | 0 | . | 0 | 0 | incon- |
| | | | | | | | sistent |
| 100001619 | | glycerophospho- | Lipid | Glycerolipid | 0 | 0 | neg |
| | | glycerol | | Metabolism | | | |
| 100001876 | | sphinganine-1- | Lipid | Sphingolipid | 0 | 0 | pos |
| | | phosphate | | Metabolism | | | |
| 100009078 | | 1-oleoyl-2- | Lipid | Phospholipid | 0 | 0 | neg |
| | | linoleoyl-GPE | | Metabolism | | | |
| | | (18:1/18:2)* | | | | | |
| 935 | | sucrose | Carbo- | Disaccharides and | 0 | 0 | pos |
| | | | hydrate | Oligosaccharides | | | |
| 100002500 | | formiminoglutamate | Amino | Histidine | 0 | 0 | pos |
| | | | Acid | Metabolism | | | |
| 100009264 | | glycochenodeoxy- | Lipid | Primary | 0 | 0 | pos |
| | | cholate | | Bile Acid | | | |
| | | glucuronide | | Metabolism | | | |
| | | (1) | | | | | |
| 100001211 | | sebacate | Lipid | Fatty Acid, | 0 | 0 | incon- |
| | | (decanedioate) | | Dicarboxylate | | | sistent |
| 100001956 | | N- | Amino | Urea cycle; | 0 | 0 | incon- |
| | | methylproline | Acid | Arginine | | | sistent |
| | | | | and Proline | | | |
| | | | | Metabolism | | | |
| 100001359 | | aconitate | Energy | TCA Cycle | 0 | 0 | pos |
| | | [cis or trans] | | | | | |
| 100002458 | | 3- | Amino | Leucine, | 0 | 0 | incon- |
| | | methylglutaconate | Acid | Isoleucine | | | sistent |
| | | | | and Valine | | | |
| | | | | Metabolism | | | |
| 100002951 | | eicosanodioate | Lipid | Fatty Acid, | 0 | 0 | neg |
| | | (C20-DC) | | Dicarboxylate | | | |
| 535 | | uridine | Nucleotide | Pyrimidine | 0 | 0 | incon- |
| | | | | Metabolism, | | | sistent |
| | | | | Uracil | | | |
| | | | | containing | | | |
| 100010955 | | perfluorooctane- | Xeno- | Chemical | 0 | 0 | neg |
| | | sulfonic acid | biotics | | | | |
| | | (PFOS) | | | | | |
| 100008916 | | 1-stearoyl- | Lipid | Phospholipid | 0 | 0 | incon- |
| | | 2-docosahexa- | | Metabolism | | | sistent |
| | | enoyl-GPC | | | | | |
| | | (18:0/22:6) | | | | | |
| 100001253 | | N- | Amino | Glutamate | 0 | 0 | incon- |
| | | acetylglutamine | Acid | Metabolism | | | sistent |
| 100006294 | | behenoyl | Lipid | Sphingolipid | 0 | 0 | incon- |
| | | sphingomyelin | | Metabolism | | | sistent |
| | | (d18:1/22:0)* | | | | | |
| 100008994 | | 1-stearoyl- | Lipid | Phospholipid | 0 | 0 | incon- |
| | | 2-linoleoyl- | | Metabolism | | | sistent |
| | | GPI | | | | | |
| | | (18:0/18:2) | | | | | |
| 100005717 | | 1-palmitoyl- | Lipid | Lysolipid | 0 | 0 | pos |
| | | GPG (16:0)* | | | | | |
| 100000295 | | tartarate | Xeno- | Food | 0 | 0 | neg |
| | | | biotics | Component/ | | | |
| | | | | Plant | | | |
| 100002390 | | 4-methyl- | Xeno- | Chemical | 0 | 0 | neg |
| | | benzenesulfonate | biotics | | | | |
| 100003006 | | 2-linoleoyl- | Lipid | Lysophospholipid | 0 | 0 | neg |
| | | GPI (18:2)* | | | | | |
| 100002417 | | 2,3- | Xeno- | Food | 0 | 0 | neg |
| | | dihydroxy- | biotics | Component/ | | | |
| | | isovalerate | | Plant | | | |
| 100006373 | | 1,2,3- | Xeno- | Chemical | 0 | 0 | neg |
| | | benzenetriol | biotics | | | | |
| | | sulfate (1) | | | | | |
| 100009075 | | 1-palmitoleoyl- | Lipid | Phospholipid | 0 | 0 | incon- |
| | | 2-linoleoyl- | | Metabolism | | | sistent |
| | | GPC | | | | | |
| | | (16:1/18:2)* | | | | | |
| 100000039 | | methionine | Amino | Methionine, | 0 | 0 | pos |
| | | sulfoxide | Acid | Cysteine, | | | |
| | | | | SAM and | | | |
| | | | | Taurine | | | |
| | | | | Metabolism | | | |
| 100009069 | | 1-(1-enyl- | Lipid | Plasmalogen | 0 | 0 | incon- |
| | | palmitoyl)- | | | | | sistent |
| | | 2-linoleoyl- | | | | | |
| | | GPE (P- | | | | | |
| | | 16:0/18:2)* | | | | | |
| X - 12729 | | X - 12730 | 0 | . | 0 | 0 | incon- |
| | | | | | | | sistent |
| X - 11795 | | X - 11805 | 0 | . | 0 | 0 | neg |
| 100000900 | | iminodiacetate | Xeno- | Chemical | 0 | 0 | neg |
| | | (IDA) | biotics | | | | |
| X - 18888 | | X - 18888 | 0 | . | 0 | 0 | neg |
| 136 | | cholate | Lipid | Primary | 0 | 0 | incon- |
| | | | | Bile Acid | | | sistent |
| | | | | Metabolism | | | |
| X - 23587 | | X - 23587 | 0 | . | 0 | 0 | pos |
| 100001198 | | myristoleate | Lipid | Long Chain | 0 | 0 | pos |
| | | (14:1n5) | | Fatty Acid | | | |
| 100020492 | X - 01911 | glucuronide | 0 | . | 0 | 0 | incon- |
| | | of piperine | | | | | sistent |
| | | metabolite | | | | | |
| | | C17H21NO3 (4)* | | | | | |
| X - 11852 | | X - 11858 | 0 | . | 0 | 0 | neg |
| 100006298 | | lignoceroyl | Lipid | Sphingolipid | 0 | 0 | neg |
| | | sphingomyelin | | Metabolism | | | |
| | | (d18:1/24:0) | | | | | |
| 891 | | margarate | Lipid | Long Chain | 0 | 0 | incon- |
| | | (17:0) | | Fatty Acid | | | sistent |
| 111 | | 3-hydroxy- | Amino | Leucine, | 0 | 0 | incon- |
| | | isobutyrate | Acid | Isoleucine | | | sistent |
| | | | | and Valine | | | |
| | | | | Metabolism | | | |
| 418 | | methylmalonate | Lipid | Fatty Acid | 0 | 0 | pos |
| | | (MMA) | | Metabolism | | | |
| | | | | (also BCAA | | | |
| | | | | Metabolism) | | | |
| 100001269 | | campesterol | Lipid | Sterol | 0 | 0 | neg |
| 100001229 | | stearidonate | Lipid | Polyun- | 0 | 0 | incon- |
| | | (18:4n3) | | saturated | | | sistent |
| | | | | Fatty Acid | | | |
| | | | | (n3 and n6) | | | |
| 1124 | | citrate | Energy | TCA Cycle | 0 | 0 | neg |
| 100020208 | X - 11305 | perfluorooctanoate | 0 | . | 0 | 0 | neg |
| | | (PFOA)* | | | | | |
| 100009215 | | 1-stearoyl- | Lipid | Phosphatidyl- | 0 | 0 | pos |
| | | 2-dihomo- | | ethanolamine | | | |
| | | linolenoyl-GPE | | (PE) | | | |
| | | (18:0/20:3n3 | | | | | |
| | | or 6)* | | | | | |
| 100000672 | | 1-myristoyl- | Lipid | Phospholipid | 0 | 0 | incon- |
| | | 2-palmitoyl- | | Metabolism | | | sistent |
| | | GPC | | | | | |
| | | (14:0/16:0) | | | | | |
| X - 12730 | | X - 12738 | 0 | . | 0 | 0 | incon- |
| | | | | | | | sistent |
| X - 18913 | | X - 18913 | 0 | . | 0 | 0 | incon- |
| | | | | | | | sistent |
| 100003470 | | pregnanediol-3- | Lipid | Steroid | 0 | 0 | neg |
| | | glucuronide | | | | | |
| 100001293 | | N- | Amino | Histidine | 0 | 0 | neg |
| | | acetylhistidine | Acid | Metabolism | | | |
| 100000898 | | glycylglycine | Peptide | Dipeptide | 0 | 0 | pos |
| 100000008 | | benzoate | Xeno- | Benzoate | 0 | 0 | pos |
| | | | biotics | Metabolism | | | |
| 432 | | nicotinamide | Cofactors | Nicotinate | 0 | 0 | pos |
| | | | and | and | | | |
| | | | Vitamins | Nicotinamide | | | |
| | | | | Metabolism | | | |
| 100009005 | | 1-(1-enyl- | Lipid | Plasmalogen | 0 | 0 | incon- |
| | | palmitoyl)- | | | | | sistent |
| | | 2-oleoyl- | | | | | |
| | | GPE (P- | | | | | |
| | | 16:0/18:1)* | | | | | |
| X - 11849 | | X - 11850 | 0 | . | 0 | 0 | neg |
| 100002806 | | gabapentin | Xeno- | Drug | 0 | 0 | pos |
| | | | biotics | | | | |
| 231 | | arginine | Amino | Urea cycle; | 0 | 0 | incon- |
| | | | Acid | Arginine | | | sistent |
| | | | | and Proline | | | |
| | | | | Metabolism | | | |
| 100020014 | X - 16947 | glucuronide | 0 | . | 0 | 0 | pos |
| | | of | | | | | |
| | | C10H18O2 | | | | | |
| | | (7)* | | | | | |
| 100001662 | | deoxycarnitine | Lipid | Carnitine | 0 | 0 | pos |
| | | | | Metabolism | | | |
| 100001092 | | trigonelline | Cofactors | Nicotinate | 0 | 0 | incon- |
| | | (N′- | and | and | | | sistent |
| | | methylnicotinate) | Vitamins | Nicotinamide | | | |
| | | | | Metabolism | | | |
| 519 | | myristate | Lipid | Long Chain | 0 | 0 | pos |
| | | (14:0) | | Fatty Acid | | | |
| 342 | | glycocholate | Lipid | Primary | 0 | 0 | incon- |
| | | | | Bile Acid | | | sistent |
| | | | | Metabolism | | | |
| 100002153 | | betonicine | Xeno- | Food | 0 | 0 | incon- |
| | | | biotics | Component/ | | | sistent |
| | | | | Plant | | | |
| 100002734 | | hydrochlorothiazide | Xeno- | Drug | 0 | 0 | incon- |
| | | | biotics | | | | sistent |
| 1026 | | phosphoethanolamine | Lipid | Phospholipid | 0 | 0 | neg |
| | | | | Metabolism | | | |
| 100009082 | | 1-linoleoyl- | Lipid | Lysolipid | 0 | 0 | neg |
| | | GPA | | | | | |
| | | (18:2)* | | | | | |
| 100003769 | | norfluoxetine | Xeno- | Drug | 0 | 0 | incon- |
| | | | biotics | | | | sistent |
| 100010966 | | 1-palmityl- | Lipid | Plasmalogen | 0 | 0 | neg |
| | | 2-stearoyl- | | | | | |
| | | GPC (O- | | | | | |
| | | 16:0/18:0)* | | | | | |
| X - 23314 | | X - 23314 | 0 | . | 0 | 0 | neg |
| 100009167 | | phosphocholine | Lipid | phospholipid | 0 | 0 | incon- |
| | | (18:0/20:5, | | | | | sistent |
| | | 16:0/22:5n6)* | | | | | |
| X - 17685 | | X - 17685 | 0 | . | 0 | 0 | incon- |
| | | | | | | | sistent |
| 100002813 | | leukotriene | Lipid | Eicosanoid | 0 | 0 | pos |
| | | B5 | | | | | |
| X - 12816 | | X - 12822 | 0 | . | 0 | 0 | neg |
| X - 10458 | | X - 11261 | 0 | . | 0 | 0 | incon- |
| | | | | | | | sistent |
| 1021 | | 5- | Amino | Glutathione | 0 | 0 | neg |
| | | oxoproline | Acid | Metabolism | | | |
| X - 17185 | | X - 17185 | 0 | . | 0 | 0 | neg |
| 100004227 | | 2- | Lipid | Fatty Acid, | 0 | 0 | incon- |
| | | aminooctanoate | | Amino | | | sistent |
| 100000773 | | 3- | Lipid | Fatty Acid, | 0 | 0 | pos |
| | | hydroxyoctanoate | | Monohydroxy | | | |
| 100000792 | | dehydroiso- | Lipid | Steroid | 0 | 0 | incon- |
| | | androsterone | | | | | sistent |
| | | sulfate | | | | | |
| | | (DHEA-S) | | | | | |
| 100001743 | | tryptophan | Amino | Tryptophan | 0 | 0 | neg |
| | | betaine | Acid | Metabolism | | | |
| 100006374 | | 1,2,3- | Xeno- | Chemical | 0 | 0 | incon- |
| | | benzenetriol | biotics | | | | sistent |
| | | sulfate (2) | | | | | |
| 100015640 | | N- | Lipid | Endocannabinoid | 0 | 0 | neg |
| | | palmitoylserine | | | | | |
| 512 | | taurine | Amino | Methionine, | 0 | 0 | incon- |
| | | | Acid | Cysteine, | | | sistent |
| | | | | SAM and | | | |
| | | | | Taurine | | | |
| | | | | Metabolism | | | |
| 926 | | caproate | Lipid | Medium | 0 | 0 | incon- |
| | | (6:0) | | Chain Fatty | | | sistent |
| | | | | Acid | | | |
| 229 | | arachidonate | Lipid | Polyun- | 0 | 0 | incon- |
| | | (20:4n6) | | saturated | | | sistent |
| | | | | Fatty Acid | | | |
| | | | | (n3 and n6) | | | |
| 100001624 | | 3-(3-hydroxy- | Amino | Phenylalanine | 0 | 0 | incon- |
| | | phenyl)propionate | Acid | and Tyrosine | | | sistent |
| | | | | Metabolism | | | |
| 100004208 | | O- | Xeno- | Benzoate | 0 | 0 | pos |
| | | methylcatechol | biotics | Metabolism | | | |
| | | sulfate | | | | | |
| 100001232 | | 5- | Lipid | Medium | 0 | 0 | neg |
| | | dodecenoate | | Chain Fatty | | | |
| | | (12:1n7) | | Acid | | | |
| 100003651 | | methionylalanine | Peptide | Dipeptide | 0 | 0 | neg |
| 100001989 | | glycocholenate | Lipid | Secondary | 0 | 0 | pos |
| | | sulfate* | | Bile Acid | | | |
| | | | | Metabolism | | | |
| X - 12543 | | X - 12680 | 0 | . | 0 | 0 | incon- |
| | | | | | | | sistent |
| 100001571 | | 1- | Lipid | Lysolipid | 0 | 0 | incon- |
| | | arachidonoyl- | | | | | sistent |
| | | GPE (20:4)* | | | | | |
| 925 | | heptanoate | Lipid | Medium | 0 | 0 | neg |
| | | (7:0) | | Chain Fatty | | | |
| | | | | Acid | | | |
| 100003178 | | isoleucylvaline | Peptide | Dipeptide | 0 | 0 | incon- |
| | | | | | | | sistent |
| 100000781 | | hexanoylcarnitine | Lipid | Fatty Acid | 0 | 0 | incon- |
| | | | | Metabolism | | | sistent |
| | | | | (Acyl | | | |
| | | | | Carnitine) | | | |
| X - 12680 | | X - 12681 | 0 | . | 0 | 0 | incon- |
| | | | | | | | sistent |
| 100009014 | | 1-(1-enyl- | Lipid | Phospholipid | 0 | 0 | incon- |
| | | palmitoyl)- | | Metabolism | | | sistent |
| | | 2-arachidonoyl- | | | | | |
| | | GPC (P- | | | | | |
| | | 16:0/20:4)* | | | | | |
| 100001073 | | androsterone | Lipid | Steroid | 0 | 0 | incon- |
| | | sulfate | | | | | sistent |
| 100001197 | | 10-undecenoate | Lipid | Medium | 0 | 0 | incon- |
| | | (11:1n1) | | Chain Fatty | | | sistent |
| | | | | Acid | | | |
| 100003442 | | paroxetine | Xeno- | Drug | 0 | 0 | neg |
| | | | biotics | | | | |
| 100001778 | | 1-linoleoyl- | Lipid | Lysolipid | 0 | 0 | incon- |
| | | GPI (18:2)* | | | | | sistent |
| 1487 | | ibuprofen | Xeno- | Drug | 0 | 0 | incon- |
| | | | biotics | | | | sistent |
| 100001034 | | indoleacetate | Amino | Tryptophan | 0 | 0 | incon- |
| | | | Acid | Metabolism | | | sistent |
| 1382 | | fluoxetine | Xeno- | Drug | 0 | 0 | incon- |
| | | | biotics | | | | sistent |
| 1648 | | taurocholate | Lipid | Primary | 0 | 0 | incon- |
| | | | | Bile Acid | | | sistent |
| | | | | Metabolism | | | |
| 100015730 | | glycosyl | Lipid | Ceramides | 0 | 0 | pos |
| | | ceramide | | | | | |
| | | (d16:1/24:1, | | | | | |
| | | d18:1/22:1)* | | | | | |
| 100015625 | | glycosyl-N- | Lipid | Ceramides | 0 | 0 | neg |
| | | behenoyl- | | | | | |
| | | sphingadienine | | | | | |
| | | (d18:2/22:0)* | | | | | |
| X - 16938 | | X - 16938 | 0 | . | 0 | 0 | neg |
| 192 | | N- | Amino | Polyamine | 0 | 0 | incon- |
| | | acetylputrescine | Acid | Metabolism | | | sistent |
| 100001086 | | N-(2- | Xeno- | Food | 0 | 0 | incon- |
| | | furoyl)glycine | biotics | Component/ | | | sistent |
| | | | | Plant | | | |
| 1230 | | estrone 3- | Lipid | Steroid | 0 | 0 | incon- |
| | | sulfate | | | | | sistent |
| 100001561 | | 2- | Lipid | Lysolipid | 0 | 0 | pos |
| | | palmitoleoyl- | | | | | |
| | | GPC (16:1)* | | | | | |
| 100015594 | | 1-stearoyl- | Lipid | Phosphatidyl- | 0 | 0 | pos |
| | | 2-adrenoyl- | | ethanolamine | | | |
| | | GPE | | (PE) | | | |
| | | (18:0/22:4)* | | | | | |
| 100000551 | | 4-methyl-2- | Amino | Leucine, | 0 | 0 | incon- |
| | | oxopentanoate | Acid | Isoleucine | | | sistent |
| | | | | and Valine | | | |
| | | | | Metabolism | | | |
| X - 10358 | | X - 10458 | 0 | . | 0 | 0 | neg |
| 100006619 | | 4-hydroxy- | Amino | Tyrosine | 0 | 0 | neg |
| | | phenylacetatoyl | Acid | Metabolism | | | |
| | | carnitine | | | | | |
| 100015836 | | ximenoylcarnitine | Lipid | Fatty Acid | 0 | 0 | neg |
| | | (C26:1)* | | Metabolism | | | |
| | | | | (Acyl | | | |
| | | | | Carnitine) | | | |
| 100001999 | | 21-hydroxy- | Lipid | Steroid | 0 | 0 | incon- |
| | | pregnenolone | | | | | sistent |
| | | disulfate | | | | | |
| 100004015 | | diltiazem | Xeno- | Drug | 0 | 0 | incon- |
| | | | biotics | | | | sistent |
| 100015789 | | sphingomyelin | Lipid | Sphingolipid | 0 | 0 | neg |
| | | (d18:2/24:2)* | | Metabolism | | | |
| 100001267 | | piperine | Xeno- | Food | 0 | 0 | incon- |
| | | | biotics | Component/ | | | sistent |
| | | | | Plant | | | |
| 100002406 | | ibuprofen | Xeno- | Drug | 0 | 0 | pos |
| | | acyl | biotics | | | | |
| | | glucuronide | | | | | |
| 818 | | malonate | Lipid | Fatty Acid | 0 | 0 | incon- |
| | | | | Synthesis | | | sistent |
| 100002135 | | 5-HEPE | Lipid | Eicosanoid | 0 | 0 | incon- |
| | | | | | | | sistent |
| 207 | | adenosine | Nucleotide | Purine | 0 | 0 | pos |
| | | 3′,5′-cyclic | | Metabolism, | | | |
| | | monophosphate | | Adenine | | | |
| | | (cAMP) | | containing | | | |
| 491 | | pyridoxal | Cofactors | Vitamin B6 | 0 | 0 | neg |
| | | | and | Metabolism | | | |
| | | | Vitamins | | | | |
| 100009225 | | 1-(1-enyl- | Lipid | Plasmalogen | 0 | 0 | neg |
| | | stearoyl)-2- | | | | | |
| | | linoleoyl- | | | | | |
| | | GPE (P- | | | | | |
| | | 18:0/18:2) | | | | | |
| 100003250 | | alpha- | Peptide | Dipeptide | 0 | 0 | incon- |
| | | glutamyltyrosine | | | | | sistent |
| 100006435 | | N-acetyl- | Carbo- | Aminosugar | 0 | 0 | pos |
| | | glucosamine/ | hydrate | Metabolism | | | |
| | | N-acetyl- | | | | | |
| | | galactosamine | | | | | |
| X - 12221 | | X - 12230 | 0 | . | 0 | 0 | incon- |
| | | | | | | | sistent |
| 100001337 | | linolenate | Lipid | Polyun- | 0 | 0 | incon- |
| | | [alpha or | | saturated | | | sistent |
| | | gamma; | | Fatty Acid | | | |
| | | (18:3n3 or 6)] | | (n3 and n6) | | | |
| 100004295 | | 2- | Xeno- | Food | 0 | 0 | incon- |
| | | piperidinone | biotics | Component/ | | | sistent |
| | | | | Plant | | | |
| X - 21441 | | X - 21441 | 0 | . | 0 | 0 | pos |
| 100002014 | | 5alpha-pregnan- | Lipid | Steroid | 0 | 0 | incon- |
| | | 3beta,20alpha-diol | | | | | sistent |
| | | monosulfate (2) | | | | | |
| 501 | | salicylate | Xeno- | Drug | 0 | 0 | incon- |
| | | | biotics | | | | sistent |
| 100006203 | X - 13848 | acesulfame | Xeno- | Food | 0 | 0 | incon- |
| | | | biotics | Component/ | | | sistent |
| | | | | Plant | | | |
| 100008996 | | 1-palmitoyl- | Lipid | Phospholipid | 0 | 0 | pos |
| | | 2-palmitoleoyl- | | Metabolism | | | |
| | | GPE | | | | | |
| | | (16:0/16:1)* | | | | | |
| 100001757 | | thymol | Xeno- | Food | 0 | 0 | incon- |
| | | sulfate | biotics | Component/ | | | sistent |
| | | | | Plant | | | |
| 100004112 | | 3-methyl | Xeno- | Benzoate | 0 | 0 | incon- |
| | | catechol | biotics | Metabolism | | | sistent |
| | | sulfate (1) | | | | | |
| 100015835 | | cerotoylcarnitine | Lipid | Fatty Acid | 0 | 0 | neg |
| | | (C26)* | | Metabolism | | | |
| | | | | (Acyl | | | |
| | | | | Carnitine) | | | |
| 100003101 | | alpha- | Cofactors | Tocopherol | 0 | 0 | pos |
| | | CEHC | and | Metabolism | | | |
| | | glucuronide | Vitamins | | | | |
| 100010939 | | palmitoyl- | Lipid | Diacylglycerol | 0 | 0 | pos |
| | | linolenoyl- | | | | | |
| | | glycerol | | | | | |
| | | (16:0/18:3) | | | | | |
| | | [2]* | | | | | |
| X - 12738 | | X - 12740 | 0 | . | 0 | 0 | incon- |
| | | | | | | | sistent |
| 100001551 | | 1- | Lipid | Lysolipid | 0 | 0 | incon- |
| | | arachidonoyl- | | | | | sistent |
| | | GPC (20:4)* | | | | | |
| 50 | | spermidine | Amino | Polyamine | 0 | 0 | incon- |
| | | | Acid | Metabolism | | | sistent |
| X - 23369 | | X - 23369 | 0 | . | 0 | 0 | pos |
| 100002871 | | 1-adrenoyl- | Lipid | Lysolipid | 0 | 0 | pos |
| | | GPC (22:4)* | | | | | |
| 100001270 | | myristoylcarnitine | Lipid | Fatty Acid | 0 | 0 | incon- |
| | | | | Metabolism | | | sistent |
| | | | | (Acyl | | | |
| | | | | Carnitine) | | | |
| 100015586 | | 1-palmityl- | Lipid | Plasmalogen | 0 | 0 | neg |
| | | 2-palmitoyl- | | | | | |
| | | GPC (0- | | | | | |
| | | 16:0/16:0)* | | | | | |
| 100009140 | | 1-myristoyl- | Lipid | Phospholipid | 0 | 0 | pos |
| | | 2-docosahexa- | | Metabolism | | | |
| | | enoyl-GPC | | | | | |
| | | (14:0/22:6)* | | | | | |
| X - 23997 | | X - 23997 | 0 | . | 0 | 0 | neg |
| 100004169 | | 2- | Xeno- | Drug | 0 | 0 | incon- |
| | | hydroxyibuprofen | biotics | | | | sistent |
| 461 | | phosphate | Energy | Oxidative | 0 | 0 | incon- |
| | | | | Phosphorylation | | | sistent |
| 100010928 | | linoleoyl- | Lipid | Diacylglycerol | 0 | 0 | pos |
| | | docosahexaenoyl- | | | | | |
| | | glycerol | | | | | |
| | | (18:2/22:6) | | | | | |
| | | [1]* | | | | | |
| 100001950 | | bilirubin | Cofactors | Hemoglobin | 0 | 0 | neg |
| | | (E,E)* | and | and Porphyrin | | | |
| | | | Vitamins | Metabolism | | | |
| 100001287 | | epiandrosterone | Lipid | Steroid | 0 | 0 | incon- |
| | | sulfate | | | | | sistent |
| 1829 | | cis- | Energy | TCA Cycle | 0 | 0 | pos |
| | | aconitate | | | | | |
| 100010869 | | 2,3- | Amino | Leucine, | 0 | 0 | neg |
| | | dihydroxy-2- | Acid | Isoleucine | | | |
| | | methylbutyrate | | and Valine | | | |
| | | | | Metabolism | | | |
| X - 23649 | | X - 23649 | 0 | . | 0 | 0 | incon- |
| | | | | | | | sistent |
| 363 | | myo-inositol | Lipid | Inositol | 0 | 0 | incon- |
| | | | | Metabolism | | | sistent |
| X - 12013 | | X - 12026 | 0 | . | 0 | 0 | neg |
| 100015792 | | sphingomyelin | Lipid | Sphingolipid | 0 | 0 | pos |
| | | (d18:1/25:0, | | Metabolism | | | |
| | | d19:0/24:1, | | | | | |
| | | d20:1/23:0, | | | | | |
| | | d19:1/24:0)* | | | | | |
| X - 17690 | | X - 17690 | 0 | . | 0 | 0 | incon- |
| | | | | | | | sistent |
| 100008919 | | 1-(1-enyl- | Lipid | Plasmalogen | 0 | 0 | incon- |
| | | stearoyl)-2- | | | | | sistent |
| | | oleoyl-GPE | | | | | |
| | | (P-18:0/18:1) | | | | | |
| 100006051 | | myristoleoyl- | Lipid | Fatty Acid | 0 | 0 | incon- |
| | | carnitine* | | Metabolism | | | sistent |
| | | | | (Acyl | | | |
| | | | | Carnitine) | | | |
| 100001423 | | 4- | Xeno- | Benzoate | 0 | 0 | incon- |
| | | hydroxyhippurate | biotics | Metabolism | | | sistent |
| X - 15469 | | X - 15469 | 0 | . | 0 | 0 | incon- |
| | | | | | | | sistent |
| 100001207 | | 4- | Amino | Histidine | 0 | 0 | incon- |
| | | imidazoleacetate | Acid | Metabolism | | | sistent |
| 100000708 | | isovalerate | Amino | Leucine, | 0 | 0 | incon- |
| | | | Acid | Isoleucine | | | sistent |
| | | | | and Valine | | | |
| | | | | Metabolism | | | |
| 100001081 | | 2- | Xeno- | Chemical | 0 | 0 | neg |
| | | pyrrolidinone | biotics | | | | |
| 100001108 | | 3- | Xeno- | Xanthine | 0 | 0 | pos |
| | | methylxanthine | biotics | Metabolism | | | |
| 100001026 | | galactonate | Carbo- | Fructose, | 0 | 0 | incon- |
| | | | hydrate | Mannose and | | | sistent |
| | | | | Galactose | | | |
| | | | | Metabolism | | | |
| X - 21735 | | X - 21735 | 0 | . | 0 | 0 | pos |
| 1668 | | taurodeoxycholate | Lipid | Secondary | 0 | 0 | incon- |
| | | | | Bile Acid | | | sistent |
| | | | | Metabolism | | | |
| 100009217 | | 1,2- | Lipid | Phosphatidyl- | 0 | 0 | neg |
| | | dilinoleoyl- | | ethanolamine | | | |
| | | GPE | | (PE) | | | |
| | | (18:2/18:2)* | | | | | |
| 100002102 | | N-acetyl- | Nucleotide | Pyrimidine | 0 | 0 | incon- |
| | | beta-alanine | | Metabolism, | | | sistent |
| | | | | Uracil | | | |
| | | | | containing | | | |
| 278 | | cysteinylglycine | Amino | Glutathione | 0 | 0 | incon- |
| | | | Acid | Metabolism | | | sistent |
| 100008955 | | tricosanoyl | Lipid | Sphingolipid | 0 | 0 | incon- |
| | | sphingomyelin | | Metabolism | | | sistent |
| | | (d18:1/23:0)* | | | | | |
| 100010923 | | linoleoyl- | Lipid | Diacylglycerol | 0 | 0 | pos |
| | | arachidonoyl- | | | | | |
| | | glycerol | | | | | |
| | | (18:2/20:4) | | | | | |
| | | [2]* | | | | | |
| 100004111 | | 4- | Xeno- | Benzoate | 0 | 0 | neg |
| | | methylcatechol | biotics | Metabolism | | | |
| | | sulfate | | | | | |
| X - 11299 | | X - 11305 | 0 | . | 0 | 0 | incon- |
| | | | | | | | sistent |
| X - 12462 | | X - 12472 | 0 | . | 0 | 0 | incon- |
| | | | | | | | sistent |
| 100000442 | | quinate | Xeno- | Food | 0 | 0 | incon- |
| | | | biotics | Component/ | | | sistent |
| | | | | Plant | | | |
| 100000656 | | 1-stearoyl- | Lipid | Lysolipid | 0 | 0 | incon- |
| | | GPI (18:0) | | | | | sistent |
| 100000964 | | alpha- | Peptide | Dipeptide | 0 | 0 | incon- |
| | | glutamyl- | | | | | sistent |
| | | glutamate | | | | | |
| 100009406 | | palmitoleoyl | Lipid | Fatty Acid | 0 | 0 | neg |
| | | carnitine | | Metabolism | | | |
| | | (C16:1) | | (Acyl | | | |
| | | | | Carnitine) | | | |
| 100010924 | | palmitoyl- | Lipid | Diacylglycerol | 0 | 0 | pos |
| | | arachidonoyl- | | | | | |
| | | glycerol | | | | | |
| | | (16:0/20:4) | | | | | |
| | | [1]* | | | | | |
| 100001121 | | pyridoxate | Cofactors | Vitamin B6 | 0 | 0 | incon- |
| | | | and | Metabolism | | | sistent |
| | | | Vitamins | | | | |
| 828 | | arabinose | Carbo- | Pentose | 0 | 0 | pos |
| | | | hydrate | Metabolism | | | |
| 100001767 | | pyrraline | Xeno- | Food | 0 | 0 | pos |
| | | | biotics | Component/ | | | |
| | | | | Plant | | | |
| 100003473 | | alliin | Xeno- | Food | 0 | 0 | neg |
| | | | biotics | Component/ | | | |
| | | | | Plant | | | |
| 100003239 | | N- | Lipid | Endocannabinoid | 0 | 0 | neg |
| | | palmitoyltaurine | | | | | |
| 100015735 | | ceramide | Lipid | Ceramides | 0 | 0 | pos |
| | | (d18:1/14:0, | | | | | |
| | | d16:1/16:0)* | | | | | |
| X - 21444 | | X - 21444 | 0 | . | 0 | 0 | incon- |
| | | | | | | | sistent |
| 251 | | biotin | Cofactors | Biotin | 0 | 0 | pos |
| | | | and | Metabolism | | | |
| | | | Vitamins | | | | |
| 100004442 | | 1- | Lipid | Lysolipid | 0 | 0 | pos |
| | | arachidonoyl- | | | | | |
| | | GPA (20:4) | | | | | |
| 100001540 | | pyroglutamine* | Amino | Glutamate | 0 | 0 | incon- |
| | | | Acid | Metabolism | | | sistent |
| 100002027 | | 4-androsten- | Lipid | Steroid | 0 | 0 | incon- |
| | | 3alpha,17alpha- | | | | | sistent |
| | | diol | | | | | |
| | | monosulfate (3) | | | | | |
| 100015840 | | dihomo- | Lipid | Fatty Acid | 0 | 0 | neg |
| | | linolenoylcarnitine | | Metabolism | | | |
| | | (20:3n3 or 6)* | | (Acyl | | | |
| | | | | Carnitine) | | | |
| 100006361 | | dopamine | Amino | Phenylalanine | 0 | 0 | incon- |
| | | sulfate (2) | Acid | and Tyrosine | | | sistent |
| | | | | Metabolism | | | |
| X - 23974 | | X - 23974 | 0 | . | 0 | 0 | incon- |
| | | | | | | | sistent |
| X - 12007 | | X - 12013 | 0 | . | 0 | 0 | incon- |
| | | | | | | | sistent |
| 100001195 | | docosatrienoate | Lipid | Polyun- | 0 | 0 | pos |
| | | (22:3n3) | | saturated | | | |
| | | | | Fatty Acid | | | |
| | | | | (n3 and n6) | | | |
| 100000263 | | imidazole | Amino | Histidine | 0 | 0 | neg |
| | | lactate | Acid | Metabolism | | | |
| 100002035 | | 5alpha-pregnan- | Lipid | Progestin | 0 | 0 | neg |
| | | 3beta-ol,20-one | | Steroids | | | |
| | | sulfate | | | | | |
| 100009154 | | 1-palmitoyl- | Lipid | Phosphatidyl- | 0 | 0 | pos |
| | | 2-gamma- | | choline (PC) | | | |
| | | linolenoyl- | | | | | |
| | | GPC | | | | | |
| | | (16:0/18:3n6)* | | | | | |
| 100000409 | | 2- | Xeno- | Food | 0 | 0 | incon- |
| | | isopropylmalate | biotics | Component/ | | | sistent |
| | | | | Plant | | | |
| 2051 | | methylsuccinate | Amino | Leucine, | 0 | 0 | incon- |
| | | | Acid | Isoleucine | | | sistent |
| | | | | and Valine | | | |
| | | | | Metabolism | | | |
| 100001257 | | N- | Amino | Alanine and | 0 | 0 | incon- |
| | | acetylasparagine | Acid | Aspartate | | | sistent |
| | | | | Metabolism | | | |
| 100006369 | | N- | Amino | Alanine and | 0 | 0 | neg |
| | | carbamoylalanine | Acid | Aspartate | | | |
| | | | | Metabolism | | | |
| X - 11483 | | X - 11491 | 0 | . | 0 | 0 | incon- |
| | | | | | | | sistent |
| 100003668 | | prolylalanine | Peptide | Dipeptide | 0 | 0 | pos |
| 100003008 | | 2-stearoyl- | Lipid | Lysolipid | 0 | 0 | incon- |
| | | GPI (18:0)* | | | | | sistent |
| 100000647 | | 1,2- | Lipid | Phospholipid | 0 | 0 | incon- |
| | | dimyristoyl- | | Metabolism | | | sistent |
| | | GPC | | | | | |
| | | (14:0/14:0) | | | | | |
| 100003915 | | palmitic | Lipid | Fatty Acid, | 0 | 0 | neg |
| | | amide | | Amide | | | |
| 100001277 | | 10- | Lipid | Long Chain | 0 | 0 | incon- |
| | | nonadecenoate | | Fatty Acid | | | sistent |
| | | (19:1n9) | | | | | |
| 100010948 | | palmitoyl- | Lipid | Diacylglycerol | 0 | 0 | pos |
| | | palmitoyl- | | | | | |
| | | glycerol | | | | | |
| | | (16:0/16:0) | | | | | |
| | | [2]* | | | | | |
| X - 23046 | | X - 23046 | 0 | . | 0 | 0 | pos |
| 100002537 | | 4-hydroxy- | Lipid | Fatty Acid, | 0 | 0 | pos |
| | | 2-oxoglutaric | | Dicarboxylate | | | |
| | | acid | | | | | |
| 100009232 | X - 12792 | thioproline | Amino | Tryptophan | 0 | 0 | incon- |
| | | | Acid | Metabolism | | | sistent |
| 100001402 | | 5-acetylamino- | Xeno- | Xanthine | 0 | 0 | incon- |
| | | 6-formylamino- | biotics | Metabolism | | | sistent |
| | | 3-methyluracil | | | | | |
| 100015882 | | glycosyl | Lipid | Ceramides | 0 | 0 | neg |
| | | ceramide | | | | | |
| | | (d18:1/20:0, | | | | | |
| | | d16:1/22:0)* | | | | | |
| 100006375 | | 3- | Xeno- | Benzoate | 0 | 0 | incon- |
| | | methoxycatechol | biotics | Metabolism | | | sistent |
| | | sulfate (1) | | | | | |
| 100001226 | | l- | Cofactors | Hemoglobin | 0 | 0 | pos |
| | | urobilinogen | and | and Porphyrin | | | |
| | | | Vitamins | Metabolism | | | |
| 100001554 | | 2- | Lipid | Lysolipid | 0 | 0 | incon- |
| | | arachidonoyl- | | | | | sistent |
| | | GPC (20:4)* | | | | | |
| X - 12830 | | X - 12844 | 0 | . | 0 | 0 | pos |
| 100003151 | | linoleoylcarnitine* | Lipid | Fatty Acid | 0 | 0 | incon- |
| | | | | Metabolism | | | sistent |
| | | | | (Acyl | | | |
| | | | | Carnitine) | | | |
| 100001332 | | salicyluric | Xeno- | Drug | 0 | 0 | incon- |
| | | glucuronide* | biotics | | | | sistent |
| 100002868 | | 1-behenoyl- | Lipid | Lysophospholipid | 0 | 0 | neg |
| | | GPC (22:0) | | | | | |
| 100001674 | | 2-arachidonoyl- | Lipid | Lysolipid | 0 | 0 | incon- |
| | | GPE (20:4)* | | | | | sistent |
| X - 17146 | | X - 17146 | 0 | . | 0 | 0 | incon- |
| | | | | | | | sistent |
| 100001429 | | 1- | Lipid | Monoacylglycerol | 0 | 0 | incon- |
| | | margaroylglycerol | | | | | sistent |
| | | (17:0) | | | | | |
| X - 12407 | | X - 12411 | 0 | . | 0 | 0 | incon- |
| | | | | | | | sistent |
| 2028 | | metoprolol | Xeno- | Drug | 0 | 0 | neg |
| | | | biotics | | | | |
| 100009079 | | 1-palmitoyl- | Lipid | Phospholipid | 0 | 0 | neg |
| | | 2-dihomo- | | Metabolism | | | |
| | | linolenoyl-GPE | | | | | |
| | | (16:0/20:3)* | | | | | |
| 100001411 | | beta-guanidino- | Xeno- | Food | 0 | 0 | pos |
| | | propanoate | biotics | Component/ | | | |
| | | | | Plant | | | |
| X - 17327 | | X - 17327 | 0 | . | 0 | 0 | incon- |
| | | | | | | | sistent |
| 100006173 | | pregnanolone/ | Lipid | Steroid | 0 | 0 | incon- |
| | | allopregnanolone | | | | | sistent |
| | | sulfate | | | | | |
| 100009378 | | 1-(1-enyl- | Lipid | Plasmalogen | 0 | 0 | pos |
| | | stearoyl)-2- | | | | | |
| | | dihomo- | | | | | |
| | | linolenoyl- | | | | | |
| | | GPE (P- | | | | | |
| | | 18:0/20:3)* | | | | | |
| 100006627 | | suberoylcarnitine | Lipid | Fatty Acid | 0 | 0 | pos |
| | | (C8-DC) | | Metabolism | | | |
| | | | | (Acyl | | | |
| | | | | Carnitine) | | | |
| X - 12472 | | X - 12511 | 0 | . | 0 | 0 | incon- |
| | | | | | | | sistent |
| 1231 | | dihomo- | Lipid | Polyun- | 0 | 0 | incon- |
| | | linoleate | | saturated | | | sistent |
| | | (20:2n6) | | Fatty Acid | | | |
| | | | | (n3 and n6) | | | |
| 100006641 | | glycochenodeoxy- | Lipid | Primary | 0 | 0 | neg |
| | | cholate | | Bile Acid | | | |
| | | sulfate | | Metabolism | | | |
| 100015788 | | sphingomyelin | Lipid | Sphingolipid | 0 | 0 | pos |
| | | (d18:2/18:1)* | | Metabolism | | | |
| 100002206 | | alpha- | Cofactors | Tocopherol | 0 | 0 | pos |
| | | CEHC | and | Metabolism | | | |
| | | | Vitamins | | | | |
| 100015737 | | ceramide | Lipid | Ceramides | 0 | 0 | pos |
| | | (d18:1/17:0, | | | | | |
| | | d17:1/18:0)* | | | | | |
| 100006367 | X - 21892 | 3- | Lipid | Fatty Acid, | 0 | 0 | pos |
| | | hydroxyhexanoate | | Monohydroxy | | | |
| 100002914 | | 3-(4-hydroxy- | Amino | Phenylalanine | 0 | 0 | neg |
| | | phenyl)propionate | Acid | and Tyrosine | | | |
| | | | | Metabolism | | | |
| 100005818 | | N-acetyl-S- | Xeno- | Food | 0 | 0 | neg |
| | | allyl-L- | biotics | Component/ | | | |
| | | cysteine | | Plant | | | |
| 1111 | | vanillylmandelate | Amino | Phenylalanine | 0 | 0 | incon- |
| | | (VMA) | Acid | and Tyrosine | | | sistent |
| | | | | Metabolism | | | |
| 132 | | 3- | Carbo- | Glycolysis, | 0 | 0 | pos |
| | | phosphoglycerate | hydrate | Gluconeo- | | | |
| | | | | genesis, and | | | |
| | | | | Pyruvate | | | |
| | | | | Metabolism | | | |
| 100001563 | | 2-myristoyl- | Lipid | Lysolipid | 0 | 0 | neg |
| | | GPC | | | | | |
| | | (14:0)* | | | | | |
| 233 | | ascorbate | Cofactors | Ascorbate | 0 | 0 | incon- |
| | | (Vitamin C) | and | and Aldarate | | | sistent |
| | | | Vitamins | Metabolism | | | |
| 100003397 | | trimethylamine | Lipid | Phospholipid | 0 | 0 | incon- |
| | | N-oxide | | Metabolism | | | sistent |
| 100008951 | | leucylphenyl- | Peptide | Dipeptide | 0 | 0 | incon- |
| | | alanine/ | | | | | sistent |
| | | isoleucyl- | | | | | |
| | | phenylalanine | | | | | |
| 100015591 | | phosphatidyl- | Lipid | Phosphatidyl- | 0 | 0 | pos |
| | | choline | | choline (PC) | | | |
| | | (16:0/20:4n3; | | | | | |
| | | 18:1/18:3n6)* | | | | | |
| 100004322 | | 2-aminophenol | Xeno- | Chemical | 0 | 0 | incon- |
| | | sulfate | biotics | | | | sistent |
| 100015793 | | sphingomyelin | Lipid | Sphingolipid | 0 | 0 | pos |
| | | (d17:2/16:0, | | Metabolism | | | |
| | | d18:2/15:0)* | | | | | |
| 100006098 | | 3- | Xeno- | Chemical | 0 | 0 | incon- |
| | | hydroxypyridine | biotics | | | | sistent |
| | | sulfate | | | | | |
| X - 15666 | | X - 15666 | 0 | . | 0 | 0 | pos |
| 100006282 | | umbelliferone | Xeno- | Food | 0 | 0 | incon- |
| | | sulfate | biotics | Component/ | | | sistent |
| | | | | Plant | | | |
| 100001315 | | p-cresol | Amino | Phenylalanine | 0 | 0 | incon- |
| | | sulfate | Acid | and Tyrosine | | | sistent |
| | | | | Metabolism | | | |
| 100002259 | | cis-4- | Lipid | Fatty Acid | 0 | 0 | incon- |
| | | decenoyl | | Metabolism | | | sistent |
| | | carnitine | | (Acyl | | | |
| | | | | Carnitine) | | | |
| 100009124 | | 1-margaroyl- | Lipid | Phospholipid | 0 | 0 | neg |
| | | 2-arachidonoyl- | | Metabolism | | | |
| | | GPC | | | | | |
| | | (17:0/20:4)* | | | | | |
| 100015833 | | arachidoylcarnitine | Lipid | Fatty Acid | 0 | 0 | neg |
| | | (C20)* | | Metabolism | | | |
| | | | | (Acyl | | | |
| | | | | Carnitine) | | | |
| 100003444 | | escitalopram | Xeno- | Drug | 0 | 0 | neg |
| | | | biotics | | | | |
| 100001386 | | heme | Cofactors | Hemoglobin | 0 | 0 | pos |
| | | | and | and Porphyrin | | | |
| | | | Vitamins | Metabolism | | | |
| X - 22147 | | dihydrocaffeate | 0 | . | 0 | 0 | pos |
| | | sulfate | | | | | |
| | | (2) | | | | | |
| 100009144 | | 1-palmitoleoyl- | Lipid | Phospholipid | 0 | 0 | pos |
| | | 2-docosahexa- | | Metabolism | | | |
| | | enoyl-GPC | | | | | |
| | | (16:1/22:6)* | | | | | |
| 100005673 | | 1-docosapenta- | Lipid | Lysolipid | 0 | 0 | incon- |
| | | enoyl-GPC | | | | | sistent |
| | | (22:5n6)* | | | | | |
| 100001161 | | valylglutamate | Peptide | Dipeptide | 0 | 0 | pos |
| 1342 | | 3- | Amino | Phenylalanine | 0 | 0 | incon- |
| | | methoxytyrosine | Acid | and Tyrosine | | | sistent |
| | | | | Metabolism | | | |
| 100002849 | | ethyl | Xeno- | Chemical | 0 | 0 | incon- |
| | | glucuronide | biotics | | | | sistent |
| 415 | | methionine | Amino | Methionine, | 0 | 0 | incon- |
| | | | Acid | Cysteine, | | | sistent |
| | | | | SAM and | | | |
| | | | | Taurine | | | |
| | | | | Metabolism | | | |
| 100009067 | | 1-stearoyl- | Lipid | Phosphatidyl- | 0 | 0 | neg |
| | | 2-docosahexa- | | inositol (PI) | | | |
| | | enoyl-GPI | | | | | |
| | | (18:0/22:6)* | | | | | |
| X - 17189 | | X - 17189 | 0 | . | 0 | 0 | incon- |
| | | | | | | | sistent |
| 100001788 | | desmethylnaproxen | Xeno- | Drug | 0 | 0 | incon- |
| | | sulfate | biotics | | | | sistent |
| 100004326 | X - 23788 | 3-acetylphenol | Xeno- | Chemical | 0 | 0 | incon- |
| | | sulfate | biotics | | | | sistent |
| 100001988 | | 5alpha-pregnan- | Lipid | Steroid | 0 | 0 | incon- |
| | | 3beta,20alpha-diol | | | | | sistent |
| | | disulfate | | | | | |
| 100001789 | | sucralose | Xeno- | Food | 0 | 0 | pos |
| | | | biotics | Component/ | | | |
| | | | | Plant | | | |
| 100003639 | | valylaspartate | Peptide | Dipeptide | 0 | 0 | neg |
| 100000997 | | 3- | Lipid | Fatty Acid, | 0 | 0 | incon- |
| | | hydroxydecanoate | | Monohydroxy | | | sistent |
| 100010850 | | 4- | Peptide | Acetylated | 0 | 0 | neg |
| | | hydroxyphenyl- | | Peptides | | | |
| | | acetylglutamine | | | | | |
| X - 24425 | | X - 24425 | 0 | . | 0 | 0 | incon- |
| | | | | | | | sistent |
| X - 17351 | | X - 17351 | 0 | . | 0 | 0 | incon- |
| | | | | | | | sistent |
| 100001617 | | undecanedioate | Lipid | Fatty Acid, | 0 | 0 | incon- |
| | | | | Dicarboxylate | | | sistent |
| 100004327 | | 1-stearoyl- | Lipid | Lysolipid | 0 | 0 | pos |
| | | GPS (18:0)* | | | | | |
| X - 12101 | | X - 12127 | 0 | . | 0 | 0 | incon- |
| | | | | | | | sistent |
| 100000299 | | xanthosine | Nucleotide | Purine | 0 | 0 | pos |
| | | | | Metabolism, | | | |
| | | | | (Hypo)Xanthine/ | | | |
| | | | | Inosine | | | |
| | | | | containing | | | |
| 180 | | linoleate | Lipid | Polyun- | 0 | 0 | incon- |
| | | (18:2n6) | | saturated | | | sistent |
| | | | | Fatty Acid | | | |
| | | | | (n3 and n6) | | | |
| X - 12329 | | X - 12339 | 0 | . | 0 | 0 | incon- |
| | | | | | | | sistent |
| 100020487 | X - 12688 | N-acetyl- | 0 | . | 0 | 0 | incon- |
| | | isoputrenine* | | | | | sistent |
| X - 24071 | | his-glu | 0 | . | 0 | 0 | pos |
| 100001112 | | 3- | Lipid | Fatty Acid, | 0 | 0 | incon- |
| | | hydroxylaurate | | Monohydroxy | | | sistent |
| 361 | | inosine | Nucleotide | Purine | 0 | 0 | pos |
| | | | | Metabolism, | | | |
| | | | | (Hypo)Xanthine/ | | | |
| | | | | Inosine | | | |
| | | | | containing | | | |
| 100001396 | | 7- | Xeno- | Xanthine | 0 | 0 | incon- |
| | | methylxanthine | biotics | Metabolism | | | sistent |
| 100001145 | | 3- | Lipid | Fatty Acid, | 0 | 0 | incon- |
| | | hydroxysebacate | | Monohydroxy | | | sistent |
| 100001268 | | glycylphenyl- | Peptide | Dipeptide | 0 | 0 | incon- |
| | | alanine | | | | | sistent |
| 100015618 | | palmitoyl- | Lipid | Diacylglycerol | 0 | 0 | pos |
| | | docosahexaenoyl- | | | | | |
| | | glycerol | | | | | |
| | | (16:0/22:6) | | | | | |
| | | [1]* | | | | | |
| 100010929 | | linoleoyl- | Lipid | Diacylglycerol | 0 | 0 | pos |
| | | docosahexaenoyl- | | | | | |
| | | glycerol | | | | | |
| | | (18:2/22:6) | | | | | |
| | | [2]* | | | | | |
| 100003163 | | isoleucylalanine | Peptide | Dipeptide | 0 | 0 | neg |
| 100002912 | | tauroursodeoxy- | Lipid | Secondary | 0 | 0 | pos |
| | | cholate | | Bile Acid | | | |
| | | | | Metabolism | | | |
| 100010942 | | linoleoyl- | Lipid | Diacylglycerol | 0 | 0 | pos |
| | | linoleoyl- | | | | | |
| | | glycerol | | | | | |
| | | (18:2/18:2) | | | | | |
| | | [2]* | | | | | |
| X - 23644 | | X - 23644 | 0 | . | 0 | 0 | incon- |
| | | | | | | | sistent |
| 100009407 | | pimeloylcarnitine/ | Lipid | Fatty Acid | 0 | 0 | neg |
| | | 3-methyladipoyl- | | Metabolism | | | |
| | | carnitine | | (Acyl | | | |
| | | (C7-DC) | | Carnitine) | | | |
| 100010927 | | linoleoyl- | Lipid | Diacylglycerol | 0 | 0 | pos |
| | | linolenoyl- | | | | | |
| | | glycerol | | | | | |
| | | (18:2/18:3) | | | | | |
| | | [2]* | | | | | |
| 35 | | S-1- | Amino | Glutamate | 0 | 0 | neg |
| | | pyrroline-5- | Acid | Metabolism | | | |
| | | carboxylate | | | | | |
| 100002968 | | quinine | Xeno- | Drug | 0 | 0 | incon- |
| | | | biotics | | | | sistent |
| 100020274 | X - 16134 | Fibrinopeptide | 0 | . | 0 | 0 | pos |
| | | A (5-16)* | | | | | |
| 71 | | 5-hydroxy- | Amino | Tryptophan | 0 | 0 | incon- |
| | | indoleacetate | Acid | Metabolism | | | sistent |
| 100009018 | | 1- | Lipid | Phospholipid | 0 | 0 | incon- |
| | | pentadecanoyl-2- | | Metabolism | | | sistent |
| | | oleoyl-GPC | | | | | |
| | | (15:0/18:1)* | | | | | |
| 100000784 | | theanine | Xeno- | Food | 0 | 0 | incon- |
| | | | biotics | Component/ | | | sistent |
| | | | | Plant | | | |
| 100003673 | | prolylglutamate | Peptide | Dipeptide | 0 | 0 | neg |
| X - 24293 | | X - 24293 | 0 | . | 0 | 0 | incon- |
| | | | | | | | sistent |
| X - 12849 | | X - 12855 | 0 | . | 0 | 0 | incon- |
| | | | | | | | sistent |
| X - 11843 | | X - 11847 | 0 | . | 0 | 0 | incon- |
| | | | | | | | sistent |
| 100003252 | | phenylalanylserine | Peptide | Dipeptide | 0 | 0 | incon- |
| | | | | | | | sistent |
| 100002070 | | 2- | Lipid | Fatty Acid, | 0 | 0 | incon- |
| | | hydroxyglutarate | | Dicarboxylate | | | sistent |
| 208 | | adenosine | Nucleotide | Purine | 0 | 0 | pos |
| | | 5′- | | Metabolism, | | | |
| | | diphosphate (ADP) | | Adenine | | | |
| | | | | containing | | | |
| 100001787 | | desmethylnaproxen | Xeno- | Drug | 0 | 0 | incon- |
| | | | biotics | | | | sistent |
| 100006293 | | sphingomyelin | Lipid | Sphingolipid | 0 | 0 | pos |
| | | (d18:1/20:2, | | Metabolism | | | |
| | | d18:2/20:1, | | | | | |
| | | d16:1/22:2)* | | | | | |
| X - 21815 | | X - 21815 | 0 | . | 0 | 0 | incon- |
| | | | | | | | sistent |
| 100015832 | | behenoylcarnitine | Lipid | Fatty Acid | 0 | 0 | pos |
| | | (C22)* | | Metabolism | | | |
| | | | | (Acyl | | | |
| | | | | Carnitine) | | | |
| 100006378 | | N- | Amino | Tryptophan | 0 | 0 | pos |
| | | acetylkynurenine | Acid | Metabolism | | | |
| | | (2) | | | | | |
| 1137 | | oleoyl | Lipid | Endocannabinoid | 0 | 0 | incon- |
| | | ethanolamide | | | | | sistent |
| 100006082 | | 4-hydroxy- | Xeno- | Chemical | 0 | 0 | incon- |
| | | chlorothalonil | biotics | | | | sistent |
| 100001526 | | malonylcarnitine | Lipid | Fatty Acid | 0 | 0 | pos |
| | | | | Synthesis | | | |
| 241 | | phenylpyruvate | Amino | Phenylalanine | 0 | 0 | pos |
| | | | Acid | and Tyrosine | | | |
| | | | | Metabolism | | | |
| 565 | | tryptophan | Amino | Tryptophan | 0 | 0 | incon- |
| | | | Acid | Metabolism | | | sistent |
| 100015790 | | sphingomyelin | Lipid | Sphingolipid | 0 | 0 | neg |
| | | (d18:2/21:0), | | Metabolism | | | |
| | | d16:2/23:0)* | | | | | |
| 100001151 | | butyrylglycine | Lipid | Fatty Acid | 0 | 0 | pos |
| | | | | Metabolism | | | |
| | | | | (also BCAA | | | |
| | | | | Metabolism) | | | |
| 100009006 | | 1-(1-enyl- | Lipid | Plasmalogen | 0 | 0 | pos |
| | | palmitoyl)- | | | | | |
| | | 2-dihomo- | | | | | |
| | | linolenoyl- | | | | | |
| | | GPC (P- | | | | | |
| | | 16:0/20:3)* | | | | | |
| 100015643 | | sphingadienine | Lipid | Sphingolipid | 0 | 0 | neg |
| | | | | Metabolism | | | |
| 100004523 | | N-delta- | Amino | Urea cycle; | 0 | 0 | incon- |
| | | acetylornithine | Acid | Arginine | | | sistent |
| | | | | and Proline | | | |
| | | | | Metabolism | | | |
| 100008906 | | 1,2-dioleoyl- | Lipid | Phospholipid | 0 | 0 | pos |
| | | GPE (18:1/18:1) | | Metabolism | | | |
| 100008939 | | isoleucylleucine/ | Peptide | Dipeptide | 0 | 0 | neg |
| | | leucylisoleucine | | | | | |
| 100001993 | | pregnen-diol | Lipid | Steroid | 0 | 0 | incon- |
| | | disulfate* | | | | | sistent |
| 100000882 | | 3- | Lipid | Fatty Acid, | 0 | 0 | neg |
| | | hydroxymyristate | | Monohydroxy | | | |
| 100001612 | | N-acetyl- | Amino | Glutamate | 0 | 0 | pos |
| | | aspartyl- | Acid | Metabolism | | | |
| | | glutamate | | | | | |
| | | (NAAG) | | | | | |
| 100005403 | | etiocholanolone | Lipid | Steroid | 0 | 0 | pos |
| | | glucuronide | | | | | |
| X - 16124 | | X - 16124 | 0 | . | 0 | 0 | incon- |
| | | | | | | | sistent |
| X - 21821 | | X - 21821 | 0 | . | 0 | 0 | incon- |
| | | | | | | | sistent |
| 100001664 | | N6- | Nucleotide | Purine | 0 | 0 | neg |
| | | succinyladenosine | | Metabolism, | | | |
| | | | | Adenine | | | |
| | | | | containing | | | |
| 100001882 | | glycosyl-N- | Lipid | Sphingolipid | 0 | 0 | neg |
| | | stearoyl- | | Metabolism | | | |
| | | sphingosine | | | | | |
| 100001990 | | taurocholenate | Lipid | Secondary | 0 | 0 | incon- |
| | | sulfate | | Bile Acid | | | sistent |
| | | | | Metabolism | | | |
| 100002953 | | 16- | Lipid | Fatty Acid, | 0 | 0 | incon- |
| | | hydroxypalmitate | | Monohydroxy | | | sistent |
| 100008991 | | 1-palmitoyl- | Lipid | Phospholipid | 0 | 0 | neg |
| | | 2-docosahexa- | | Metabolism | | | |
| | | enoyl-GPE | | | | | |
| | | (16:0/22:6)* | | | | | |
| 100001103 | | glutamate, | Amino | Glutamate | 0 | 0 | pos |
| | | gamma- | Acid | Metabolism | | | |
| | | methyl | | | | | |
| | | ester | | | | | |
| 100009045 | | phenylacetyl- | Amino | Phenylalanine | 0 | 0 | pos |
| | | glutamate | Acid | and Tyrosine | | | |
| | | | | Metabolism | | | |
| 100000939 | | 1,6- | Xeno- | Food | 0 | 0 | neg |
| | | anhydroglucose | biotics | Component/ | | | |
| | | | | Plant | | | |
| 100002679 | | gamma- | Amino | Glutamate | 0 | 0 | pos |
| | | carboxyglutamate | Acid | Metabolism | | | |
| 826 | | xylose | Carbo- | Pentose | 0 | 0 | pos |
| | | | hydrate | Metabolism | | | |
| 100004171 | | carboxyibuprofen | Xeno- | Drug | 0 | 0 | incon- |
| | | | biotics | | | | sistent |
| 100002009 | | 5alpha-pregnan- | Lipid | Steroid | 0 | 0 | incon- |
| | | 3beta,20beta-diol | | | | | sistent |
| | | monosulfate (1) | | | | | |
| 100005714 | | 1-linolenoyl- | Lipid | Lysophospholipid | 0 | 0 | pos |
| | | GPE (18:3)* | | | | | |
| 100001216 | | delta- | Cofactors | Tocopherol | 0 | 0 | pos |
| | | tocopherol | and | Metabolism | | | |
| | | | Vitamins | | | | |
| 1504 | | oleamide | Lipid | Fatty Acid, | 0 | 0 | neg |
| | | | | Amide | | | |
| 100015687 | | phosphatidyl- | Lipid | Phosphatidyl- | 0 | 0 | pos |
| | | ethanolamine | | ethanolamine | | | |
| | | (P-18:1/20:4, | | (PE) | | | |
| | | P-16:0/22:5n3)* | | | | | |
| 100006295 | | sphingomyelin | Lipid | Sphingolipid | 0 | 0 | neg |
| | | (d18:1/22:1, | | Metabolism | | | |
| | | d18:2/22:0, | | | | | |
| | | d16:1/24:1) | | | | | |
| 100010926 | | linoleoyl- | Lipid | Diacylglycerol | 0 | 0 | pos |
| | | linolenoyl- | | | | | |
| | | glycerol | | | | | |
| | | (18:2/18:3) | | | | | |
| | | [1]* | | | | | |
| 100003630 | | alpha- | Peptide | Dipeptide | 0 | 0 | pos |
| | | glutamylglycine | | | | | |
| 100002735 | | ranitidine | Xeno- | Drug | 0 | 0 | incon- |
| | | | biotics | | | | sistent |
| 100001167 | | pro- | Amino | Urea cycle; | 0 | 0 | incon- |
| | | hydroxy-pro | Acid | Arginine | | | sistent |
| | | | | and Proline | | | |
| | | | | Metabolism | | | |
| 100010895 | | 2′-O- | Nucleotide | Pyrimidine | 0 | 0 | neg |
| | | methylcytidine | | Metabolism, | | | |
| | | | | Cytidine | | | |
| | | | | containing | | | |
| 143 | | 4-hydroxy- | Lipid | Fatty Acid, | 0 | 0 | pos |
| | | 2-nonenal | | Oxidized | | | |
| X - 17167 | | X - 17167 | 0 | . | 0 | 0 | incon- |
| | | | | | | | sistent |
| 100004054 | | margaroylcarnitine* | Lipid | Fatty Acid | 0 | 0 | pos |
| | | | | Metabolism | | | |
| | | | | (Acyl | | | |
| | | | | Carnitine) | | | |
| 100015596 | | 1-(1-enyl- | Lipid | Plasmalogen | 0 | 0 | neg |
| | | stearoyl)-2- | | | | | |
| | | docosapentaenoyl- | | | | | |
| | | GPE (P- | | | | | |
| | | 18:0/22:5n3)* | | | | | |
| X - 12740 | | X - 12748 | 0 | . | 0 | 0 | incon- |
| | | | | | | | sistent |
| 100004509 | | S- | Xeno- | Food | 0 | 0 | neg |
| | | allylcysteine | biotics | Component/ | | | |
| | | | | Plant | | | |
| 100006089 | | isoeugenol | Xeno- | Food | 0 | 0 | incon- |
| | | sulfate | biotics | Component/ | | | sistent |
| | | | | Plant | | | |
| 100006360 | | dopamine | Amino | Phenylalanine | 0 | 0 | pos |
| | | sulfate (1) | Acid | and Tyrosine | | | |
| | | | | Metabolism | | | |
| 215 | | adenosine 5′- | Cofactors | Nicotinate and | 0 | 0 | neg |
| | | diphosphoribose | and | Nicotinamide | | | |
| | | (ADP-ribose) | Vitamins | Metabolism | | | |
| 980 | | pentadecanoate | Lipid | Long Chain | 0 | 0 | pos |
| | | (15:0) | | Fatty Acid | | | |
| 249 | | carnosine | Amino | Histidine | 0 | 0 | neg |
| | | | Acid | Metabolism | | | |
| 100005367 | | N- | Xeno- | Food | 0 | 0 | neg |
| | | acetylalliin | biotics | Component/ | | | |
| | | | | Plant | | | |
| 100015831 | | linolenoyl- | Lipid | Fatty Acid | 0 | 0 | neg |
| | | carnitine | | Metabolism | | | |
| | | (C18:3)* | | (Acyl | | | |
| | | | | Carnitine) | | | |
| 100015727 | | ceramide | Lipid | Ceramides | 0 | 0 | neg |
| | | (d16:1/24:1, | | | | | |
| | | d18:1/22:1)* | | | | | |
| 100009019 | | 1-stearyl-2- | Lipid | Plasmalogen | 0 | 0 | pos |
| | | arachidonoyl- | | | | | |
| | | GPC (O- | | | | | |
| | | 18:0/20:4)* | | | | | |
| 100006129 | | vanillactate | Amino | Tyrosine | 0 | 0 | neg |
| | | | Acid | Metabolism | | | |
| 100005383 | | N- | Xeno- | Bacterial/ | 0 | 0 | neg |
| | | methylpipecolate | biotics | Fungal | | | |
| 100009042 | | 5- | Amino | Tryptophan | 0 | 0 | neg |
| | | hydroxyindole | Acid | Metabolism | | | |
| | | sulfate | | | | | |
| 100002227 | | 4-cholesten- | Lipid | Sterol | 0 | 0 | neg |
| | | 3-one | | | | | |
| 100004635 | | methionine | Amino | Methionine, | 0 | 0 | incon- |
| | | sulfone | Acid | Cysteine, | | | sistent |
| | | | | SAM and | | | |
| | | | | Taurine | | | |
| | | | | Metabolism | | | |
| 100009275 | | methylsuccinoyl- | Amino | Leucine, | 0 | 0 | pos |
| | | carnitine (1) | Acid | Isoleucine | | | |
| | | | | and Valine | | | |
| | | | | Metabolism | | | |
| 1099 | | guanosine | Nucleotide | Purine | 0 | 0 | incon- |
| | | | | Metabolism, | | | sistent |
| | | | | Guanine | | | |
| | | | | containing | | | |
| 100015744 | | ceramide | Lipid | Ceramides | 0 | 0 | pos |
| | | (d18:2/24:1, | | | | | |
| | | d18:1/24:2)* | | | | | |
| 100001132 | | pyroglutamylvaline | Peptide | Dipeptide | 0 | 0 | neg |
| 100001063 | | 7- | Lipid | Secondary | 0 | 0 | neg |
| | | ketodeoxycholate | | Bile Acid | | | |
| | | | | Metabolism | | | |
| 100015837 | | arachidonoyl- | Lipid | Fatty Acid | 0 | 0 | pos |
| | | carnitine (C20:4) | | Metabolism | | | |
| | | | | (Acyl | | | |
| | | | | Carnitine) | | | |
| 100006184 | | 2- | Xeno- | Chemical | 0 | 0 | neg |
| | | methoxyresorcinol | biotics | | | | |
| | | sulfate | | | | | |
| 100003260 | | carboxyethyl- | Amino | Glutamate | 0 | 0 | pos |
| | | GABA | Acid | Metabolism | | | |
| 100001002 | | EDTA | Xeno- | Chemical | 0 | 0 | neg |
| | | | biotics | | | | |
| 100009038 | | myristoyl | Lipid | Sphingolipid | 0 | 0 | pos |
| | | dihydro- | | Metabolism | | | |
| | | sphingomyelin | | | | | |
| | | (d18:0/14:0)* | | | | | |
| 100003210 | | valylleucine | Peptide | Dipeptide | 0 | 0 | neg |
| 100015791 | | sphingomyelin | Lipid | Sphingolipid | 0 | 0 | pos |
| | | (d18:2/23:1)* | | Metabolism | | | |
| 100003679 | | prolylserine | Peptide | Dipeptide | 0 | 0 | neg |
| 100005972 | | alpha-CEHC | Cofactors | Tocopherol | 0 | 0 | neg |
| | | sulfate | and | Metabolism | | | |
| | | | Vitamins | | | | |
| 100001733 | X - 12824 | hexanoylglutamine | Lipid | Fatty Acid | 0 | 0 | incon- |
| | | | | Metabolism | | | sistent |
| | | | | (Acyl | | | |
| | | | | Glutamine) | | | |
| 1215 | | N-acetyl- | Carbo- | Aminosugar | 0 | 0 | pos |
| | | glucosaminyl- | hydrate | Metabolism | | | |
| | | asparagine | | | | | |
| 100009157 | | 1-palmitoleoyl- | Lipid | Phosphatidyl- | 0 | 0 | neg |
| | | 2-arachidonoyl- | | choline (PC) | | | |
| | | GPC | | | | | |
| | | (16:1/20:4)* | | | | | |
| 100002067 | | pregn | Lipid | Steroid | 0 | 0 | incon- |
| | | steroid | | | | | sistent |
| | | monosulfate* | | | | | |
| 100010896 | | 2′-O- | Nucleotide | Pyrimidine | 0 | 0 | neg |
| | | methyluridine | | Metabolism, | | | |
| | | | | Uracil | | | |
| | | | | containing | | | |
| 100001431 | | 1-pentadecanoyl- | Lipid | Monoacylglycerol | 0 | 0 | pos |
| | | glycerol (15:0) | | | | | |
| 100002344 | | 13- | Lipid | Fatty Acid, | 0 | 0 | neg |
| | | methylmyristate | | Branched | | | |
| | | (i15:0) | | | | | |
| 100015850 | | adrenoylcarnitine | Lipid | Fatty Acid | 0 | 0 | pos |
| | | (C22:4)* | | Metabolism | | | |
| | | | | (Acyl | | | |
| | | | | Carnitine) | | | |
| 100003606 | | tyrosylglutamine | Peptide | Dipeptide | 0 | 0 | incon- |
| | | | | | | | sistent |
| X - 17325 | | X - 17325 | 0 | . | 0 | 0 | pos |
| 100002128 | | 17alpha-hydroxy- | Lipid | Steroid | 0 | 0 | neg |
| | | pregnenolone | | | | | |
| | | sulfate | | | | | |
| 100015605 | | 1-palmitoleoyl- | Lipid | Phosphatidyl- | 0 | 0 | pos |
| | | 2-eicosapenta- | | choline (PC) | | | |
| | | enoyl-GPC | | | | | |
| | | (16:1/20:5)* | | | | | |
| 213 | | N6- | Nucleotide | Purine | 0 | 0 | neg |
| | | methyladenosine | | Metabolism, | | | |
| | | | | Adenine | | | |
| | | | | containing | | | |
| 117 | | homovanillate (HVA) | Amino | Tyrosine | 0 | 0 | pos |
| | | | Acid | Metabolism | | | |
| 100001469 | | N1-Methyl- | Cofactors | Nicotinate and | 0 | 0 | neg |
| | | 4-pyridone-3- | and | Nicotinamide | | | |
| | | carboxamide | Vitamins | Metabolism | | | |
| 100005418 | | 17alpha-hydroxy- | Lipid | Pregnenolone | 0 | 0 | pos |
| | | pregnanolone | | Steroids | | | |
| | | glucuronide | | | | | |
| 100009227 | | 1-linoleoyl- | Lipid | Lysophospholipid | 0 | 0 | pos |
| | | GPG (18:2)* | | | | | |
| 1023 | | sarcosine | Amino | Glycine, | 0 | 0 | pos |
| | | | Acid | Serine and | | | |
| | | | | Threonine | | | |
| | | | | Metabolism | | | |
| 100001266 | | N- | Amino | Urea cycle; | 0 | 0 | pos |
| | | acetylarginine | Acid | Arginine | | | |
| | | | | and Proline | | | |
| | | | | Metabolism | | | |
| 100009184 | | 1-stearoyl- | Lipid | Phosphatidyl- | 0 | 0 | neg |
| | | 2-dihomo- | | inositol (PI) | | | |
| | | linolenoyl- | | | | | |
| | | GPI | | | | | |
| | | (18:0/20:3n3 | | | | | |
| | | or 6)* | | | | | |
| 100002003 | | 21-hydroxy- | Lipid | Pregnenolone | 0 | 0 | pos |
| | | pregnenolone | | Steroids | | | |
| | | monosulfate (1) | | | | | |
| 100005716 | | 1-oleoyl- | Lipid | Lysolipid | 0 | 0 | neg |
| | | GPG (18:1)* | | | | | |
| 100008905 | | 1,2- | Lipid | Phospholipid | 0 | 0 | neg |
| | | dioleoyl-GPC | | Metabolism | | | |
| | | (18:1/18:1)* | | | | | |
| 100006271 | | ethyl | Xeno- | Chemical | 0 | 0 | pos |
| | | paraben | biotics | | | | |
| | | sulfate | | | | | |
| 100015755 | | ceramide | Lipid | Ceramides | 0 | 0 | pos |
| | | (d18:1/20:0, | | | | | |
| | | d16:/22:0, | | | | | |
| | | d20:1/18:0)* | | | | | |
| 100006108 | | phenylacetyl- | Amino | Phenylalanine | 0 | 0 | neg |
| | | carnitine | Acid | and Tyrosine | | | |
| | | | | Metabolism | | | |
| 100009181 | | 1-stearoyl- | Lipid | Phosphatidyl- | 0 | 0 | neg |
| | | 2-oleoyl-GPI | | inositol (PI) | | | |
| | | (18:0/18:1)* | | | | | |
| 100015688 | | 1-stearoyl- | Lipid | Phosphatidyl- | 0 | 0 | neg |
| | | 2-(hydroxy- | | choline (PC) | | | |
| | | linoleoyl)-GPC | | | | | |
| | | (18:0/18:2(OH))* | | | | | |
| 100001129 | | O- | Amino | Glycine, | 0 | 0 | neg |
| | | acetylhomoserine | Acid | Serine and | | | |
| | | | | Threonine | | | |
| | | | | Metabolism | | | |
| 100002017 | | 5alpha-androstan- | Lipid | Steroid | 0 | 0 | pos |
| | | 3alpha,17beta-diol | | | | | |
| | | disulfate | | | | | |
| 100004056 | | N- | Amino | Methionine, | 0 | 0 | neg |
| | | methyltaurine | Acid | Cysteine, | | | |
| | | | | SAM and | | | |
| | | | | Taurine | | | |
| | | | | Metabolism | | | |
| 100015624 | | N-behenoyl- | Lipid | Sphingolipid | 0 | 0 | neg |
| | | sphingadienine | | Metabolism | | | |
| | | (d18:2/22:0)* | | | | | |
| 100002015 | | 5alpha-pregnan- | Lipid | Steroid | 0 | 0 | neg |
| | | 3(alpha or | | | | | |
| | | beta),20beta-diol | | | | | |
| | | disulfate | | | | | |
| 100002952 | | docosadioate | Lipid | Fatty Acid, | 0 | 0 | pos |
| | | (C22-DC) | | Dicarboxylate | | | |
| 1488 | | arachidonoyl | Lipid | Endocannabinoid | 0 | 0 | neg |
| | | ethanolamide | | | | | |
| 100000639 | | 1-stearoyl- | Lipid | Phosphatidyl- | 0 | 0 | neg |
| | | 2-oleoyl-GPS | | serine (PS) | | | |
| | | (18:0/18:1) | | | | | |
| 100005834 | | 9- | Lipid | Fatty Acid, | 0 | 0 | neg |
| | | hydroxystearate | | Monohydroxy | | | |
| 100001721 | | N2- | Amino | Lysine | 0 | 0 | pos |
| | | acetyllysine | Acid | Metabolism | | | |
| 100001279 | | hyocholate | Lipid | Secondary | 0 | 0 | pos |
| | | | | Bile Acid | | | |
| | | | | Metabolism | | | |
| 100008979 | | 1-oleoyl-2- | Lipid | Phospholipid | 0 | 0 | neg |
| | | linoleoyl-GPI | | Metabolism | | | |
| | | (18:1/18:2)* | | | | | |
| 100015731 | | N-palmitoyl- | Lipid | Sphingolipid | 0 | 0 | pos |
| | | heptadeca- | | Metabolism | | | |
| | | sphingosine | | | | | |
| | | (d17:1/16:0)* | | | | | |
| 100000565 | | 15-HETE | Lipid | Eicosanoid | 0 | 0 | pos |
| 100015787 | | sphingomyelin | Lipid | Sphingolipid | 0 | 0 | pos |
| | | (d18:1/19:0, | | Metabolism | | | |
| | | d19:1/18:0)* | | | | | |
| 100004318 | | indolin-2-one | Xeno- | Food | 0 | 0 | pos |
| | | | biotics | Component/ | | | |
| | | | | Plant | | | |
| 100003109 | | 2-oxindole- | Xeno- | Food | 0 | 0 | neg |
| | | 3-acetate | biotics | Component/ | | | |
| | | | | Plant | | | |
| 100004634 | | 3-methoxytyramine | Amino | Tyrosine | 0 | 0 | pos |
| | | sulfate | Acid | Metabolism | | | |
| 100015689 | | 1-palmitoyl- | Lipid | Phosphatidyl- | 0 | 0 | neg |
| | | 2-(hydroxy- | | choline (PC) | | | |
| | | linoleoyl)-GPC | | | | | |
| | | (16:0/18:2(OH))* | | | | | |
| 100015834 | | lignoceroyl- | Lipid | Fatty Acid | 0 | 0 | neg |
| | | carnitine | | Metabolism | | | |
| | | (C24)* | | (Acyl | | | |
| | | | | Carnitine) | | | |
| 100006296 | | sphingomyelin | Lipid | Sphingolipid | 0 | 0 | pos |
| | | (d18:1/22:2, | | Metabolism | | | |
| | | d18:2/22:1, | | | | | |
| | | d16:1/24:2)* | | | | | |
| 1022 | | picolinate | Amino | Tryptophan | 0 | 0 | pos |
| | | | Acid | Metabolism | | | |

| | | TWINSUK | TWINSUK | TWINSUK | Health Nucleus |
|---|---|---|---|---|---|
| | | v1 p (after | v2 p (after | v3 p (after | p (after |
| | | controlling | controlling | controlling | controlling |
| | | for age, sex, and | for age, sex, and | for age, sex, and | for age, sex, and |
| | rank of | first genetic | first genetic | first genetic | first genetic |
| Metabolite | impor- | principal | principal | principal | principal |
| ID | tance | component) | component) | component) | component) |
| 1134 | 1 | 8.20E−24 | 3.40E−36 | 5.49E−31 | 2.70E−11 |
| 100001412 | 2 | 7.36E−14 | 9.89E−28 | 1.90E−26 | 3.88E−06 |
| 100009051 | 3 | 1.07E−17 | 3.71E−20 | 5.60E−20 | 5.81E−10 |
| 561 | 4 | 3.95E−08 | 2.85E−07 | 6.72E−28 | 1.05E−23 |
| 212 | 5 | 1.08E−08 | 1.96E−18 | 1.21E−28 | 3.35E−05 |
| 100001384 | 6 | 3.11E−10 | 2.25E−19 | 3.13E−20 | 5.82E−11 |
| 100001006 | 7 | 5.17E−11 | 1.63E−18 | 3.38E−22 | 8.76E−07 |
| 100005353 | 8 | 4.19E−10 | 9.27E−19 | 2.64E−24 | 4.15E−04 |
| 566 | 9 | 1.90E−14 | 1.68E−19 | 5.13E−18 | 3.45E−05 |
| 100009007 | 10 | 1.59E−05 | 2.91E−12 | 3.99E−20 | 6.25E−17 |
| 100005352 | 11 | 4.93E−07 | 9.63E−15 | 7.75E−16 | 2.11E−16 |
| 100001948 | 12 | 5.39E−12 | 2.81E−16 | 1.77E−18 | 9.58E−07 |
| 100008917 | 13 | 2.47E−06 | 6.60E−15 | 6.50E−15 | 1.51E−14 |
| 100001162 | 14 | 4.53E−12 | 2.37E−14 | 5.37E−13 | 3.16E−09 |
| 98 | 15 | 6.09E−11 | 7.81E−18 | 4.38E−14 | 5.60E−05 |
| 803 | 16 | 1.29E−07 | 5.83E−16 | 3.62E−14 | 4.73E−07 |
| 1084 | 17 | 1.36E−12 | 7.25E−13 | 4.00E−12 | 7.49E−07 |
| 100008981 | 18 | 7.26E−08 | 1.23E−12 | 1.33E−19 | 2.78E−04 |
| 100001395 | 19 | 2.78E−06 | 4.74E−12 | 1.54E−14 | 9.57E−11 |
| 100004046 | 20 | 6.76E−07 | 3.45E−17 | 6.21E−14 | 2.66E−05 |
| 100002106 | 21 | 1.32E−15 | 1.70E−12 | 4.30E−08 | 1.50E−06 |
| 100001415 | 22 | 9.77E−10 | 1.14E−14 | 9.01E−13 | 3.11E−05 |
| 100009009 | 23 | 8.40E−08 | 3.40E−10 | 9.83E−18 | 1.43E−06 |
| 100008985 | 24 | 1.67E−10 | 5.53E−11 | 7.34E−14 | 1.00E−06 |
| 1110 | 25 | 3.58E−09 | 5.18E−15 | 3.08E−12 | 1.15E−04 |
| 811 | 26 | 5.28E−08 | 8.14E−12 | 1.77E−13 | 2.33E−07 |
| 100009015 | 27 | 1.91E−05 | 5.59E−10 | 7.17E−19 | 4.76E−06 |
| 100000491 | 28 | 2.29E−07 | 1.12E−10 | 1.07E−17 | 1.90E−04 |
| 100009055 | 29 | 4.39E−10 | 5.28E−13 | 9.25E−10 | 3.84E−07 |
| 917 | 30 | 1.89E−05 | 5.90E−11 | 6.64E−13 | 5.44E−08 |
| 1102 | 31 | 4.25E−05 | 2.33E−15 | 4.21E−11 | 2.12E−05 |
| 815 | 32 | 4.14E−07 | 5.34E−12 | 8.78E−11 | 5.51E−06 |
| 100002990 | 33 | 4.99E−08 | 6.85E−10 | 1.30E−09 | 6.75E−08 |
| 100008903 | 34 | 9.23E−08 | 1.28E−08 | 2.64E−14 | 1.12E−04 |
| 397 | 35 | 7.53E−07 | 1.17E−12 | 3.55E−10 | 4.03E−05 |
| 100009053 | 36 | 7.77E−06 | 1.64E−10 | 1.28E−13 | 2.34E−04 |
| 100009052 | 37 | 5.98E−10 | 1.69E−09 | 1.29E−07 | 1.34E−06 |
| 100001104 | 38 | 3.33E−07 | 1.11E−12 | 4.28E−08 | 1.62E−05 |
| 100000007 | 39 | 8.70E−10 | 1.50E−07 | 3.70E−08 | 1.18E−07 |
| 100002989 | 40 | 2.75E−08 | 5.33E−09 | 4.76E−10 | 8.39E−06 |
| 234 | 41 | 2.69E−06 | 4.97E−07 | 3.95E−06 | 3.24E−13 |
| 100002253 | 42 | 8.13E−07 | 8.05E−10 | 6.54E−12 | 4.66E−04 |
| 100009054 | 43 | 3.23E−05 | 2.11E−08 | 2.84E−12 | 1.12E−04 |
| 182 | 44 | 3.84E−07 | 2.91E−09 | 3.50E−08 | 3.60E−05 |
| 100001509 | 45 | 9.20E−09 | 1.29E−06 | 2.20E−06 | 1.64E−07 |
| 572 | 46 | 9.55E−09 | 2.02E−07 | 7.71E−07 | 2.88E−04 |
| 100009143 | 47 | 7.57E−07 | 1.03E−09 | 3.46E−06 | 1.95E−04 |
| 100001586 | 48 | 5.46E−05 | 2.51E−06 | 2.45E−06 | 2.03E−06 |
| 273 | 49 | 1.41E−05 | 1.05E−05 | 6.33E−08 | 1.35E−04 |
| X - 12063 | 50 | 4.73E−34 | 1.23E−40 | 1.14E−29 | NA |
| X - 22822 | 51 | 1.84E−14 | 9.49E−29 | 1.73E−22 | NA |
| X - 11564 | 52 | 1.37E−13 | 6.26E−26 | 8.26E−25 | NA |
| X - 15492 | 53 | 3.23E−16 | 7.61E−20 | 9.08E−20 | NA |
| X - 13529 | 54 | 3.86E−13 | 6.01E−19 | 6.22E−17 | NA |
| X - 15497 | 55 | 1.13E−09 | 1.18E−14 | 2.88E−16 | NA |
| 100009020 | 56 | NA | NA | NA | 5.92E−13 |
| X - 15503 | 57 | 1.67E−06 | 1.35E−14 | 1.15E−17 | NA |
| X - 11444 | 58 | 8.00E−09 | 3.27E−11 | 9.40E−17 | NA |
| X - 12026 | 59 | 4.20E−08 | 2.15E−15 | 1.51E−12 | NA |
| 100005985 | 60 | 4.98E−20 | 8.02E−16 | 8.83E−08 | 9.30E−04 |
| 821 | 61 | 7.34E−09 | 8.42E−16 | 2.87E−18 | 4.01E−03 |
| X - 11261 | 62 | 5.48E−10 | 8.77E−07 | 4.79E−17 | NA |
| 100000265 | 63 | 5.60E−09 | 2.55E−14 | 1.01E−17 | 1.39E−03 |
| 100006379 | 64 | 8.22E−10 | 7.45E−18 | 5.29E−14 | 8.54E−02 |
| 100002514 | 65 | 6.01E−09 | 1.69E−15 | 1.26E−14 | 1.27E−03 |
| 344 | 66 | 5.10E−12 | 4.12E−15 | 6.63E−14 | 1.83E−01 |
| 381 | 67 | 9.89E−09 | 3.39E−14 | 3.81E−15 | 2.72E−03 |
| 100000010 | 68 | 5.91E−09 | 2.71E−16 | 1.20E−12 | 3.19E−03 |
| 1254 | 69 | 4.72E−08 | 7.48E−16 | 6.30E−13 | 1.30E−03 |
| 100019794 | 70 | 6.90E−04 | 1.76E−11 | 2.83E−13 | NA |
| 880 | 71 | 1.26E−05 | 2.48E−09 | 3.76E−20 | 1.52E−02 |
| 100010930 | 72 | 2.58E−09 | 4.10E−11 | 3.19E−13 | 9.84E−04 |
| 100001425 | 73 | 6.35E−06 | 1.45E−10 | 3.05E−11 | NA |
| X - 17166 | 74 | 9.31E−08 | 7.10E−10 | 5.73E−10 | NA |
| 376 | 75 | 7.35E−08 | 8.45E−15 | 1.42E−12 | 1.64E−01 |
| 100005849 | 76 | 1.44E−05 | 5.21E−11 | 6.77E−11 | NA |
| 1242 | 77 | 6.01E−07 | 9.76E−13 | 5.74E−13 | 1.47E−03 |
| X - 12846 | 78 | 6.75E−06 | 1.11E−10 | 3.73E−10 | NA |
| 893 | 79 | 5.27E−04 | 1.96E−12 | 4.41E−16 | 1.65E−02 |
| X - 15486 | 80 | 4.36E−08 | 9.29E−06 | 1.29E−11 | NA |
| 100001264 | 81 | 7.68E−05 | 5.20E−09 | 8.47E−12 | 5.22E−08 |
| 100001272 | 82 | 4.03E−02 | 2.45E−07 | 2.20E−09 | 2.12E−14 |
| X - 12170 | 83 | 2.37E−06 | 6.83E−09 | 1.50E−09 | NA |
| 892 | 84 | 4.14E−05 | 6.58E−13 | 8.99E−14 | 3.40E−01 |
| 100009130 | 85 | 2.42E−03 | 1.58E−08 | 1.04E−15 | 2.35E−05 |
| 100009037 | 86 | 4.28E−06 | 6.25E−09 | 2.08E−14 | 3.47E−03 |
| 1082 | 87 | 3.66E−10 | 2.19E−10 | 5.36E−10 | 4.51E−02 |
| 244 | 88 | 1.20E−05 | 1.49E−13 | 2.70E−11 | 7.10E−02 |
| 100001557 | 89 | 2.68E−03 | 9.82E−08 | 1.53E−12 | 1.24E−08 |
| X - 13835 | 90 | 3.60E−08 | 3.10E−10 | 1.49E−05 | NA |
| 100001977 | 91 | 5.61E−08 | 1.55E−08 | 1.64E−11 | 1.47E−03 |
| X - 17299 | 92 | 9.28E−07 | 2.72E−08 | 2.67E−08 | NA |
| 100009139 | 93 | NA | NA | NA | 1.30E−07 |
| X - 18901 | 94 | 9.08E−06 | 1.48E−06 | 5.08E−10 | NA |
| 100004329 | 95 | 9.31E−18 | 4.17E−07 | 1.75E−02 | 2.77E−02 |
| 358 | 96 | 2.82E−07 | 1.17E−09 | 4.77E−10 | 1.61E−02 |
| 533 | 97 | 3.87E−07 | 1.06E−12 | 3.55E−08 | 1.97E−01 |
| X - 23639 | 98 | 2.40E−08 | 1.47E−08 | 5.47E−05 | NA |
| 460 | 99 | 3.60E−07 | 1.45E−09 | 3.14E−10 | 3.93E−02 |
| 100009122 | 100 | NA | NA | NA | 3.04E−07 |
| 100020254 | 101 | 8.91E−06 | 6.72E−06 | 6.38E−10 | NA |
| 100002544 | 102 | NA | NA | NA | 3.43E−07 |
| 100001468 | 103 | 4.80E−06 | 6.86E−13 | 2.45E−08 | 2.33E−01 |
| 100001856 | 104 | 1.40E−05 | 2.77E−10 | 2.66E−09 | 1.93E−03 |
| 100002875 | 105 | 1.56E−02 | 1.65E−05 | 2.84E−11 | 3.07E−09 |
| 100008952 | 106 | 5.96E−06 | 2.56E−10 | 2.77E−10 | 6.20E−02 |
| 100001256 | 107 | 7.43E−10 | 3.87E−07 | 2.47E−09 | 3.83E−02 |
| 100001734 | 108 | 1.50E−03 | 4.05E−11 | 3.12E−10 | 1.77E−03 |
| X - 23026 | 109 | 5.20E−05 | 1.83E−09 | 1.96E−06 | NA |
| 240 | 110 | 1.39E−07 | 4.92E−09 | 6.77E−08 | 3.23E−03 |
| 100001485 | 111 | 1.68E−03 | 1.48E−01 | 4.15E−14 | 3.40E−08 |
| 100001566 | 112 | 7.16E−03 | 1.39E−07 | 4.30E−09 | 9.09E−08 |
| 100009135 | 113 | NA | NA | NA | 8.53E−07 |
| 482 | 114 | 4.32E−03 | 7.45E−07 | 2.36E−07 | 1.38E−09 |
| X - 11315 | 115 | 1.80E−03 | 7.13E−06 | 8.79E−11 | NA |
| 1087 | 116 | 1.51E−02 | 7.20E−10 | 4.65E−10 | 4.94E−04 |
| 100001397 | 117 | 1.28E−02 | 1.49E−07 | 8.31E−09 | 2.00E−07 |
| X - 11491 | 118 | 1.25E−04 | 3.29E−09 | 6.16E−06 | NA |
| 100001393 | 119 | 3.73E−07 | 6.51E−08 | 5.05E−08 | 5.12E−03 |
| X - 17145 | 120 | 4.29E−04 | 1.24E−07 | 8.85E−08 | NA |
| 100001292 | 121 | 5.81E−07 | 1.87E−05 | 4.43E−07 | NA |
| X - 11880 | 122 | 8.29E−05 | 2.11E−08 | 2.99E−06 | NA |
| 100009162 | 123 | 2.80E−02 | 2.70E−06 | 2.99E−11 | 4.55E−06 |
| 100009148 | 124 | 1.42E−06 | 1.98E−07 | 8.34E−10 | 1.38E−01 |
| 100009160 | 125 | NA | NA | NA | 3.30E−06 |
| X - 18249 | 126 | 5.68E−06 | 2.29E−08 | 3.90E−04 | NA |
| X - 11381 | 127 | 2.95E−06 | 4.01E−06 | 6.23E−06 | NA |
| X - 21752 | 128 | 6.97E−03 | 1.55E−08 | 6.83E−07 | NA |
| X - 16944 | 129 | 1.69E−07 | 1.17E−04 | 3.85E−06 | NA |
| 235 | 130 | 3.50E−03 | 6.31E−09 | 2.45E−10 | 7.77E−02 |
| 100001276 | 131 | 4.94E−05 | 8.09E−09 | 1.42E−06 | 7.87E−04 |
| X - 24435 | 132 | 9.14E−03 | 6.83E−08 | 2.07E−07 | NA |
| 823 | 133 | 2.02E−01 | 2.94E−02 | 3.95E−12 | 2.92E−08 |
| 100009008 | 134 | 6.20E−02 | 1.05E−04 | 5.26E−13 | 2.37E−04 |
| 100009147 | 135 | 2.77E−03 | 6.17E−07 | 5.61E−12 | 8.96E−02 |
| X - 12306 | 136 | 4.21E−03 | 3.38E−04 | 1.28E−10 | NA |
| 1526 | 137 | 7.63E−05 | 2.41E−08 | 5.35E−07 | 1.09E−03 |
| 1162 | 138 | 3.86E−05 | 1.90E−09 | 6.26E−08 | 4.00E−01 |
| 112 | 139 | 1.04E−02 | 4.29E−07 | 1.52E−11 | 3.80E−02 |
| 100001851 | 140 | 5.72E−03 | 4.46E−08 | 2.94E−09 | 4.00E−03 |
| 100001399 | 141 | 1.76E−03 | 1.50E−07 | 3.44E−07 | 3.43E−05 |
| 480 | 142 | 4.06E−03 | 1.97E−08 | 2.55E−07 | 2.09E−04 |
| 1268 | 143 | 2.70E−04 | 6.75E−02 | 5.16E−14 | 6.31E−03 |
| 100010922 | 144 | 4.39E−05 | 2.27E−08 | 1.27E−06 | 5.62E−03 |
| X - 12844 | 145 | 6.26E−07 | 4.59E−06 | 4.31E−04 | NA |
| 340 | 146 | 4.50E−04 | 2.14E−06 | 4.38E−08 | 3.54E−04 |
| X - 16580 | 147 | 2.01E−04 | 5.40E−05 | 1.35E−07 | NA |
| 100009142 | 148 | 2.41E−04 | 1.42E−07 | 2.78E−07 | 2.83E−03 |
| X - 21736 | 149 | 4.65E−04 | 3.68E−05 | 1.27E−07 | NA |
| 100000406 | 150 | 7.11E−05 | 6.16E−08 | 6.87E−06 | 1.26E−03 |
| 100001051 | 151 | 2.35E−05 | 1.73E−06 | 1.22E−07 | 9.27E−03 |
| 100001565 | 152 | 8.27E−03 | 1.11E−06 | 2.96E−08 | 1.82E−04 |
| X - 11805 | 153 | 3.94E−04 | 3.01E−08 | 5.11E−04 | NA |
| 563 | 154 | 6.40E−04 | 2.68E−04 | 4.39E−07 | 1.59E−06 |
| 100001295 | 155 | NA | NA | NA | 1.87E−05 |
| 100001416 | 156 | 2.88E−04 | 1.22E−09 | 3.27E−05 | 1.37E−02 |
| 100001083 | 157 | 3.74E−04 | 3.81E−05 | 2.21E−06 | 8.30E−06 |
| 100008993 | 158 | 2.36E−04 | 3.34E−09 | 3.14E−05 | 1.80E−02 |
| 1002 | 159 | 3.12E−05 | 1.80E−04 | 1.60E−09 | 6.02E−02 |
| 100001054 | 160 | 1.26E−02 | 3.16E−06 | 9.48E−05 | 1.83E−07 |
| 100009030 | 161 | 6.04E−04 | 1.30E−04 | 3.70E−07 | 2.58E−05 |
| 197 | 162 | NA | NA | NA | 3.04E−05 |
| 100001556 | 163 | 2.54E−01 | 4.46E−04 | 2.21E−07 | 3.58E−08 |
| 100000665 | 164 | 1.85E−02 | 4.82E−05 | 1.25E−09 | 1.15E−03 |
| 297 | 165 | 4.57E−04 | 8.96E−09 | 5.01E−06 | 7.77E−02 |
| 100006121 | 166 | 3.59E−05 | 2.24E−06 | 4.65E−08 | 7.40E−01 |
| 100000924 | 167 | 1.63E−05 | 2.10E−05 | 7.34E−06 | 1.21E−03 |
| 100002028 | 168 | 1.77E−06 | 8.03E−07 | 4.83E−04 | 4.47E−03 |
| 100008984 | 169 | 2.13E−05 | 1.96E−05 | 1.74E−03 | 5.05E−06 |
| 100019978 | 170 | 3.64E−05 | 2.23E−06 | 1.37E−03 | NA |
| 100009132 | 171 | NA | NA | NA | 5.08E−05 |
| 100009350 | 172 | NA | NA | NA | 5.14E−05 |
| X - 15245 | 173 | 5.49E−02 | 1.15E−04 | 2.22E−08 | NA |
| 100002107 | 174 | 9.07E−01 | 8.91E−04 | 7.16E−10 | 1.26E−05 |
| 100008928 | 175 | 8.44E−06 | 8.15E−07 | 1.47E−05 | 7.24E−02 |
| 100002103 | 176 | 2.61E−02 | 4.02E−05 | 1.39E−07 | NA |
| 100009066 | 177 | 1.88E−03 | 5.75E−08 | 4.68E−06 | 1.79E−02 |
| 100002018 | 178 | 7.57E−06 | 5.40E−09 | 2.19E−03 | 1.11E−01 |
| 1528 | 179 | 3.59E−04 | 2.08E−08 | 3.60E−04 | 4.82E−03 |
| 100001456 | 180 | 2.87E−03 | 8.34E−06 | 5.54E−09 | 1.52E−01 |
| X - 17179 | 181 | 1.19E−04 | 3.85E−07 | 1.11E−02 | NA |
| 100001869 | 182 | 1.17E−06 | 2.94E−05 | 1.89E−03 | 6.83E−04 |
| 100004542 | 183 | 2.59E−06 | 1.60E−05 | 2.79E−06 | 4.56E−01 |
| 100001552 | 184 | 4.57E−07 | 1.46E−04 | 1.43E−06 | 6.07E−01 |
| 1025 | 185 | 8.05E−02 | 1.78E−04 | 3.06E−09 | 2.06E−03 |
| 100001435 | 186 | 2.11E−05 | 4.56E−07 | 6.05E−03 | 2.02E−03 |
| 100000257 | 187 | 2.50E−06 | 1.30E−05 | 3.74E−03 | 1.04E−03 |
| 100001618 | 188 | 1.44E−03 | 8.09E−08 | 1.40E−06 | 8.40E−01 |
| 100001925 | 189 | 4.47E−03 | 1.42E−06 | 3.80E−06 | 7.30E−03 |
| 100009153 | 190 | 1.52E−05 | 8.62E−07 | 3.57E−04 | 3.77E−02 |
| 100008977 | 191 | 1.35E−03 | 2.58E−06 | 1.97E−04 | 3.72E−04 |
| 100001126 | 192 | 6.28E−03 | 1.68E−01 | 1.51E−11 | 1.63E−02 |
| X - 14838 | 193 | 3.15E−05 | 1.41E−05 | 6.08E−03 | NA |
| 338 | 194 | 4.15E−06 | 3.65E−05 | 2.27E−05 | 1.23E−01 |
| 100008992 | 195 | 1.67E−04 | 3.99E−06 | 4.21E−04 | 2.99E−03 |
| 100001254 | 196 | 4.76E−04 | 1.62E−05 | 8.53E−05 | 1.34E−03 |
| 100009004 | 197 | 2.84E−02 | 1.39E−03 | 4.63E−10 | 4.87E−02 |
| 100001652 | 198 | 2.90E−01 | 1.19E−01 | 8.07E−08 | 3.44E−07 |
| 100004243 | 199 | 4.97E−03 | 1.76E−07 | 6.99E−04 | 2.08E−03 |
| 100000961 | 200 | 1.42E−04 | 4.98E−04 | 1.83E−05 | 1.11E−03 |
| 100002769 | 201 | 1.29E−05 | 3.28E−04 | 1.99E−05 | 2.77E−02 |
| X - 23593 | 202 | 1.23E−02 | 3.94E−04 | 2.37E−06 | NA |
| 100008914 | 203 | 1.09E−04 | 1.53E−03 | 1.60E−02 | 1.07E−06 |
| 1090 | 204 | 1.06E−05 | 1.80E−04 | 2.74E−03 | 5.57E−04 |
| X - 21626 | 205 | 3.36E−03 | 1.37E−04 | 2.91E−05 | NA |
| 100001208 | 206 | 6.84E−02 | 4.98E−09 | 1.07E−05 | 9.00E−01 |
| 93 | 207 | 1.62E−02 | 2.55E−06 | 1.93E−07 | 6.87E−01 |
| 100001567 | 208 | 2.17E−01 | 2.26E−02 | 2.08E−07 | 5.99E−06 |
| 1104 | 209 | 3.36E−02 | 3.92E−03 | 9.45E−06 | 5.18E−06 |
| 100008998 | 210 | 1.89E−03 | 1.86E−03 | 9.60E−04 | 2.12E−06 |
| X - 12100 | 211 | 7.63E−03 | 4.63E−02 | 7.81E−08 | NA |
| X - 14056 | 212 | 2.14E−02 | 1.28E−04 | 1.27E−05 | NA |
| 100008915 | 213 | 2.55E−01 | 2.13E−04 | 1.41E−08 | 1.58E−02 |
| 100008976 | 214 | 2.94E−04 | 9.42E−06 | 2.24E−04 | 2.28E−02 |
| X - 21339 | 215 | 8.49E−02 | 2.83E−06 | 2.57E−04 | NA |
| 100003001 | 216 | 1.85E−02 | 6.99E−06 | 6.95E−05 | 5.23E−03 |
| 100002876 | 217 | 2.82E−01 | 7.15E−03 | 1.21E−07 | 2.04E−04 |
| 504 | 218 | 1.15E−04 | 6.68E−05 | 1.55E−05 | 4.46E−01 |
| 100008929 | 219 | 1.59E−01 | 3.03E−08 | 2.34E−02 | NA |
| X - 16132 | 220 | 1.28E−02 | 3.36E−05 | 2.71E−04 | NA |
| X - 11530 | 221 | 1.07E−05 | 6.08E−05 | 1.89E−01 | NA |
| X - 12216 | 222 | 6.47E−03 | 1.09E−02 | 1.89E−06 | NA |
| X - 17337 | 223 | 4.13E−07 | 2.45E−02 | 1.32E−02 | NA |
| 100002063 | 224 | 1.06E−01 | 2.28E−04 | 3.28E−05 | 1.01E−04 |
| 1538 | 225 | 4.53E−06 | 2.91E−04 | 1.26E−01 | 7.55E−04 |
| 100008921 | 226 | 2.12E−01 | 8.88E−02 | 1.98E−09 | 3.55E−03 |
| 100001577 | 227 | 8.96E−04 | 2.56E−06 | 1.23E−03 | 5.31E−02 |
| X - 16123 | 228 | 4.96E−04 | 1.83E−05 | 3.28E−02 | NA |
| 100000846 | 229 | 8.03E−05 | 1.70E−08 | 2.59E−01 | 7.57E−01 |
| 100004083 | 230 | 1.99E−05 | 1.54E−03 | 6.99E−03 | 1.32E−03 |
| 1221 | 231 | 1.12E−05 | 5.56E−03 | 4.22E−03 | 1.09E−03 |
| 100001951 | 232 | 6.71E−04 | 6.09E−03 | 2.72E−02 | 3.75E−06 |
| 806 | 233 | 2.29E−03 | 8.98E−04 | 8.94E−06 | 2.40E−02 |
| X - 24061 | 234 | 1.95E−01 | 1.11E−03 | 2.58E−06 | NA |
| 100001553 | 235 | 5.32E−01 | 2.15E−02 | 1.37E−03 | 3.02E−08 |
| 100002293 | 236 | 9.07E−04 | 3.79E−06 | 2.35E−03 | 6.11E−02 |
| 100001320 | 237 | 4.99E−01 | 6.47E−01 | 6.27E−07 | 2.81E−06 |
| 100000616 | 238 | 8.16E−03 | 1.04E−05 | 1.33E−04 | 5.21E−02 |
| 100001590 | 239 | 1.19E−05 | 2.42E−05 | 7.48E−03 | 2.99E−01 |
| 100001765 | 240 | 2.28E−01 | 3.50E−05 | 3.72E−07 | 2.36E−01 |
| X - 11522 | 241 | 1.25E−05 | 4.36E−04 | 1.63E−01 | NA |
| 100001776 | 242 | 2.95E−02 | 4.98E−04 | 6.58E−06 | 9.45E−03 |
| 100000840 | 243 | 3.67E−05 | 9.76E−06 | 9.27E−02 | 2.77E−02 |
| 923 | 244 | 1.79E−03 | 7.64E−05 | 8.62E−06 | 9.23E−01 |
| 100001446 | 245 | 1.79E−03 | 1.15E−03 | 8.03E−07 | 7.12E−01 |
| 100001271 | 246 | 3.17E−01 | 9.18E−01 | 2.94E−04 | 1.57E−08 |
| 100001731 | 247 | 1.25E−01 | 1.19E−04 | 2.84E−06 | 4.03E−02 |
| 100002061 | 248 | 1.71E−01 | 3.66E−04 | 1.13E−07 | 2.74E−01 |
| 100000282 | 249 | 1.66E−03 | 2.78E−04 | 5.32E−05 | 8.10E−02 |
| 100000841 | 250 | 1.41E−01 | 2.02E−01 | 5.23E−06 | 2.87E−05 |
| 100002154 | 251 | 3.25E−03 | 1.42E−03 | 4.10E−06 | 2.46E−01 |
| 100008954 | 252 | 4.53E−01 | 5.09E−03 | 7.59E−07 | 2.81E−03 |
| 100001609 | 253 | 2.40E−05 | 1.04E−03 | 4.26E−03 | 4.63E−02 |
| 100001102 | 254 | 1.66E−03 | 3.52E−04 | 4.09E−05 | 2.13E−01 |
| 100001263 | 255 | 4.41E−01 | 3.16E−01 | 1.48E−05 | 2.51E−06 |
| 2050 | 256 | 6.95E−01 | 1.04E−02 | 3.55E−06 | 2.23E−04 |
| 100002784 | 257 | 2.13E−01 | 2.42E−04 | 9.74E−06 | 1.23E−02 |
| 100002877 | 258 | 1.32E−01 | 8.34E−03 | 2.87E−07 | 2.26E−02 |
| 100001007 | 259 | 6.00E−04 | 1.94E−05 | 3.21E−02 | 2.72E−02 |
| 100005466 | 260 | 6.65E−04 | 5.26E−05 | 3.43E−03 | 8.90E−02 |
| 100001593 | 261 | 1.54E−02 | 3.05E−04 | 6.58E−06 | 4.50E−01 |
| 100001740 | 262 | 5.70E−01 | 6.91E−04 | 1.69E−07 | 2.14E−01 |
| 100001398 | 263 | 1.29E−02 | 3.81E−05 | 8.08E−02 | 3.77E−04 |
| 100006056 | 264 | 1.13E−05 | 1.59E−03 | 1.80E−03 | 5.11E−01 |
| 100001579 | 265 | 5.27E−02 | 1.06E−03 | 1.05E−05 | 3.43E−02 |
| 100002528 | 266 | 2.76E−02 | 5.18E−04 | 4.47E−05 | 4.19E−02 |
| 100000657 | 267 | 3.84E−01 | 4.69E−02 | 7.05E−07 | 2.79E−03 |
| 100009394 | 268 | 1.78E−02 | 2.23E−07 | 2.98E−02 | 4.98E−01 |
| 302 | 269 | 2.66E−01 | 2.63E−03 | 9.17E−06 | 1.44E−02 |
| 1052 | 270 | 1.94E−02 | 3.67E−05 | 8.68E−03 | 1.69E−02 |
| 888 | 271 | 9.76E−04 | 1.78E−05 | 2.20E−02 | 3.00E−01 |
| X - 24021 | 272 | 1.22E−01 | 1.20E−01 | 3.13E−06 | NA |
| 100000611 | 273 | 2.94E−01 | 4.61E−02 | 4.09E−04 | 3.65E−05 |
| 1094 | 274 | 3.39E−01 | 2.57E−03 | 2.09E−05 | 1.39E−02 |
| 100001433 | 275 | 4.58E−03 | 2.42E−05 | 3.89E−03 | 6.75E−01 |
| 100001710 | 276 | 2.10E−03 | 1.39E−05 | 3.28E−02 | 5.24E−01 |
| 452 | 277 | 1.25E−02 | 9.08E−06 | 1.23E−02 | 4.23E−01 |
| 100009002 | 278 | 5.52E−04 | 2.26E−05 | 2.61E−01 | 2.63E−01 |
| 100001570 | 279 | 1.71E−01 | 2.48E−02 | 2.97E−05 | 7.28E−03 |
| 100004552 | 280 | 9.11E−01 | 3.16E−02 | 1.80E−06 | 1.93E−02 |
| 252 | 281 | 1.56E−03 | 2.04E−05 | 4.59E−01 | 6.91E−02 |
| 100002113 | 282 | 1.91E−03 | 1.10E−01 | 2.17E−05 | 2.24E−01 |
| 100001155 | 283 | 1.32E−01 | 1.33E−03 | 4.40E−05 | 2.16E−01 |
| 144 | 284 | 1.88E−01 | 9.44E−04 | 2.82E−05 | 3.52E−01 |
| 100001843 | 285 | 9.88E−02 | 3.07E−01 | 9.69E−08 | 7.34E−01 |
| 100002241 | 286 | 1.31E−02 | 2.29E−05 | 1.07E−02 | 7.72E−01 |
| 1004 | 287 | 7.78E−02 | 3.06E−05 | 4.40E−03 | 2.43E−01 |
| 100009149 | 288 | 5.38E−01 | 2.02E−02 | 3.51E−05 | NA |
| 1083 | 289 | 7.97E−01 | 2.34E−01 | 3.30E−03 | 7.50E−06 |
| 100006292 | 290 | 2.31E−05 | 4.78E−03 | 2.81E−01 | 1.93E−01 |
| 100009138 | 291 | 1.81E−02 | 1.29E−01 | 7.45E−01 | 4.19E−06 |
| 100002060 | 292 | 8.05E−01 | 6.45E−03 | 1.44E−05 | 1.23E−01 |
| 100003678 | 293 | 7.41E−02 | 3.13E−01 | 4.65E−05 | NA |
| X - 13737 | 294 | 6.48E−01 | 6.50E−02 | 3.61E−05 | NA |
| 100001461 | 295 | 1.11E−01 | 2.00E−01 | 5.90E−02 | 1.60E−05 |
| 100009166 | 296 | 5.31E−01 | 1.75E−01 | 1.42E−02 | 2.16E−05 |
| 100001153 | 297 | 2.68E−05 | 2.77E−01 | 2.91E−02 | 1.42E−01 |
| 100004499 | 298 | 2.76E−01 | 5.33E−03 | 2.71E−05 | 7.98E−01 |
| 1239 | 299 | 2.49E−01 | 6.44E−03 | 5.14E−05 | 5.36E−01 |
| 100001400 | 300 | 7.20E−01 | 1.39E−01 | 4.83E−02 | 9.27E−06 |
| 100009036 | 301 | 3.73E−01 | 6.03E−03 | 3.65E−05 | 6.44E−01 |
| 100000936 | 302 | 7.04E−01 | 3.11E−01 | 2.27E−02 | 2.68E−05 |
| 100001262 | 303 | 3.91E−01 | 8.91E−01 | 4.73E−06 | 1.56E−01 |
| 100008918 | 304 | 9.85E−01 | 8.43E−02 | 1.36E−05 | 2.74E−01 |
| 100010901 | 305 | 2.05E−01 | 8.75E−01 | 1.47E−05 | 9.64E−01 |
| 100000036 | 306 | 7.17E−01 | 4.13E−01 | 3.08E−01 | 5.48E−05 |
| 267 | 307 | 9.75E−01 | 9.39E−01 | 3.86E−05 | 5.64E−01 |
| 100009331 | 308 | NA | NA | NA | 1.60E−04 |
| 100003677 | 309 | NA | NA | NA | 1.73E−04 |
| 100009134 | 310 | NA | NA | NA | 2.93E−04 |
| X - 11787 | 311 | 5.82E−04 | 2.52E−04 | 2.16E−04 | NA |
| 100015684 | 312 | NA | NA | NA | 3.22E−04 |
| 100002873 | 313 | NA | NA | NA | 3.54E−04 |
| 100010935 | 314 | NA | NA | NA | 3.55E−04 |
| 100009361 | 315 | NA | NA | NA | 4.28E−04 |
| X - 24309 | 316 | 5.45E−04 | 7.90E−04 | 2.47E−04 | NA |
| 100002749 | 317 | 1.85E−03 | 7.16E−05 | 2.18E−04 | 2.50E−03 |
| 189 | 318 | 2.94E−04 | 1.91E−04 | 2.95E−04 | 4.66E−03 |
| 100015838 | 319 | NA | NA | NA | 5.81E−04 |
| 100005396 | 320 | NA | NA | NA | 5.83E−04 |
| 100010940 | 321 | NA | NA | NA | 6.21E−04 |
| 100009347 | 322 | NA | NA | NA | 6.79E−04 |
| 100006614 | 323 | NA | NA | NA | 7.91E−04 |
| X - 21628 | 324 | 1.12E−02 | 6.28E−05 | 7.58E−04 | NA |
| 100001127 | 325 | 1.04E−03 | 6.48E−05 | 1.30E−02 | NA |
| 100001413 | 326 | NA | NA | NA | 1.04E−03 |
| 100009026 | 327 | NA | NA | NA | 1.08E−03 |
| 100010937 | 328 | NA | NA | NA | 1.16E−03 |
| 100010936 | 329 | NA | NA | NA | 1.20E−03 |
| 310 | 330 | 1.17E−03 | 3.56E−04 | 5.79E−03 | 8.98E−04 |
| 100000943 | 331 | 8.11E−04 | 2.59E−03 | 3.31E−04 | 3.49E−03 |
| 100003926 | 332 | 1.18E−04 | 1.95E−03 | 3.85E−04 | 5.02E−02 |
| 141 | 333 | NA | NA | NA | 1.61E−03 |
| X - 11441 | 334 | 2.80E−04 | 9.47E−05 | 1.61E−01 | NA |
| 100002927 | 335 | NA | NA | NA | 1.68E−03 |
| 100000269 | 336 | 1.18E−01 | 3.37E−04 | 1.45E−04 | 1.48E−03 |
| 100009027 | 337 | NA | NA | NA | 1.72E−03 |
| 100010917 | 338 | NA | NA | NA | 1.76E−03 |
| 1001 | 339 | 2.13E−02 | 3.00E−03 | 6.24E−04 | 3.76E−04 |
| 100000453 | 340 | 7.14E−02 | 1.73E−04 | 1.73E−03 | 7.63E−04 |
| 100000827 | 341 | 7.19E−04 | 6.79E−05 | 4.36E−04 | 9.85E−01 |
| 882 | 342 | 2.26E−03 | 1.73E−03 | 2.75E−03 | NA |
| 100001033 | 343 | NA | NA | NA | 2.26E−03 |
| 100001327 | 344 | 2.95E−02 | 2.45E−04 | 1.70E−03 | NA |
| 100001193 | 345 | 3.37E−03 | 3.99E−04 | 2.47E−04 | 9.31E−02 |
| 100010916 | 346 | NA | NA | NA | 2.42E−03 |
| 849 | 347 | 1.53E−01 | 1.71E−04 | 2.01E−03 | 7.03E−04 |
| 100001452 | 348 | 7.26E−04 | 1.05E−04 | 1.59E−03 | 3.48E−01 |
| 100009375 | 349 | NA | NA | NA | 2.55E−03 |
| 1140 | 350 | 8.25E−03 | 1.26E−04 | 5.03E−02 | 9.66E−04 |
| X - 24241 | 351 | 1.21E−02 | 2.55E−03 | 6.53E−04 | NA |
| X - 16935 | 352 | 2.27E−01 | 1.21E−03 | 7.44E−05 | NA |
| 100015786 | 353 | NA | NA | NA | 2.80E−03 |
| 100000445 | 354 | 6.40E−03 | 2.12E−04 | 6.68E−03 | 7.71E−03 |
| X - 12822 | 355 | 3.26E−02 | 6.57E−03 | 1.20E−04 | NA |
| X - 21737 | 356 | 2.28E−02 | 2.03E−03 | 5.99E−04 | NA |
| X - 15728 | 357 | 2.04E−03 | 5.92E−04 | 2.41E−02 | NA |
| 100015620 | 358 | NA | NA | NA | 3.27E−03 |
| X - 12798 | 359 | 2.00E−03 | 8.38E−03 | 2.38E−03 | NA |
| 1547 | 360 | NA | NA | NA | 3.45E−03 |
| 275 | 361 | 1.84E−02 | 5.58E−03 | 8.40E−05 | 2.11E−02 |
| 100015683 | 362 | NA | NA | NA | 3.68E−03 |
| 100000715 | 363 | NA | NA | NA | 3.70E−03 |
| 100004299 | 364 | 4.48E−04 | 9.39E−05 | 5.06E−03 | 9.99E−01 |
| X - 18914 | 365 | 1.45E−04 | 2.74E−03 | 1.62E−01 | NA |
| 100000015 | 366 | 3.08E−03 | 1.39E−03 | 6.16E−04 | 1.04E−01 |
| 2048 | 367 | 6.00E−04 | 9.94E−04 | 6.47E−04 | 8.81E−01 |
| 100003637 | 368 | 4.65E−02 | 5.03E−03 | 3.73E−04 | NA |
| X - 11372 | 369 | 2.03E−02 | 1.52E−03 | 2.85E−03 | NA |
| 100002254 | 370 | NA | NA | NA | 4.68E−03 |
| 100000580 | 371 | 4.33E−03 | 1.82E−03 | 1.78E−01 | 3.60E−04 |
| 100009028 | 372 | NA | NA | NA | 4.86E−03 |
| 100001510 | 373 | 3.41E−03 | 1.45E−04 | 1.39E−03 | 8.68E−01 |
| 100003520 | 374 | 3.05E−02 | 6.91E−05 | 6.20E−02 | NA |
| X - 11442 | 375 | 3.09E−03 | 1.16E−04 | 3.88E−01 | NA |
| X - 11838 | 376 | 6.81E−02 | 3.54E−04 | 5.93E−03 | NA |
| 100000776 | 377 | 1.29E−03 | 1.89E−03 | 2.69E−01 | 1.18E−03 |
| 100001278 | 378 | 3.90E−02 | 9.14E−05 | 6.14E−03 | 4.12E−02 |
| 100000711 | 379 | 4.63E−03 | 3.91E−03 | 4.64E−04 | 1.18E−01 |
| 100002717 | 380 | 2.66E−02 | 6.16E−04 | 1.55E−03 | 4.28E−02 |
| 100009035 | 381 | 2.16E−02 | 1.37E−03 | 1.19E−04 | 3.27E−01 |
| X - 14364 | 382 | 1.26E−02 | 3.79E−04 | 4.97E−02 | NA |
| 100006370 | 383 | NA | NA | NA | 6.40E−03 |
| 100009360 | 384 | NA | NA | NA | 6.53E−03 |
| 100002462 | 385 | NA | NA | NA | 6.69E−03 |
| 100001768 | 386 | 2.15E−01 | 5.71E−05 | 2.82E−04 | 5.97E−01 |
| 100000054 | 387 | 7.18E−04 | 1.77E−01 | 1.00E−02 | 1.69E−03 |
| 100003434 | 388 | 8.98E−01 | 1.04E−03 | 1.13E−03 | 2.04E−03 |
| 100001739 | 389 | 3.88E−04 | 8.27E−03 | 3.11E−02 | 2.41E−02 |
| 100001872 | 390 | NA | NA | NA | 7.06E−03 |
| 100000966 | 391 | 1.33E−02 | 2.69E−04 | 1.19E−03 | 5.90E−01 |
| 100009343 | 392 | NA | NA | NA | 7.56E−03 |
| X - 22162 | 393 | 2.08E−02 | 9.71E−03 | 2.17E−03 | NA |
| 100002173 | 394 | 5.99E−02 | 4.36E−03 | 1.20E−04 | 1.13E−01 |
| 100001274 | 395 | NA | NA | NA | 7.88E−03 |
| X - 11847 | 396 | 1.01E−02 | 1.28E−02 | 3.82E−03 | NA |
| 391 | 397 | 4.14E−02 | 8.98E−04 | 1.91E−04 | 6.11E−01 |
| 100010925 | 398 | NA | NA | NA | 8.64E−03 |
| 100015962 | 399 | 4.05E−02 | 3.12E−03 | 1.04E−03 | 5.52E−02 |
| 331 | 400 | 1.53E−01 | 8.07E−01 | 1.42E−04 | 4.15E−04 |
| 100006430 | 401 | 4.21E−02 | 1.29E−03 | 5.90E−03 | 2.27E−02 |
| 100000258 | 402 | 3.03E−02 | 1.65E−04 | 2.08E−03 | 7.64E−01 |
| 179 | 403 | NA | NA | NA | 9.53E−03 |
| X - 16946 | 404 | 8.43E−04 | 1.52E−03 | 7.02E−01 | NA |
| 100009397 | 405 | NA | NA | NA | 9.81E−03 |
| 100009346 | 406 | NA | NA | NA | 9.83E−03 |
| 100000802 | 407 | 5.26E−03 | 2.30E−03 | 1.21E−02 | 7.08E−02 |
| 100015759 | 408 | NA | NA | NA | 1.02E−02 |
| 100015593 | 409 | NA | NA | NA | 1.03E−02 |
| 100001178 | 410 | 7.09E−01 | 1.21E−03 | 2.21E−04 | 5.98E−02 |
| 100001580 | 411 | NA | NA | NA | 1.08E−02 |
| 100005351 | 412 | 9.39E−01 | 1.42E−02 | 1.24E−03 | 9.05E−04 |
| 100009376 | 413 | NA | NA | NA | 1.11E−02 |
| 100005850 | 414 | 4.79E−01 | 6.31E−02 | 1.67E−03 | 3.42E−04 |
| X - 21448 | 415 | 2.66E−03 | 8.56E−04 | 6.65E−01 | NA |
| 503 | 416 | 2.01E−01 | 4.57E−02 | 1.62E−02 | 1.17E−04 |
| 100006260 | 417 | 2.29E−01 | 1.97E−02 | 7.38E−05 | 7.04E−02 |
| 100001615 | 418 | 1.02E−02 | 4.35E−04 | 2.44E−02 | 2.33E−01 |
| 100004541 | 419 | NA | NA | NA | 1.28E−02 |
| 356 | 420 | 3.12E−02 | 2.55E−02 | 3.10E−02 | 1.09E−03 |
| 100001511 | 421 | 7.53E−05 | 2.86E−03 | 2.94E−01 | 4.43E−01 |
| 100001562 | 422 | 3.67E−01 | 2.70E−01 | 1.92E−03 | 1.54E−04 |
| 100001810 | 423 | 8.03E−02 | 5.35E−03 | 2.10E−04 | 3.40E−01 |
| 100006651 | 424 | NA | NA | NA | 1.34E−02 |
| 100003179 | 425 | 2.77E−01 | 3.78E−02 | 2.49E−04 | NA |
| X - 14939 | 426 | 1.28E−02 | 1.22E−01 | 1.69E−03 | NA |
| 100001613 | 427 | 3.01E−02 | 3.21E−03 | 5.24E−04 | 7.59E−01 |
| 100002356 | 428 | 1.11E−01 | 8.32E−03 | 7.14E−04 | 6.25E−02 |
| 100009333 | 429 | NA | NA | NA | 1.43E−02 |
| 100001040 | 430 | 6.55E−03 | 7.86E−03 | 7.08E−02 | 1.14E−02 |
| 100001597 | 431 | 6.23E−04 | 5.88E−03 | 9.49E−02 | 1.21E−01 |
| 100005864 | 432 | 3.30E−01 | 4.95E−02 | 1.42E−02 | 1.84E−04 |
| 100000467 | 433 | 1.35E−01 | 1.37E−02 | 1.68E−04 | 1.41E−01 |
| 100009332 | 434 | NA | NA | NA | 1.45E−02 |
| 100002397 | 435 | 1.42E−01 | 3.14E−03 | 7.14E−03 | NA |
| 100001182 | 436 | 5.14E−02 | 1.23E−03 | 9.65E−04 | 7.91E−01 |
| 100000963 | 437 | 4.45E−02 | 4.87E−04 | 3.00E−02 | 7.86E−02 |
| X - 16071 | 438 | 9.91E−04 | 5.37E−03 | 6.52E−01 | NA |
| 171 | 439 | 4.29E−01 | 1.57E−02 | 1.40E−01 | 6.05E−05 |
| X - 09789 | 440 | 1.65E−02 | 1.46E−02 | 1.56E−02 | NA |
| 100015681 | 441 | NA | NA | NA | 1.55E−02 |
| X - 12442 | 442 | 7.71E−02 | 1.22E−03 | 4.16E−02 | NA |
| 100001055 | 443 | 3.05E−02 | 2.25E−02 | 5.33E−03 | 1.70E−02 |
| 100009209 | 444 | NA | NA | NA | 1.62E−02 |
| 100015610 | 445 | NA | NA | NA | 1.66E−02 |
| X - 23782 | 446 | 1.14E−01 | 3.57E−03 | 1.20E−02 | NA |
| 100009145 | 447 | 2.15E−03 | 1.56E−03 | 5.27E−02 | 4.71E−01 |
| 1080 | 448 | 7.87E−05 | 1.37E−01 | 4.63E−01 | NA |
| 209 | 449 | NA | NA | NA | 1.71E−02 |
| X - 11308 | 450 | 2.35E−01 | 5.02E−03 | 4.51E−03 | NA |
| 100002126 | 451 | 9.99E−01 | 2.19E−02 | 2.11E−02 | 2.05E−04 |
| 1123 | 452 | 9.07E−02 | 7.96E−04 | 2.48E−02 | 6.61E−02 |
| 100006116 | 453 | 2.90E−02 | 1.28E−02 | 1.08E−02 | 3.28E−02 |
| 913 | 454 | NA | NA | NA | 1.92E−02 |
| 100002869 | 455 | NA | NA | NA | 1.94E−02 |
| X - 11478 | 456 | 2.56E−02 | 3.27E−01 | 8.78E−04 | NA |
| 827 | 457 | 5.25E−02 | 2.30E−04 | 1.32E−01 | 9.02E−02 |
| 62 | 458 | NA | NA | NA | 1.98E−02 |
| 100000707 | 459 | 6.65E−01 | 1.75E−01 | 1.34E−03 | 1.01E−03 |
| 407 | 460 | 1.05E−02 | 4.12E−03 | 1.91E−02 | 1.92E−01 |
| 932 | 461 | 2.88E−03 | 6.14E−04 | 2.17E−01 | 4.62E−01 |
| 100015967 | 462 | NA | NA | NA | 2.09E−02 |
| 250 | 463 | 3.10E−03 | 1.53E−03 | 5.37E−01 | 8.04E−02 |
| 100009233 | 464 | NA | NA | NA | 2.17E−02 |
| 100008957 | 465 | 3.18E−04 | 5.51E−01 | 4.41E−02 | 3.15E−02 |
| 100004646 | 466 | NA | NA | NA | 2.24E−02 |
| 272 | 467 | NA | NA | NA | 2.24E−02 |
| 100001466 | 468 | NA | NA | NA | 2.25E−02 |
| X - 17178 | 469 | 4.05E−01 | 1.23E−01 | 2.30E−04 | NA |
| 537 | 470 | 9.22E−03 | 5.30E−03 | 1.92E−01 | 2.84E−02 |
| 100015839 | 471 | NA | NA | NA | 2.29E−02 |
| 100000016 | 472 | 3.93E−04 | 5.17E−02 | 2.08E−02 | 7.50E−01 |
| X - 21796 | 473 | 4.48E−02 | 3.14E−01 | 9.59E−04 | NA |
| 100004575 | 474 | NA | NA | NA | 2.40E−02 |
| 100000774 | 475 | 8.39E−03 | 3.09E−02 | 5.83E−03 | 2.23E−01 |
| 100009335 | 476 | NA | NA | NA | 2.44E−02 |
| 1506 | 477 | NA | NA | NA | 2.44E−02 |
| 100001594 | 478 | NA | NA | NA | 2.45E−02 |
| 2049 | 479 | 1.75E−02 | 1.87E−02 | 1.25E−03 | 9.01E−01 |
| 100001756 | 480 | 4.30E−02 | 2.78E−02 | 1.26E−01 | 2.52E−03 |
| 100001391 | 481 | 4.63E−01 | 1.81E−01 | 8.60E−05 | 5.42E−02 |
| 100015968 | 482 | NA | NA | NA | 2.51E−02 |
| 194 | 483 | 5.79E−01 | 7.63E−02 | 3.91E−04 | 2.29E−02 |
| 100001992 | 484 | 5.43E−02 | 9.83E−03 | 1.73E−01 | 4.35E−03 |
| 254 | 485 | 2.59E−01 | 2.34E−02 | 1.41E−03 | 4.95E−02 |
| 100004089 | 486 | 2.09E−01 | 4.72E−02 | 3.91E−02 | 1.18E−03 |
| 100001541 | 487 | 3.18E−03 | 3.18E−02 | 3.95E−02 | 1.17E−01 |
| 100009150 | 488 | NA | NA | NA | 2.64E−02 |
| 100001994 | 489 | 1.42E−01 | 3.18E−03 | 2.50E−01 | 4.53E−03 |
| 100020004 | 490 | 3.61E−04 | 8.56E−02 | 6.55E−01 | NA |
| 100005371 | 491 | 4.30E−03 | 3.47E−02 | 2.79E−01 | 1.32E−02 |
| 100015745 | 492 | NA | NA | NA | 2.73E−02 |
| 100004182 | 493 | NA | NA | NA | 2.75E−02 |
| 100000743 | 494 | 8.80E−05 | 3.57E−02 | 2.44E−01 | 7.44E−01 |
| 100015845 | 495 | NA | NA | NA | 2.76E−02 |
| X - 21353 | 496 | 1.56E−01 | 1.08E−02 | 1.28E−02 | NA |
| X - 12206 | 497 | 1.45E−01 | 2.30E−02 | 6.87E−03 | NA |
| 100010918 | 498 | NA | NA | NA | 2.85E−02 |
| X - 16570 | 499 | 2.78E−02 | 1.77E−02 | 4.74E−02 | NA |
| 100000463 | 500 | 1.94E−02 | 2.42E−02 | 1.74E−03 | 8.68E−01 |
| 100005372 | 501 | 6.66E−01 | 2.92E−02 | 1.60E−03 | 2.28E−02 |
| X - 21834 | 502 | 2.08E−02 | 3.71E−02 | 3.22E−02 | NA |
| 100000011 | 503 | 3.42E−02 | 4.44E−03 | 1.30E−02 | 4.00E−01 |
| 100001403 | 504 | 3.11E−01 | 6.66E−02 | 9.99E−02 | 3.93E−04 |
| 100002171 | 505 | 3.09E−02 | 6.17E−02 | 6.20E−03 | 7.07E−02 |
| 100004555 | 506 | NA | NA | NA | 3.09E−02 |
| 100002152 | 507 | 6.60E−01 | 2.20E−02 | 5.17E−02 | 1.24E−03 |
| X - 22816 | 508 | 1.06E−01 | 1.20E−01 | 2.40E−03 | NA |
| X - 12127 | 509 | 5.67E−02 | 3.36E−02 | 1.62E−02 | NA |
| 100001022 | 510 | 9.84E−03 | 5.82E−01 | 3.06E−03 | 5.57E−02 |
| 100001445 | 511 | 2.27E−01 | 4.02E−03 | 3.44E−02 | NA |
| 100001596 | 512 | 1.39E−02 | 2.06E−02 | 2.86E−01 | 1.24E−02 |
| 100010950 | 513 | NA | NA | NA | 3.21E−02 |
| 100001106 | 514 | 3.48E−01 | 8.51E−02 | 6.69E−01 | 5.51E−05 |
| 100003674 | 515 | 2.22E−03 | 4.43E−02 | 1.07E−01 | 1.04E−01 |
| 100002183 | 516 | 5.79E−04 | 8.84E−02 | 1.80E−01 | 1.30E−01 |
| 100000014 | 517 | 1.26E−01 | 5.77E−03 | 1.23E−02 | 1.45E−01 |
| 100002167 | 518 | 8.04E−03 | 9.30E−02 | 1.08E−02 | 1.64E−01 |
| 100002204 | 519 | 1.31E−02 | 1.13E−02 | 1.89E−02 | 5.21E−01 |
| 100000862 | 520 | 1.15E−02 | 1.66E−02 | 2.35E−01 | NA |
| 100001064 | 521 | 6.05E−01 | 4.13E−01 | 5.20E−04 | 1.24E−02 |
| 100005986 | 522 | 1.92E−01 | 1.85E−01 | 7.15E−01 | 6.52E−05 |
| X -12459 | 523 | 6.99E−02 | 1.06E−02 | 6.24E−02 | NA |
| 100003686 | 524 | 1.45E−01 | 1.01E−02 | 1.76E−03 | 6.89E−01 |
| 100001784 | 525 | NA | NA | NA | 3.75E−02 |
| 409 | 526 | 4.23E−01 | 4.06E−02 | 1.09E−03 | 1.10E−01 |
| 1442 | 527 | 2.07E−03 | 6.89E−02 | 6.69E−02 | 2.25E−01 |
| 100001604 | 528 | 2.44E−01 | 1.84E−03 | 3.76E−02 | 1.32E−01 |
| 100003200 | 529 | 1.76E−02 | 1.13E−03 | 1.69E−01 | 7.42E−01 |
| 100000706 | 530 | 2.38E−02 | 6.76E−03 | 5.73E−02 | 2.81E−01 |
| 100000437 | 531 | 3.02E−01 | 4.89E−02 | 3.70E−01 | 4.75E−04 |
| 1135 | 532 | 1.16E−01 | 3.94E−02 | 4.90E−02 | 1.17E−02 |
| X - 12212 | 533 | 1.06E−03 | 1.96E−01 | 3.14E−01 | NA |
| 100001251 | 534 | 2.37E−01 | 1.80E−03 | 7.13E−02 | 8.91E−02 |
| 1492 | 535 | NA | NA | NA | 4.13E−02 |
| 158 | 536 | 2.55E−01 | 1.19E−02 | 4.68E−03 | 2.05E−01 |
| 100002405 | 537 | 5.14E−02 | 2.17E−01 | 6.58E−03 | NA |
| 279 | 538 | 1.37E−02 | 3.05E−01 | 1.89E−01 | 4.07E−03 |
| 100005384 | 539 | 9.40E−02 | 4.92E−03 | 5.59E−01 | 1.28E−02 |
| 100008956 | 540 | 3.98E−01 | 2.40E−01 | 5.42E−04 | 6.60E−02 |
| X - 21729 | 541 | 6.21E−02 | 1.88E−01 | 6.81E−03 | NA |
| 100009271 | 542 | NA | NA | NA | 4.30E−02 |
| 100010958 | 543 | NA | NA | NA | 4.33E−02 |
| 100001501 | 544 | 2.97E−02 | 2.95E−03 | 2.37E−01 | 1.73E−01 |
| 100000584 | 545 | 3.28E−02 | 2.36E−03 | 1.50E−01 | 3.45E−01 |
| 100002137 | 546 | 5.53E−03 | 2.80E−01 | 5.87E−02 | NA |
| X - 11440 | 547 | 3.73E−01 | 1.82E−02 | 1.33E−02 | NA |
| 848 | 548 | 1.33E−01 | 2.27E−03 | 8.49E−02 | 1.62E−01 |
| 100001437 | 549 | NA | NA | NA | 4.54E−02 |
| 362 | 550 | NA | NA | NA | 4.55E−02 |
| 100000660 | 551 | 8.86E−01 | 1.51E−01 | 3.09E−04 | 1.08E−01 |
| 100001658 | 552 | 4.55E−02 | 2.96E−02 | 1.23E−01 | 2.71E−02 |
| 100010919 | 553 | NA | NA | NA | 4.62E−02 |
| 1053 | 554 | 2.66E−03 | 2.57E−01 | 8.02E−02 | 8.48E−02 |
| 100008980 | 555 | 9.13E−01 | 7.64E−01 | 7.90E−04 | 8.86E−03 |
| 1235 | 556 | 1.84E−01 | 4.88E−01 | 1.50E−04 | 3.73E−01 |
| 100006125 | 557 | NA | NA | NA | 4.74E−02 |
| 100001409 | 558 | NA | NA | NA | 4.74E−02 |
| 100000101 | 559 | 5.12E−03 | 5.15E−01 | 4.10E−02 | NA |
| 825 | 560 | 1.98E−02 | 7.73E−04 | 8.16E−01 | 4.16E−01 |
| 100006314 | 561 | 7.63E−01 | 8.25E−02 | 3.10E−04 | 2.69E−01 |
| 100015915 | 562 | NA | NA | NA | 4.81E−02 |
| 1105 | 563 | 7.26E−02 | 1.09E−01 | 1.32E−03 | 5.46E−01 |
| 100001870 | 564 | 6.54E−02 | 6.64E−03 | 5.14E−02 | 3.08E−01 |
| 100001527 | 565 | 5.28E−01 | 3.67E−02 | 1.38E−03 | 2.63E−01 |
| 100002761 | 566 | 7.63E−02 | 3.18E−02 | 5.68E−02 | NA |
| X - 23705 | 567 | 4.09E−01 | 7.15E−02 | 5.14E−03 | NA |
| X - 21411 | 568 | 6.28E−01 | 2.18E−01 | 1.12E−03 | NA |
| 100015587 | 569 | NA | NA | NA | 5.36E−02 |
| 100003549 | 570 | 2.49E−01 | 4.90E−03 | 1.36E−01 | NA |
| 100003696 | 571 | 4.87E−01 | 1.19E−02 | 1.97E−01 | 8.16E−03 |
| 424 | 572 | 1.91E−01 | 1.84E−03 | 3.91E−01 | 6.93E−02 |
| 799 | 573 | 9.42E−01 | 7.44E−02 | 1.72E−02 | 8.38E−03 |
| 100001300 | 574 | 7.05E−04 | 6.33E−02 | 3.02E−01 | 7.67E−01 |
| 100001795 | 575 | 1.14E−01 | 4.30E−02 | 3.39E−03 | 6.53E−01 |
| X - 24097 | 576 | 2.12E−01 | 7.48E−03 | 1.21E−01 | NA |
| 100000626 | 577 | 1.53E−01 | 7.89E−04 | 1.64E−01 | 5.81E−01 |
| 100000042 | 578 | 7.89E−03 | 1.73E−02 | 1.15E−01 | 7.80E−01 |
| 100000096 | 579 | 1.39E−02 | 4.86E−02 | 5.91E−02 | 3.17E−01 |
| 878 | 580 | 9.29E−04 | 3.50E−01 | 7.94E−02 | 5.03E−01 |
| 100006190 | 581 | 6.95E−02 | 1.76E−01 | 5.58E−03 | 1.99E−01 |
| 100000803 | 582 | 1.60E−01 | 7.29E−03 | 1.93E−01 | NA |
| 100001383 | 583 | 1.16E−02 | 1.62E−02 | 8.20E−01 | 8.94E−02 |
| X - 13866 | 584 | 9.84E−03 | 4.40E−02 | 5.33E−01 | NA |
| 100002945 | 585 | 2.80E−01 | 1.08E−01 | 1.44E−01 | 3.30E−03 |
| 100003901 | 586 | 4.03E−01 | 1.07E−01 | 4.05E−01 | 8.25E−04 |
| 100008904 | 587 | 5.58E−03 | 3.99E−03 | 8.25E−01 | 8.00E−01 |
| 100010944 | 588 | NA | NA | NA | 6.22E−02 |
| 100015966 | 589 | NA | NA | NA | 6.22E−02 |
| 100000487 | 590 | 1.59E−01 | 4.06E−03 | 3.79E−01 | NA |
| 100001335 | 591 | 5.99E−01 | 2.40E−03 | 1.89E−02 | 5.85E−01 |
| 100001620 | 592 | 4.98E−01 | 2.82E−03 | 8.84E−02 | 1.30E−01 |
| 100001614 | 593 | 1.23E−01 | 1.92E−02 | 1.82E−02 | 3.87E−01 |
| 1113 | 594 | 6.29E−01 | 6.84E−02 | 1.55E−03 | 2.58E−01 |
| 100001791 | 595 | 1.05E−01 | 3.64E−02 | 7.05E−03 | 6.68E−01 |
| 100006115 | 596 | 1.71E−02 | 3.46E−02 | 3.55E−02 | 8.73E−01 |
| X - 23293 | 597 | 8.21E−02 | 2.04E−02 | 1.70E−01 | NA |
| X - 11850 | 598 | 9.79E−02 | 6.16E−02 | 4.73E−02 | NA |
| X - 21286 | 599 | 4.05E−01 | 1.53E−02 | 4.68E−02 | NA |
| 100008999 | 600 | 3.83E−02 | 8.66E−02 | 7.26E−01 | 8.13E−03 |
| 100009146 | 601 | NA | NA | NA | 6.69E−02 |
| 339 | 602 | 1.12E−02 | 1.34E−01 | 4.18E−02 | 3.19E−01 |
| 100003700 | 603 | 4.01E−02 | 8.06E−02 | 1.01E−01 | NA |
| 100001148 | 604 | 1.77E−02 | 2.63E−02 | 8.94E−02 | 5.44E−01 |
| 1258 | 605 | 1.57E−02 | 1.16E−01 | 1.81E−01 | NA |
| 100000870 | 606 | 1.36E−01 | 9.40E−02 | 8.83E−02 | 2.07E−02 |
| 1128 | 607 | 2.06E−02 | 5.27E−02 | 4.48E−02 | 4.84E−01 |
| 100001392 | 608 | 3.08E−01 | 2.70E−03 | 7.72E−02 | 3.72E−01 |
| 100009234 | 609 | NA | NA | NA | 7.02E−02 |
| 100002021 | 610 | 7.04E−01 | 2.30E−01 | 2.26E−04 | 6.68E−01 |
| 100002122 | 611 | 7.67E−01 | 2.05E−02 | 2.13E−02 | 7.58E−02 |
| 100004284 | 612 | NA | NA | NA | 7.20E−02 |
| X - 18899 | 613 | 2.23E−02 | 4.02E−01 | 4.27E−02 | NA |
| 100001569 | 614 | 8.88E−01 | 3.68E−01 | 8.82E−02 | 9.72E−04 |
| 100010934 | 615 | NA | NA | NA | 7.34E−02 |
| 100002094 | 616 | 1.68E−01 | 1.10E−02 | 1.51E−01 | 1.04E−01 |
| X - 11452 | 617 | 6.44E−02 | 9.91E−02 | 6.33E−02 | NA |
| 100004328 | 618 | 1.84E−03 | 2.54E−01 | 3.70E−01 | 1.77E−01 |
| 100001621 | 619 | 5.29E−01 | 6.04E−03 | 1.36E−01 | NA |
| 100010949 | 620 | NA | NA | NA | 7.60E−02 |
| 1537 | 621 | 9.13E−01 | 1.37E−01 | 3.40E−04 | 7.90E−01 |
| 100001987 | 622 | 5.13E−02 | 2.42E−02 | 5.69E−01 | 4.85E−02 |
| 100009345 | 623 | NA | NA | NA | 7.66E−02 |
| 100006092 | 624 | 5.50E−01 | 8.10E−04 | 1.31E−01 | 5.92E−01 |
| 100015760 | 625 | 5.57E−02 | 9.55E−01 | 1.44E−01 | 4.60E−03 |
| 100009344 | 626 | NA | NA | NA | 7.75E−02 |
| 1628 | 627 | 5.63E−01 | 9.20E−01 | 1.05E−04 | 6.90E−01 |
| 100006290 | 628 | 3.99E−02 | 1.46E−01 | 1.27E−01 | 5.15E−02 |
| 100001462 | 629 | NA | NA | NA | 7.87E−02 |
| 100001247 | 630 | 2.90E−01 | 4.99E−03 | 1.36E−01 | 1.94E−01 |
| X - 17438 | 631 | 1.13E−02 | 5.45E−02 | 7.98E−01 | NA |
| 100001417 | 632 | 1.53E−01 | 6.96E−02 | 6.76E−03 | 5.40E−01 |
| 100006642 | 633 | NA | NA | NA | 8.11E−02 |
| 100009220 | 634 | NA | NA | NA | 8.16E−02 |
| 100001250 | 635 | NA | NA | NA | 8.18E−02 |
| 100001777 | 636 | 3.05E−01 | 4.30E−01 | 5.41E−03 | 6.40E−02 |
| 100008990 | 637 | 2.84E−01 | 1.29E−02 | 2.33E−01 | 5.41E−02 |
| 100001868 | 638 | 9.86E−01 | 7.71E−03 | 1.46E−01 | 4.17E−02 |
| 100003892 | 639 | 2.64E−02 | 2.27E−02 | 1.42E−01 | 5.65E−01 |
| 500 | 640 | 4.03E−02 | 4.91E−02 | 2.97E−01 | NA |
| 100020492 | 641 | 1.28E−01 | 1.97E−01 | 2.55E−02 | NA |
| 100002049 | 642 | NA | NA | NA | 8.72E−02 |
| 100001170 | 643 | 2.07E−01 | 3.79E−01 | 5.36E−03 | 1.40E−01 |
| X - 22776 | 644 | 1.24E−02 | 3.81E−01 | 1.43E−01 | NA |
| 800 | 645 | 2.58E−01 | 3.56E−02 | 1.60E−02 | 4.07E−01 |
| 100003271 | 646 | 2.89E−01 | 2.14E−02 | 1.80E−01 | 6.04E−02 |
| 266 | 647 | 1.52E−03 | 9.02E−01 | 6.77E−02 | 7.44E−01 |
| 100009364 | 648 | NA | NA | NA | 9.23E−02 |
| 100002105 | 649 | NA | NA | NA | 9.28E−02 |
| 100020204 | 650 | 2.41E−02 | 3.72E−02 | 9.01E−01 | NA |
| 100015641 | 651 | NA | NA | NA | 9.35E−02 |
| 399 | 652 | 2.39E−02 | 4.59E−01 | 7.51E−02 | NA |
| 100006264 | 653 | 1.02E−01 | 7.50E−03 | 3.34E−01 | 3.05E−01 |
| 100001313 | 654 | 5.62E−01 | 9.90E−02 | 2.69E−03 | 5.21E−01 |
| 100010959 | 655 | NA | NA | NA | 9.43E−02 |
| 100001651 | 656 | 3.76E−01 | 3.39E−01 | 2.90E−02 | 2.16E−02 |
| X - 14314 | 657 | 1.56E−01 | 4.54E−02 | 1.20E−01 | NA |
| 100005391 | 658 | 1.71E−03 | 7.83E−01 | 9.07E−01 | 6.95E−02 |
| 330 | 659 | 7.41E−01 | 1.32E−01 | 1.81E−02 | 4.94E−02 |
| 100001048 | 660 | 6.40E−02 | 7.12E−02 | 1.35E−01 | 1.43E−01 |
| 100002029 | 661 | 6.79E−02 | 6.41E−03 | 5.07E−01 | 4.02E−01 |
| 100006126 | 662 | 7.30E−02 | 3.12E−02 | 1.78E−01 | 2.26E−01 |
| 100006191 | 663 | 3.32E−01 | 1.52E−02 | 2.14E−02 | 8.81E−01 |
| 100020497 | 664 | 7.08E−02 | 1.80E−01 | 7.72E−02 | NA |
| 100006171 | 665 | 8.54E−01 | 1.90E−02 | 6.37E−03 | 9.59E−01 |
| 100002488 | 666 | 2.82E−02 | 4.23E−02 | 8.69E−02 | 9.61E−01 |
| 100009338 | 667 | NA | NA | NA | 1.00E−01 |
| 100000447 | 668 | 2.14E−02 | 2.08E−01 | 4.95E−01 | 4.63E−02 |
| 100008989 | 669 | 8.74E−02 | 2.55E−01 | 9.30E−03 | 5.29E−01 |
| 100003432 | 670 | 5.33E−01 | 2.34E−02 | 4.46E−01 | 2.13E−02 |
| 100002719 | 671 | 3.12E−01 | 1.03E−02 | 4.43E−02 | 8.41E−01 |
| 55 | 672 | 4.55E−02 | 1.76E−01 | 2.19E−01 | 6.85E−02 |
| 100002249 | 673 | NA | NA | NA | 1.05E−01 |
| 100001786 | 674 | NA | NA | NA | 1.05E−01 |
| 100006726 | 675 | NA | NA | NA | 1.06E−01 |
| 1869 | 676 | 7.16E−01 | 3.40E−03 | 4.50E−01 | 1.18E−01 |
| 100009161 | 677 | NA | NA | NA | 1.08E−01 |
| 100001793 | 678 | 1.26E−01 | 3.12E−01 | 4.21E−03 | 8.13E−01 |
| X - 21258 | 679 | 4.33E−01 | 1.43E−01 | 2.06E−02 | NA |
| X - 11438 | 680 | 4.36E−01 | 1.43E−02 | 2.10E−01 | NA |
| 100001150 | 681 | 1.51E−02 | 6.19E−01 | 1.22E−01 | 1.27E−01 |
| 1141 | 682 | 5.35E−01 | 3.08E−01 | 2.67E−01 | 3.33E−03 |
| 1539 | 683 | 7.54E−03 | 7.78E−02 | 7.66E−01 | 3.32E−01 |
| 100001294 | 684 | 3.40E−01 | 7.36E−03 | 9.60E−01 | 6.47E−02 |
| 100009125 | 685 | NA | NA | NA | 1.13E−01 |
| 1224 | 686 | 2.53E−02 | 3.40E−01 | 5.52E−01 | 3.51E−02 |
| 100001212 | 687 | 1.05E−01 | 7.58E−02 | 1.14E−01 | 1.88E−01 |
| X - 22764 | 688 | 6.34E−01 | 1.49E−01 | 1.64E−02 | NA |
| 100005389 | 689 | 8.95E−02 | 8.57E−01 | 2.49E−01 | 9.94E−03 |
| X - 07765 | 690 | 1.26E−01 | 4.50E−02 | 2.88E−01 | NA |
| 1384 | 691 | 4.74E−02 | 1.56E−01 | 9.38E−01 | 2.78E−02 |
| 100008930 | 692 | 2.98E−01 | 4.95E−03 | 1.75E−01 | 7.50E−01 |
| 100003119 | 693 | NA | NA | NA | 1.19E−01 |
| X - 24242 | 694 | 2.17E−01 | 2.72E−01 | 2.88E−02 | NA |
| 100002185 | 695 | NA | NA | NA | 1.20E−01 |
| 439 | 696 | 3.87E−01 | 2.39E−01 | 6.75E−03 | 3.45E−01 |
| 100003000 | 697 | 5.94E−01 | 1.68E−01 | 3.05E−02 | 7.18E−02 |
| 100001806 | 698 | 2.21E−01 | 1.05E−01 | 4.75E−01 | 2.00E−02 |
| 100001859 | 699 | 9.23E−01 | 4.94E−01 | 3.28E−01 | 1.55E−03 |
| 100020478 | 700 | 8.32E−01 | 5.44E−02 | 4.16E−02 | NA |
| 100009337 | 701 | 1.66E−02 | 5.54E−02 | 8.67E−01 | 2.95E−01 |
| 100002726 | 702 | 5.07E−01 | 2.69E−02 | 1.45E−01 | NA |
| 100001310 | 703 | NA | NA | NA | 1.26E−01 |
| 100000436 | 704 | 7.24E−01 | 5.35E−01 | 1.48E−03 | 4.37E−01 |
| 100010947 | 705 | NA | NA | NA | 1.26E−01 |
| 100009021 | 706 | NA | NA | NA | 1.27E−01 |
| 100008920 | 707 | 7.72E−01 | 3.01E−01 | 4.74E−03 | 2.41E−01 |
| 536 | 708 | 1.69E−01 | 3.15E−02 | 7.64E−01 | 6.53E−02 |
| 1383 | 709 | 2.08E−01 | 3.42E−01 | 1.27E−02 | 3.02E−01 |
| 100001657 | 710 | 2.97E−01 | 1.74E−01 | 9.53E−01 | 5.52E−03 |
| 100000043 | 711 | 2.41E−01 | 5.88E−02 | 2.10E−02 | 9.35E−01 |
| 100009043 | 712 | NA | NA | NA | 1.31E−01 |
| 100002773 | 713 | NA | NA | NA | 1.32E−01 |
| 100001755 | 714 | 2.24E−01 | 5.57E−02 | 7.65E−02 | 3.23E−01 |
| 1161 | 715 | NA | NA | NA | 1.33E−01 |
| 100001296 | 716 | 1.44E−01 | 7.37E−01 | 3.31E−01 | 9.20E−03 |
| 100005999 | 717 | 3.72E−02 | 6.18E−01 | 1.05E−01 | NA |
| 1489 | 718 | 2.94E−01 | 6.89E−01 | 4.28E−01 | 3.77E−03 |
| 100005350 | 719 | 6.82E−01 | 5.43E−01 | 9.90E−03 | 9.04E−02 |
| 100002026 | 720 | 2.61E−01 | 7.14E−02 | 6.45E−01 | 2.82E−02 |
| X - 22771 | 721 | 2.84E−02 | 2.80E−01 | 3.18E−01 | NA |
| 798 | 722 | 4.40E−01 | 8.07E−02 | 1.67E−02 | 6.07E−01 |
| 100002024 | 723 | NA | NA | NA | 1.41E−01 |
| 181 | 724 | 2.18E−02 | 4.81E−01 | 7.94E−02 | 4.76E−01 |
| 100003594 | 725 | 5.23E−02 | 7.45E−02 | 1.26E−01 | 8.18E−01 |
| 1024 | 726 | 6.94E−01 | 5.27E−02 | 1.09E−01 | 1.03E−01 |
| 100003640 | 727 | NA | NA | NA | 1.42E−01 |
| X - 12230 | 728 | 3.54E−01 | 4.77E−02 | 1.71E−01 | NA |
| 100009000 | 729 | 4.17E−01 | 6.92E−01 | 5.83E−03 | 2.49E−01 |
| 100001405 | 730 | 3.67E−01 | 9.15E−01 | 6.16E−01 | 2.15E−03 |
| 100009025 | 731 | 1.46E−01 | 7.88E−01 | 1.29E−02 | 3.06E−01 |
| 100015723 | 732 | NA | NA | NA | 1.48E−01 |
| 100001655 | 733 | 6.02E−02 | 1.39E−02 | 8.93E−01 | 6.64E−01 |
| 100000987 | 734 | 2.78E−01 | 1.81E−01 | 8.85E−01 | 1.12E−02 |
| 796 | 735 | NA | NA | NA | 1.50E−01 |
| 100006438 | 736 | 3.99E−01 | 1.73E−01 | 2.43E−01 | 3.02E−02 |
| X - 12411 | 737 | 8.17E−02 | 1.58E−01 | 2.65E−01 | NA |
| 100010941 | 738 | NA | NA | NA | 1.51E−01 |
| X - 11858 | 739 | 5.90E−02 | 3.18E−01 | 1.85E−01 | NA |
| 100019975 | 740 | 8.08E−01 | 3.98E−02 | 1.09E−01 | NA |
| 100001723 | 741 | NA | NA | NA | 1.52E−01 |
| X - 21442 | 742 | 1.40E−01 | 1.84E−01 | 1.38E−01 | NA |
| 100001797 | 743 | 2.34E−01 | 1.46E−02 | 2.43E−01 | 6.57E−01 |
| X - 19141 | 744 | 4.49E−01 | 1.34E−01 | 5.97E−02 | NA |
| 355 | 745 | 6.65E−01 | 1.52E−01 | 1.54E−01 | 3.54E−02 |
| 100009141 | 746 | 6.61E−02 | 1.19E−01 | 9.79E−01 | 7.18E−02 |
| 100002911 | 747 | 2.47E−02 | 3.60E−01 | 1.84E−01 | 3.38E−01 |
| 100001472 | 748 | NA | NA | NA | 1.53E−01 |
| 100009131 | 749 | 8.61E−01 | 5.24E−01 | 3.95E−03 | 3.20E−01 |
| 100001334 | 750 | NA | NA | NA | 1.56E−01 |
| 100009126 | 751 | NA | NA | NA | 1.57E−01 |
| 564 | 752 | 3.63E−02 | 1.14E−01 | 2.39E−01 | 6.24E−01 |
| 1518 | 753 | 1.34E−02 | 4.06E−01 | 3.34E−01 | 3.40E−01 |
| 100004110 | 754 | 2.03E−01 | 1.50E−01 | 4.39E−01 | 4.64E−02 |
| 1256 | 755 | 3.04E−01 | 1.21E−01 | 2.49E−02 | 6.79E−01 |
| 100009272 | 756 | NA | NA | NA | 1.58E−01 |
| 100000808 | 757 | 1.68E−01 | 2.63E−01 | 5.00E−01 | 3.06E−02 |
| 100001181 | 758 | 8.53E−01 | 2.54E−01 | 3.24E−02 | 9.90E−02 |
| 100001564 | 759 | NA | NA | NA | 1.63E−01 |
| 2029 | 760 | 4.52E−01 | 1.81E−01 | 9.51E−03 | 9.16E−01 |
| 100001654 | 761 | 1.72E−01 | 3.49E−02 | 6.14E−01 | 1.96E−01 |
| 100006005 | 762 | NA | NA | NA | 1.65E−01 |
| 498 | 763 | 4.03E−01 | 2.11E−01 | 3.71E−01 | 2.66E−02 |
| 100001481 | 764 | 2.66E−01 | 8.62E−02 | 5.48E−01 | 6.67E−02 |
| 100015846 | 765 | NA | NA | NA | 1.72E−01 |
| 100001314 | 766 | 7.36E−01 | 3.20E−01 | 1.32E−02 | 2.81E−01 |
| 100001605 | 767 | 1.14E−01 | 2.02E−01 | 1.15E−01 | 3.38E−01 |
| X - 21341 | 768 | 1.53E−01 | 3.64E−01 | 9.32E−02 | NA |
| 100001635 | 769 | NA | NA | NA | 1.74E−01 |
| 100002129 | 770 | 2.28E−01 | 1.08E−01 | 4.31E−02 | 8.64E−01 |
| 2054 | 771 | 6.60E−01 | 2.82E−01 | 7.74E−02 | 6.60E−02 |
| X - 12847 | 772 | 8.22E−02 | 9.53E−02 | 7.19E−01 | NA |
| 1114 | 773 | 1.79E−01 | 3.56E−01 | 6.46E−01 | 2.48E−02 |
| 100001502 | 774 | 3.45E−01 | 9.87E−01 | 3.05E−02 | 9.97E−02 |
| 100009123 | 775 | NA | NA | NA | 1.79E−01 |
| 100016069 | 776 | 1.50E−02 | 6.45E−01 | 6.12E−01 | NA |
| 313 | 777 | 1.46E−01 | 1.73E−02 | 4.36E−01 | 9.86E−01 |
| X - 17010 | 778 | 6.56E−02 | 3.56E−01 | 2.62E−01 | NA |
| 1629 | 779 | 4.86E−01 | 6.25E−01 | 5.06E−03 | 7.30E−01 |
| 100002568 | 780 | NA | NA | NA | 1.86E−01 |
| 100002008 | 781 | NA | NA | NA | 1.86E−01 |
| X - 11470 | 782 | 5.94E−01 | 5.74E−01 | 1.90E−02 | NA |
| 100002732 | 783 | 1.37E−01 | 2.60E−01 | 2.56E−01 | 1.35E−01 |
| 100004251 | 784 | 5.82E−01 | 2.66E−02 | 4.36E−01 | NA |
| 100001323 | 785 | 4.69E−01 | 2.56E−01 | 1.55E−02 | 7.19E−01 |
| 444 | 786 | 6.66E−01 | 1.68E−02 | 3.75E−01 | 3.20E−01 |
| 100009076 | 787 | 2.64E−02 | 1.34E−01 | 4.55E−01 | 8.34E−01 |
| 100002196 | 788 | 6.11E−01 | 6.98E−01 | 3.05E−01 | 1.04E−02 |
| X - 21319 | 789 | 2.61E−01 | 6.58E−01 | 4.12E−02 | NA |
| 100001550 | 790 | NA | NA | NA | 1.93E−01 |
| 100015751 | 791 | NA | NA | NA | 1.93E−01 |
| 100010962 | 792 | NA | NA | NA | 1.94E−01 |
| 100000787 | 793 | 5.94E−01 | 8.82E−01 | 6.75E−01 | 3.99E−03 |
| 445 | 794 | 2.13E−01 | 9.85E−01 | 7.17E−03 | 9.49E−01 |
| 100003240 | 795 | NA | NA | NA | 1.96E−01 |
| 100001866 | 796 | NA | NA | NA | 1.96E−01 |
| X - 14568 | 797 | 4.87E−01 | 4.12E−02 | 3.78E−01 | NA |
| 100001619 | 798 | NA | NA | NA | 1.97E−01 |
| 100001876 | 799 | 7.95E−01 | 4.80E−02 | 7.50E−01 | 5.42E−02 |
| 100009078 | 800 | NA | NA | NA | 1.99E−01 |
| 935 | 801 | 9.07E−01 | 4.24E−03 | 4.39E−01 | 9.42E−01 |
| 100002500 | 802 | NA | NA | NA | 2.01E−01 |
| 100009264 | 803 | NA | NA | NA | 2.01E−01 |
| 100001211 | 804 | 4.48E−02 | 5.97E−02 | 7.87E−01 | 7.74E−01 |
| 100001956 | 805 | 9.19E−01 | 4.57E−01 | 7.73E−02 | 5.09E−02 |
| 100001359 | 806 | NA | NA | NA | 2.04E−01 |
| 100002458 | 807 | 3.24E−01 | 3.36E−01 | 2.32E−01 | 6.97E−02 |
| 100002951 | 808 | NA | NA | NA | 2.05E−01 |
| 535 | 809 | 1.47E−01 | 1.22E−01 | 1.58E−01 | 6.21E−01 |
| 100010955 | 810 | NA | NA | NA | 2.05E−01 |
| 100008916 | 811 | 2.34E−01 | 6.32E−01 | 9.49E−02 | 1.26E−01 |
| 100001253 | 812 | 4.81E−01 | 7.16E−02 | 9.82E−01 | 5.27E−02 |
| 100006294 | 813 | 4.46E−02 | 7.39E−01 | 1.13E−01 | 4.84E−01 |
| 100008994 | 814 | 5.58E−02 | 9.62E−01 | 1.79E−01 | 1.94E−01 |
| 100005717 | 815 | NA | NA | NA | 2.08E−01 |
| 100000295 | 816 | 4.87E−01 | 1.94E−01 | 9.44E−02 | 2.12E−01 |
| 100002390 | 817 | NA | NA | NA | 2.11E−01 |
| 100003006 | 818 | NA | NA | NA | 2.12E−01 |
| 100002417 | 819 | 3.17E−01 | 3.53E−01 | 5.57E−02 | 3.27E−01 |
| 100006373 | 820 | NA | NA | NA | 2.13E−01 |
| 100009075 | 821 | 1.74E−02 | 5.53E−01 | 3.14E−01 | 6.88E−01 |
| 100000039 | 822 | 1.19E−01 | 6.97E−01 | 3.59E−02 | 7.03E−01 |
| 100009069 | 823 | 6.31E−01 | 4.62E−01 | 6.96E−02 | 1.03E−01 |
| X - 12729 | 824 | 9.96E−01 | 1.14E−01 | 8.63E−02 | NA |
| X - 11795 | 825 | 6.21E−01 | 1.50E−01 | 1.06E−01 | NA |
| 100000900 | 826 | NA | NA | NA | 2.15E−01 |
| X - 18888 | 827 | 4.24E−01 | 6.32E−02 | 3.76E−01 | NA |
| 136 | 828 | 7.66E−01 | 1.59E−02 | 2.96E−01 | 6.14E−01 |
| X - 23587 | 829 | 5.73E−01 | 4.61E−02 | 3.89E−01 | NA |
| 100001198 | 830 | 7.64E−01 | 1.59E−02 | 4.03E−01 | 4.67E−01 |
| 100020492 | 831 | 6.59E−02 | 3.89E−01 | 4.13E−01 | NA |
| X - 11852 | 832 | 6.86E−02 | 8.37E−01 | 1.85E−01 | NA |
| 100006298 | 833 | NA | NA | NA | 2.21E−01 |
| 891 | 834 | 6.26E−01 | 4.42E−01 | 1.87E−01 | 4.63E−02 |
| 111 | 835 | 1.51E−01 | 7.70E−02 | 6.62E−01 | 3.17E−01 |
| 418 | 836 | NA | NA | NA | 2.22E−01 |
| 100001269 | 837 | NA | NA | NA | 2.23E−01 |
| 100001229 | 838 | 1.43E−01 | 9.20E−01 | 2.71E−02 | 6.91E−01 |
| 1124 | 839 | 3.03E−01 | 8.15E−01 | 7.61E−01 | 1.39E−02 |
| 100020208 | 840 | 6.95E−01 | 6.18E−01 | 2.71E−02 | NA |
| 100009215 | 841 | NA | NA | NA | 2.27E−01 |
| 100000672 | 842 | 7.73E−01 | 5.42E−01 | 4.33E−01 | 1.51E−02 |
| X - 12730 | 843 | 1.88E−01 | 3.87E−01 | 1.66E−01 | NA |
| X - 18913 | 844 | 1.68E−01 | 1.93E−01 | 3.77E−01 | NA |
| 100003470 | 845 | 1.91E−01 | 9.79E−01 | 4.69E−01 | 3.25E−02 |
| 100001293 | 846 | 6.26E−01 | 4.94E−01 | 7.40E−01 | 1.24E−02 |
| 100000898 | 847 | 7.98E−01 | 2.12E−02 | 7.34E−01 | NA |
| 100000008 | 848 | 3.96E−01 | 6.50E−01 | 1.02E−01 | 1.10E−01 |
| 432 | 849 | 7.89E−01 | 8.96E−01 | 1.99E−02 | 2.05E−01 |
| 100009005 | 850 | 4.77E−01 | 7.21E−01 | 2.46E−01 | 3.56E−02 |
| X - 11849 | 851 | 2.41E−01 | 1.52E−01 | 3.54E−01 | NA |
| 100002806 | 852 | 3.45E−01 | 3.44E−01 | 1.10E−01 | NA |
| 231 | 853 | 9.46E−02 | 3.13E−01 | 5.06E−01 | 2.06E−01 |
| 100020014 | 854 | 5.48E−02 | 5.82E−01 | 4.14E−01 | NA |
| 100001662 | 855 | 2.17E−02 | 8.40E−01 | 5.25E−01 | 3.35E−01 |
| 100001092 | 856 | 1.39E−02 | 4.24E−01 | 7.64E−01 | 7.15E−01 |
| 519 | 857 | 8.28E−01 | 2.39E−02 | 7.64E−01 | 2.15E−01 |
| 342 | 858 | 3.47E−01 | 5.77E−01 | 2.58E−02 | 6.30E−01 |
| 100002153 | 859 | 7.20E−01 | 6.80E−01 | 1.93E−02 | 3.50E−01 |
| 100002734 | 860 | 3.61E−01 | 4.14E−01 | 9.45E−02 | NA |
| 1026 | 861 | NA | NA | NA | 2.42E−01 |
| 100009082 | 862 | 2.61E−01 | 3.50E−01 | 2.39E−01 | 1.60E−01 |
| 100003769 | 863 | 5.01E−01 | 2.82E−01 | 1.02E−01 | NA |
| 100010966 | 864 | NA | NA | NA | 2.45E−01 |
| X - 23314 | 865 | 5.13E−01 | 1.38E−01 | 2.08E−01 | NA |
| 100009167 | 866 | 4.16E−02 | 9.95E−01 | 8.88E−02 | 9.95E−01 |
| X - 17685 | 867 | 4.48E−01 | 9.92E−02 | 3.40E−01 | NA |
| 100002813 | 868 | NA | NA | NA | 2.48E−01 |
| X - 12816 | 869 | 8.54E−01 | 7.55E−02 | 2.37E−01 | NA |
| X - 10458 | 870 | 1.12E−01 | 4.95E−01 | 2.77E−01 | NA |
| 1021 | 871 | 2.74E−01 | 9.53E−01 | 4.23E−01 | 3.52E−02 |
| X - 17185 | 872 | 5.70E−01 | 1.27E−01 | 2.22E−01 | NA |
| 100004227 | 873 | 3.98E−01 | 7.22E−01 | 1.54E−01 | 9.41E−02 |
| 100000773 | 874 | 2.87E−01 | 7.53E−01 | 2.00E−02 | 9.80E−01 |
| 100000792 | 875 | 1.12E−01 | 2.12E−01 | 6.07E−01 | 3.00E−01 |
| 100001743 | 876 | 1.36E−01 | 9.66E−02 | 4.54E−01 | 7.56E−01 |
| 100006374 | 877 | 6.73E−01 | 9.57E−01 | 9.62E−02 | 7.64E−02 |
| 100015640 | 878 | NA | NA | NA | 2.63E−01 |
| 512 | 879 | 4.36E−01 | 2.57E−01 | 9.82E−02 | 4.33E−01 |
| 926 | 880 | 3.43E−02 | 9.58E−01 | 7.41E−01 | 1.97E−01 |
| 229 | 881 | 1.02E−01 | 2.83E−01 | 2.30E−01 | 7.20E−01 |
| 100001624 | 882 | 1.66E−01 | 3.04E−01 | 2.87E−01 | 3.31E−01 |
| 100004208 | 883 | 6.67E−01 | 2.66E−01 | 3.84E−01 | 7.11E−02 |
| 100001232 | 884 | 4.62E−01 | 3.53E−01 | 1.80E−01 | 1.65E−01 |
| 100003651 | 885 | NA | NA | NA | 2.65E−01 |
| 100001989 | 886 | 1.86E−01 | 4.07E−01 | 3.81E−01 | 1.72E−01 |
| X - 12543 | 887 | 8.53E−02 | 6.93E−01 | 3.18E−01 | NA |
| 100001571 | 888 | 2.19E−01 | 5.17E−01 | 5.99E−01 | 7.45E−02 |
| 925 | 889 | 5.63E−01 | 5.00E−01 | 4.13E−02 | 4.39E−01 |
| 100003178 | 890 | 9.97E−02 | 5.69E−01 | 3.40E−01 | NA |
| 100000781 | 891 | 8.48E−01 | 9.67E−01 | 5.28E−01 | 1.27E−02 |
| X - 12680 | 892 | 1.46E−01 | 2.22E−01 | 6.31E−01 | NA |
| 100009014 | 893 | 2.70E−01 | 6.85E−01 | 8.39E−02 | 3.81E−01 |
| 100001073 | 894 | 5.68E−01 | 7.83E−02 | 1.42E−01 | 9.56E−01 |
| 100001197 | 895 | 4.83E−01 | 3.16E−02 | 6.06E−01 | 6.69E−01 |
| 100003442 | 896 | NA | NA | NA | 2.81E−01 |
| 100001778 | 897 | 5.70E−01 | 9.33E−01 | 1.32E−02 | 8.85E−01 |
| 1487 | 898 | 1.32E−01 | 7.03E−01 | 9.02E−02 | 7.46E−01 |
| 100001034 | 899 | 6.94E−01 | 6.32E−01 | 5.60E−02 | 2.62E−01 |
| 1382 | 900 | 7.29E−01 | 9.31E−02 | 3.38E−01 | NA |
| 1648 | 901 | 1.94E−01 | 5.75E−01 | 9.29E−02 | 6.47E−01 |
| 100015730 | 902 | NA | NA | NA | 2.87E−01 |
| 100015625 | 903 | NA | NA | NA | 2.88E−01 |
| X - 16938 | 904 | 4.22E−01 | 2.36E−01 | 2.41E−01 | NA |
| 192 | 905 | 8.79E−01 | 1.32E−01 | 7.65E−02 | 7.82E−01 |
| 100001086 | 906 | 6.34E−01 | 6.96E−01 | 2.30E−01 | 6.93E−02 |
| 1230 | 907 | 1.17E−01 | 3.51E−01 | 3.11E−01 | 5.56E−01 |
| 100001561 | 908 | 8.44E−02 | 1.74E−01 | 7.52E−01 | 6.59E−01 |
| 100015594 | 909 | NA | NA | NA | 2.93E−01 |
| 100000551 | 910 | 9.96E−01 | 9.73E−01 | 4.30E−01 | 1.81E−02 |
| X - 10358 | 911 | 6.10E−01 | 7.01E−02 | 6.21E−01 | NA |
| 100006619 | 912 | NA | NA | NA | 2.99E−01 |
| 100015836 | 913 | NA | NA | NA | 3.01E−01 |
| 100001999 | 914 | 4.09E−01 | 1.26E−01 | 7.16E−01 | 2.23E−01 |
| 100004015 | 915 | 4.47E−01 | 5.80E−01 | 1.07E−01 | NA |
| 100015789 | 916 | NA | NA | NA | 3.05E−01 |
| 100001267 | 917 | 7.15E−01 | 6.88E−01 | 8.08E−01 | 2.18E−02 |
| 100002406 | 918 | NA | NA | NA | 3.06E−01 |
| 818 | 919 | 6.61E−02 | 7.20E−01 | 6.08E−01 | 3.07E−01 |
| 100002135 | 920 | 2.14E−01 | 4.12E−01 | 3.37E−01 | NA |
| 207 | 921 | NA | NA | NA | 3.10E−01 |
| 491 | 922 | NA | NA | NA | 3.10E−01 |
| 100009225 | 923 | NA | NA | NA | 3.10E−01 |
| 100003250 | 924 | 3.55E−01 | 5.40E−01 | 1.58E−01 | NA |
| 100006435 | 925 | NA | NA | NA | 3.13E−01 |
| X - 12221 | 926 | 9.21E−01 | 2.30E−01 | 1.47E−01 | NA |
| 100001337 | 927 | 1.66E−01 | 8.34E−02 | 7.22E−01 | 9.99E−01 |
| 100004295 | 928 | 2.30E−01 | 3.81E−01 | 5.11E−01 | 2.31E−01 |
| X - 21441 | 929 | 6.80E−01 | 7.59E−02 | 6.36E−01 | NA |
| 100002014 | 930 | 2.46E−01 | 9.76E−01 | 2.66E−01 | 1.65E−01 |
| 501 | 931 | 4.35E−01 | 9.09E−02 | 8.07E−01 | 3.34E−01 |
| 100006203 | 932 | 6.93E−01 | 1.08E−01 | 6.02E−01 | 2.39E−01 |
| 100008996 | 933 | NA | NA | NA | 3.23E−01 |
| 100001757 | 934 | 3.04E−01 | 7.71E−01 | 7.52E−02 | 6.24E−01 |
| 100004112 | 935 | 6.26E−01 | 7.59E−01 | 4.19E−01 | 5.56E−02 |
| 100015835 | 936 | NA | NA | NA | 3.26E−01 |
| 100003101 | 937 | NA | NA | NA | 3.26E−01 |
| 100010939 | 938 | NA | NA | NA | 3.27E−01 |
| X - 12738 | 939 | 4.18E−01 | 2.87E−01 | 3.07E−01 | NA |
| 100001551 | 940 | 4.41E−02 | 5.77E−01 | 9.52E−01 | 5.07E−01 |
| 50 | 941 | 4.05E−01 | 6.50E−01 | 3.63E−01 | 1.33E−01 |
| X - 23369 | 942 | 1.81E−01 | 8.06E−01 | 2.60E−01 | NA |
| 100002871 | 943 | 2.50E−01 | 5.35E−01 | 2.15E−01 | 4.45E−01 |
| 100001270 | 944 | 8.51E−01 | 8.31E−01 | 7.00E−01 | 2.57E−02 |
| 100015586 | 945 | NA | NA | NA | 3.37E−01 |
| 100009140 | 946 | NA | NA | NA | 3.37E−01 |
| X - 23997 | 947 | 3.17E−01 | 2.16E−01 | 5.65E−01 | NA |
| 100004169 | 948 | 9.75E−02 | 6.91E−01 | 5.78E−01 | 3.50E−01 |
| 461 | 949 | 3.12E−01 | 3.85E−01 | 4.76E−01 | 2.39E−01 |
| 100010928 | 950 | NA | NA | NA | 3.43E−01 |
| 100001950 | 951 | 7.14E−02 | 7.55E−01 | 7.76E−01 | 3.37E−01 |
| 100001287 | 952 | 7.82E−01 | 9.31E−01 | 2.17E−02 | 9.12E−01 |
| 1829 | 953 | 5.19E−01 | 1.22E−01 | 6.65E−01 | NA |
| 100010869 | 954 | NA | NA | NA | 3.49E−01 |
| X - 23649 | 955 | 2.57E−01 | 4.31E−01 | 3.85E−01 | NA |
| 363 | 956 | 7.11E−01 | 7.98E−02 | 9.57E−01 | 2.78E−01 |
| X - 12013 | 957 | 6.10E−01 | 3.55E−01 | 2.01E−01 | NA |
| 100015792 | 958 | NA | NA | NA | 3.52E−01 |
| X - 17690 | 959 | 1.50E−01 | 5.55E−01 | 5.28E−01 | NA |
| 100008919 | 960 | 3.98E−01 | 9.25E−01 | 4.31E−02 | 9.89E−01 |
| 100006051 | 961 | 7.35E−01 | 3.40E−01 | 3.80E−01 | 1.68E−01 |
| 100001423 | 962 | 9.24E−01 | 1.95E−01 | 9.53E−02 | 9.34E−01 |
| X - 15469 | 963 | 6.07E−01 | 5.02E−01 | 1.49E−01 | NA |
| 100001207 | 964 | 1.63E−01 | 2.91E−01 | 6.17E−01 | 5.52E−01 |
| 100000708 | 965 | 2.11E−01 | 4.81E−01 | 4.17E−01 | 3.84E−01 |
| 100001081 | 966 | 2.33E−01 | 4.40E−01 | 4.47E−01 | NA |
| 100001108 | 967 | 9.61E−01 | 1.69E−01 | 8.43E−01 | 1.21E−01 |
| 100001026 | 968 | 3.01E−01 | 3.22E−01 | 9.21E−01 | 1.87E−01 |
| X - 21735 | 969 | 2.24E−01 | 4.73E−01 | 4.43E−01 | NA |
| 1668 | 970 | 7.27E−01 | 9.92E−01 | 3.54E−02 | 6.76E−01 |
| 100009217 | 971 | NA | NA | NA | 3.62E−01 |
| 100002102 | 972 | 2.54E−01 | 1.50E−01 | 7.92E−01 | 5.81E−01 |
| 278 | 973 | 6.06E−01 | 1.83E−01 | 1.76E−01 | 9.21E−01 |
| 100008955 | 974 | 4.89E−01 | 9.70E−01 | 8.14E−01 | 4.76E−02 |
| 100010923 | 975 | NA | NA | NA | 3.68E−01 |
| 100004111 | 976 | 2.62E−01 | 6.99E−01 | 4.23E−01 | 2.40E−01 |
| X - 11299 | 977 | 6.05E−01 | 3.84E−01 | 2.18E−01 | NA |
| X - 12462 | 978 | 1.77E−01 | 6.28E−01 | 4.60E−01 | NA |
| 100000442 | 979 | 7.93E−01 | 2.86E−01 | 4.35E−01 | 1.92E−01 |
| 100000656 | 980 | 3.82E−01 | 8.98E−01 | 2.17E−01 | 2.56E−01 |
| 100000964 | 981 | 4.07E−01 | 5.49E−01 | 2.32E−01 | NA |
| 100009406 | 982 | NA | NA | NA | 3.74E−01 |
| 100010924 | 983 | NA | NA | NA | 3.74E−01 |
| 100001121 | 984 | 1.37E−01 | 8.03E−01 | 4.79E−01 | 3.76E−01 |
| 828 | 985 | NA | NA | NA | 3.76E−01 |
| 100001767 | 986 | 4.68E−01 | 1.64E−01 | 4.76E−01 | 5.63E−01 |
| 100003473 | 987 | 6.75E−01 | 3.45E−01 | 1.02E−01 | 8.72E−01 |
| 100003239 | 988 | NA | NA | NA | 3.81E−01 |
| 100015735 | 989 | NA | NA | NA | 3.84E−01 |
| X - 21444 | 990 | 9.01E−01 | 1.05E−01 | 6.04E−01 | NA |
| 251 | 991 | NA | NA | NA | 3.85E−01 |
| 100004442 | 992 | 9.15E−01 | 4.12E−02 | 9.54E−01 | 6.13E−01 |
| 100001540 | 993 | 1.70E−01 | 6.74E−01 | 2.52E−01 | 7.68E−01 |
| 100002027 | 994 | 2.84E−01 | 9.54E−02 | 9.25E−01 | 8.85E−01 |
| 100015840 | 995 | NA | NA | NA | 3.89E−01 |
| 100006361 | 996 | 7.54E−02 | 3.75E−01 | 9.86E−01 | 8.58E−01 |
| X - 23974 | 997 | 9.30E−01 | 3.50E−01 | 1.91E−01 | NA |
| X - 12007 | 998 | 6.13E−01 | 5.74E−01 | 1.78E−01 | NA |
| 100001195 | 999 | NA | NA | NA | 3.98E−01 |
| 100000263 | 1000 | 9.41E−01 | 1.13E−01 | 5.63E−01 | 4.36E−01 |
| 100002035 | 1001 | NA | NA | NA | 4.03E−01 |
| 100009154 | 1002 | NA | NA | NA | 4.04E−01 |
| 100000409 | 1003 | 5.75E−01 | 1.84E−01 | 5.41E−01 | 4.85E−01 |
| 2051 | 1004 | 2.52E−01 | 3.16E−01 | 5.50E−01 | 6.36E−01 |
| 100001257 | 1005 | 3.38E−01 | 3.27E−01 | 6.23E−01 | NA |
| 100006369 | 1006 | NA | NA | NA | 4.11E−01 |
| X - 11483 | 1007 | 2.34E−01 | 4.00E−01 | 7.44E−01 | NA |
| 100003668 | 1008 | NA | NA | NA | 4.12E−01 |
| 100003008 | 1009 | 3.63E−01 | 9.03E−01 | 4.08E−01 | 2.18E−01 |
| 100000647 | 1010 | 2.77E−01 | 5.57E−01 | 4.60E−01 | NA |
| 100003915 | 1011 | NA | NA | NA | 4.15E−01 |
| 100001277 | 1012 | 8.77E−01 | 6.23E−01 | 7.56E−01 | 7.27E−02 |
| 100010948 | 1013 | NA | NA | NA | 4.18E−01 |
| X - 23046 | 1014 | 1.39E−01 | 6.69E−01 | 7.83E−01 | NA |
| 100002537 | 1015 | NA | NA | NA | 4.20E−01 |
| 100009232 | 1016 | 5.69E−02 | 9.95E−01 | 8.10E−01 | 6.91E−01 |
| 100001402 | 1017 | 5.93E−01 | 1.82E−01 | 5.39E−01 | 5.61E−01 |
| 100015882 | 1018 | NA | NA | NA | 4.27E−01 |
| 100006375 | 1019 | 8.75E−01 | 5.75E−02 | 6.93E−01 | 9.59E−01 |
| 100001226 | 1020 | NA | NA | NA | 4.29E−01 |
| 100001554 | 1021 | 1.54E−01 | 7.41E−01 | 3.09E−01 | 9.64E−01 |
| X - 12830 | 1022 | 2.01E−01 | 7.05E−01 | 5.60E−01 | NA |
| 100003151 | 1023 | 8.46E−01 | 4.40E−01 | 5.10E−01 | 1.81E−01 |
| 100001332 | 1024 | 9.75E−01 | 4.34E−01 | 5.72E−01 | 1.42E−01 |
| 100002868 | 1025 | NA | NA | NA | 4.31E−01 |
| 100001674 | 1026 | 7.43E−01 | 4.51E−01 | 3.11E−01 | 3.34E−01 |
| X - 17146 | 1027 | 2.15E−01 | 8.07E−01 | 4.67E−01 | NA |
| 100001429 | 1028 | 4.98E−01 | 1.78E−01 | 9.26E−01 | NA |
| X - 12407 | 1029 | 5.31E−01 | 7.99E−01 | 1.96E−01 | NA |
| 2028 | 1030 | NA | NA | NA | 4.37E−01 |
| 100009079 | 1031 | NA | NA | NA | 4.38E−01 |
| 100001411 | 1032 | NA | NA | NA | 4.38E−01 |
| X - 17327 | 1033 | 3.44E−01 | 3.21E−01 | 7.64E−01 | NA |
| 100006173 | 1034 | 2.04E−01 | 4.68E−01 | 4.30E−01 | 9.05E−01 |
| 100009378 | 1035 | NA | NA | NA | 4.45E−01 |
| 100006627 | 1036 | NA | NA | NA | 4.49E−01 |
| X - 12472 | 1037 | 4.52E−01 | 5.03E−01 | 4.08E−01 | NA |
| 1231 | 1038 | 9.75E−01 | 3.95E−01 | 4.54E−01 | 2.47E−01 |
| 100006641 | 1039 | NA | NA | NA | 4.57E−01 |
| 100015788 | 1040 | NA | NA | NA | 4.59E−01 |
| 100002206 | 1041 | NA | NA | NA | 4.60E−01 |
| 100015737 | 1042 | NA | NA | NA | 4.62E−01 |
| 100006367 | 1043 | 4.70E−01 | 8.41E−01 | 1.27E−01 | 9.12E−01 |
| 100002914 | 1044 | NA | NA | NA | 4.64E−01 |
| 100005818 | 1045 | NA | NA | NA | 4.65E−01 |
| 1111 | 1046 | 3.83E−01 | 9.89E−01 | 9.07E−01 | 1.37E−01 |
| 132 | 1047 | NA | NA | NA | 4.70E−01 |
| 100001563 | 1048 | NA | NA | NA | 4.71E−01 |
| 233 | 1049 | 5.57E−01 | 2.79E−01 | 6.73E−01 | NA |
| 100003397 | 1050 | 6.26E−01 | 5.75E−01 | 1.58E−01 | 8.66E−01 |
| 100008951 | 1051 | 5.19E−01 | 6.53E−01 | 7.08E−01 | 2.07E−01 |
| 100015591 | 1052 | NA | NA | NA | 4.72E−01 |
| 100004322 | 1053 | 1.72E−01 | 9.94E−01 | 3.01E−01 | 9.97E−01 |
| 100015793 | 1054 | NA | NA | NA | 4.76E−01 |
| 100006098 | 1055 | 7.46E−01 | 5.57E−01 | 4.34E−01 | 2.87E−01 |
| X - 15666 | 1056 | 3.45E−01 | 4.84E−01 | 6.52E−01 | NA |
| 100006282 | 1057 | 7.59E−01 | 4.00E−01 | 2.26E−01 | 7.78E−01 |
| 100001315 | 1058 | 2.65E−01 | 6.12E−01 | 3.47E−01 | 9.99E−01 |
| 100002259 | 1059 | 3.53E−01 | 3.87E−01 | 6.07E−01 | 6.79E−01 |
| 100009124 | 1060 | NA | NA | NA | 4.87E−01 |
| 100015833 | 1061 | NA | NA | NA | 4.88E−01 |
| 100003444 | 1062 | 1.48E−01 | 8.08E−01 | 9.83E−01 | NA |
| 100001386 | 1063 | NA | NA | NA | 4.92E−01 |
| X - 22147 | 1064 | 5.51E−01 | 2.60E−01 | 8.55E−01 | NA |
| 100009144 | 1065 | NA | NA | NA | 4.97E−01 |
| 100005673 | 1066 | 3.29E−01 | 9.86E−01 | 9.75E−01 | 1.95E−01 |
| 100001161 | 1067 | NA | NA | NA | 4.99E−01 |
| 1342 | 1068 | 2.86E−01 | 9.13E−01 | 3.73E−01 | 6.74E−01 |
| 100002849 | 1069 | 8.14E−01 | 4.53E−01 | 2.16E−01 | 8.28E−01 |
| 415 | 1070 | 3.65E−01 | 5.83E−01 | 4.30E−01 | 7.21E−01 |
| 100009067 | 1071 | NA | NA | NA | 5.13E−01 |
| X - 17189 | 1072 | 9.00E−01 | 2.75E−01 | 5.45E−01 | NA |
| 100001788 | 1073 | 6.15E−01 | 8.81E−01 | 4.25E−01 | 3.02E−01 |
| 100004326 | 1074 | 5.61E−01 | 5.84E−01 | 4.28E−01 | 4.98E−01 |
| 100001988 | 1075 | 6.76E−01 | 8.70E−01 | 3.61E−01 | 3.31E−01 |
| 100001789 | 1076 | NA | NA | NA | 5.18E−01 |
| 100003639 | 1077 | NA | NA | NA | 5.21E−01 |
| 100000997 | 1078 | 7.96E−01 | 9.92E−01 | 2.61E−01 | 3.61E−01 |
| 100010850 | 1079 | NA | NA | NA | 5.22E−01 |
| X - 24425 | 1080 | 3.99E−01 | 4.46E−01 | 8.04E−01 | NA |
| X - 17351 | 1081 | 2.46E−01 | 7.20E−01 | 8.10E−01 | NA |
| 100001617 | 1082 | 2.45E−01 | 6.11E−01 | 9.63E−01 | NA |
| 100004327 | 1083 | NA | NA | NA | 5.24E−01 |
| X - 12101 | 1084 | 9.35E−01 | 6.52E−01 | 2.37E−01 | NA |
| 100000299 | 1085 | NA | NA | NA | 5.27E−01 |
| 180 | 1086 | 7.57E−01 | 7.94E−01 | 1.30E−01 | 9.93E−01 |
| X - 12329 | 1087 | 6.82E−01 | 6.47E−01 | 3.39E−01 | NA |
| 100020487 | 1088 | 8.98E−01 | 3.65E−01 | 4.60E−01 | NA |
| X - 24071 | 1089 | 3.82E−01 | 4.04E−01 | 9.87E−01 | NA |
| 100001112 | 1090 | 6.31E−01 | 7.94E−01 | 3.43E−01 | 4.88E−01 |
| 361 | 1091 | 2.37E−01 | 8.61E−01 | 5.47E−01 | 7.55E−01 |
| 100001396 | 1092 | 9.53E−01 | 6.70E−01 | 8.09E−01 | 1.63E−01 |
| 100001145 | 1093 | 1.75E−01 | 9.03E−01 | 5.90E−01 | 9.04E−01 |
| 100001268 | 1094 | 8.73E−01 | 7.94E−01 | 2.26E−01 | NA |
| 100015618 | 1095 | NA | NA | NA | 5.41E−01 |
| 100010929 | 1096 | NA | NA | NA | 5.44E−01 |
| 100003163 | 1097 | NA | NA | NA | 5.49E−01 |
| 100002912 | 1098 | NA | NA | NA | 5.49E−01 |
| 100010942 | 1099 | NA | NA | NA | 5.54E−01 |
| X - 23644 | 1100 | 6.43E−01 | 7.53E−01 | 3.53E−01 | NA |
| 100009407 | 1101 | NA | NA | NA | 5.55E−01 |
| 100010927 | 1102 | NA | NA | NA | 5.58E−01 |
| 35 | 1103 | NA | NA | NA | 5.59E−01 |
| 100002968 | 1104 | 4.91E−01 | 9.78E−01 | 3.65E−01 | NA |
| 100020274 | 1105 | 8.80E−01 | 6.49E−01 | 3.10E−01 | NA |
| 71 | 1106 | 3.65E−01 | 9.47E−01 | 2.93E−01 | 9.91E−01 |
| 100009018 | 1107 | 3.38E−01 | 7.62E−01 | 6.92E−01 | NA |
| 100000784 | 1108 | 7.55E−01 | 4.37E−01 | 5.43E−01 | 5.62E−01 |
| 100003673 | 1109 | NA | NA | NA | 5.66E−01 |
| X - 24293 | 1110 | 7.11E−01 | 8.84E−01 | 2.91E−01 | NA |
| X - 12849 | 1111 | 3.14E−01 | 9.80E−01 | 5.96E−01 | NA |
| X - 11843 | 1112 | 4.73E−01 | 6.06E−01 | 6.39E−01 | NA |
| 100003252 | 1113 | 8.17E−01 | 8.35E−01 | 2.73E−01 | NA |
| 100002070 | 1114 | 4.59E−01 | 7.20E−01 | 3.56E−01 | 9.13E−01 |
| 208 | 1115 | NA | NA | NA | 5.73E−01 |
| 100001787 | 1116 | 3.52E−01 | 5.86E−01 | 9.14E−01 | NA |
| 100006293 | 1117 | NA | NA | NA | 5.74E−01 |
| X - 21815 | 1118 | 5.43E−01 | 3.98E−01 | 8.90E−01 | NA |
| 100015832 | 1119 | NA | NA | NA | 5.77E−01 |
| 100006378 | 1120 | NA | NA | NA | 5.77E−01 |
| 1137 | 1121 | 1.54E−01 | 9.06E−01 | 8.70E−01 | 9.21E−01 |
| 100006082 | 1122 | 2.15E−01 | 8.37E−01 | 7.84E−01 | 7.88E−01 |
| 100001526 | 1123 | NA | NA | NA | 5.86E−01 |
| 241 | 1124 | 6.12E−01 | 8.92E−01 | 9.01E−01 | 2.47E−01 |
| 565 | 1125 | 6.96E−01 | 8.66E−01 | 4.82E−01 | 4.19E−01 |
| 100015790 | 1126 | NA | NA | NA | 5.93E−01 |
| 100001151 | 1127 | NA | NA | NA | 5.94E−01 |
| 100009006 | 1128 | NA | NA | NA | 5.94E−01 |
| 100015643 | 1129 | NA | NA | NA | 5.98E−01 |
| 100004523 | 1130 | 2.67E−01 | 8.63E−01 | 5.96E−01 | 9.53E−01 |
| 100008906 | 1131 | NA | NA | NA | 6.04E−01 |
| 100008939 | 1132 | NA | NA | NA | 6.14E−01 |
| 100001993 | 1133 | 8.36E−01 | 6.50E−01 | 5.95E−01 | 4.52E−01 |
| 100000882 | 1134 | NA | NA | NA | 6.18E−01 |
| 100001612 | 1135 | NA | NA | NA | 6.20E−01 |
| 100005403 | 1136 | 3.61E−01 | 5.73E−01 | 8.20E−01 | 8.94E−01 |
| X - 16124 | 1137 | 7.54E−01 | 4.39E−01 | 7.34E−01 | NA |
| X - 21821 | 1138 | 4.69E−01 | 6.33E−01 | 8.23E−01 | NA |
| 100001664 | 1139 | NA | NA | NA | 6.26E−01 |
| 100001882 | 1140 | NA | NA | NA | 6.33E−01 |
| 100001990 | 1141 | 8.39E−01 | 4.68E−01 | 7.82E−01 | 5.26E−01 |
| 100002953 | 1142 | 5.73E−01 | 3.92E−01 | 9.46E−01 | 7.61E−01 |
| 100008991 | 1143 | 9.95E−01 | 9.67E−01 | 1.73E−01 | 9.73E−01 |
| 100001103 | 1144 | NA | NA | NA | 6.36E−01 |
| 100009045 | 1145 | NA | NA | NA | 6.36E−01 |
| 100000939 | 1146 | NA | NA | NA | 6.37E−01 |
| 100002679 | 1147 | NA | NA | NA | 6.41E−01 |
| 826 | 1148 | NA | NA | NA | 6.41E−01 |
| 100004171 | 1149 | 3.04E−01 | 7.64E−01 | 7.31E−01 | 9.98E−01 |
| 100002009 | 1150 | 9.55E−01 | 5.88E−01 | 9.47E−01 | 3.21E−01 |
| 100005714 | 1151 | NA | NA | NA | 6.43E−01 |
| 100001216 | 1152 | NA | NA | NA | 6.44E−01 |
| 1504 | 1153 | NA | NA | NA | 6.45E−01 |
| 100015687 | 1154 | NA | NA | NA | 6.46E−01 |
| 100006295 | 1155 | NA | NA | NA | 6.48E−01 |
| 100010926 | 1156 | NA | NA | NA | 6.55E−01 |
| 100003630 | 1157 | 4.89E−01 | 7.23E−01 | 8.00E−01 | NA |
| 100002735 | 1158 | 7.28E−01 | 6.77E−01 | 5.78E−01 | NA |
| 100001167 | 1159 | 8.85E−01 | 5.00E−01 | 6.56E−01 | 6.54E−01 |
| 100010895 | 1160 | NA | NA | NA | 6.63E−01 |
| 143 | 1161 | NA | NA | NA | 6.63E−01 |
| X - 17167 | 1162 | 8.84E−01 | 7.50E−01 | 4.43E−01 | NA |
| 100004054 | 1163 | NA | NA | NA | 6.66E−01 |
| 100015596 | 1164 | NA | NA | NA | 6.69E−01 |
| X - 12740 | 1165 | 3.67E−01 | 8.76E−01 | 9.29E−01 | NA |
| 100004509 | 1166 | 5.40E−01 | 8.99E−01 | 4.50E−01 | 9.20E−01 |
| 100006089 | 1167 | 7.29E−01 | 3.47E−01 | 9.15E−01 | 9.17E−01 |
| 100006360 | 1168 | NA | NA | NA | 6.82E−01 |
| 215 | 1169 | NA | NA | NA | 6.83E−01 |
| 980 | 1170 | NA | NA | NA | 6.91E−01 |
| 249 | 1171 | NA | NA | NA | 6.93E−01 |
| 100005367 | 1172 | 9.99E−01 | 6.66E−01 | 5.34E−01 | 6.67E−01 |
| 100015831 | 1173 | NA | NA | NA | 7.01E−01 |
| 100015727 | 1174 | NA | NA | NA | 7.09E−01 |
| 100009019 | 1175 | NA | NA | NA | 7.20E−01 |
| 100006129 | 1176 | NA | NA | NA | 7.23E−01 |
| 100005383 | 1177 | NA | NA | NA | 7.24E−01 |
| 100009042 | 1178 | NA | NA | NA | 7.31E−01 |
| 100002227 | 1179 | NA | NA | NA | 7.40E−01 |
| 100004635 | 1180 | 8.82E−01 | 9.75E−01 | 5.62E−01 | 6.24E−01 |
| 100009275 | 1181 | NA | NA | NA | 7.41E−01 |
| 1099 | 1182 | 6.34E−01 | 8.03E−01 | 8.08E−01 | NA |
| 100015744 | 1183 | NA | NA | NA | 7.47E−01 |
| 100001132 | 1184 | NA | NA | NA | 7.50E−01 |
| 100001063 | 1185 | NA | NA | NA | 7.51E−01 |
| 100015837 | 1186 | NA | NA | NA | 7.52E−01 |
| 100006184 | 1187 | NA | NA | NA | 7.69E−01 |
| 100003260 | 1188 | NA | NA | NA | 7.71E−01 |
| 100001002 | 1189 | NA | NA | NA | 7.73E−01 |
| 100009038 | 1190 | NA | NA | NA | 7.81E−01 |
| 100003210 | 1191 | NA | NA | NA | 7.82E−01 |
| 100015791 | 1192 | NA | NA | NA | 7.85E−01 |
| 100003679 | 1193 | NA | NA | NA | 7.93E−01 |
| 100005972 | 1194 | NA | NA | NA | 7.97E−01 |
| 100001733 | 1195 | 6.82E−01 | 9.58E−01 | 8.07E−01 | 7.95E−01 |
| 1215 | 1196 | NA | NA | NA | 8.05E−01 |
| 100009157 | 1197 | NA | NA | NA | 8.08E−01 |
| 100002067 | 1198 | 9.28E−01 | 9.18E−01 | 6.71E−01 | 7.52E−01 |
| 100010896 | 1199 | NA | NA | NA | 8.11E−01 |
| 100001431 | 1200 | NA | NA | NA | 8.15E−01 |
| 100002344 | 1201 | NA | NA | NA | 8.21E−01 |
| 100015850 | 1202 | NA | NA | NA | 8.24E−01 |
| 100003606 | 1203 | 9.12E−01 | 8.58E−01 | 7.28E−01 | NA |
| X - 17325 | 1204 | 6.98E−01 | 9.73E−01 | 8.63E−01 | NA |
| 100002128 | 1205 | NA | NA | NA | 8.38E−01 |
| 100015605 | 1206 | NA | NA | NA | 8.44E−01 |
| 213 | 1207 | NA | NA | NA | 8.44E−01 |
| 117 | 1208 | NA | NA | NA | 8.46E−01 |
| 100001469 | 1209 | NA | NA | NA | 8.55E−01 |
| 100005418 | 1210 | NA | NA | NA | 8.58E−01 |
| 100009227 | 1211 | NA | NA | NA | 8.68E−01 |
| 1023 | 1212 | NA | NA | NA | 8.72E−01 |
| 100001266 | 1213 | NA | NA | NA | 8.77E−01 |
| 100009184 | 1214 | NA | NA | NA | 8.80E−01 |
| 100002003 | 1215 | NA | NA | NA | 8.85E−01 |
| 100005716 | 1216 | NA | NA | NA | 8.87E−01 |
| 100008905 | 1217 | NA | NA | NA | 8.89E−01 |
| 100006271 | 1218 | NA | NA | NA | 9.00E−01 |
| 100015755 | 1219 | NA | NA | NA | 9.01E−01 |
| 100006108 | 1220 | NA | NA | NA | 9.03E−01 |
| 100009181 | 1221 | NA | NA | NA | 9.13E−01 |
| 100015688 | 1222 | NA | NA | NA | 9.15E−01 |
| 100001129 | 1223 | NA | NA | NA | 9.32E−01 |
| 100002017 | 1224 | NA | NA | NA | 9.32E−01 |
| 100004056 | 1225 | NA | NA | NA | 9.37E−01 |
| 100015624 | 1226 | NA | NA | NA | 9.43E−01 |
| 100002015 | 1227 | NA | NA | NA | 9.46E−01 |
| 100002952 | 1228 | NA | NA | NA | 9.48E−01 |
| 1488 | 1229 | NA | NA | NA | 9.49E−01 |
| 100000639 | 1230 | NA | NA | NA | 9.54E−01 |
| 100005834 | 1231 | NA | NA | NA | 9.55E−01 |
| 100001721 | 1232 | NA | NA | NA | 9.61E−01 |
| 100001279 | 1233 | NA | NA | NA | 9.61E−01 |
| 100008979 | 1234 | NA | NA | NA | 9.61E−01 |
| 100015731 | 1235 | NA | NA | NA | 9.66E−01 |
| 100000565 | 1236 | NA | NA | NA | 9.71E−01 |
| 100015787 | 1237 | NA | NA | NA | 9.77E−01 |
| 100004318 | 1238 | NA | NA | NA | 9.77E−01 |
| 100003109 | 1239 | NA | NA | NA | 9.83E−01 |
| 100004634 | 1240 | NA | NA | NA | 9.83E−01 |
| 100015689 | 1241 | NA | NA | NA | 9.84E−01 |
| 100015834 | 1242 | NA | NA | NA | 9.90E−01 |
| 100006296 | 1243 | NA | NA | NA | 9.94E−01 |
| 1022 | 1244 | NA | NA | NA | 9.96E−01 |

TABLE 12 A

| | | | | | | TWINSUK/Health | | | | | | |
|---|---|---|---|---|---|---|---|---|---|---|---|---|
| | | | | | | Nucleus insulin | | | | | | |
| | | | | | | resistance p | | | | TWINSUK normal | TWINSUK over- | TWINSUK obese |
| Metabolite | TWINSUK | TWINSUK | TWINSUK | Health | | (after control- | TWINSUK normal | TWINSUK | TWINSUK | weight direction | weight direction | direction |
| ID | v1 r2 | v2 r2 | v3 r2 | Nucleus r2 | Mean r2 | ling for BMI) | weight p v1 | overweight p v1 | obese p v3 | of effect v1 | of effect v1 | of effect v3 |
| 1134 | 0.123 | 0.162 | 0.136 | 0.219 | 0.179 | 0.0680 | 0.0001 | 0.0502 | 0.0059 | pos | pos | pos |
| 100001412 | 0.070 | 0.126 | 0.110 | 0.033 | 0.068 | 0.3923 | 0.0393 | 0.7432 | 0.0008 | pos | pos | pos |
| 100009051 | 0.104 | 0.090 | 0.096 | 0.057 | 0.077 | 0.0000 | 0.0040 | 0.1187 | 0.0637 | pos | pos | pos |
| 561 | 0.044 | 0.038 | 0.139 | 0.345 | 0.210 | 0.0143 | 0.1008 | 0.3477 | 0.2596 | pos | pos | pos |
| 212 | 0.039 | 0.092 | 0.118 | 0.044 | 0.063 | 0.0820 | 0.0631 | 0.2722 | 0.0031 | pos | pos | pos |
| 100001384 | 0.047 | 0.094 | 0.128 | 0.086 | 0.088 | 0.0392 | 0.1017 | 0.2960 | 0.0344 | neg | neg | neg |
| 100001006 | 0.062 | 0.107 | 0.139 | 0.077 | 0.090 | 0.0084 | 0.2226 | 0.0714 | 0.0044 | neg | neg | neg |
| 100005353 | 0.028 | 0.085 | 0.126 | 0.033 | 0.056 | 0.5156 | 0.3013 | 0.3297 | 0.0571 | neg | pos | neg |
| 566 | 0.074 | 0.088 | 0.084 | 0.131 | 0.106 | 0.0539 | 0.0502 | 0.0734 | 0.1447 | pos | pos | pos |
| 100009007 | 0.017 | 0.058 | 0.104 | 0.194 | 0.127 | 0.0482 | 0.3673 | 0.0120 | 0.0603 | pos | neg | neg |
| 100005352 | 0.023 | 0.080 | 0.098 | 0.068 | 0.067 | 0.0030 | 0.5998 | 0.8899 | 0.0477 | neg | neg | neg |
| 100001948 | 0.067 | 0.087 | 0.078 | 0.089 | 0.083 | 0.5326 | 0.1017 | 0.0212 | 0.0313 | pos | pos | pos |
| 100008917 | 0.019 | 0.064 | 0.101 | 0.159 | 0.110 | 0.1538 | 0.1739 | 0.0730 | 0.1838 | neg | neg | neg |
| 100001162 | 0.058 | 0.072 | 0.082 | 0.183 | 0.127 | 0.3506 | 0.0760 | 0.0788 | 0.0661 | pos | pos | pos |
| 98 | 0.056 | 0.082 | 0.063 | 0.053 | 0.060 | 0.0002 | 0.2466 | 0.2188 | 0.0226 | pos | pos | pos |
| 803 | 0.033 | 0.073 | 0.063 | 0.146 | 0.101 | 0.0044 | 0.9031 | 0.5666 | 0.1509 | neg | neg | pos |
| 1084 | 0.059 | 0.062 | 0.052 | 0.066 | 0.062 | 0.0099 | 0.8149 | 0.0115 | 0.0123 | pos | pos | pos |
| 100008981 | 0.039 | 0.057 | 0.086 | 0.080 | 0.071 | 0.1844 | 0.0038 | 0.2554 | 0.0331 | neg | neg | neg |
| 100001395 | 0.022 | 0.059 | 0.074 | 0.074 | 0.063 | 0.2483 | 0.0699 | 0.4531 | 0.0341 | neg | pos | neg |
| 100004046 | 0.036 | 0.083 | 0.063 | 0.130 | 0.095 | 0.6391 | 0.0695 | 0.1333 | 0.0183 | pos | pos | pos |
| 100002106 | 0.095 | 0.056 | 0.039 | 0.027 | 0.045 | 0.0217 | 0.0146 | 0.0215 | 0.3551 | pos | pos | pos |
| 100001415 | 0.057 | 0.070 | 0.058 | 0.042 | 0.052 | 0.9077 | 0.1123 | 0.4849 | 0.0046 | pos | pos | pos |
| 100009009 | 0.033 | 0.054 | 0.094 | 0.100 | 0.080 | 0.0900 | 0.2588 | 0.1759 | 0.0347 | neg | neg | neg |
| 100008985 | 0.066 | 0.048 | 0.068 | 0.028 | 0.044 | 0.0000 | 0.0004 | 0.4929 | 0.3368 | pos | pos | pos |
| 1110 | 0.056 | 0.071 | 0.058 | 0.035 | 0.049 | 0.2195 | 0.0644 | 0.1359 | 0.0340 | pos | pos | pos |
| 811 | 0.046 | 0.058 | 0.070 | 0.078 | 0.068 | 0.0002 | 0.0105 | 0.4585 | 0.1311 | pos | pos | pos |
| 100009015 | 0.012 | 0.039 | 0.100 | 0.036 | 0.043 | 0.1071 | 0.8010 | 0.9525 | 0.6949 | neg | neg | neg |
| 100000491 | 0.037 | 0.052 | 0.074 | 0.053 | 0.054 | 0.0026 | 0.3477 | 0.1989 | 0.0126 | pos | pos | pos |
| 100009055 | 0.060 | 0.054 | 0.048 | 0.076 | 0.065 | 0.0000 | 0.0323 | 0.0347 | 0.4324 | pos | pos | pos |
| 917 | 0.017 | 0.047 | 0.061 | 0.056 | 0.049 | 0.1422 | 0.7212 | 0.0176 | 0.2998 | neg | neg | neg |
| 1102 | 0.027 | 0.078 | 0.051 | 0.061 | 0.057 | 0.0006 | 0.5949 | 0.6817 | 0.0466 | pos | pos | pos |
| 815 | 0.040 | 0.058 | 0.049 | 0.066 | 0.057 | 0.0000 | 0.2147 | 0.8559 | 0.0898 | pos | pos | pos |
| 100002990 | 0.045 | 0.043 | 0.044 | 0.086 | 0.065 | 0.0006 | 0.2025 | 0.0298 | 0.4527 | pos | pos | pos |
| 100008903 | 0.028 | 0.043 | 0.069 | 0.070 | 0.058 | 0.2898 | 0.0305 | 0.8352 | 0.0622 | neg | neg | neg |
| 397 | 0.041 | 0.056 | 0.042 | 0.137 | 0.092 | 0.2557 | 0.1253 | 0.2563 | 0.5778 | pos | pos | pos |
| 100009053 | 0.053 | 0.061 | 0.067 | 0.039 | 0.049 | 0.0000 | 0.1240 | 0.5378 | 0.6210 | pos | pos | pos |
| 100009052 | 0.051 | 0.043 | 0.034 | 0.062 | 0.052 | 0.0001 | 0.6923 | 0.0444 | 0.7884 | pos | pos | pos |
| 100001104 | 0.030 | 0.056 | 0.034 | 0.062 | 0.051 | 0.0026 | 0.6925 | 0.1250 | 0.1962 | neg | pos | pos |
| 100000007 | 0.053 | 0.035 | 0.035 | 0.129 | 0.085 | 0.5871 | 0.4609 | 0.1296 | 0.6934 | pos | pos | pos |
| 100002989 | 0.040 | 0.040 | 0.047 | 0.082 | 0.062 | 0.0022 | 0.8656 | 0.0382 | 0.7658 | pos | pos | pos |
| 234 | 0.030 | 0.028 | 0.027 | 0.215 | 0.122 | 0.0626 | 0.0055 | 0.1845 | 0.2293 | pos | neg | pos |
| 100002253 | 0.022 | 0.055 | 0.056 | 0.023 | 0.033 | 0.9673 | 0.0387 | 0.1894 | 0.6396 | neg | neg | pos |
| 100009054 | 0.054 | 0.044 | 0.053 | 0.034 | 0.042 | 0.0002 | 0.0122 | 0.8539 | 0.4452 | pos | neg | pos |
| 182 | 0.061 | 0.076 | 0.057 | 0.044 | 0.054 | 0.0061 | 0.0196 | 0.0100 | 0.0108 | pos | pos | pos |
| 100001509 | 0.041 | 0.046 | 0.051 | 0.119 | 0.082 | 0.3741 | 0.7154 | 0.1058 | 0.0626 | pos | pos | pos |
| 572 | 0.046 | 0.036 | 0.039 | 0.101 | 0.071 | 0.0000 | 0.2328 | 0.0698 | 0.4976 | pos | pos | pos |
| 100009143 | 0.038 | 0.042 | 0.038 | 0.011 | 0.025 | 0.0239 | 0.0263 | 0.6714 | 0.1422 | pos | pos | pos |
| 100001586 | 0.025 | 0.033 | 0.024 | 0.026 | 0.027 | 0.0801 | 0.3708 | 0.0099 | 0.9946 | pos | pos | pos |
| 273 | 0.020 | 0.022 | 0.036 | 0.011 | 0.019 | 0.5039 | 0.0619 | 0.1576 | 0.4033 | neg | neg | neg |

TABLE 12B

| | | TWINSUK/ | | | | | TWINSUK/ | TWINSUK/ | | | | | |
|---|---|---|---|---|---|---|---|---|---|---|---|---|---|
| | | Health | TWINSUK/ | TWINSUK/ | TWINSUK/ | TWINSUK/ | Health | Health | TWINSUK/ | TWINSUK/ | | | TWINSUK/ |
| | TWINSUK/ | Nucleus | Health | Health | Health | Health | Nucleus | Nucleus | Health | Health | TWINSUK/ | TWINSUK/ | Health |
| | Health | Android/ | Nucleus | Nucleus | Nucleus | Nucleus | Diastolic | Systolic | Nucleus | Nucleus | Health | Health | Nucleus |
| Metabolite | Nucleus | gynoid | Percent | Subcutaneous | Visceral | Waist/hip | blood | blood | Insulin | Total | Nucleus | Nucleus | Total |
| ID | BMI r2 | ratio r2 | fat r2 | fat r2 | fat r2 | ratio r2 | pressure r2 | pressure r2 | resistance r2 | cholesterol r2 | HDL r2 | LDL r2 | triglycerides r2 |
| 1134 | 0.164 | 0.139 | <0.01 | 0.102 | 0.102 | 0.075 | 0.047 | 0.047 | 0.037 | <0.01 | 0.056 | <0.01 | 0.092 |
| 100001412 | 0.088 | 0.029 | 0.018 | 0.031 | 0.031 | 0.019 | 0.029 | 0.037 | 0.016 | <0.01 | <0.01 | <0.01 | 0.025 |
| 100009051 | 0.098 | 0.075 | 0.033 | 0.060 | 0.060 | 0.068 | 0.037 | 0.025 | 0.035 | 0.160 | <0.01 | 0.064 | 0.250 |
| 561 | 0.115 | 0.133 | 0.022 | 0.041 | 0.041 | 0.086 | 0.026 | 0.022 | 0.116 | <0.01 | 0.067 | 0.055 | 0.051 |
| 212 | 0.075 | 0.047 | <0.01 | 0.043 | 0.043 | 0.059 | 0.021 | 0.027 | 0.036 | <0.01 | 0.012 | <0.01 | 0.039 |
| 100001384 | 0.086 | 0.048 | 0.046 | 0.024 | 0.024 | 0.011 | <0.01 | <0.01 | 0.069 | 0.028 | 0.043 | 0.035 | <0.01 |
| 100001006 | 0.090 | 0.083 | 0.014 | 0.021 | 0.021 | 0.056 | <0.01 | <0.01 | 0.046 | <0.01 | 0.044 | <0.01 | 0.054 |
| 100005353 | 0.042 | 0.017 | 0.029 | <0.01 | <0.01 | <0.01 | <0.01 | <0.01 | 0.020 | 0.076 | 0.028 | 0.046 | <0.01 |
| 566 | 0.088 | 0.099 | <0.01 | 0.085 | 0.085 | 0.073 | 0.018 | 0.022 | 0.082 | <0.01 | 0.043 | <0.01 | 0.040 |
| 100009007 | 0.071 | 0.124 | 0.016 | 0.044 | 0.044 | 0.050 | <0.01 | <0.01 | 0.050 | 0.023 | 0.233 | <0.01 | 0.116 |
| 100005352 | 0.062 | 0.047 | 0.025 | <0.01 | <0.01 | <0.01 | <0.01 | <0.01 | 0.058 | 0.024 | 0.040 | <0.01 | 0.012 |
| 100001948 | 0.098 | 0.074 | <0.01 | 0.031 | 0.031 | 0.063 | 0.013 | 0.017 | 0.045 | <0.01 | 0.063 | <0.01 | 0.060 |
| 100008917 | 0.065 | 0.095 | <0.01 | 0.020 | 0.020 | 0.028 | 0.015 | <0.01 | 0.045 | 0.043 | 0.171 | 0.033 | 0.078 |
| 100001162 | 0.099 | 0.105 | <0.01 | 0.028 | 0.028 | 0.050 | 0.022 | 0.020 | 0.075 | <0.01 | 0.044 | <0.01 | 0.056 |
| 98 | 0.060 | 0.046 | <0.01 | 0.028 | 0.028 | 0.026 | 0.025 | 0.029 | 0.071 | <0.01 | 0.012 | <0.01 | 0.022 |
| 803 | 0.066 | 0.052 | <0.01 | 0.043 | 0.043 | 0.051 | 0.016 | 0.025 | 0.068 | <0.01 | 0.026 | <0.01 | 0.013 |
| 1084 | 0.073 | 0.056 | <0.01 | 0.047 | 0.047 | 0.088 | 0.013 | 0.020 | 0.069 | <0.01 | <0.01 | <0.01 | 0.036 |
| 100008981 | 0.056 | 0.053 | 0.015 | 0.023 | 0.023 | 0.017 | <0.01 | <0.01 | 0.049 | <0.01 | 0.069 | <0.01 | <0.01 |
| 100001395 | 0.049 | <0.01 | 0.038 | <0.01 | <0.01 | 0.011 | <0.01 | <0.01 | 0.029 | 0.063 | 0.068 | 0.017 | <0.01 |
| 100004046 | 0.069 | 0.129 | <0.01 | 0.052 | 0.052 | 0.061 | 0.017 | 0.015 | 0.019 | <0.01 | 0.058 | <0.01 | 0.015 |
| 100002106 | 0.068 | 0.012 | 0.069 | 0.039 | 0.039 | 0.015 | 0.011 | <0.01 | 0.014 | 0.164 | <0.01 | 0.095 | 0.024 |
| 100001415 | 0.073 | 0.037 | 0.011 | 0.021 | 0.021 | 0.019 | 0.011 | 0.020 | 0.011 | <0.01 | 0.017 | <0.01 | 0.016 |
| 100009009 | 0.057 | 0.061 | 0.024 | 0.028 | 0.028 | 0.050 | <0.01 | <0.01 | 0.048 | 0.026 | 0.177 | <0.01 | 0.095 |
| 100008985 | 0.051 | 0.035 | 0.024 | 0.030 | 0.030 | 0.030 | 0.030 | 0.026 | 0.026 | 0.126 | <0.01 | 0.028 | 0.236 |
| 1110 | 0.066 | 0.035 | <0.01 | 0.044 | 0.044 | 0.044 | 0.011 | 0.015 | 0.029 | <0.01 | 0.015 | <0.01 | 0.022 |
| 811 | 0.053 | 0.055 | <0.01 | 0.044 | 0.044 | 0.033 | 0.022 | 0.017 | 0.043 | <0.01 | <0.01 | <0.01 | 0.038 |
| 100009015 | 0.025 | 0.035 | 0.018 | <0.01 | <0.01 | 0.015 | <0.01 | <0.01 | 0.014 | 0.026 | 0.074 | <0.01 | 0.063 |
| 100000491 | 0.060 | 0.050 | <0.01 | 0.025 | 0.025 | 0.059 | 0.022 | 0.038 | 0.088 | <0.01 | 0.032 | <0.01 | 0.025 |
| 100009055 | 0.076 | 0.083 | 0.024 | 0.083 | 0.083 | 0.098 | 0.035 | 0.029 | 0.076 | 0.066 | 0.099 | 0.031 | 0.376 |
| 917 | 0.037 | 0.042 | 0.023 | <0.01 | <0.01 | <0.01 | <0.01 | <0.01 | <0.01 | <0.01 | 0.014 | <0.01 | 0.025 |
| 1102 | 0.046 | 0.053 | <0.01 | 0.024 | 0.024 | 0.050 | 0.034 | 0.044 | 0.075 | <0.01 | <0.01 | 0.015 | 0.025 |
| 815 | 0.018 | 0.010 | <0.01 | 0.030 | 0.030 | 0.059 | 0.013 | <0.01 | 0.088 | <0.01 | <0.01 | <0.01 | <0.01 |
| 100002990 | 0.063 | 0.082 | 0.015 | 0.070 | 0.070 | 0.077 | 0.031 | 0.025 | 0.040 | 0.069 | 0.122 | 0.038 | 0.417 |
| 100008903 | 0.042 | 0.026 | 0.013 | 0.020 | 0.020 | 0.014 | <0.01 | <0.01 | 0.013 | 0.054 | 0.075 | 0.025 | <0.01 |
| 397 | 0.068 | 0.098 | <0.01 | 0.049 | 0.049 | 0.063 | 0.018 | 0.019 | 0.087 | <0.01 | 0.027 | <0.01 | 0.030 |
| 100009053 | 0.056 | 0.058 | 0.020 | 0.049 | 0.049 | 0.056 | 0.031 | 0.039 | 0.061 | 0.025 | 0.036 | <0.01 | 0.283 |
| 100009052 | 0.072 | 0.101 | 0.011 | 0.068 | 0.068 | 0.094 | 0.022 | 0.025 | 0.084 | 0.025 | 0.109 | <0.01 | 0.336 |
| 100001104 | 0.042 | 0.063 | <0.01 | 0.022 | 0.022 | 0.075 | 0.026 | 0.025 | 0.068 | <0.01 | <0.01 | <0.01 | 0.024 |
| 100000007 | 0.075 | 0.074 | <0.01 | 0.047 | 0.047 | 0.034 | <0.01 | <0.01 | 0.019 | <0.01 | 0.034 | <0.01 | <0.01 |
| 100002989 | 0.059 | 0.090 | <0.01 | 0.080 | 0.080 | 0.074 | 0.027 | 0.024 | 0.034 | 0.038 | 0.138 | 0.020 | 0.398 |
| 234 | 0.070 | 0.079 | 0.023 | 0.017 | 0.017 | 0.022 | <0.01 | <0.01 | 0.088 | <0.01 | 0.052 | 0.043 | 0.029 |
| 100002253 | 0.035 | 0.060 | 0.014 | 0.026 | 0.026 | 0.014 | <0.01 | <0.01 | <0.01 | <0.01 | 0.013 | <0.01 | 0.043 |
| 100009054 | 0.060 | 0.041 | 0.025 | 0.052 | 0.052 | 0.028 | 0.018 | 0.024 | 0.034 | 0.019 | 0.023 | <0.01 | 0.223 |
| 182 | 0.084 | 0.058 | 0.034 | 0.040 | 0.040 | 0.049 | 0.013 | 0.013 | 0.039 | <0.01 | 0.042 | <0.01 | 0.049 |
| 100001509 | 0.083 | 0.087 | <0.01 | 0.044 | 0.044 | 0.066 | 0.017 | 0.017 | 0.083 | <0.01 | 0.048 | 0.016 | 0.017 |
| 572 | 0.063 | 0.052 | <0.01 | 0.022 | 0.022 | 0.021 | 0.016 | 0.028 | 0.063 | <0.01 | 0.020 | <0.01 | 0.060 |
| 100009143 | 0.029 | 0.015 | 0.033 | 0.021 | 0.021 | 0.035 | 0.020 | 0.016 | <0.01 | 0.075 | <0.01 | 0.027 | 0.159 |
| 100001586 | 0.032 | 0.020 | <0.01 | <0.01 | <0.01 | <0.01 | <0.01 | 0.013 | 0.014 | 0.024 | <0.01 | 0.036 | 0.038 |
| 273 | 0.025 | <0.01 | 0.021 | <0.01 | <0.01 | <0.01 | <0.01 | <0.01 | <0.01 | <0.01 | <0.01 | 0.011 | <0.01 |

Of particular interest was the association with cortisone, a metabolite of the steroid hormone cortisol. The results show lower levels among the obese individuals, which is consistent with previous reports. There are, however some inconsistent relationships between cortisol and metabolic parameters in the literature. Additionally, each of the 49 metabolites in just those of normal weight, overweight or obese separately were examined. The directionality of the effect was found to be largely consistent with those seen in the group as a whole (Table 12A or Table 12B).

Modelling the Metabolome of Obesity—

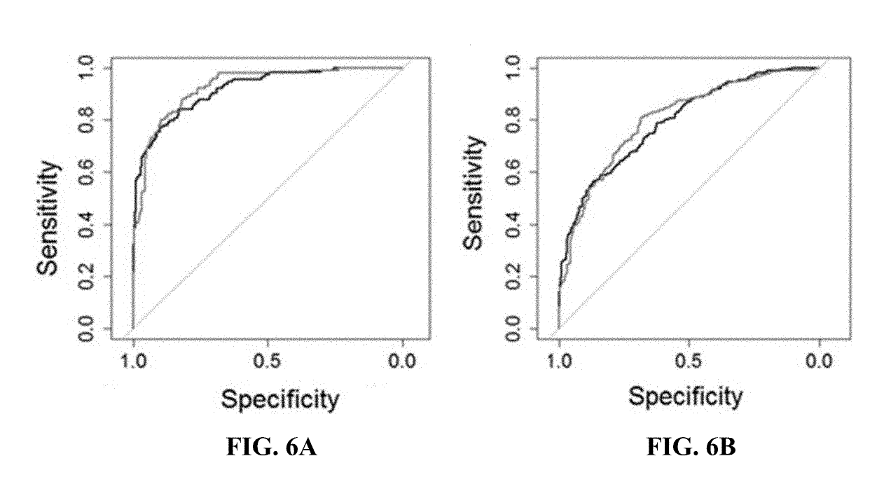

Ridge regression was used to build a model that would predict BMI from the 49 BMI-associated metabolites (see FIG. 3). This method was chosen to focus on the most stringently associated metabolites and to remove effects of co-linearity, and similar results were observed using lasso regression. Data for the first visit of the TWINSUK cohort and the Health Nucleus cohort was combined and the model was trained with 10-fold cross-validation on a random half of the population. In the test set of the other half of the data, it was found that the model could explain 39.1% of the variation in BMI (FIG. 3A). In predicting whether participants were obese (BMI>=30) or normal weight (BMI 18.5-25), the model had an area under the curve (AUC) of 0.922, specificity of 89.1% and sensitivity of 80.2% (FIG. 6). The model based on the metabolite signature was thereafter used as a tool to define mBMI, the predicted BMI on the basis of metabolome.

Richer models using the full set of available metabolites (n=650 measured in both cohorts) improved the accuracy of the model (47-49% of the variance explained) and could be considered as the optimal approach by accepting the additional cost of a full untargeted metabolome as compared to the more targeted panel of 49 metabolites. This performance should be contrasted to the results of models using routine clinical laboratory determinations: regression analysis predicting BMI from age, sex, HDL, LDL, total cholesterol and total triglycerides explained 31% of the variation in BMI, whereas a model using age, sex and the 49 metabolite signature explained 43% of the variation. In fact, even though mBMI was modeled by training on BMI, this metabolite signature had a better correlation than BMI with most health-related phenotypes measured here (Table 2).

Identification and Characterization of Metabolic BMI Outliers—

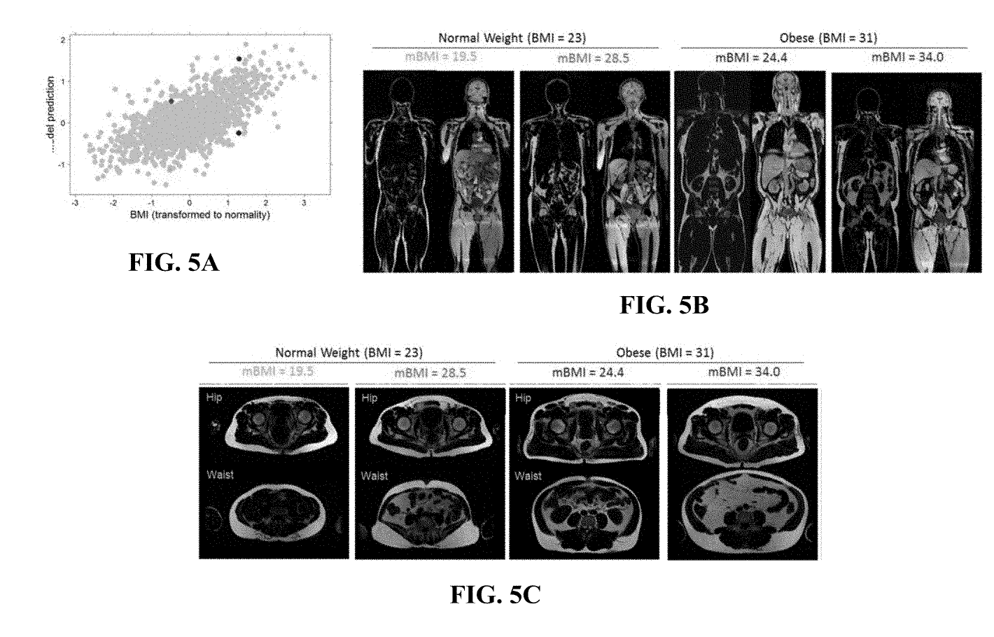

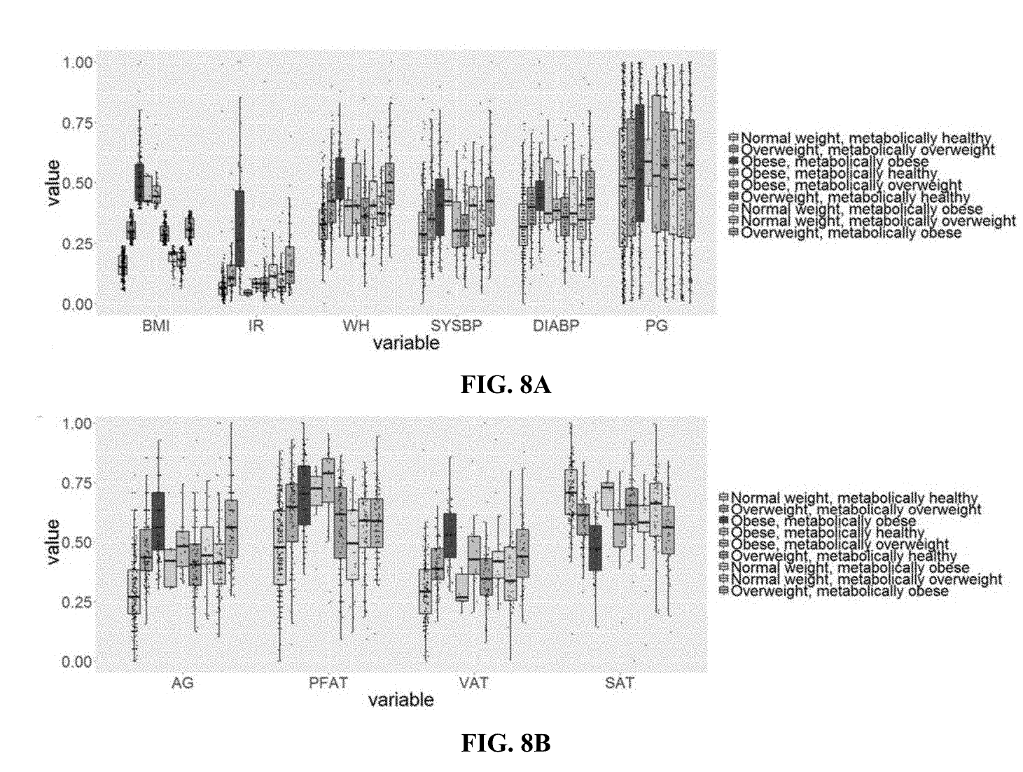

Having established a model to predict BMI using the metabolome, the participants were split into 5 groups (FIG. 3A, FIG. 8). Three groups included individuals whose metabolome accurately predicted their BMI, as defined by having a residual between −0.5 and 0.5 m a regression analysis of mBMI with age, sex and BMI included as predictors. These criteria delineated ˜80% of individuals as having an mBMI relatively concordant with actual BMI. Three groups included individuals whose metabolome accurately predicted their BMI (residual between −0.5 and 0.5): they were characterized as having a normal BMI (18.5-25), overweight (25-30), or obese (>30). Two groups were characterized as outliers: these included individuals whose metabolome predicted a substantially higher mBMI than the actual BMI (mBMI>>BMI, residual >0.5) or a substantially lower mBMI than the actual BMI (mBMI<<BMI, residual >0.5). While these two outlier groups had the same weight range distribution, they had very different values for many of the phenotypes of metabolic health collected from these cohorts (FIG. 3B and FIG. 3C). Individuals with an mBMI prediction that was substantially higher than their actual BMI had levels of insulin resistance, blood pressure, waist/hip ratio, android/gynoid ratio, percent body fat, percent visceral fat, and percent subcutaneous fat that were similar to obese individuals with obese metabolomes. Individuals with an mBMI prediction that was substantially lower than their actual BMI had levels for these traits that were similar to those of normal-weight individuals with healthy metabolomes. Evaluating these data from a more clinical perspective, with individuals separated into clinical categories such as normal BMI with obese metabolome and obese BMI with healthy metabolome, generally confirmed these effects (FIG. 5 and FIG. 8). These findings suggest that the metabolome can be used as clinically meaningful instrument, where obesity is analyzed in the context of its metabolome perturbation. Thus, these results are important in the frame of the current debate on the “healthy” obese and for the identification of individuals with normal BMIS but poor metabolic health.

Having characterized these outliers, metabolome differences were revisited. As expected, those with mBMI substantially higher than BMI significantly differed in their metabolite levels from those with mBMI substantially lower than BMI for most of the 49 BMI-associated metabolites. However, two of the BMI-associated metabolites did not differ between these two groups: asparagine and cortisone. The association between each of the BMI-associated metabolites and insulin resistance was additionally investigated, which as many previously reported markers of obesity have also been markers of diabetes. Insulin resistance measurements were taken for 515 unrelated, European-ancestry participants. After controlling for BMI, it was found that 12 of the 49 BMI-associated metabolites were significantly associated (correcting for 49 tests requires p<0.001) with insulin resistance, all with positive direction of effect: tyrosine, alanine, kynurenate, gamma-glutamyltyrosine, 1-oleoyl-3-linoleoyl-glycerol (18:1/18:2), and six phospholipids, and glucose (see Tables 11, 12A, Table 12B).

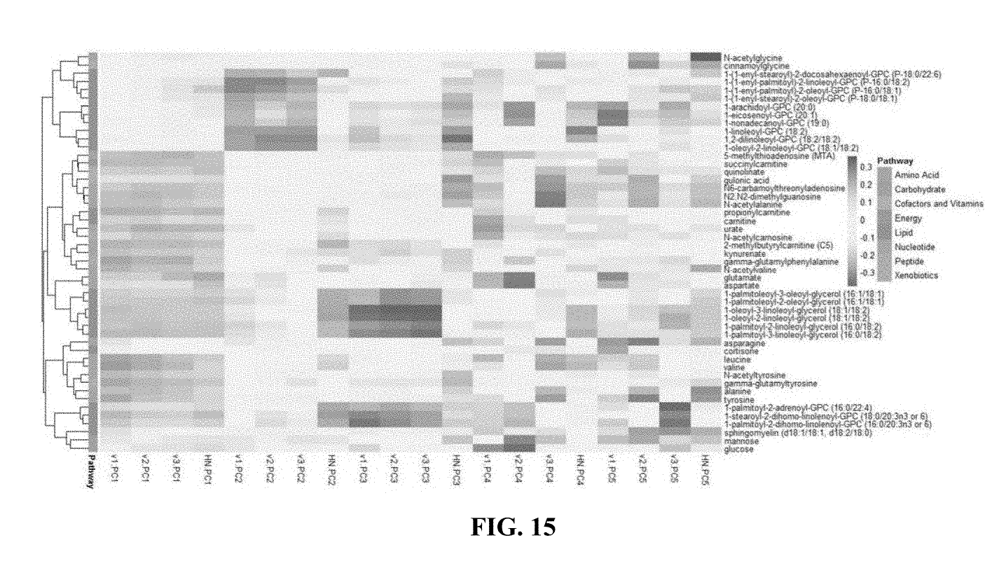

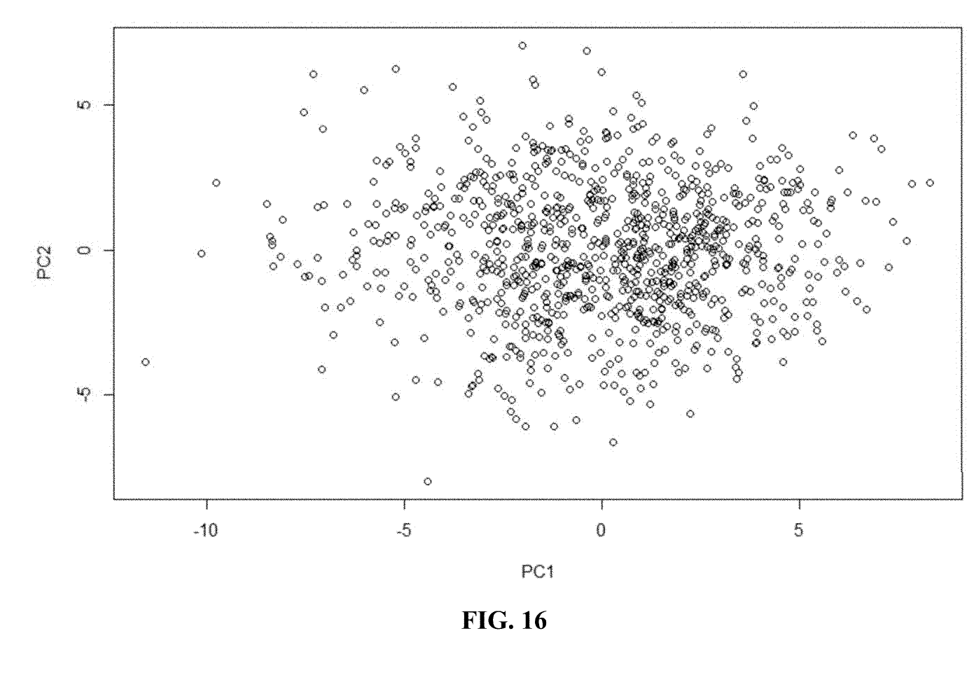

Principal component analysis of the main 49 BMI-associated metabolites indicated that the first principal component, which was most heavily influenced by nucleotides and amino acids, explained 19.7, 19.8, and 21.4% of the overall variation in these metabolites at time points 1, 2 and 3 and 22.5% in the Health Nucleus. The first principal component also explained 17.9%, 28.6%, and 29.9% of the variation in BMI at these time points and 48.9% in the Health Nucleus. This difference in explanatory power across cohorts likely reflected differences in cohort composition, especially the difference in sex ratios across studies. The TWINSUK cohort was 96.7% female, while the Health Nucleus cohort was 32.9% female; when restricting to females within the Health Nucleus cohort, the first principal component only explained 32.9% of the variation in BMI. The first principal component was not only useful for distinguishing obese from non-obese: even among those who were obese, this component respectively explained 4.0, 13.5, 10.9, and 16.1% of the variation in obese BMI. This first principal component was a robust and reliable predictor of BMI, with the majority of the 49 metabolites having strong influences on this component at all three visits. Some of the most important contributors to this axis included metabolites involved in nucleotide metabolism, such as urate and pseudouridine, and diverse amino acids, especially branched-chain amino acids. The subsequent axes were more prone to changing their contributing metabolites across visits, but they were consistently strongly influenced by key metabolites as shown in FIG. 15: in general, the second and third axes reflected glycerol phospholipids and glycerophosphocholines, with axis 2 additionally reflecting various amino acids, especially tryptophan metabolism; axis 4 reflected amino acids, especially branched-chain amino acids and aromatic amino acids; and axis 5 reflected mannose, glucose, glycerol and glycerol lipids, and diverse amino acids. Clear subgroups of individuals did not appear from the principal components as distributions were continuous (FIG. 16).

BMI Changes Over Time—

BMI data from TWINSUK were available for all three-time points for 1,458 participants. On average, participants gained 0.91 BMI between the first and second visits, when the mean age increased from 51 to 58, and lost 0.09 BMI between the second and third visits, when the mean age increased to 64 (FIG. 2). Some of this variation was related to the age of the participants and to their menopause status: the 209 women who remained premenopausal at the second visit gained 1.57 BMI, the 146 who progressed from premenopausal at the first visit to post menopause at the second visit gained on average 1.42 BMI, and the 648 women who were already postmenopausal at the first visit gained on average only 0.54 BMI between the first and second visit. Over the full course of the study, 1,044 participants (71.6%) always stayed within 3 BMI of their starting weight, 253 (17.4%) gained more than 3 BMI, and 77 (5.3%) lost more than 3 BMI (FIG. 2).

Predictors of Changes in BMI—

BMI change over the course of the study as a phenotype in analyses was used to identify metabolites or demographic factors that could predict weight change in the 695 TWINSUK participants with weight at all three time points who were unrelated and genetically of European ethnicity. It was found that age at the start of the study was by far the most significant predictor of weight change, explaining 9.4% of the variation in slope of BMI change. Menopause status at the beginning and end of the study explained an additional 1.5% of the variation and time between visits explained 0.5% more, while initial BMI and sex were not predictors of change in BMI over time. No single metabolite at time point 1 was significantly (p<5.5×10-5) associated with the slope of subsequent BMI change after controlling for initial BMI, age and time between visits. Likewise, the BMI prediction made using 49 metabolites from visit 1 was not significantly associated with subsequent weight change. The lack of association with change in BMI thus show that the perturbation in metabolite patterns was likely a consequence of the BMI changes as opposed to a contributing factor.

Metabolite Recovery after BMI Change—

To confirm this direction of effect, a study was conducted to investigate whether longitudinal changes in weight were reflected by longitudinal changes in metabolite levels within the same person. It was found that when tracking an individual's weight across visits, their metabolite changes generally reflected their weight changes. For example, 73% of the 41 participants who were classified as having gained weight between time point 1 and 2 and then losing weight between time points 2 and 3 had metabolome BMI model predictions increase at time point 2 and then decrease again at time point 3, demonstrating that metabolite changes associated with a BMI change could be reversed. Overall, participants who had substantial weight change between time points (as defined in the methods and FIG. 2) had metabolome BMI model prediction changes consistent with the expectation for that weight change at both time points 63% of the time, and the complete opposite from expectation only 6% of the time.

MC4R Variant Carriers with Low Polygenic Risk Scores—

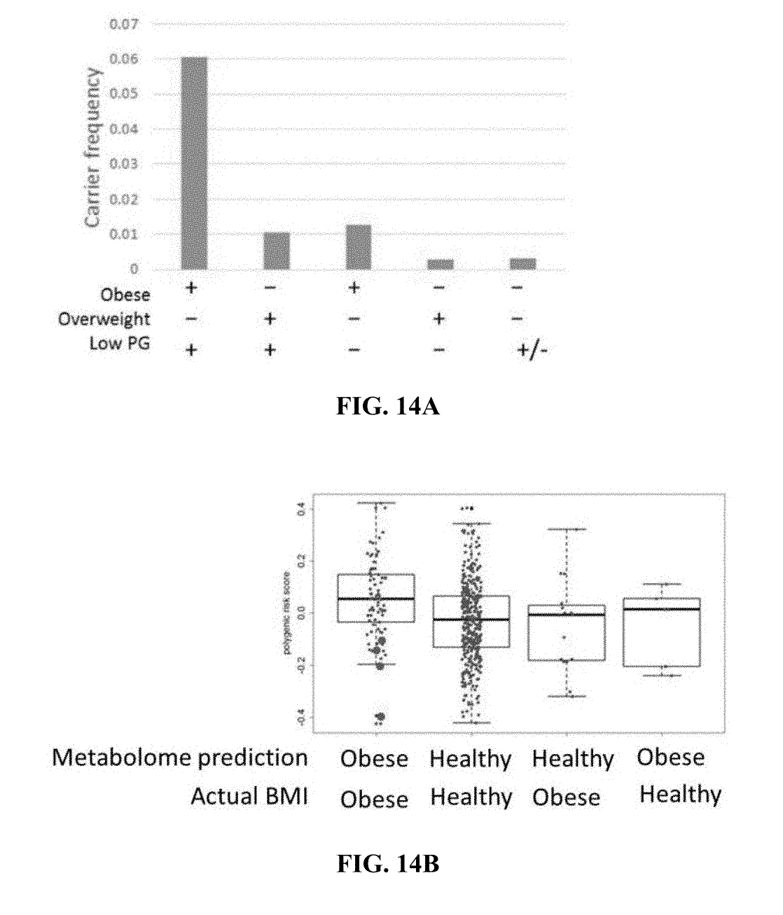

Members of the study populations who were carrying rare (MAF<0.01%) coding variants in the known obesity gene MC4R were identified. Specifically, eight such carriers were identified in the subset of unrelated participants, with an enrichment in participants who were obese despite a low polygenic risk score. Out of 31 participants who were obese with polygenic risk scores in the lowest quartile, 6.1% were MC4R variant carriers, while the carrier frequency was just 0.3% in those of normal weight (FIG. 14). Of four obese MC4R variant carriers, two had a dizygotic twin who was also a carrier of the variant. In both cases, both twins were obese despite having polygenic risk scores in the bottom quartile. Both sets of twins were predicted to be obese from their metabolome. Three of the four unrelated obese carriers of MC4R variants were also predicted to be obese from their metabolomes, and their metabolomes were indistinguishable from other obese participants who were not MC4R carriers.

Evolution of Obesity and Metabolome Clinical Profiles—

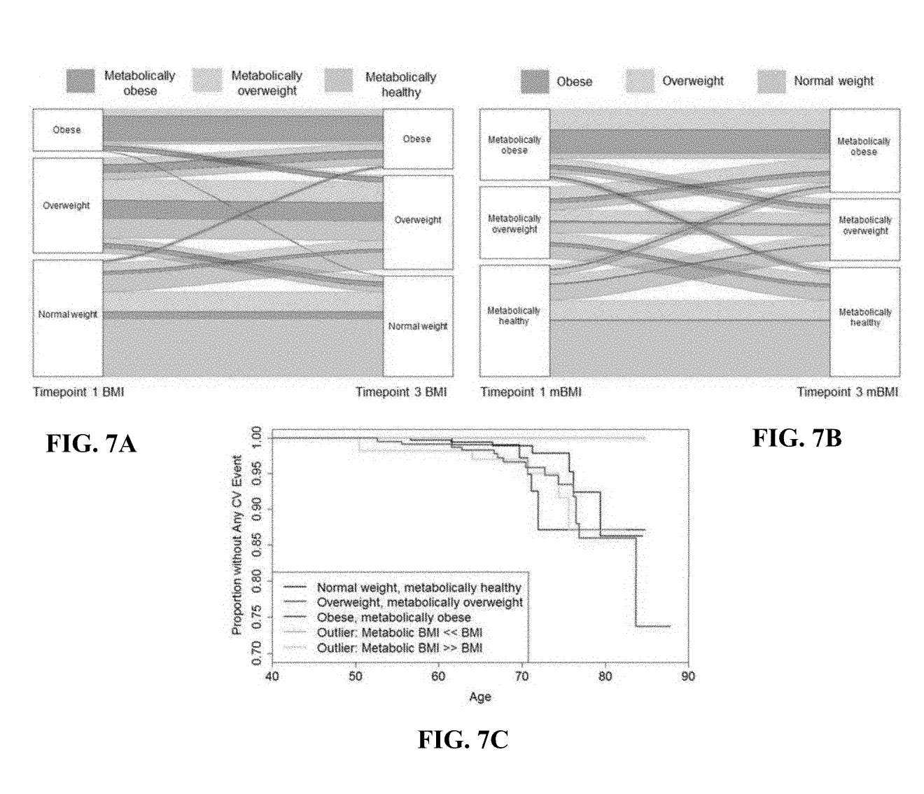

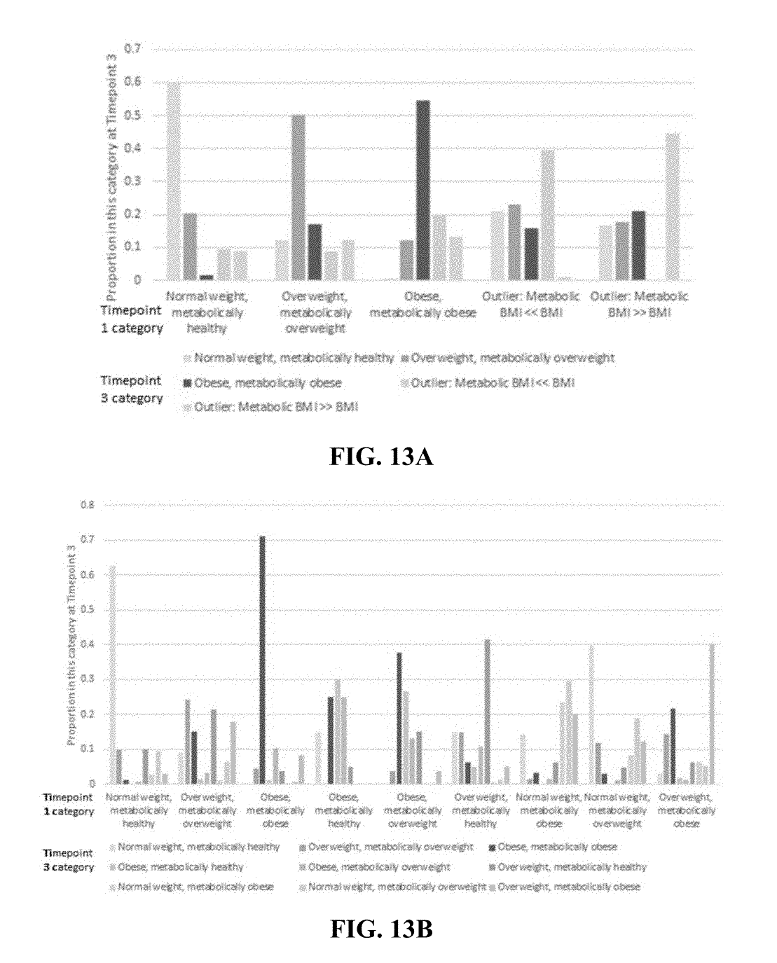

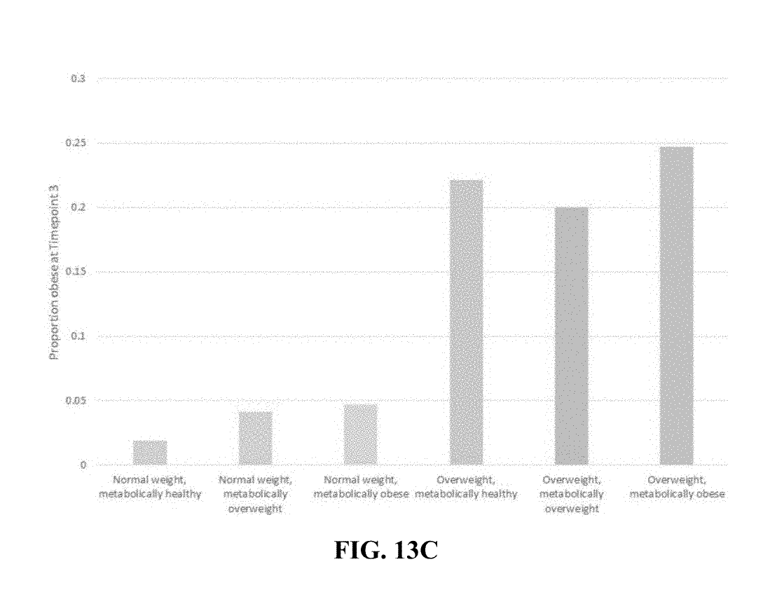

Given recent research showing that obese individuals who are metabolically healthy may remain at higher risk of negative health outcomes than are normal weight individuals who are metabolically healthy, a study was conducted to address whether the outlier groups were more likely to become obese over time. Focusing on the 1,458 individuals from TWINSUK who had weight measurements at all three time points, it was found that those who had a mBMI that was higher than their BMI were marginally more likely to gain weight and convert to an obese phenotype (BMI>30) over the 8-18 years of follow up. For example, 32.8% of those of normal weight but with an overweight or obese metabolome converted to being overweight or obese by time point 3 compared to 24.8% of those who were of normal weight and had a healthy metabolome (p=0.02, FIG. 7 and FIG. 13A-13C). The mBMI states of the individuals remained fairly stable with time (FIG. 7 and FIG. 13A-13C). For example, 68% of the individuals who began the study with an obese metabolome ended the study with an obese metabolome. In summary, these results are consistent with a favorable long-term health benefit for the overweight and obese individuals with a healthy metabolome.

Cardiovascular Disease Outcomes—

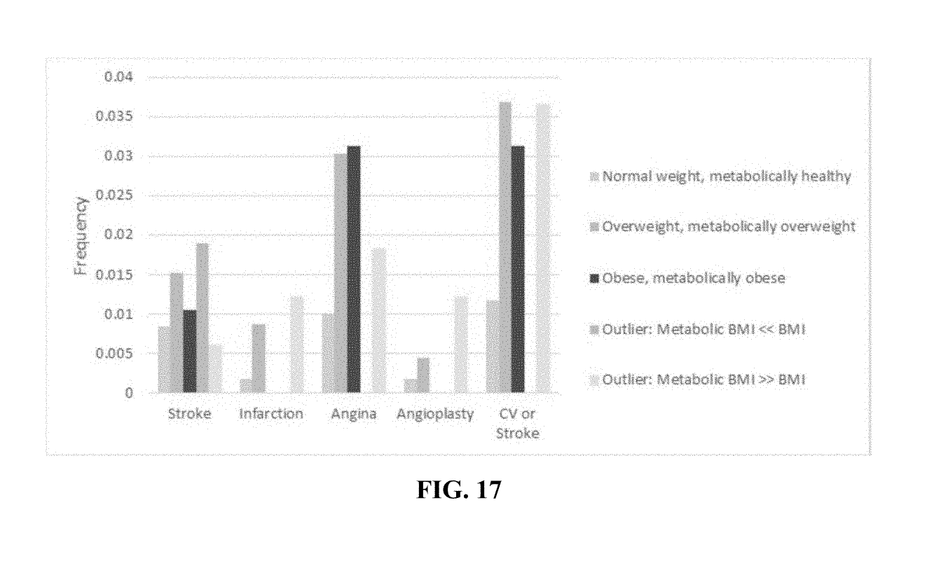

Obesity is a well-recognized risk factor for cardiovascular disease and ischemic stroke. The longitudinal nature of the TWINSUK study allowed the collection of clinical endpoints in these unselected participants. The age of participants at the first visit ranged from 33 to 74 years old (median 51); and 42 to 88 years old (median 65) at the last visit. During the follow up (median 13 years), the study recorded 53 cardiovascular events (myocardial infarct, angina, angioplasty) or strokes for 1573 individuals. Participants with a healthy metabolome (normal BMI or obese) had 2 events per hundred individuals. Individuals with an obese metabolic profile had 3.7 (normal BMI) and 4.2 events (in obese individuals) per hundred individuals. Separated analysis of the various endpoints confirmed the trends, more accentuated for cardiovascular than for diagnosis of stroke (FIG. 17). A formal survival analysis was then performed for participants to have any cardiovascular event after the first time point, and it was found those with healthier metabolomes to have fewer/later cardiac events, p-value 0.02 (FIG. 7).

Correlations Between Twins—

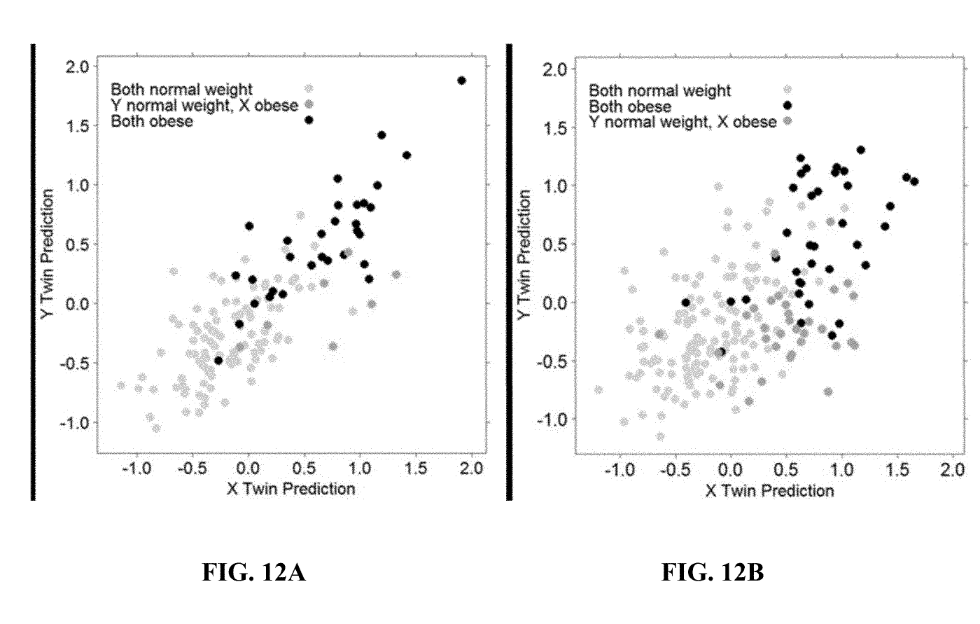

Because twin studies are important to analyze the heritability of traits, the BMI model predictions and obesity status of 350 sets of twins were reassessed, wherein either both twins had normal BMI (n=244), both twins were obese (n=67), or one was obese and the other had normal BMI (n=39). To keep the categories clear, individuals with BMIs between 25 and 30 (overweight) and their twins were excluded. As asserted by the model's high specificity and sensitivity, the metabolite-based obesity predictions tended to reflect the actual obesity statuses of the individuals. This was even the case when only one twin was obese: the obese twin was generally predicted by their metabolome to be obese, while the normal weight twin was not (FIG. 12). The correlations between the metabolite-based obesity predictions was also substantially higher between the monozygotic twins than the dizygotic twins, as expected. Interestingly, 3 sets of twins were identified, where both twins were predicted from the metabolome to be of normal weight, but both were obese, and 8 sets of twins where the reverse was true.

These outliers were thought to represent the healthy obese and normal weight, metabolically unhealthy individuals described above.

Genetic Analyses

Known Genetics of Obesity—

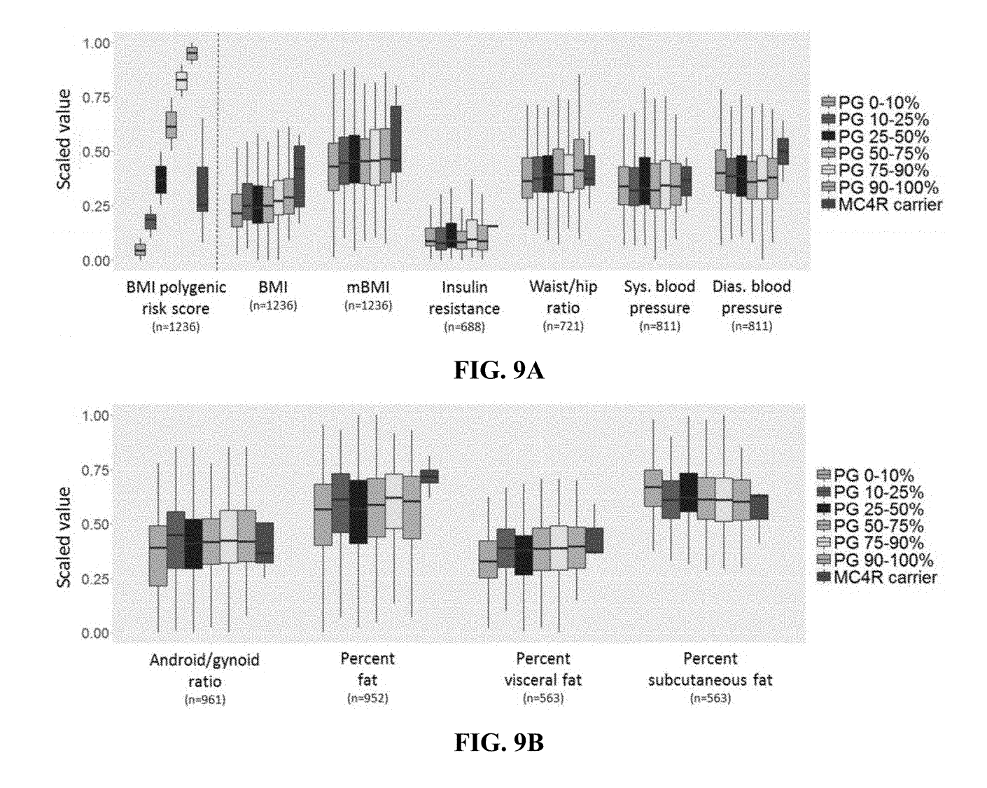

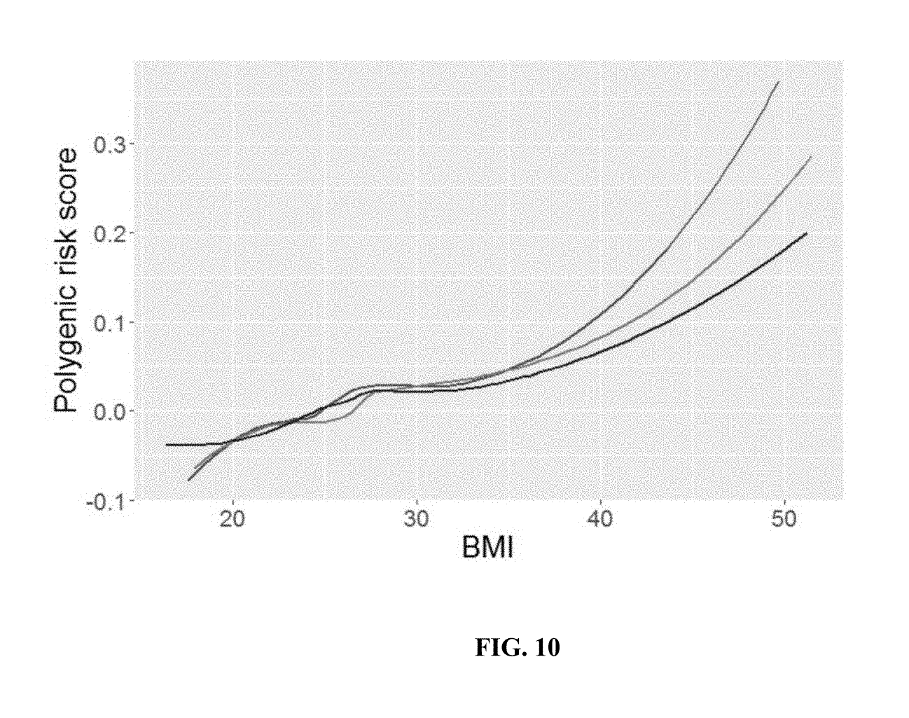

The study first investigated the known genetic factors contributing to high BMI. Polygenic scores for BMI were calculated using known associations from the considerable literature of obesity and BMI GWAS. As previously reported, it was found that polygenic risk score only explained 2.2% of the variation in BMI at each of the three TWINSUK time points and in Health Nucleus for unrelated participants of European ancestry (FIG. 10). A study was conducted to investigate whether unique individuals with the highest polygenic risk would have a significant perturbation of the metabolome and anthropomorphic, insulin resistance and DEXA measurements (FIG. 9). While the data did not support a strong role for polygenic risk, there were trends for higher polygenic risk scores to be associated with a higher android/gynoid ratio (p=0.04) and waist/hip ratio (p=0.04). However, there was no statistical association between the polygenic score and mBMI (p=0.16). Overall, the data suggest that the genetics of BMI could reflect an association with anthropomorphic traits (larger-framed individuals) rather than a unique association with obesity as a disease trait. Members of the study populations who were carrying rare (MAF<0.01%) coding variants in the known obesity gene MC4R were specifically identified.

Specifically, the study identified 8 such carriers in the subset of unrelated participants (Table 2). Each variant was observed in one unrelated individual, and 5 of the 8 had already been annotated as causing obesity in HGMD or ClinVar (Table 2). As a group, MC4R carriers had significantly higher BMI (p=0.02) than did non-carriers as well as non-significant trends toward a higher diastolic blood pressure, insulin resistance, and percent body fat (FIG. 9). However, not all rare variants may be deleterious, and the metabolic impact could have been greater for the true subset of functional variants. The BMI data in the participants supported a pathogenic role for five of the variants (Met292fs, Arg236Cys, Ser180Pro, Ala175T, and Thr11Ala), but did not corroborate a role of Ile170V, which is defined in HGMD and ClinVar as pathogenic. Importantly, of the five sets of twins who both carried the same MC4R variant, three sets included twins who were both overweight and obese. In the two cases where a carrier's twin did not have the MC4R variant, their BMI was lower than their twin's. An enrichment of MC4R variant carriers was observed among obese individuals with low polygenic risk scores (supplemental results, FIG. 14). Out of 31 participants who were obese with polygenic risk scores in the lowest quartile, 6.1% were MC4R variant carriers, while the carrier frequency was just 0.3% in those of normal weight.

Genetics of the Healthy Obese—

Additional support for the decoupling of the genetics of high BMI versus the basis of obesity and predicted mBMI was derived from the analysis of outliers. Individuals with an mBMI that was substantially lower than their actual BMI had a higher polygenic risk score for BMI than did other groups. In contrast, those whose mBMI was substantially higher than their actual BMI had low polygenic risk scores (FIG. 3B; p=0.006 for a difference between these two groups). This result would also support the notion that the polygenic risk score for BMI may capture an anthropomorphic phenotype rather than a disease phenotype.

Genetics of metabolome differences—

The study further investigated whether obese individuals with different genetic backgrounds had different metabolomes from other obese individuals. First, metabolites that could distinguish individuals with different BMI polygenic risk scores or MC4R variant carriers were searched. Linear regression showed no significant associations between any single metabolites and polygenic risk or MC4R carrier status either in the entire population or in only the obese individuals. This result implies that metabolites are unlikely to be intermediate phenotypes that explain the underlying genetics of obesity. To check for more specific signals beyond the compiled polygenic risk score, separate analyses of each of the 97 variants that are used to calculate the polygenic risk score were also performed. There was no evidence for any of these known GWAS variants to be more strongly associated with a metabolite than with BMI itself, though the power for discovery was limited given the very small effect sizes of most individual GWAS variants. In summary, although it is known that there is a strong genetic component to metabolite levels, most of the metabolic perturbations that occur in the obese state are a response to obesity as opposed to shared genetic mechanisms.

### DISCUSSION

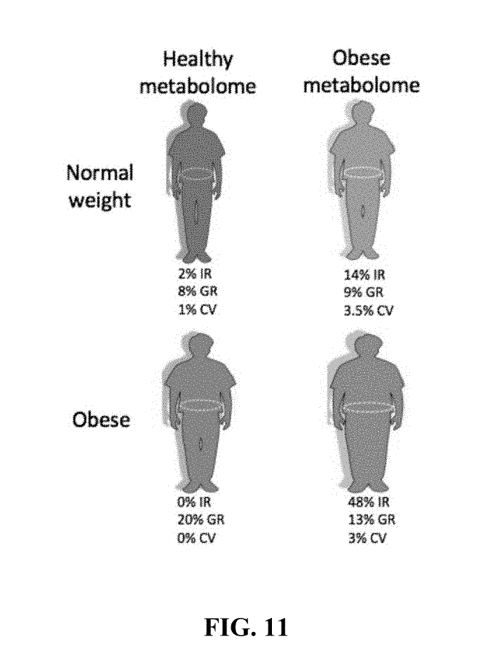

The results of the present study highlight the profound disruption of the metabolome that is caused by obesity and identifies a metabolome signature that serves to examine metabolic health beyond anthropomorphic measurements (FIG. 11). Nearly one third of the approximately 1000 metabolites measured in the study were associated with BMI, and 49 were selected as a strong signature for the study of the relationship between BMI, obesity, metabolic disease and the genetics of BMI. Consistent with previous studies and earlier work in the TWINSUK cohort, branched-chain and aromatic amino acids, and metabolites involved in nucleotide metabolism, such as urate and pseudouridine, are strongly perturbed by obesity. The underlying reason for the perturbation of branched-chain amino acid metabolism in obese individuals and those with insulin resistance is thought to be related to differences in the amino acid catabolism in adipose tissue. The single metabolite with the most significant association with BMI was urate, as previously reported.

It is well known that uric acid increases with obesity, due to insulin resistance reducing the kidneys' ability to eliminate uric acid, but previous work has not emphasized the power of urate to predict BMI. It was also found that 23 of the lipids in the assay were definitively associated with BMI, with an enrichment of associations found for glycerol lipids. These results are consistent with previous studies showing that sphingomyelins and diacylglycerols increase with BMI while lysophosphocholines decrease with BMI, with other various phosphatidylcholines having effects in both directions. Other previously reported metabolite associations with BMI, including positive associations with choline, cysteine, pantothenate, fructose, palmitate, stearate, fructose, and xylose, and negative associations with citrulline, methionine, and uridine are not apparent in the large study. These metabolites have largely been implicated in studies specifically addressing diabetes in the setting of obesity, and their effects may be limited to that context. Given this landscape, it will be of interest to perform studies that more specifically dissect the associations of metabolites with various traits. For example, few of the BMI-associated metabolites were associated with insulin resistance after controlling for BMI, despite the overlap between obese patients and patients with insulin resistance. As previously observed, the metabolome abnormalities associated with high BMI corrected with loss of weight. However, the present study found that metabolite levels did not provide predictive power for future weight changes. Overall, the metabolome perturbations appear as a consequence of changes in weight as opposed to being a contributing factor.

The present study does not support a strong association between metabolome changes and the genetics of BMI defined by a 97-variant polygenic risk score. This may be explained by the fact that known BMI GWAS loci explain only a small fraction (˜3%) of BMI heritability. However, as discussed below, the BMI polygenic risk may also influence body build and not only obesity. Taken together, it does not appear that metabolites are intermediate phenotypes between the genetics of BMI and obesity itself. The study also identified individuals who carried rare functional variants in the known obesity gene MC4R. The carriers of these variants were often obese individuals, but their metabolome was not categorically different from that of other obese individuals. The lack of metabolome differences for carriers of variants in this gene is not surprising given that MC4R variants cause obesity by increasing appetite. However, the results did not show that obese carriers of MC4R variants often had low polygenic risk scores for obesity; out of 31 participants who were obese with polygenic risk scores in the lowest quartile, 6.1% were MC4R variant carriers, while the carrier frequency was just 0.3% in those of normal weight. Polygenic risk scores are calculated using common variants and association statistics from existing genome-wide association studies. Their impact on phenotype is generally modest, and the present study demonstrates part of why this is true: rare variants with larger effects on phenotypes are not captured in polygenic risk scores.

The present study shows the potential to sequence obese individuals who are outliers with low polygenic risk scores because of the apparent enrichment for monogenic contributions. As of the completion of the study, a large consortium provided additional detail on the role of variants in pathways that implicate energy intake and expenditure in obesity. Finally, the metabolome signature identified individuals whose predicted mBMI was either substantially higher or lower than their actual BMI. These individuals include the metabolically healthy obese, but also emphasize the importance of the metabolome in unhealthy individuals with a normal BMI. These profiles were surprisingly stable over the prolonged follow-up. This suggests that there is a durable benefit of maintaining a healthy metabolome signature and points to an ongoing risk for the individuals that have an unhealthy metabolome despite stability of BMI. An abnormal metabolome signature, irrespective of BMI, was associated in the present study with three-fold increase in cardiovascular events. Thus, while these findings are in line with the known relationships between metabolically healthy obese status and health-related traits like metabolic syndrome and body fat, the relationship was extended to a broader category of metabolically healthy and unhealthy individuals on the basis of the disparity between mBMI and BMI. The fact that the metabolically healthy obese have a high BMI polygenic risk score also supports the concept that some of the genetic studies may capture anthropomorphic associations—body size—rather than obesity sensu stricto. These findings are in line with previous studies identifying genetic variants specifically associated with the metabolically healthy obese, or favorable adiposity. While the common variants associated with favorable adiposity thus far have had subtle effects, a thorough investigation of the full genomes mBMI/BMI outliers can be expected to identify rare variants with large effects on healthy obesity and unhealthy metabolome with normal weight.

The biological differences between these outlier categories would benefit from further study as well. For example, differences in waist/hip ratio, percent visceral fat, and blood pressure between mBMI/BMI outliers were observed despite having the same BMI distribution (FIG. 5 and FIG. 11). Furthermore, while most of the 49 BMI-associated metabolites were significantly different between the outlier groups, it was found that cortisone and asparagine levels did not differ. The specificity of this association in the cohort may help shed light on the inconsistent relationships between cortisol and obesity that have been reported. This study highlights the health risks of the perturbed metabolome. The study also decouples the genetics of BMI from metabolic health and serves to prioritize a subset of individuals for genetic analysis. The assessment of the metabolome and genome of BMI lays groundwork for future studies of the heterogeneity of obesity and treatment of its endophenotypes. Specifically, the metabolome signature can act as a biomarker of response to the new therapeutics that target patients with MC4R mutations. Metabolic profiling could help select patients for clinical trials beyond genetic sequencing, thus expanding drug utility.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

We claim:

1. A method of diagnosing obesity or a disease related thereto in a subject, comprising,
obtaining a biological sample from the subject;
detecting, in the biological sample, levels or activities of at least 3 metabolites selected from the metabolites of Table 1 or derivatives thereof and calculating a metabolomic body mass index (mBMI) value for the subject based on the detection, wherein the metabolites of Table 1 are listed in the order of effect on the mBMI value;
TABLE 1

| S/N | Metabolite |
|---|---|
| 1 | Urate |
| 2 | 5-methylthioadenosine (MTA) |
| 3 | Glutamate |
| 4 | N2,N2-dimethylguanosine |
| 5 | 1-nonadecanoyl-GPC (19:0) |
| 6 | N-acetylglycine |
| 7 | 1-arachidoyl-GPC (20:0) |
| 8 | 1-stearoyl-2-dihomo-linolenoyl-GPC (18:0/20:3n3 or 6) |
| 9 | 1-(1-enyl-palmitoyl)-2-oleoyl-GPC (P-16:0/18:1) |
| 10 | 1-oleoyl-2-linoleoyl-GPC (18:1/18:2) |
| 11 | Valine |
| 12 | 1-(1-enyl-stearoyl)-2-docosahexaenoyl-GPC (P-18:0/22:6) |
| 13 | Succinylcarnitine |
| 14 | Kynurenate |
| 15 | 1-(1-enyl-palmitoyl)-2-linoleoyl-GPC (P-16:0/18:2) |
| 16 | gamma-glutamylphenylalanine |
| 17 | N-acetylcamosine |
| 18 | 1-eicosenoyl-GPC (20:1) |
| 19 | Mannose |
| 20 | sphingomyelin (d18:1/18:1, d18:2/18:0) |
| 21 | gamma-glutamyltyrosine |
| 22 | N-acetylalanine |
| 23 | 1-(1-enyl-stearoyl)-2-oleoyl-GPC (P-18:0/18:1) |
| 24 | N6-carbamoylthreonyladenosine |
| 25 | 1-linoleoyl-GPC (18:2) |
| 26 | Propionylcarnitine |
| 27 | 1,2-dilinoleoyl-GPC (18:2/18:2) |
| 28 | 1-palmitoyl-2-dihomo-linolenoyl-GPC (16:0/20:3n3 or 6) |
| 29 | 1-palmitoleoyl-2-oleoyl-glycerol (16:1/18:1) |
| 30 | Alanine |
| 31 | Aspartate |
| 32 | 1-palmitoyl-3-linoleoyl-glycerol (16:0/18:2) |
| 33 | Asparagine |
| 34 | N-acetylvaline |
| 35 | N-acetyltyrosine |
| 36 | Leucine |
| 37 | 1-palmitoleoyl-3-oleoyl-glycerol (16:1/18:1) |
| 38 | Tyrosine |
| 39 | Cinnamoylglycine |
| 40 | 1-oleoyl-2-linoleoyl-glycerol (18:1/18:2) |
| 41 | 1-palmitoyl-2-linoleoyl-glycerol (16:0/18:2) |
| 42 | 1-oleoyl-3-linoleoyl-glycerol (18:1/18:2) |
| 43 | Carnitine |
| 44 | 1-palmitoyl-2-adrenoyl-GPC(16:0/22:4) |
| 45 | Quinolinate |
| 46 | 2-methylbutyrylcarnitine (C5) |
| 47 | Glucose |
| 48 | Cortisone |
| 49 | gulonic acid |
| 50 | Adenine |
| 51 | sphingomyelin (d18:2/14:0, d18:1/14:1) |
| 52 | Pseudouridine |
| 53 | sphingomyelin (d18:2/16:0, d18:1/16:1) |
| 54 | Kynurenine |
| 55 | 3-phenylpropionate (hydrocinnamate) |
| 56 | arachidate (20:0) |
| 57 | Glycerol |
| 58 | 1-oleoyl-2-docosahexaenoyl-GPC (18:1/22:6) |
| 59 | hydantoin-5-propionic acid |
| 60 | 2-aminoadipate |
| 61 | 1-margaroyl-2-linoleoyl-GPC (17:0/18:2) |
| 62 | 1-oleoyl-GPC (18:1) |
| 63 | palmitoleoyl-linoleoyl-glycerol (16:1/18:2) [1] |
| 64 | N1-methyladenosine |
| 65 | 2-linoleoyl-GPC (18:2) |
| 66 | 1-margaroyl-GPC (17:0) |
| 67 | 3-hydroxy-3-methylglutarate |
| 68 | beta-cryptoxanthin |
| 69 | 1-(1-enyl-palmitoyl)-GPC (P-16:0) |
| 70 | 1-(1-enyl-palmitoyl)-2-palmitoyl-GPC (P-16:0/16:0) |
| 71 | N6-acetyllysine |
| 72 | N-acetylleucine |
| 73 | 1-stearoyl-2-oleoyl-GPE (18:0/18:1) |
| 74 | Phenylalanine |
| 75 | 1-(1-enyl-stearoyl)-2-docosahexaenoyl-GPE (P-18:0/22:6) |
| 76 | erucate (22:1n9) |
| 77 | Hypotaurine |
| 78 | N-acetylphenylalanine |
| 79 | Orotidine |
| 80 | docosahexaenoate (DHA; 22:6n3) |
| 81 | Lactate |
| 82 | N-acetylserine |
| 83 | 1-palmitoyl-2-arachidonoyl-GPI (16:0/20:4) |
| 84 | 1-docosahexaenoyl-GPC (22:6) |
| 85 | 3-(4-hydroxyphenyl)lactate |
| 86 | N-acetylisoleucine |
| 87 | 1,3,7-trimethylurate |
| 88 | Proline |
| 89 | 1-palmitoyl-2-linoleoyl-GPI (16:0/18:2) |
| 90 | linoleoyl-arachidonoyl-glycerol (18:2/20:4) [1] |
| 91 | 1-palmitoyl-2-oleoyl-GPE (16:0/18:1) |
| 92 | 2-docosahexaenoyl-GPC (22:6) |
| 93 | Glycine |
| 94 | Isovalerylcarnitine |
| 95 | 1-palmitoyl-2-oleoyl-GPI (16:0/18:1) |
| 96 | Ribitol |
| 97 | 1-methylhistidine |
| 98 | 1-stearoyl-2-docosapentaenoyl-GPC (18:0/22:5n6) |
| 99 | 1,7-dimethylurate |
| 100 | gamma-CEHC glucuronide |
| 101 | Butyrylcarnitine |
| 102 | lactosyl-N-palmitoyl-sphingosine |
| 103 | Glutamine |
| 104 | 1-linolenoylglycerol (18:3) |
| 105 | 4-androsten-3beta,17beta-diol monosulfate (1) |
| 106 | 1-stearoyl-2-meadoyl-GPC (18:0/20:3n9) |
| 107 | 1-palmitoyl-2-arachidonoyl-GPC (16:0/20:4) |
| 108 | 1-stearoyl-2-arachidonoyl-GPC (18:0/20:4) |
| 109 | cyclo(leu-pro) |
| 110 | gamma-tocopherol/beta-tocopherol |
| 111 | indolepropionate |
| 112 | glucuronate |
| 113 | 1-stearoyl-2-arachidonoyl-GPE (18:0/20:4) |
| 114 | bilirubin (E,Z or Z,E) |
| 115 | 1-stearoyl-2-docosahexaenoyl-GPE (18:0/22:6) |
| 116 | 1-palmitoyl-2-palmitoleoyl-GPC (16:0/16:1) |
| 117 | methyl indole-3-acetate |
| 118 | 2-linoleoyl-GPE (18:2) |
| 119 | 1-(1-enyl-stearoyl)-GPE (P-18:0) |
| 120 | 1-oleoylglycerol (18:1) |
| 121 | dimethylglycine |
| 122 | 1-stearoyl-2-linoleoyl-GPE (18:0/18:2) |
| 123 | bilirubin (Z,Z) |
| 124 | creatine |
| 125 | argininate |
| 126 | N-acetyltryptophan |
| 127 | homoarginine |
| 128 | ribonate |
| 129 | glycohyocholate |
| 130 | 7-alpha-hydroxy-3-oxo-4-cholestenoate (7-Hoca) |
| 131 | glycerate |
| 132 | sulfate |
| 133 | X - 12100 |
| 134 | X - 22822 |
| 135 | X - 11787 |
| 136 | X - 15492 |
| 137 | 1-carboxyethylvaline |
| 138 | X - 15503 |
| 139 | X - 11299 |
| 140 | X - 11452 |
| 141 | 1-carboxyethylphenylalanine |
| 142 | X - 12040 |
| 143 | hydroxy-CMPF |
| 144 | X - 15486 |
| 145 | 5,6-dihydrouridine |
| 146 | 3-methylglutarylcarnitine (1) |
| 147 | X - 11372 |
| 148 | X - 12847 |
| 149 | X - 12329 |
| 150 | X - 13835 |
| 151 | X - 18901 |
| 152 | X - 17166 |
| 153 | glycine conjugate of C10H14O2 (1) |
| 154 | X - 12206 |
| 155 | X - 23026 |
| 156 | X - 11522 |
| 157 | X - 23639 |
| 158 | X - 21752 |
| 159 | X - 11905 |
| 160 | X - 18249 |
| 161 | X - 17299 |
| 162 | X - 11838 |
| 163 | X - 24435 |
| 164 | X - 12101 |
| 165 | X - 17145 |
| 166 | X - 21736 |
| 167 | X - 16580 |
| 168 | 5-methylthioribose |
| 169 | X - 16944 |
| 170 | X - 17179 |
| 171 | X - 17337 |
| 172 | bradykinin, des-arg(9) |
| 173 | X - 12846 |
| 174 | X - 12221 |
| 175 | octadecenedioate (C18:1-DC) |
| 176 | X - 23593 |
| 177 | X - 11429 |
| 178 | X - 14056 |
| 179 | X - 14838 |
| 180 | X - 16123 |
| 181 | X - 21626 |
| 182 | X - 16132 |
| 183 | 1-palmityl-2-oleoyl-GPC (O-16:0/18:1) |
| 184 | 1-myristoyl-2-arachidonoyl-GPC (14:0/20:4) |
| 185 | 1-pentadecanoyl-2-arachidonoyl-GPC (15:0/20:4) |
| 186 | 4-hydroxyglutamate |
| 187 | 1-(1-enyl-stearoyl)-2-linoleoyl-GPC (P-18:0/18:2) |
| 188 | 1-(1-enyl-palmitoyl)-2-palmitoleoyl-GPC (P-16:0/16:1) |
| 189 | gamma-glutamyltryptophan |
| 190 | S-adenosylhomocysteine (SAH) |
| 191 | 1-linoleoyl-2-docosahexaenoyl-GPC (18:2/22:6) |
| 192 | 1-oleoyl-2-dihomo-linoleoyl-GPC (18:1/20:2) |
| 193 | C-glycosyltryptophan |
| 194 | guanidinoacetate |
| 195 | isoleucine |
| 196 | gamma-glutamylisoleucine |
| 197 | gamma-glutamylleucine |
| 198 | nonadecanoate (19:0) |
| 199 | beta-alanine |
| 200 | 1-(1-enyl-palmitoyl)-2-docosahexaenoyl-GPC (P-16:0/22:6) |
| 201 | N1-Methyl-2-pyridone-5-carboxamide |
| 202 | urea |
| 203 | pyruvate |
| 204 | 1-stearyl-GPC (O-18:0) |
| 205 | gamma-glutamylvaline |
| 206 | 2-hydroxyphenylacetate |
| 207 | 1-palmitoleoylglycerol (16:1) |
| 208 | palmitoyl sphingomyelin (d18:1/16:0) |
| 209 | 1-oleoyl-2-dihomo-linolenoyl-GPC (18:1/20:3) |
| 210 | allantoin |
| 211 | N-acetylneuraminate |
| 212 | 1-palmitoyl-2-stearoyl-GPC (16:0/18:0) |
| 213 | pipecolate |
| 214 | 1-methylimidazoleacetate |
| 215 | 5alpha-androstan-3alpha,17beta-diol monosulfate (1) |
| 216 | 7-methylguanine |
| 217 | sphingosine |
| 218 | 1-palmitoyl-2-docosahexaenoyl-GPC (16:0/22:6) |
| 219 | 1-stearoyl-GPC (18:0) |
| 220 | erythritol |
| 221 | 1-dihomo-linoleoyl-GPC (20:2) |
| 222 | 2-oleoyl-GPC (18:1) |
| 223 | 1-dihomo-linolenylglycerol (20:3) |
| 224 | 2-palmitoyl-GPE (16:0) |
| 225 | 1-myristoylglycerol (14:0) |
| 226 | gamma-glutamylalanine |
| 227 | 2-docosahexaenoyl-GPE (22:6) |
| 228 | 1-(1-enyl-oleoyl)-GPC (P-18:1) |
| 229 | mannitol/sorbitol |
| 230 | alpha-ketoglutarate |
| 231 | 1-palmitoyl-GPE (16:0) |
| 232 | hexadecadienoate (16:2n6) |
| 233 | 1-(1-enyl-stearoyl)-GPC (P-18:0) |
| 234 | 3-methyladipate |
| 235 | 1-dihomo-linolenoyl-GPC (20:3n3 or 6) |
| 236 | erythronate |
| 237 | 1,2-dipalmitoyl-GPC (16:0/16:0) |
| 238 | palmitoyl dihydro sphingomyelin (d18:0/16:0) |
| 239 | 5-methyluridine (ribothymidine) |
| 240 | 2-hydroxybutyrate/2-hydroxyisobutyrate |
| 241 | 1-eicosapentaenoyl-GPE (20:5) |
| 242 | 1-palmitoyl-GPC (16:0) |
| 243 | N-acetylcitrulline |
| 244 | 2-aminoheptanoate |
| 245 | indoleacetylglutamine |
| 246 | eicosapentaenoate (EPA; 20:5n3) |
| 247 | phenylalanylphenylalanine |
| 248 | ergothioneine |
| 249 | gluconate |
| 250 | 1-myristoyl-2-linoleoyl-GPC (14:0/18:2) |
| 251 | stearoyl sphingomyelin (d18:1/18:0) |
| 252 | gamma-glutamyl-epsilon-lysine |
| 253 | oxalate (ethanedioate) |
| 254 | glutarylcarnitine (C5) |
| 255 | N-acetylmethionine |
| 256 | dihydroorotate |
| 257 | palmitoleate (16:1n7) |
| 258 | deoxycholate |
| 259 | 1-methylurate |
| 260 | 2-oxoarginine |
| 261 | tartronate (hydroxymalonate) |
| 262 | 1-stearoyl-2-arachidonoyl-GPI (18:0/20:4) |
| 263 | 2-hydroxypalmitate |
| 264 | N-formylphenylalanine |
| 265 | isobutyrylglycine |
| 266 | 1-(1-enyl-stearoyl)-2-arachidonoyl-GPC (P-18:0/20:4) |
| 267 | leucylleucine |
| 268 | 1-docosahexaenoyl-GPE (22:6) |
| 269 | gamma-glutamyl-alpha-lysine |
| 270 | serotonin |
| 271 | 1-stearoyl-GPE (18:0) |
| 272 | caprate (10:0) |
| 273 | succinate |
| 274 | thyroxine |
| 275 | phosphocholine (16:0/22:5n3, 18:1/20:4) |
| 276 | cysteine sulfinic acid |
| 277 | 1-(1-enyl-palmitoyl)-2-arachidonoyl-GPE (P-16:0/20:4) |
| 278 | 7-methylurate |
| 279 | sphingomyelin (d18:1/20:1, d18:2/20:0) |
| 280 | 1-arachidonylglycerol (20:4) |
| 281 | 2-hydroxyadipate |
| 282 | 3-methyl-2-oxobutyrate |
| 283 | 6-oxopiperidine-2-carboxylic acid |
| 284 | 4-hydroxyphenylacetate |
| 285 | 1-linoleoyl-GPE (18:2) |
| 286 | xanthine |
| 287 | 1-docosapentaenoyl-GPC (22:5n3) |
| 288 | 1-margaroyl-2-oleoyl-GPC (17:0/18:1) |
| 289 | 1-palmityl-GPC (O-16:0) |
| 290 | 3,7-dimethylurate |
| 291 | choline phosphate |
| 292 | dodecanedioate |
| 293 | 2-methylbutyrylglycine |
| 294 | 2-hydroxystearate |
| 295 | N-acetyltaurine |
| 296 | N-acetylglutamate |
| 297 | 3-methyl-2-oxovalerate |
| 298 | X - 15245 |
| 299 | 2-methylcitrate/homocitrate |
| 300 | PC(O-16:0/16:0) |
| 301 | X - 21339 |
| 302 | lysoPE(O-16:0) |
| 303 | X - 11537 |
| 304 | X - 11530 |
| 305 | 1-oleoyl-2-eicosapentaenoyl-GPC (18:1/20:5) |
| 306 | X - 13737 |
| 307 | prolylproline |

and
diagnosing the subject as having obesity if the mBMI value of the subject is modulated compared to a reference standard.

2. A method of diagnosing obesity or a disease related thereto in a subject, comprising,
obtaining a biological sample from the subject;
detecting, in the biological sample, levels or activities of at least 3 metabolites selected from the metabolites of Table 2 or derivatives thereof and computing a metabolomic body mass index (mBMI) value for the subject based on the detection, wherein the metabolites of Table 2 are listed in the order of effect on the mBMI value;
TABLE 2

| S/N | Metabolite |
|---|---|
| 1 | Urate |
| 2 | Glutamate |
| 3 | 1-(1-enyl-palmitoyl)-2-oleoyl-GPC (P-16:0/18:1) |
| 4 | 1-stearoyl-2-dihomo-linolenoyl-GPC (18:0/20:3n3 or |
| | 6) |
| 5 | 1-eicosenoyl-GPC (20:1) |
| 6 | N2,N2-dimethylguanosine |
| 7 | 1-arachidoyl-GPC (20:0) |
| 8 | 1-(1-enyl-stearoyl)-2-oleoyl-GPC (P-18:0/18:1) |
| 9 | N-acetylglycine |
| 10 | 5-methylthioadenosine (MTA) |
| 11 | Valine |
| 12 | Propionylcarnitine |
| 13 | Succinylcarnitine |
| 14 | 1-nonadecanoyl-GPC (19:0) |
| 15 | 1-linoleoyl-GPC (18:2) |
| 16 | Aspartate |
| 17 | Mannose |
| 18 | N-acetylvaline |
| 19 | Kynurenate |
| 20 | sphingomyelin (d18:1/18:1, d18:2/18:0) |
| 21 | 1-palmitoyl-2-dihomo-linolenoyl-GPC (16:0/20:3n3 |
| | or 6) |
| 22 | 1-(1-enyl-palmitoyl)-2-linoleoyl-GPC (P-16:0/18:2) |
| 23 | Alanine |
| 24 | 1-palmitoyl-3-linoleoyl-glycerol (16:0/18:2) |
| 25 | N-acetylcarnosine |
| 26 | Asparagine |
| 27 | 1-oleoyl-2-linoleoyl-GPC (18:1/18:2) |
| 28 | N6-carbamoylthreonyladenosine |
| 29 | 1-(1-enyl-stearoyl)-2-docosahexaenoyl-GPC (P- |
| | 18:0/22:6) |
| 30 | 1-oleoyl-3-linoleoyl-glycerol (18:1/18:2) |
| 31 | N-acetylalanine |
| 32 | gamma-glutamylphenylalanine |
| 33 | Carnitine |
| 34 | Tyrosine |
| 35 | gamma-glutamyltyrosine |
| 36 | 1-palmitoyl-2-linoleoyl-glycerol (16:0/18:2) |
| 37 | Leucine |
| 38 | 1-oleoyl-2-linoleoyl-glycerol (18:1/18:2) |
| 39 | 1,2-dilinoleoyl-GPC (18:2/18:2) |
| 40 | N-acetyltyrosine |
| 41 | 2-methylbutyrylcarnitine (C5) |
| 42 | 1-palmitoleoyl-2-oleoyl-glycerol (16:1/18:1) |
| 43 | Cinnamoylglycine |
| 44 | Quinolinate |
| 45 | 1-palmitoleoyl-3-oleoyl-glycerol (16:1/18:1) |
| 46 | gulonic acid |
| 47 | 1-palmitoyl-2-adrenoyl-GPC (16:0/22:4) |
| 48 | Glucose |
| 49 | Cortisone |

and
diagnosing subject as having obesity if the mBMI value of the subject is modulated compared to a reference standard.

3. A method of diagnosing obesity or a disease related thereto in a subject, comprising,
obtaining a biological sample from the subject;
detecting, in the biological sample, levels or activities of at least 3 metabolites selected from the metabolites of Table 4 or derivatives thereof and computing a metabolomic body mass index (mBMI) value for the subject based on the detection, wherein the metabolites of Table 4 are listed in order of effect on the mBMI value;
TABLE 4

| S/N | Metabolite |
|---|---|
| 1 | 1-(1-enyl-stearoyl)-2-docosahexaenoyl-GPC |
| | (P-18:0/22:6) |
| 2 | sphingomyelin (d18:1/18:1, d18:2/18:0) |
| 3 | urate |

and
diagnosing subject as having obesity if the mBMI value of the subject is modulated compared to a reference standard.

4. A method of diagnosing obesity or a disease related thereto in a subject, comprising,
obtaining a biological sample from the subject;
detecting, in the biological sample, levels or activities of at least 3 metabolites selected from the metabolites of Table 5 or derivatives thereof and computing a metabolomic body mass index (mBMI) value for the subject based on the detection, wherein the metabolites of Table 5 are listed in order of effect on the mBMI value;
TABLE 5

| S/N | Metabolite |
|---|---|
| 1 | N-acetylglycine |
| 2 | 1-(1-enyl-stearoyl)-2-docosahexaenoyl-GPC |
| | (P-18:0/22:6) |
| 3 | sphingomyelin (d18:1/18:1, d18:2/18:0) |
| 4 | cortisone |
| 5 | mannose |
| 6 | urate |

and
diagnosing subject as having obesity if the mBMI value of the subject is modulated compared to a reference standard.

5. A method of diagnosing obesity or a disease related thereto in a subject, comprising,
obtaining a biological sample from the subject;
detecting, in the biological sample, levels or activities of at least 3 metabolites selected from the metabolites of Table 6 or derivatives thereof and computing a metabolomic body mass index (mBMI) value for the subject based on the detection, wherein the metabolites of Table 6 are listed order of effect on the mBMI value;
TABLE 6

| S/N | Metabolite |
|---|---|
| 1 | cortisone |
| 2 | N-acetylglycine |
| 3 | 1-nonadecanoyl-GPC (19:0) |
| 4 | asparagine |
| 5 | glucose |
| 6 | mannose |
| 7 | sphingomyelin (d18:1/18:1, d18:2/18:0) |
| 8 | aspartate |
| 9 | alanine |
| 10 | 1-stearoyl-2-dihomo-linolenoyl-GPC |
| | (18:0/20:3n3 or 6) |
| 11 | glutamate |
| 12 | kynurenate |
| 13 | urate |

and
diagnosing subject as having obesity if the mBMI value of the subject is modulated compared to a reference standard.

6. A method of diagnosing obesity or a disease related thereto in a subject, comprising,
obtaining a biological sample from the subject;
detecting, in the biological sample, levels or activities of at least 3 metabolites selected from the metabolites of Table 7 or derivatives thereof and computing a metabolomic body mass index (mBMI) value for the subject based on the detection, wherein the metabolites of Table 7 are listed in order of effect on the mBMI value;
TABLE 7

| S/N | Metabolite |
|---|---|
| 1 | urate |
| 2 | 1-stearoyl-2-dihomo-linolenoyl-GPC |
| | (18:0/20:3n3 or 6)* |
| 3 | alanine |
| 4 | N-acetyltyrosine |
| 5 | glutamate |
| 6 | 1-palmitoleoyl-3-oleoyl-glycerol (16:1/18:1)* |
| 7 | 1-(1-enyl-palmitoyl)-2-oleoyl-GPC (P-16:0/18:1)* |
| 8 | 1-(1-enyl-stearoyl)-2-oleoyl-GPC (P-18:0/18:1) |
| 9 | 1-(1-enyl-stearoyl)-2-docosahexaenoyl-GPC |
| | (P-18:0/22:6)* |
| 10 | 1-arachidoyl-GPC (20:0) |
| 11 | N-acetylglycine |
| 12 | sphingomyelin (d18:1/18:1, d18:2/18:0) |
| 13 | mannose |
| 14 | cortisone |

and
diagnosing subject as having obesity if the mBMI value of the subject is modulated compared to a reference standard.

7. The method of any one of claim 1, wherein the biological sample comprises a blood sample.

8. The method of any one of claim 1, wherein the levels and/or activities of the metabolites is determined using a chemical analytical method selected from the group consisting of HPLC, thin layer chromatography (TLC), electrochemical analysis, Mass Spectroscopy (MS), refractive index spectroscopy (RI), Ultra-Violet spectroscopy (UV), fluorescent analysis, radiochemical analysis, Near-Infra Red spectroscopy (Near-IR), Nuclear Magnetic Resonance spectroscopy (NMR), fluorescence spectroscopy, dual polarization interferometry, computational methods, Light Scattering analysis (LS), gas chromatography (GC), GC coupled with MS, and direct injection (DI) coupled with LC-MS/MS or a combination thereof.

9. The method of any one of claim 1, wherein the disease related to obesity is selected from coronary artery disease, hypertension, stroke, peripheral vascular disease, insulin resistance, glucose intolerance, diabetes mellitus, hyperglycemia, hyperlipidemia, hypercholesteremia, hypertriglyceridemia, hyperinsulinemia, atherosclerosis, cellular proliferation and endothelial dysfunction, diabetic dyslipidemia, lipodystrophy and metabolic syndrome, type II diabetes, diabetic complications including diabetic neuropathy, nephropathy, retinopathy or cataracts, heart failure, inflammation, thrombosis, congestive heart failure, asthmatic or pulmonary disease related to obesity, and cardiovascular disease related to obesity or a combination thereof.

10. The method of any one of claim 1, wherein the derivative of metabolite is selected from salts, amides, esters, enol ethers, enol esters, acetals, ketals, acids, bases, solvates, hydrates, and polymorphs or a combination thereof.

11. The method of any one of claim 1, wherein the modulation comprises an increase or a decrease.

12. The method of any one of claim 1, wherein the reference standard comprises the subject's BMI.

13. The method of claim 12, wherein if the subject's mBMI>the subject's BMI, then the subject is diagnosed as being overweight or having obesity with metabolic consequences for health.

14. The method of any one of claim 1, further comprising determining a secondary parameter selected from blood pressure, waist/hip ratio, android/gynoid ratio, % body fat, % visceral fat, % subcutaneous fat and insulin resistance or a combination thereof.

15. The method of claim 14, comprising generating a composite score of the mBMI and the secondary parameter and comparing the composite score to a reference standard.

16. The method of claim 15, wherein the reference standard comprises a positive reference standard comprising a composite score of the mBMI and the secondary parameter for an obese subject and/or a negative reference standard comprising a composite score of the mBMI and the secondary parameter for a non-obese or healthy subject.

17. A method of diagnosing obesity in a subject and treating the diagnosed subject with a therapy for obesity, comprising,
(a) detecting levels and/or activities of at least three markers of Table 1 or Table 2 or derivatives thereof in a biological sample obtained from the subject and computing a metabolomic body mass index (mBMI) value for the subject based on the detection, wherein the at least 3 metabolites of Table 1 comprises, in the order of rank of relative correlation to the obesity, urate, 5-methylthioadenosine, and glutamate; and wherein the at least 3 metabolites of Table 2 comprises, in the order of rank of relative correlation to the obesity, urate; glutamate; and 1-(1-enyl-palmitoyl)-2-oleoyl-GPC (P-16:0/18:1);
(b) diagnosing subject with obesity if the mBMI value of the subject is modulated compared to a reference standard; and
(c) administering an effective amount of a therapy selected from the group consisting of anti-obesity pharmacotherapy, surgery, and lifestyle therapy.

18. A method for screening a test agent for treating obesity, comprising,
(a) detecting levels and/or activities of at least three metabolites of Table 1 or Table 2 or derivatives thereof in a biological sample obtained from the subject to compute a first metabolomic body mass index (mBMI) value, wherein the at least 3 metabolites of Table 1 comprises, in the order of rank of relative correlation to the subject's obesity, urate, 5-methylthioadenosine, and glutamate; and wherein the at least 3 metabolites of Table 2 comprises, in the order of rank of relative correlation to the obesity, urate; glutamate; and 1-(1-enyl-palmitoyl)-2-oleoyl-GPC (P-16:0/18:1);
(b) administering a composition comprising the test agent to the subject;
(c) detecting levels and/or activities of the metabolites of step (a) in the biological sample obtained from the subject to compute a second mBMI value; and
(d) selecting a test agent if the second mBMI value is modulated compared to the first mBMI value for the subject.

19. A kit for determining a lipid or fat content of a biological sample, comprising: reagents for detecting a metabolite profile of the biological sample; vessels for holding the biological sample; optionally together with instructions for performing the detection, wherein the metabolite profile comprises at least three of the metabolites of Table 1 or Table 2 or derivatives thereof, wherein the at least 3 metabolites of Table 1 comprises: urate, 5-methylthioadenosine, and glutamate or derivatives thereof; and wherein the at least 3 metabolites of Table 2 comprises: urate, glutamate and 1-(1-enyl-palmitoyl)-2-oleoyl-GPC (P-16:0/18:1) or derivatives thereof.
